(19) United States
(12) Patent Application Publication
(10) Pub. No.: US 2024/0197715 A1
(43) Pub. Date: Jun. 20, 2024

(54) PHARMACEUTICAL COMBINATIONS AND USES THEREOF

(71) Applicant: NOVARTIS AG, Basel (CH)

(72) Inventors: Rita ANDRAOS-REY, Attenschwiller (FR); Sandra BLUM-KALLERT, Basel (CH); Marta CORTES-CROS, Basel (CH); Ruben DE KANTER, Basel (CH); Stephane FERRETTI, Huningue (FR); Isabel Cristina Colaco Farias JACO, Binningen (CH); Jacques HAMON, Habsheim (FR); Jürgen Hans-Hermann HINRICHS, Schopfheim (DE); Laurent LABORDE, Steinbrunn-le-Bas (FR); Henrik MOEBITZ, Arlesheim (CH); Michele MOSCHETTA, Basel (CH); Joseph SCHOEPFER, Riehen (CH); Ross Sinclair STRANG, Hagenthal-le-Bas (FR)

(21) Appl. No.: 18/510,836

(22) Filed: Nov. 16, 2023

Related U.S. Application Data
(60) Provisional application No. 63/582,920, filed on Sep. 15, 2023.
(60) Provisional application No. 63/508,015, filed on Jun. 14, 2023.
(60) Provisional application No. 63/486,206, filed on Feb. 21, 2023.
(60) Provisional application No. 63/384,313, filed on Nov. 18, 2022.

(51) Int. Cl. A61K 31/4745; A61K 31/4535; A61K 31/4709; A61K 31/495; A61K 31/506; A61K 31/519; A61K 31/52; A61K 31/5377; A61K 31/7048; A61K 33/243; A61P 35/00

(52) U.S. Cl. CPC A61K 31/4745; A61K 31/4535; A61K 31/4709; A61K 31/495; A61K 31/506; A61K 31/519; A61K 31/52; A61K 31/5377; A61K 31/7048; A61K 33/243; A61P 35/00

(57) ABSTRACT

The present invention provides a pharmaceutical combination comprising a WRN inhibitor in combination with at least one additional therapeutically active agent. It also provides a method of treating cancer, involving administering to a subject in need thereof the WRN inhibitor in combination with the at least one additional therapeutically active agent. For example, the WRN inhibitor compound, or a pharmaceutically acceptable salt thereof, is of formula (I):

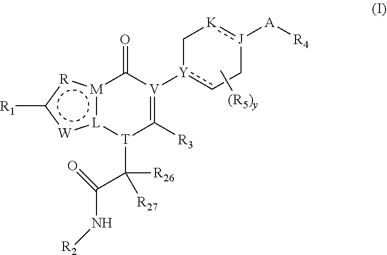

wherein R₁, R₂, R₃, R₄, R₅, R₂₈, R₂₇, y, R, M, W, L, V, T, Y, J, K and A are as described herein.

### CROSS-REFERENCE TO RELATED APPLICATIONS

This application claims benefit of U.S. Provisional Application No. 63/384,313, filed Nov. 18, 2022; U.S. Provisional Application No. 63/486,206, filed Feb. 21, 2023; U.S. Provisional Application No. 63/508,015, filed Jun. 14, 2023; and U.S. Provisional Application No. 63/582,920, filed Sep. 15, 2023. The contents of these provisional applications are hereby incorporated by reference in their entireties.

### FIELD OF THE DISCLOSURE

The present invention relates to a pharmaceutical combination comprising a WRN inhibitor in combination with at least one additional therapeutically active agent. The present invention also relates to methods of treating cancer involving administering to a subject in need thereof the WRN inhibitor in combination with the at least one additional therapeutically active agent.

### BACKGROUND

The advent of targeted therapies for cancer has increased patient lifespan for various malignancies and helped to appreciate the complexity of tumors through the study of drug resistance mechanisms. The fact that clinical responses to targeted agents are generally incomplete and/or transient results from a multitude of factors that can be broadly put into two classes: toxicities that prevent optimal dosing of drugs and consequently limit target engagement (Brana and Siu 2012, Chapman, Solit et al. 2014), and the ability of cancers to adapt and maintain their proliferative potential against perturbations (Druker 2008, Chandarlapaty 2012, Doebele, Pilling et al. 2012, Duncan, Whittle et al. 2012, Katayama, Shaw et al. 2012, Lito, Rosen et al. 2013, Sullivan and Flaherty 2013, Solit and Rosen 2014). Combinations of drugs can address both these factors by improving overall efficacies and at the same time targeting tumor robustness and complexity to counter resistance (Robert, Karaszewska et al. 2015, Turner, Ro et al. 2015). It is not yet clear how many drugs are required, and which processes need to be targeted in combination to overcome specific types of cancer. But it is almost certain that different pathways or drivers need to be inhibited, most likely requiring two or more drugs (Bozic, Reiter et al. 2013). In spite of numerous treatment options for patients with specific types of cancer, there remains a need for effective and safe combination therapies that can be administered for the treatment of cancer.

### SUMMARY

It is an object of the present invention to provide for a medicament to improve treatment of a cancer, in particular to improve treatment of cancer through inhibition of cell growth (proliferation) and/or induction of apoptosis (cell death). It is another object of the present invention to find novel combination therapies, for example those which selectively synergize the inhibition of proliferation and/or the induction of apoptosis.

Therefore, according to a first aspect of the invention, there is hereby provided a method of treating cancer, in particular cancer characterized as microsatellite instability-high (MSI-H) or mismatch repair deficient (dMMR), in a subject in need thereof, the method comprising administering to the subject a therapeutically effective amount of a WRN inhibitor in combination with a therapeutically effective amount of at least one therapeutically active agent selected from the group consisting of a PD-1 inhibitor, a chemotherapy agent, a Wee1 inhibitor, an ATR inhibitor, a DNA-PK inhibitor, ionising radiation based therapy selected from i) external beam radiation, ii) brachytherapy and iii) a radiopharmaceutical, a MEK inhibitor, an MDM2 inhibitor, a G4-quadruplex stabilizer, an ATM inhibitor, a CHK1 or CHK2 inhibitor and a PARP inhibitor.

Therefore, according to a second aspect of the invention, there is hereby provided a WRN inhibitor for use in the treatment of cancer, in particular cancer characterized as microsatellite instability-high (MSI-H) or mismatch repair deficient (dMMR), wherein the treatment further comprises administration of at least one therapeutically active agent selected from the group consisting of a PD-1 inhibitor, a chemotherapy agent, a Wee1 inhibitor, an ATR inhibitor, a DNA-PK inhibitor, ionising radiation based therapy selected from i) external beam radiation, ii) brachytherapy and iii) a radiopharmaceutical, a MEK inhibitor, an MDM2 inhibitor, a G4-quadruplex stabilizer, an ATM inhibitor, a CHK1 or CHK2 inhibitor and a PARP inhibitor.

Therefore, according to a third aspect of the invention, there is hereby provided a therapeutically active agent for use in the treatment of cancer, in particular cancer characterized as microsatellite instability-high (MSI-H) or mismatch repair deficient (dMMR), wherein the treatment further comprises administration of a WRN inhibitor, and wherein the therapeutically active agent is selected from the group consisting of a PD-1 inhibitor, a chemotherapy agent, a Wee1 inhibitor, an ATR inhibitor, a DNA-PK inhibitor, ionising radiation based therapy selected from i) external beam radiation, ii) brachytherapy and iii) a radiopharmaceutical, a MEK inhibitor, an MDM2 inhibitor, a G4-quadruplex stabilizer, an ATM inhibitor, a CHK1 or CHK2 inhibitor and a PARP inhibitor.

Therefore, according to a fourth aspect of the invention, there is hereby provided a combination comprising i) a WRN inhibitor, and ii) at least one therapeutically active agent selected from the group consisting of a Wee1 inhibitor, an ATR inhibitor, a DNA-PK inhibitor, ionising radiation selected from i) external beam radiation, ii) brachytherapy and iii) a radiopharmaceutical, a MEK inhibitor, an MDM2 inhibitor, a G4-quadruplex stabilizer, an ATM inhibitor, a CHK1 or CHK2 inhibitor, and a PARP inhibitor.

Therefore, according to a fifth aspect of the invention, there is hereby provided a method of treating colorectal cancer, in particular colorectal cancer characterized as microsatellite instability-high (MSI-H) or mismatch repair deficient (dMMR), the method comprising administering to a subject a therapeutically effective amount of a WRN inhibitor in combination with a therapeutically effective amount of a G4-quadruplex stabilizer.

Therefore, according to a sixth aspect of the invention, there is hereby provided a method of treating colorectal cancer, in particular colorectal cancer characterized as microsatellite instability-high (MSI-H) or mismatch repair deficient (dMMR), the method comprising administering to a subject a therapeutically effective amount of a WRN inhibitor in combination with a therapeutically effective amount of a MEK inhibitor.

Therefore, according to a seventh aspect of the invention, there is hereby provided a method of treating colorectal cancer, in particular colorectal cancer characterized as microsatellite instability-high (MSI-H) or mismatch repair deficient (dMMR), the method comprising administering to a subject a therapeutically effective amount of a WRN inhibitor in combination with a therapeutically effective amount of a topoisomerase inhibitor.

Therefore, according to an eighth aspect of the invention, there is hereby provided a method of treating colorectal or gastric cancer, in particular colorectal or gastric cancer characterized as microsatellite instability-high (MSI-H) or mismatch repair deficient (dMMR), the method comprising administering to a subject a therapeutically effective amount of a WRN inhibitor in combination with a therapeutically effective amount of an ATR inhibitor.

Therefore, according to a ninth aspect of the invention, there is hereby provided a method of treating gastric cancer or colorectal cancer, in particular gastric cancer characterized as microsatellite instability-high (MSI-H) or mismatch repair deficient (dMMR), or colorectal cancer characterized as microsatellite instability-high (MSI-H) or mismatch repair deficient (dMMR), the method comprising administering to a subject a therapeutically effective amount of a WRN inhibitor in combination with a therapeutically effective amount of ionising radiation based therapy selected from i) external beam radiation, ii) brachytherapy and ii) a radiopharmaceutical.

Therefore, according to a tenth aspect of the invention, there is hereby provided a method of treating colorectal cancer, in particular colorectal cancer characterized as microsatellite instability-high (MSI-H) or mismatch repair deficient (dMMR), the method comprising administering to a subject a therapeutically effective amount of a WRN inhibitor in combination with a therapeutically effective amount of a chemotherapy agent.

Therefore, according to an eleventh aspect of the invention, there is hereby provided a method of treating colorectal cancer, in particular colorectal cancer characterized as microsatellite instability-high (MSI-H) or mismatch repair deficient (dMMR), the method comprising administering to a subject a therapeutically effective amount of a WRN inhibitor in combination with a therapeutically effective amount of an MDM2 inhibitor.

Therefore, according to a twelfth aspect of the invention, there is hereby provided a method of treating colorectal cancer, in particular colorectal cancer characterized as microsatellite instability-high (MSI-H) or mismatch repair deficient (dMMR), the method comprising administering to a subject a therapeutically effective amount of a WRN inhibitor in combination with a therapeutically effective amount of an ATM inhibitor.

Therefore, according to a thirteenth aspect of the invention, there is hereby provided a method of treating colorectal cancer, in particular colorectal cancer characterized as microsatellite instability-high (MSI-H) or mismatch repair deficient (dMMR), the method comprising administering to a subject a therapeutically effective amount of a WRN inhibitor in combination with a therapeutically effective amount of a DNA-PK inhibitor.

Therefore, according to a fourteenth aspect of the invention, there is hereby provided a method of treating colorectal cancer, in particular colorectal cancer characterized as microsatellite instability-high (MSI-H) or mismatch repair deficient (dMMR), the method comprising administering to a subject a therapeutically effective amount of a WRN inhibitor in combination with a therapeutically effective amount of a WEE1 inhibitor.

Therefore, according to a fifteenth aspect of the invention, there is hereby provided a method of treating colorectal cancer, in particular colorectal cancer characterized as microsatellite instability-high (MSI-H) or mismatch repair deficient (dMMR), the method comprising administering to a subject a therapeutically effective amount of a WRN inhibitor in combination with a therapeutically effective amount of a CHK1 or CHK2 inhibitor, including a dual CHK1 and CHK2 inhibitor.

Therefore, according to a sixteenth aspect of the invention, there is hereby provided a combination comprising i) a WRN inhibitor and ii) temozolomide, and optionally iii) irinotecan.

Therefore, according to a seventeenth aspect of the invention, there is hereby provided temozolomide for use in the treatment of cancer, for example microsatellite instability-high (MSI-H), mismatch repair deficient (dMMR) or microsatellite stable (MSS) cancer, in particular MSS cancer, wherein the treatment further comprises administration of:
a WRN inhibitor, or
a WRN inhibitor in combination with irinotecan.

Therefore, according to an eighteenth aspect of the invention, there is hereby provided a WRN inhibitor for use in the treatment of cancer, for example microsatellite instability-high (MSI-H), mismatch repair deficient (dMMR) or microsatellite stable (MSS) cancer, in particular MSS cancer, wherein the treatment further comprises administration of:
temozolomide, or
temozolomide and irinotecan.

Therefore, according to a nineteenth aspect of the invention, there is hereby provided irinotecan for use in the treatment of cancer, for example microsatellite instability-high (MSI-H), mismatch repair deficient (dMMR) or microsatellite stable (MSS) cancer, in particular MSS cancer, wherein the treatment further comprises administration of:
a WRN inhibitor, or in particular,
a WRN inhibitor and temozolomide.

Therefore, according to a twentieth aspect of the invention, there is hereby provided temozolomide, a WRN inhibitor, or irinotecan, for use according to any of the sixteenth to nineteenth aspects of the invention, wherein temozolomide is administered:
as a pre-treatment before administration of the WRN inhibitor, or before administration of irinotecan, or before administration of both the WRN inhibitor and irinotecan, without subsequent combination treatment including temozolide, or
in combination, without pre-treatment with temozolomide, or
as a pre-treatment before administration of the WRN inhibitor, or before administration of irinotecan, or before administration of both the WRN inhibitor and irinotecan, followed by combination treatment including temozolomide,

wherein said combination or combination treatment is described in the sixteenth to nineteenth aspects, and said use is for the treatment of cancer which is MSI-H, dMMR or MSS, in particular dMMR or MSS cancer, more particularly MSS cancer.

Therefore, according to a twenty-first aspect of the invention, there is hereby provided a WRN inhibitor for use in the treatment of dMMR or MSS cancer, in particular MSS cancer.

Therefore, according to a twenty-second aspect of the invention, there is hereby provided a method of treating cancer in a subject in need thereof, for example microsatellite instability-high (MSI-H), mismatch repair deficient (dMMR) or microsatellite stable (MSS) cancer, in particular MSS cancer, the method comprising administering to the subject a therapeutically effective amount of temozolomide, wherein the treatment further comprises administration of:
a therapeutically effective amount of a WRN inhibitor, or
a therapeutically effective amount of a WRN inhibitor in combination with a therapeutically effective amount of irinotecan.

Therefore, according to a twenty-third aspect of the invention, there is hereby provided a method of treating cancer in a subject in need thereof, for example microsatellite instability-high (MSI-H), mismatch repair deficient (dMMR) or microsatellite stable (MSS) cancer, in particular MSS cancer, the method comprising administering to the subject a therapeutically effective amount of a WRN inhibitor, wherein the treatment further comprises administration of:
a therapeutically effective amount of temozolomide, or
a therapeutically effective amount of temozolomide and a therapeutically effective amount of irinotecan.

Therefore, according to a twenty-fourth aspect of the invention, there is hereby provided a method of treating cancer in a subject in need thereof, for example microsatellite instability-high (MSI-H), mismatch repair deficient (dMMR) or microsatellite stable (MSS) cancer, in particular MSS cancer, the method comprising administering to the subject a therapeutically effective amount of irinotecan, wherein the treatment further comprises administration of:
a therapeutically effective amount of a WRN inhibitor, or in particular
a therapeutically effective amount of a WRN inhibitor and a therapeutically effective amount or temozolomide.

Therefore, according to a twenty-fifth aspect of the invention, there is hereby provided a WRN inhibitor for use in the treatment of cancer, wherein the treatment further comprises administration of an alkylating agent, in particular temozolomide, and the cancer is:
MSS cancer,
pMMR/MSS MGMT-defective, or pMMR/MSS MGMT-deficient, or pMMR/MSS MGMT-silenced, or MGMT-methylated pMMR/MSS cancer. For example, colorectal cancer,
MGMT-methylated glioblastoma,
MGMT-hypermethylated, CRC, or
MGMT-hypermethylated, RAS-mutated or RAS wild-type CRC,

and optionally wherein the treatment further comprises administration of a chemotherapy, for example irinotecan.

Therefore, according to a twenty-sixth aspect of the invention, there is hereby provided an alkylating agent for use in the treatment of cancer, wherein the treatment further comprises administration of a WRN inhibitor, and the cancer is:
MSS cancer,
pMMR/MSS MGMT-defective, or pMMR/MSS MGMT-deficient, or pMMR/MSS MGMT-silenced, or MGMT-methylated pMMR/MSS cancer, such as colorectal cancer
MGMT-methylated glioblastoma,
MGMT-hypermethylated, CRC, or
MGMT-hypermethylated, RAS-mutated or RAS wild-type CRC.

and optionally wherein the treatment further comprises administration of a chemotherapy, for example irinotecan.

Therefore, according to a twenty-seventh aspect of the invention, there is provided a method of treating cancer, in particular cancer characterized as microsatellite instability-high (MSI-H) or mismatch repair deficient (dMMR), in a subject in need thereof, the method comprising administering to the subject a therapeutically effective amount of a WRN inhibitor in combination with a therapeutically effective amount of a DNA polymerase alpha inhibitor, for example aphidicolin, for example for the treatment of colorectal cancer.

Therefore, according to a twenty-eighth aspect of the invention, there is provided a method of treating cancer, in particular cancer characterized as MSS or mismatch repair deficient (dMMR), in particular MSS, in a subject in need thereof, the method comprising administering to the subject a therapeutically effective amount of a WRN inhibitor in combination with a therapeutic agent which can (or is capable of):
sensitise the cancer cells, for example for an improved response to the WRN inhibitor,
prime the cancer cells, for example, such priming may include triggering hypermutation status in cancer cells,
create or increase MMR deficiency in cancer cells,
create or increase MSI-H status in cancer cells,
increase MMR heterogeneity of cancer cells, and/or
create or increase resistance in cancer cells to temozolomide,

In particular to
create or increase MMR deficiency in cancer cells, or
create or increase MSI-H status in cancer cells,

and optionally wherein the treatment further comprises administration of a chemotherapy, for example irinotecan.

Therefore, according to a twenty-ninth aspect of the invention, there is provided a WRN inhibitor for use in the treatment of cancer, in particular cancer characterized as MSS or mismatch repair deficient (dMMR), in particular MSS, wherein the treatment further comprises administration of a therapeutic agent agent which can (or is capable of):
sensitise the cancer cells, for example for an improved response to a WRN inhibitor,
prime the cancer cells, for example, such priming may include triggering hypermutation status in cancer cells,
create or increase MMR deficiency in cancer cells,
create or increase MSI-H status in cancer cells,
increase MMR heterogeneity of cancer cells, and/or
create or increase resistance in cancer cells to temozolomide.

In particular to
create or increase MMR deficiency in cancer cells, or
create or increase MSI-H status in cancer cells

and optionally wherein the treatment further comprises administration of a chemotherapy, for example irinotecan.

Therefore, according to a thirtieth aspect of the invention, there is provided a method of treating cancer in a subject in need thereof, wherein the subject has microsatellite stable cancer (MSS), and wherein the patient is administered:
a) an agent which:
   sensitises the cancer cells, for example for an improved response to treatment with a WRN inhibitor,
   primes the cancer cells, for example, such priming may include triggering hypermutation status in cancer cells,
   creates or increases MMR deficiency in cancer cells,
   creates or increases MSI-H status in cancer cells, or
   increases MMR heterogeneity of cancer cells, and

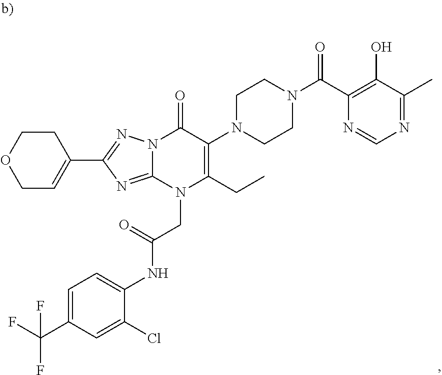 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl]

or a pharmaceutically acceptable salt thereof,

and optionally,
c) irinotecan.

### BRIEF DESCRIPTION OF THE DRAWINGS

FIG. 1: SW48 CRC MSI^{high} cell line was treated with either monotherapy of compound A, Irinotecan or a combination of compound A with Irinotecan, at the indicated concentrations. Compound treatment was refreshed on day 7 and treatment was removed around day 15. Cells were left untreated for the remaining days of the experiment, with media refreshment around day 21. Proliferation was monitored using Incucyte® S3 live-cells analysis instrument (Sartorius).

FIG. 2: SW48 CRC MSI^{high} cell line was treated with either monotherapy of compound A, ATRi (BAY1895344) or a combination of compound A with ATRi, at the indicated concentrations. Compound treatment was refreshed on day 7 and treatment was removed around day 15. Cells were left untreated for the remaining days of the experiment, with media refreshment around day 21. Proliferation was monitored using Incucyte® S3 live-cells analysis instrument (Sartorius).

FIG. 3: SW48 CRC MSI^{high} cell line was treated with either monotherapy of compound A, DNA-PKi (AZD7648) or a combination of compound A with DNA-PKi, at the indicated concentrations. Compound treatment was refreshed on day 7 and treatment was removed around day 15. Cells were left untreated for the remaining days of the experiment, with media refreshment around day 21. Proliferation was monitored using Incucyte® S3 live-cells analysis instrument (Sartorius).

FIG. 4: HCT116 CRC MSI^{high} cell line was treated with either monotherapy of compound A, Adavosertib (AZD1775) or a combination of compound A with Adavosertib, at the indicated concentrations. Compound treatment was refreshed on day 7 and treatment was removed around day 15. Cells were left untreated for the remaining days of the experiment, with media refreshment around day 21. Proliferation was monitored using Incucyte® S3 live-cells analysis instrument (Sartorius).

FIG. 5: SW48 CRC MSI^{high} cell line was treated with either monotherapy of compound A, HDM201 or a combination of compound A with HDM201, at the indicated concentrations. Compound treatment was refreshed on day 7 and treatment was removed around day 15. Cells were left untreated for the remaining days of the experiment, with media refreshment around day 21. Proliferation was monitored using Incucyte® S3 live-cells analysis instrument (Sartorius).

FIGS. 6 and 7: SW48 CRC MSI^{high} cell line was treated with either monotherapy of compound A, or ionizing radiation (IR) or a combination of compound A with IR, at the indicated concentrations. Cells were irradiated and compound was refreshed on day 7 and treatment was removed around day 15. Cells were left untreated for the remaining days of the experiment, with media refreshment around day 21. FIG. 6: Proliferation was monitored using Incucyte® S3 live-cells analysis instrument (Sartorius).

FIG. 7: % of confluence at Day 40, end of treatment, was plotted for each condition comparing compound A treatment (no IR) with compound A on irradiated cells at the rate of (+3Gy)/min

FIG. 8: SW48 CRC MSI^{high} cell line was treated with either monotherapy of compound A, 5FU or a combination of compound A with 5FU, at the indicated concentrations. Compound treatment was refreshed on day 7 and treatment was removed around day 15. Cells were left untreated for the remaining days of the experiment, with media refreshment around day 21. Proliferation was monitored using Incucyte® S3 live-cells analysis instrument (Sartorius).

FIG. 9: SW48 CRC MSI^{high} cell line was treated with either monotherapy of compound A, Epirubicin or a combination of compound A with Epirubicin, at the indicated concentrations. Compound treatment was refreshed on day 7 and treatment was removed around day 15. Cells were left untreated for the remaining days of the experiment, with media refreshment around day 21. Proliferation was monitored using Incucyte® S3 live-cells analysis instrument (Sartorius).

FIG. 10: HCT116 CRC MSI^{high} cell line was treated with either monotherapy of compound A, Epirubicin or a combination of compound A with Epirubicin, at the indicated concentrations. Compound treatment was refreshed on day 7 and treatment was removed around day 15. Cells were left untreated for the remaining days of the experiment, with media refreshment around day 21. Proliferation was monitored using Incucyte® S3 live-cells analysis instrument (Sartorius).

FIG. 11: SW48 and HCT116 CRC MSI^{high} cell lines were treated with either monotherapy of compound A, Trametinib or a combination of compound A with Trametinib, at the indicated concentrations. Compound treatment was refreshed on day 7 and treatment was removed around day 15. Cells were left untreated for the remaining days of the experiment, with media refreshment around day 21. Proliferation was monitored using Incucyte® S3 live-cells analysis instrument (Sartorius).

FIG. 12: Figure showing proliferation of SW48 colorectal carcinoma cells in nude mice (Charles River, Germany) following administration of i) Vehicle 5 ml/kg (p.o., qd), ii) Trametinib 0.3 mg/kg (p.o, qd), iii) Compound A 40 mg/kg (p.o., qd), and iv) a combination of Trametinib 0.3 mg/kg (p.o, qd) and Compound A 40 mg/kg (p.o., qd).

FIG. 13: SW48 CRC MSI^{high} cell line was treated with either monotherapy of compound A, Docetaxel or a combination of compound A with Docetaxel, at the indicated concentrations. Compound treatment was refreshed on day 7 and treatment was removed around day 15. Cells were left untreated for the remaining days of the experiment, with media refreshment around day 21. Proliferation was monitored using Incucyte® S3 live-cells analysis instrument (Sartorius).

FIG. 14: SW48 CRC MSI^{high} cell line was treated with either monotherapy of compound A, Carboplatin or a combination of compound A with Carboplatin, at the indicated concentrations. Compound treatment was refreshed on day 7 and treatment was removed around day 15. Cells were left untreated for the remaining days of the experiment, with media refreshment around day 21. Proliferation was monitored using Incucyte® S3 live-cells analysis instrument (Sartorius).

FIG. 15: Figure showing proliferation of SW48 colorectal carcinoma cells in nude mice (Charles River, Germany) following administration of i) Vehicle 5 ml/kg (p.o., qd), ii) BAY-1895344 50 mg/kg (p.o, twice a day on administration days, 3 administration days followed by 4 off days) iii) Compound A 20 mg/kg (p.o., qd), and iv) a combination of BAY-1895344 50 mg/kg (p.o, twice a day on administration days, 3 administration days followed by 4 off days) and Compound A 20 mg/kg (p.o., qd)

FIG. 16: Figure showing proliferation of HX-2861 MSI^{high} colorectal cancer tumors in nude mice (Charles River, Germany) following administration of i) Vehicle 5 ml/kg (p.o., qd), ii) BAY-1895344 25 mg/kg (p.o, twice a day on administration days, 3 administration days followed by 4 off days) iii) Compound A 40 mg/kg (p.o., qd), and iv) a combination of BAY-1895344 25 mg/kg (p.o, twice a day on administration days, 3 administration days followed by 4 off days and Compound A 40 mg/kg (p.o., qd).

FIG. 17: Figure showing proliferation of IM95 gastric carcinoma cells in SCID-BEIGE mice (Charles River, Germany) following administration of i) Vehicle 5 ml/kg (p.o., qd), ii) 5 Gy irradiation performed with a XRad320™ from Precision X-Ray and an adjustable X-ray Collimator (#XD1601-0000, Precision X-Ray), iii) Compound A 30 mg/kg (p.o., qd), and iv) a combination of 5 Gy irradiation performed with a XRad320™ from Precision X-Ray and an adjustable X-ray Collimator (#XD1601-0000, Precision X-Ray) and Compound A 30 mg/kg.

FIG. 18: Figure showing proliferation of SW48 colorectal carinoma cells in nude mice (Charles River, Germany) following administration of i) Vehicle 5 ml/kg (p.o., qd), ii) Irinotecan 60 mg/kg (i.v. qw), iii) Compound A 20 mg/kg (p.o., qd), and iv) a combination of Irinotecan 60 mg/kg (i.v. qw) and Compound A 20 mg/kg.

FIGS. 19 and 20: Potential effects of the combination of WRN inhibitors with a G4 stabilizer pyridostatin were tested in a clonogenic assay in SW48 as well as HCT116 colorectal MSI-high cancer cells. FIGS. 19 and 20 depict the images of the stained plates with the colonies with a range of WRN inhibitor compound B from 1.95 nM to 2000 nM in 2-fold increments, with or without pyridostatin. Graphs are a quantification of the signal either relative to DMSO of WRNi alone (top graph each figure), or relative to DMSO within the plate (i.e. in the presence of pyridostatin).

FIG. 21: SW48 CRC MSI^{high} cell line was treated with either monotherapy of compound A, CHK1/2i (AZD7762) or a combination of compound A with CHK1/2i, at the indicated concentrations. Compound treatment was refreshed on day 7 and treatment was removed around day 15. Cells were left untreated for the remaining days of the experiment, with media refreshment around day 21. Proliferation was monitored using Incucyte® S3 live-cells analysis instrument (Sartorius).

FIG. 22: In vitro viability of the colorectal cancer cell line HCT116 was assessed using the CellTiterGlo following 4-day treatment with the WRN inhibitor Compound B combined with doxorubicin. Growth inhibition %: 0-99=delayed proliferation, 100=growth arrest/stasis, 101-200=reduction in cell number/cell death.

FIG. 23: In vitro viability of the colorectal cancer cell line SW48 was assessed using the CellTiterGlo following 4-day treatment with the WRN inhibitor Compound B combined with 5-fluorouracil (5-FU). Growth inhibition %: 0-99=delayed proliferation, 100=growth arrest/stasis, 101-200=reduction in cell number/cell death.

FIG. 24: In vitro viability of the colorectal cancer cell line SW48 was assessed using the CellTiterGlo following 4-day treatment with the WRN inhibitor Compound B combined with Aphidicolin. Growth inhibition %: 0-99=delayed proliferation, 100=growth arrest/stasis, 101-200=reduction in cell number/cell death.

FIG. 25: In vitro viability of the colorectal cancer cell line SW48 was assessed using the CellTiterGlo following 4-day treatment with the WRN inhibitor Compound B combined with QAP1. Growth inhibition %: 0-99=delayed proliferation, 100=growth arrest/stasis, 101-200=reduction in cell number/cell death.

FIG. 26: In vitro viability of the colorectal cancer cell line SW48 was assessed using the CellTiterGlo following 4-day treatment with the WRN inhibitor Compound B combined with Bleomycin. Growth inhibition %: 0-99=delayed proliferation, 100=growth arrest/stasis, 101-200=reduction in cell number/cell death.

FIG. 27: In vitro viability of the colorectal cancer cell line SW48 was assessed using the CellTiterGlo following 4-day treatment with the WRN inhibitor Compound B combined with cisplatin. Growth inhibition %: 0-99=delayed proliferation, 100=growth arrest/stasis, 101-200=reduction in cell number/cell death.

FIG. 28: In vitro viability of the colorectal cancer cell line SW48 was assessed using the CellTiterGlo following 4-day treatment with the WRN inhibitor Compound B combined with Doxorubicin. Growth inhibition %: 0-99=delayed proliferation, 100=growth arrest/stasis, 101-200=reduction in cell number/cell death.

FIG. 29: In vitro viability of the colorectal cancer cell line SW48 was assessed using the CellTiterGlo following 4-day treatment with the WRN inhibitor Compound B combined with Gemcitabin. Growth inhibition %: 0-99=delayed proliferation, 100=growth arrest/stasis, 101-200=reduction in cell number/cell death.

FIG. 30: In vitro viability of the colorectal cancer cell line SW48 was assessed using the CellTiterGlo following 4-day treatment with the WRN inhibitor Compound B combined with HDM201. Growth inhibition %: 0-99=delayed proliferation, 100=growth arrest/stasis, 101-200=reduction in cell number/cell death.

FIG. 31: In vitro viability of the colorectal cancer cell line SW48 was assessed using the CellTiterGlo following 4-day treatment with the WRN inhibitor Compound B combined with camptothecin. Growth inhibition %: 0-99=delayed proliferation, 100=growth arrest/stasis, 101-200=reduction in cell number/cell death.

FIG. 32: In vitro viability of the colorectal cancer cell line SW48 was assessed using the CellTiterGlo following 4-day treatment with the WRN inhibitor Compound B combined with KU-60019. Growth inhibition %: 0-99=delayed proliferation, 100=growth arrest/stasis, 101-200=reduction in cell number/cell death.

FIG. 33: In vitro viability of the colorectal cancer cell line SW48 was assessed using the CellTiterGlo following 4-day treatment with the WRN inhibitor Compound B combined with NU7441 (KU-57788). Growth inhibition %: 0-99=delayed proliferation, 100=growth arrest/stasis, 101-200=reduction in cell number/cell death.

FIG. 34: HCT116 CRC MSI^{high} cell lines were treated with either monotherapy of compound A, Mitomycin C or a combination of compound A with Mitomycin C, at the indicated concentrations. Compound treatment was refreshed on day 7 and treatment was removed around day 15. Cells were left untreated for the remaining days of the experiment, with media refreshment around day 21. Proliferation was monitored using Incucyte® S3 live-cells analysis instrument (Sartorius).

FIG. 35: Figure shows the mean tumor volume in nude mice bearing SW620 (CCL-227, ATCC) [MSS colorectal carcinoma (CRC)] xenografts, following treatment with irinotecan alone, or irinotecan and Compound A.

FIG. 36: Figure shows the mean tumor volume in nude mice bearing SW620 (CCL-227, ATCC) [MSS colorectal carcinoma (CRC)] xenografts, following pre-treatment with temozolomide, followed by treatment with temozolomide, irinotecan and Compound A.

FIG. 37: Figure shows the mean tumor volume in nude mice bearing SW620 (CCL-227, ATCC) [MSS colorectal carcinoma (CRC)] xenografts, following pre-treatment with temozolomide, followed by treatment with irinotecan alone, or irinotecan and Compound A.

FIG. 38: Figure shows the efficacy of treatment of parental SW620 tumors with irinotecan, compound A and temozolomide combinations.

FIG. 39: Figure shows the efficacy of treatment of parental SW620 tumors with irinotecan, Compound A and temozolomide combinations.

FIG. 40: Figure shows the change in body weight following treatment of SW620 tumors with irinotecan, Compound A and temozolomide combinations.

FIG. 41: Figure shows the efficacy of treatment of parental T84 tumors with Compound A, irinotecan and temozolomide (TMZ) combinations.

FIG. 42: Figure shows the efficacy of treatment of parental T84 tumors with Compound A, irinotecan and temozolomide (TMZ) combinations.

FIG. 43: Figure shows change in body weight on treatment with Compound A, irinotecan and temozolomide (TMZ) combinations.

FIG. 44: Figure shows the efficacy of treatment of parental SHP77 tumors with irinotecan and temozolomide (TMZ).

FIG. 45: Figure shows the efficacy of treatment of parental SHP77 tumors with irinotecan, TMZ and Compound A combinations.

FIG. 46: Figure shows change in body weight on treatment with temozolomide and irinotecan

FIG. 47: Figure shows change in body weight on treatment with temozolomide, irinotecan and Compound A.

### DETAILED DESCRIPTION

As mentioned above, an object of the present invention is to find novel combination therapies for the treatment of cancer, in particular cancer characterized as microsatellite instability-high (MSI-H) or mismatch repair deficient (dMMR). In another object, the combination therapies synergize in inhibiting proliferation and/or in inducing apoptosis.

Another object of the invention is to find novel therapies for the treatment of cancer characterized as microsatellite stable (MSS).

Therefore, according to a first aspect of the invention, there is hereby provided a method of treating cancer, in particular cancer characterized as microsatellite instability-high (MSI-H) or mismatch repair deficient (dMMR), in a subject in need thereof, the method comprising administering to the subject a therapeutically effective amount of a WRN inhibitor in combination with a therapeutically effective amount of at least one therapeutically active agent selected from the group consisting of a PD-1 inhibitor, a chemotherapy agent, a Wee1 inhibitor, an ATR inhibitor, a DNA-PK inhibitor, ionising radiation based therapy selected from i) external beam radiation, ii) brachytherapy and iii) a radiopharmaceutical, a MEK inhibitor, an MDM2 inhibitor, a G4-quadruplex stabilizer, an ATM inhibitor, a CHK1 or CHK2 inhibitor and a PARP inhibitor.

Therefore, according to a second aspect of the invention, there is hereby provided a WRN inhibitor for use in the treatment of cancer, in particular cancer characterized as microsatellite instability-high (MSI-H) or mismatch repair deficient (dMMR), wherein the treatment further comprises administration of at least one therapeutically active agent selected from the group consisting of a PD-1 inhibitor, a chemotherapy agent, a Wee1 inhibitor, an ATR inhibitor, a DNA-PK inhibitor, ionising radiation based therapy selected from i) external beam radiation, ii) brachytherapy and iii) a radiopharmaceutical, a MEK inhibitor, an MDM2 inhibitor, a G4-quadruplex stabilizer, an ATM inhibitor, a CHK1 or CHK2 inhibitor and a PARP inhibitor.

Therefore, according to a third aspect of the invention, there is hereby provided a therapeutically active agent for use in the treatment of cancer, in particular cancer characterized as microsatellite instability-high (MSI-H) or mismatch repair deficient (dMMR), wherein the treatment further comprises administration of a WRN inhibitor, and wherein the therapeutically active agent is selected from the group consisting of a PD-1 inhibitor, a chemotherapy agent, a Wee1 inhibitor, an ATR inhibitor, a DNA-PK inhibitor, ionising radiation based therapy selected from i) external beam radiation, ii) brachytherapy and iii) a radiopharmaceutical, a MEK inhibitor, an MDM2 inhibitor, a G4-quadruplex stabilizer, an ATM inhibitor, a CHK1 or CHK2 inhibitor and a PARP inhibitor.

Therefore, according to a fourth aspect of the invention, there is hereby provided a combination comprising i) a WRN inhibitor, and ii) at least one therapeutically active agent selected from the group consisting of a Wee1 inhibitor, an ATR inhibitor, a DNA-PK inhibitor, ionising radiation selected from i) external beam radiation, ii) brachytherapy and iii) a radiopharmaceutical, a MEK inhibitor, an MDM2 inhibitor, a G4-quadruplex stabilizer, an ATM inhibitor, a CHK1 or CHK2 inhibitor, and a PARP inhibitor.

In an embodiment of any of the first to fourth aspects of the invention, the WRN inhibitor is a compound of formula (I), or a pharmaceutically acceptable salt thereof:

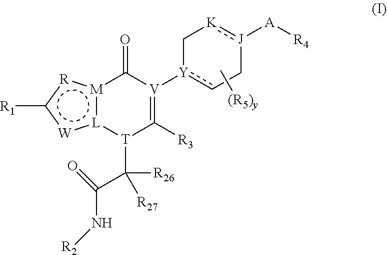

wherein
R, M, W, L, V and T are independently selected from C, CH and N, to form subformulae 1a, 1b, 1c, 1d, 1e and 1f:

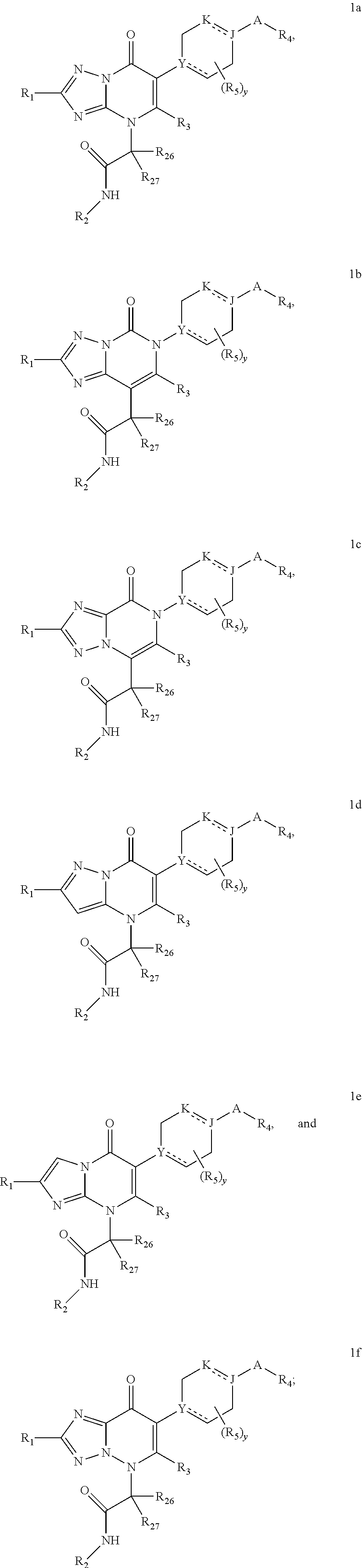

A is a linker selected from —C(O)—, —S(O)—, —S(O)₂—, and

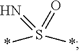

Y is N, C or CH;
y is 0, 1, 2, 3 or 4;
Y [symbol] means Y is linked via a single bond to the adjacent carbon atom when Y is CH, or Y is linked via a double bond to the adjacent atom when Y is C, and when Y [symbol] is a single bond, Y is carbon unsubstituted or substituted by OH or F;
when Y is N, Y [symbol] is a single bond;
K [symbol] means K is linked via a single or double bond to the adjacent atom;
wherein:
   when K [symbol] is a double bond, Y [symbol] is a single bond, K is CH, J is C, and A is a linker selected from —C(O)—, —S(O)—, —S(O)₂—, and

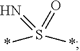

or
when K [symbol] is a single bond, K is selected from —CH₂—, —CH₂CH₂—, —NH— and a bond (to form a 5-membered ring:

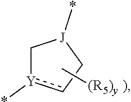

J is N, and A is a linker selected from —C(O)—, —S(O)—, —S(O)₂—, and

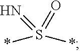

or
when K [symbol] is a single bond, K is —CH₂—, J is CH, and A is a linker selected from —S(O)—, —S(O)₂—, and

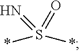

R₅ is independently selected from:
   (C₁-C₄)alkyl,
   (C₃-C₅)cycloalkyl,
   and wherein two R₅ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S,
   when K [symbol] J is a carbon-nitrogen single bond, a R₅ substituent on K and on the adjacent carbon atom may join to form ring C:

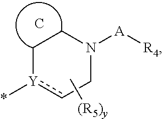

wherein ring C is a fused (C₃-C₆)cycloalkyl ring, a fused (C₃-C₆)heterocyclyl ring or a fused phenyl ring, wherein said fused (C₃-C₆)heterocyclyl ring contains ring carbon atoms and one ring heteroatom selected from O, N and S,
   when K [symbol] J is a carbon-carbon single bond, Y is N and Y [symbol] is a single bond, and A is a linker selected from —S(O)—, —S(O)₂—, and

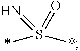

a R₅ substituent on K and on the adjacent carbon atom may join to form ring C:

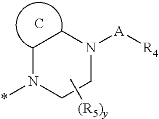

and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—;
and wherein one or more H atoms on the ring:

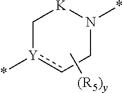

may be replaced by deuterium;
R₁ is:
cycloalkenyl, wherein said cycloalkenyl is a partially unsaturated monocyclic ring containing 5 or 6 ring carbon atoms, and said cycloalkenyl is unsubstituted or substituted by 1, 2, 3 or 4, preferably 1 or 2, R₃₃, wherein R₃₃ is halo, and wherein said cycloalkenyl or halo-substituted cycloalkenyl is substituted by 0, 1 or 2 R₁₅ substituents, or R₁ is heterocyclyl, wherein said heterocyclyl is a 5 or 6 membered fully saturated or partially unsaturated group comprising ring carbon atoms and 1 or 2 ring heteroatoms independently selected from N, O and S, and wherein said heterocyclyl is unbridged or bridged, and said bridge is 1 or 2 carbon atoms, wherein said heterocyclyl is unsubstituted or substituted by 1, 2, 3 or 4, preferably 1 or 2, R₃₃, wherein R₃₃ is halo, and wherein said heterocyclyl or halo-substituted heterocyclyl is substituted by 0, 1 or 2 substituents independently selected from R₁₅, R₁₆, R₁₇, R₁₈, R₁₉, R₂₀, R₂₂ and R₂₃,
or said heterocyclyl or halo-substituted heterocyclyl is fused to a cyclopropyl ring, wherein said cyclopropyl ring is unsubstituted or substituted by 1, 2 or 3 F,
or said heterocyclyl or halo-substituted heterocyclyl has 2 substitutents at the same ring carbon atom which join to form a cyclopropyl spiro ring,
or said heterocyclyl or halo-substituted heterocyclyl is fused with a (C₃-C₅)heterocycloalkyl ring, wherein said (C₃-C₅)heterocycloalkyl ring contains ring carbon atoms and 1 ring O atom;
or R₁ is heteroaryl, wherein said heteroaryl is a 5 or 6 membered fully unsaturated monocyclic group comprising ring carbon atoms and 1, 2, 3 or 4 ring heteroatoms independently selected from N, O and S, preferably 1 or 2 ring heteroatoms, wherein the total number of ring S atoms does not exceed 1, and the total number of ring O atoms does not exceed 1, wherein said heteroaryl is unsubstituted or substituted by 1, 2 or 3 substituents independently selected from R₂₁ and R₃₀, wherein R₂₁ and R₃₀ are independently selected from halo and (C₁-C₄)alkyl, wherein said (C₁-C₄)alkyl is unsubstituted or substituted by 1, 2 or 3 halo,
or R₁ is phenyl, wherein said phenyl is unsubstituted or substituted by 1, 2, 3 or 4, preferably 1 or 2, R₃₃, wherein R₃₃ is halo, and wherein said phenyl or halo-substituted phenyl is substituted by 0, 1 or 2 R₁₅ substituents,
or R₁ is (C₂-C₄)alkynyl or (C₂-C₄)alkenyl, wherein said (C₂-C₄)alkynyl and (C₂-C₄)alkenyl are unsubstituted or substituted by (C₁-C₄)alkyl-O—C(O)—, or morpholinyl;
each R₁₅, R₁₆, R₁₇, R₁₈, R₁₉, R₂₀, R₂₂ and R₂₃ is independently selected from:
   halo
   (C₁-C₄)alkyl-O— unsubstituted or substituted by 1, 2 or 3 halo;
   (C₁-C₄)alkyl unsubstituted or substituted by OH, —O—(C₁-C₂)alkyl or 1, 2 or 3 halo,
   HOC(O)—(CH₂)ₙ—,
   H₃C—C(O)(CH₂)ₙ—,
   (C₁-C₄)alkyl-O—C(O)(CH₂)ₙ,
   ═O
   azetidinyl or pyrrolidinyl, wherein said azetidinyl and pyrrolidinyl are linked to the rest of the molecule via the N atom, and are each unsubstituted or substituted by 1 or 2 F,
   R₂₅(R₂₄)N—, wherein R₂₄ is H or (C₁-C₄)alkyl unsubstituted or substituted by 1, 2 or 3 halo, R₂₅ is H or (C₁-C₄)alkyl unsubstituted or substituted by 1, 2 or 3 halo,
   OH
wherein n is 0, 1 or 2,
R₂₈ is CH₃, H or deuterium;
R₂₇ is CH₃, H or deuterium;
or R₂₈ and R₂₇ join, together with the carbon atom to which they are attached, to form a cyclopropyl ring;
R₂ is the moiety:

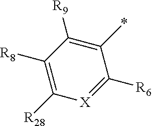

R₆ is selected from:
   H,
   halo,
   (C₁-C₄)alkyl unsubstituted or substituted by 1, 2 or 3 halo,
   (C₃-C₅)cycloalkyl unsubstituted or substituted by 1, 2 or 3 halo,
   —O—(C₁-C₄)alkyl unsubstituted or substituted by 1, 2 or 3 halo,
   OH, and
   CN;
R₈ is selected from H, halo, and (C₁-C₄)alkyl unsubstituted or substituted by 1, 2 or 3 halo,
R₉ is selected from H, O—CH₃, OH, CN, CH₃ and halo;
R₂₈ is selected from:
   SF₅,
   H,
   —C(O)H,
   halo,
   (C₁-C₄)alkyl unsubstituted or substituted by 1, 2 or 3 halo,
   (C₁-C₄)alkynyl,
   (C₁-C₄)alkenyl,
   (C₃-C₅)cycloalkyl unsubstituted or substituted by 1, 2 or 3 halo, and
OCF₃;
X is selected from C—R₇ and N, wherein R₇ is H or halo, or R₇ can join, together with R₂₈ or R₆, and the atoms to which they are attached, to form a fused (C₄-C₆)cycloalkyl ring, wherein said fused (C₄-C₆)cycloalkyl ring is unsubstituted or substituted by 1, 2 or 3 halo, or
R₂ is selected from:

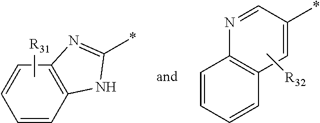

wherein
R₃₁ is selected from H, halo and CH₃,
R₃₂ is selected from H, halo and CH₃,
R₃ is:
   cyclopropyl,
   O—CH₃,
   N(CH₃)₂,
   S—CH₃,
   (C₁-C₄)alkyl unsubstituted or substituted by 1, 2 or 3 substituents independently selected from halo and OH;
R₄ is selected from:

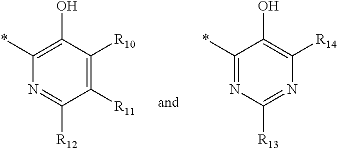

wherein
R₁₀, R₁₁, R₁₂, R₁₃ and R₁₄ are independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   —S—(C₁-C₃)alkyl,
   —O—(C₁-C₄)alkyl unsubstituted or substituted by 1, 2 or 3 halo substituents,
   OH,
   (C₃-C₅)cycloalkyl, wherein said (C₃-C₅)cycloalkyl is unsubstituted or substituted by 1 or 2 halo,
   —O—(C₃-C₅)cycloalkyl,
   —NR₃₄R₃₅ wherein R₃₄ and R₃₅ are independently selected from:
      H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
   CN,
   —(C₂-C₄)alkenyl,
   —(C₂-C₄)alkynyl,
   —C(O)H, and
   —C(O)(C₁-C₄)alkyl;
and
* indicates a point of attachment,
or the WRN inhibitor is a compound of formula (1g), or a pharmaceutically acceptable salt thereof:

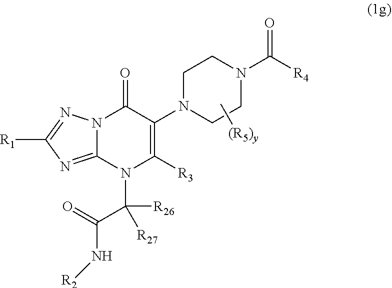

wherein
R₁ is selected from:

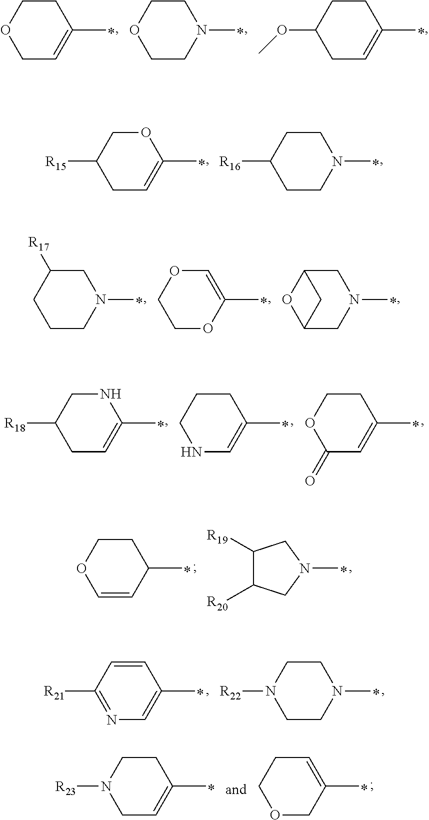

R₁₅ is H or F;
R₁₆ is H or R₂₅(R₂₄)N—;
R₁₇ is H or F;
R₁₈ is H or F;
R₁₉ is H or F;
R₂₀ is H or F;
R₂₁ is H or CHs;
R₂₂ is H, CF₃, CHF₂CH₂, HOC(O)—CH₂, H₃C—C(O)—, (H₃C)₃C—O—C(O);
R₂₃ is H, CF₃, CHF₂CH₂—, (H₃C)₃C—O—C(O)—;
R₂₄ is CHs;
R₂₅ is CHF₂CH₂—;
R₂₆ is CH₃, H or deuterium;
R₂₇ is H or deuterium;
R₂ is the moiety.

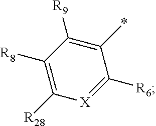

wherein Re is selected from H, Cl, CH₃, F, and Br,
R₈ is selected from H, Cl, F and CF₃;
R₉ is selected from H, CH₃ and Cl;
R₂₈ is selected from CF₃, CF₂H, —CH₂CH₃, Cl, SF₅, Br and —C(O)H;
X is selected from C—R₇ and N;
R₇ is selected from H and F;
R₃ is selected from CH₃, CH₂CH₃, cyclopropyl and hydroxyethyl;
R₄ is selected from:

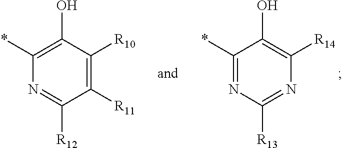

wherein
R₁₀ is selected from H, F, Cl, CH₃ and OCF₃;
R₁₁ is selected from H, Cl, F, and CH₃;
R₁₂ is selected from H, Cl and CH₃;
R₁₃ is selected from H and CH₃;
R₁₄ is selected from H, CH₃, —CH₂CH₃, cyclopropyl, —OCHF₂, OCF₃ and Cl;
y is 0, 1 or 2;
R₅ is CH₃; or alternatively, two R₅ groups on adjacent carbon atoms join, along with the carbon atoms to which they are attached, to form a fused cyclobutyl ring:

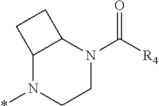

and
* indicates a point of attachment.

In an embodiment of any of the first to fourth aspects of the invention, the WRN inhibitor is a compound selected from:

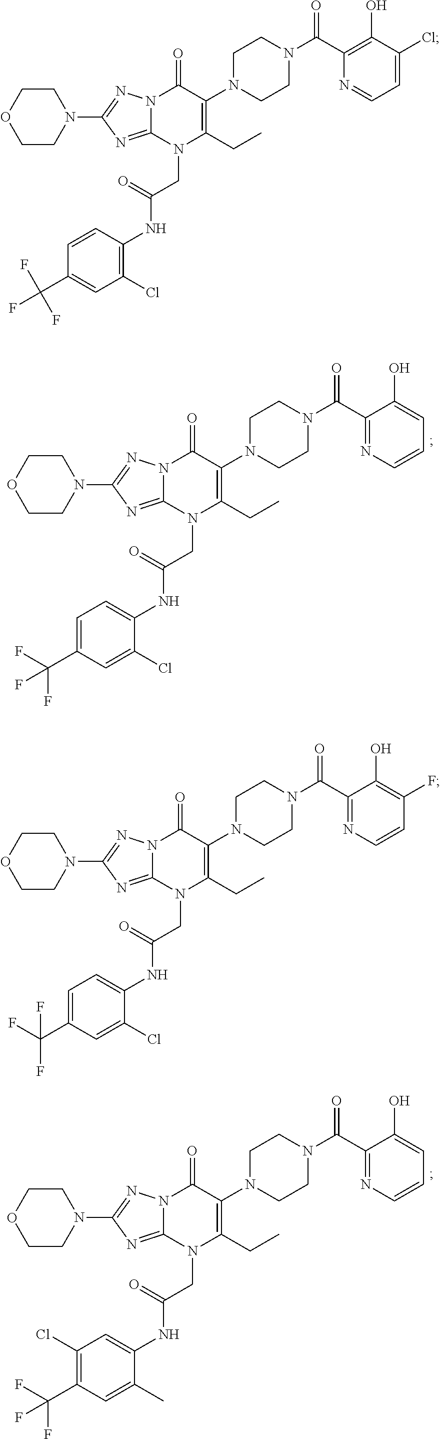 [SMILES: CCc1c(N2CCN(C(=O)c3nccc(Cl)c3O)CC2)c(=O)n2nc(N3CCOCC3)nc2n1CC(=O)Nc1ccc(C(F)(F)F)cc1Cl ; CCc1c(N2CCN(C(=O)c3ncccc3O)CC2)c(=O)n2nc(N3CCOCC3)nc2n1CC(=O)Nc1ccc(C(F)(F)F)cc1Cl ; CCc1c(N2CCN(C(=O)c3nccc(F)c3O)CC2)c(=O)n2nc(N3CCOCC3)nc2n1CC(=O)Nc1ccc(C(F)(F)F)cc1Cl ; CCc1c(N2CCN(C(=O)c3ncccc3O)CC2)c(=O)n2nc(N3CCOCC3)nc2n1CC(=O)Nc1cc(Cl)c(C(F)(F)F)cc1C]

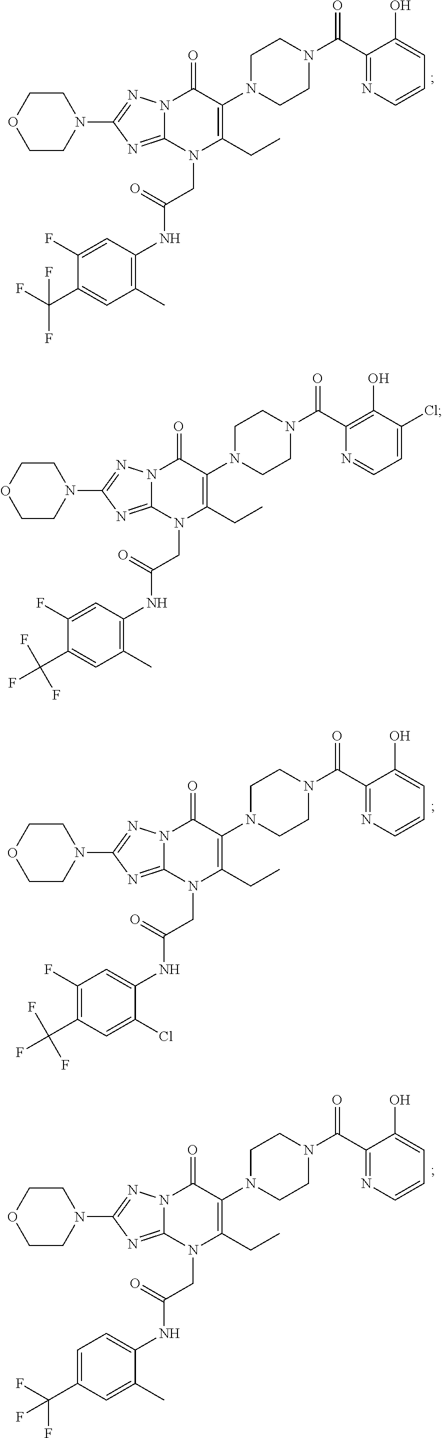 [SMILES: CCc1c(N2CCN(C(=O)c3ncccc3O)CC2)c(=O)n2nc(N3CCOCC3)nc2n1CC(=O)Nc1cc(F)c(C(F)(F)F)cc1C ; CCc1c(N2CCN(C(=O)c3nccc(Cl)c3O)CC2)c(=O)n2nc(N3CCOCC3)nc2n1CC(=O)Nc1cc(F)c(C(F)(F)F)cc1C ; CCc1c(N2CCN(C(=O)c3ncccc3O)CC2)c(=O)n2nc(N3CCOCC3)nc2n1CC(=O)Nc1cc(F)c(C(F)(F)F)cc1Cl ; CCc1c(N2CCN(C(=O)c3ncccc3O)CC2)c(=O)n2nc(N3CCOCC3)nc2n1CC(=O)Nc1ccc(C(F)(F)F)cc1C]

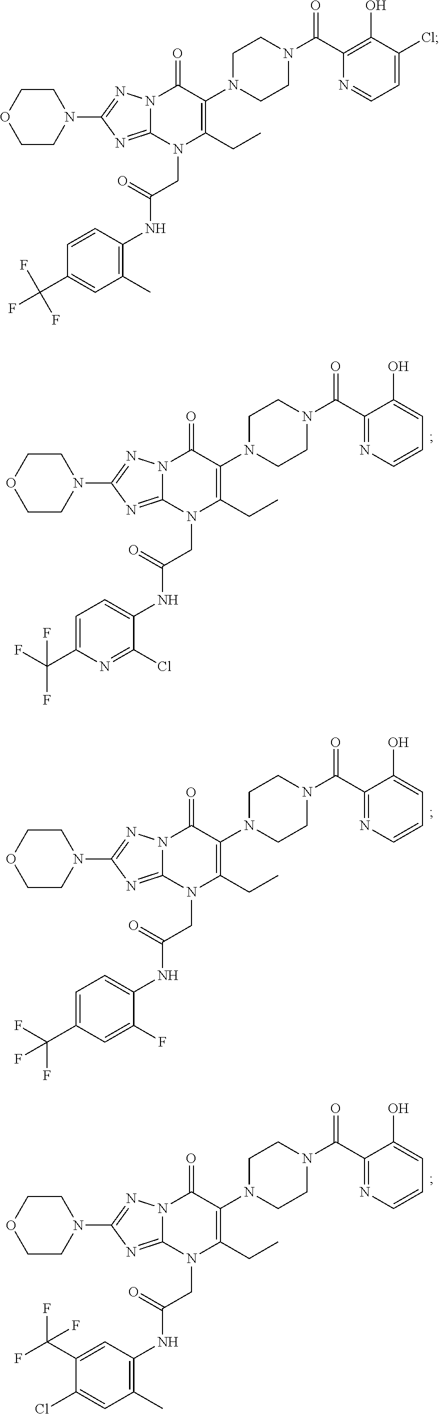 [SMILES: CCc1c(N2CCN(C(=O)c3nccc(Cl)c3O)CC2)c(=O)n2nc(N3CCOCC3)nc2n1CC(=O)Nc1ccc(C(F)(F)F)cc1C ; CCc1c(N2CCN(C(=O)c3ncccc3O)CC2)c(=O)n2nc(N3CCOCC3)nc2n1CC(=O)Nc1ccc(C(F)(F)F)nc1Cl ; CCc1c(N2CCN(C(=O)c3ncccc3O)CC2)c(=O)n2nc(N3CCOCC3)nc2n1CC(=O)Nc1ccc(C(F)(F)F)cc1F ; CCc1c(N2CCN(C(=O)c3ncccc3O)CC2)c(=O)n2nc(N3CCOCC3)nc2n1CC(=O)Nc1cc(C(F)(F)F)c(Cl)cc1C]

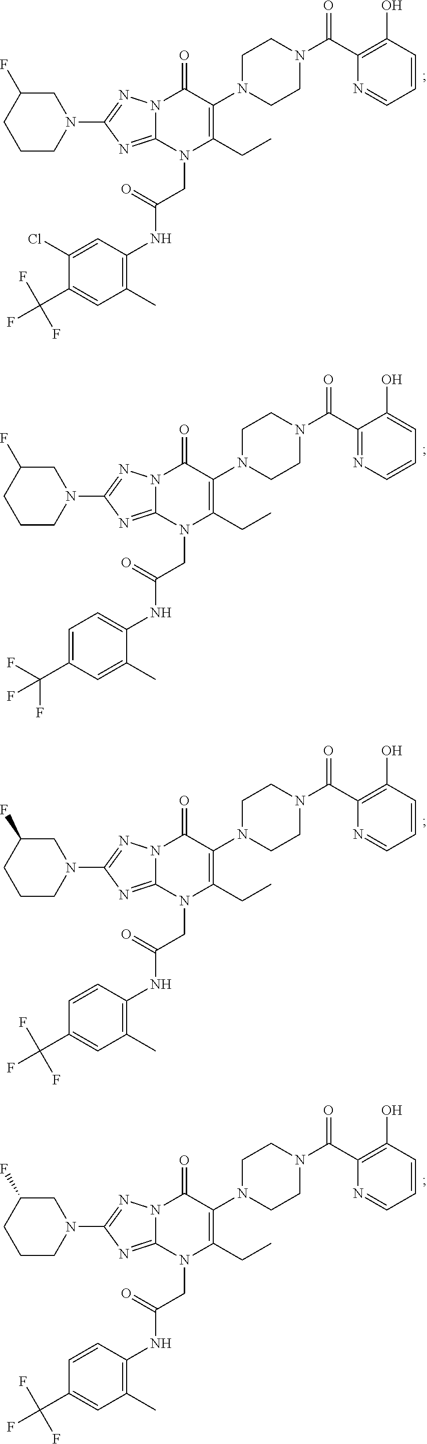 [SMILES: CCc1c(N2CCN(C(=O)c3ncccc3O)CC2)c(=O)n2nc(N3CCCC(F)C3)nc2n1CC(=O)Nc1cc(Cl)c(C(F)(F)F)cc1C ; CCc1c(N2CCN(C(=O)c3ncccc3O)CC2)c(=O)n2nc(N3CCCC(F)C3)nc2n1CC(=O)Nc1ccc(C(F)(F)F)cc1C ; CCc1c(N2CCN(C(=O)c3ncccc3O)CC2)c(=O)n2nc(N3CCC[C@@H](F)C3)nc2n1CC(=O)Nc1ccc(C(F)(F)F)cc1C ; CCc1c(N2CCN(C(=O)c3ncccc3O)CC2)c(=O)n2nc(N3CCC[C@H](F)C3)nc2n1CC(=O)Nc1ccc(C(F)(F)F)cc1C]

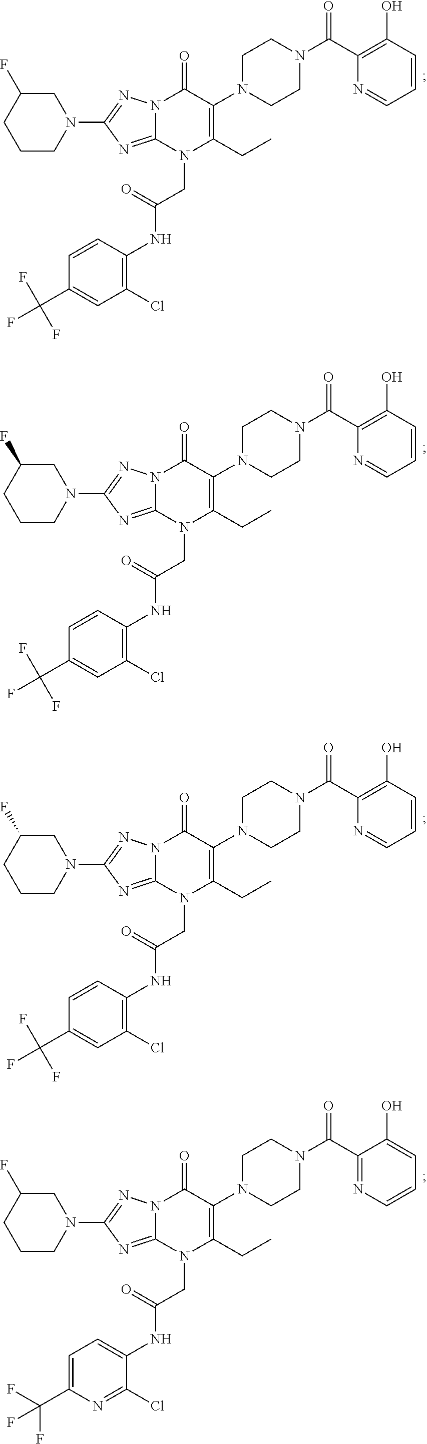 [SMILES: CCc1c(N2CCN(C(=O)c3ncccc3O)CC2)c(=O)n2nc(N3CCCC(F)C3)nc2n1CC(=O)Nc1ccc(C(F)(F)F)cc1Cl ; CCc1c(N2CCN(C(=O)c3ncccc3O)CC2)c(=O)n2nc(N3CCC[C@@H](F)C3)nc2n1CC(=O)Nc1ccc(C(F)(F)F)cc1Cl ; CCc1c(N2CCN(C(=O)c3ncccc3O)CC2)c(=O)n2nc(N3CCC[C@H](F)C3)nc2n1CC(=O)Nc1ccc(C(F)(F)F)cc1Cl ; CCc1c(N2CCN(C(=O)c3ncccc3O)CC2)c(=O)n2nc(N3CCCC(F)C3)nc2n1CC(=O)Nc1ccc(C(F)(F)F)nc1Cl]

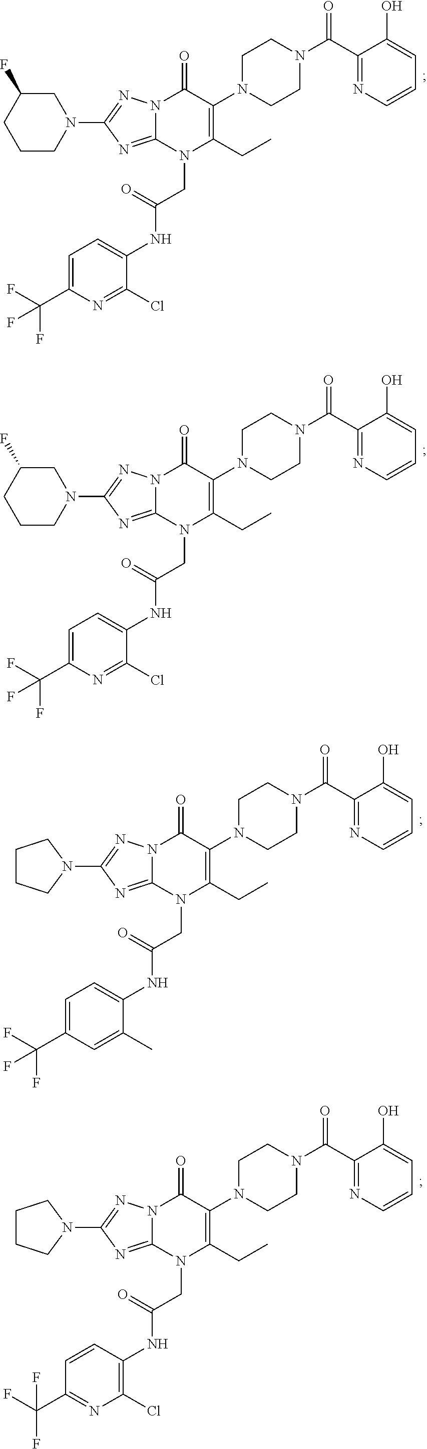 [SMILES: CCc1c(N2CCN(C(=O)c3ncccc3O)CC2)c(=O)n2nc(N3CCC[C@@H](F)C3)nc2n1CC(=O)Nc1ccc(C(F)(F)F)nc1Cl ; CCc1c(N2CCN(C(=O)c3ncccc3O)CC2)c(=O)n2nc(N3CCC[C@H](F)C3)nc2n1CC(=O)Nc1ccc(C(F)(F)F)nc1Cl ; CCc1c(N2CCN(C(=O)c3ncccc3O)CC2)c(=O)n2nc(N3CCCC3)nc2n1CC(=O)Nc1ccc(C(F)(F)F)cc1C ; CCc1c(N2CCN(C(=O)c3ncccc3O)CC2)c(=O)n2nc(N3CCCC3)nc2n1CC(=O)Nc1ccc(C(F)(F)F)nc1Cl]

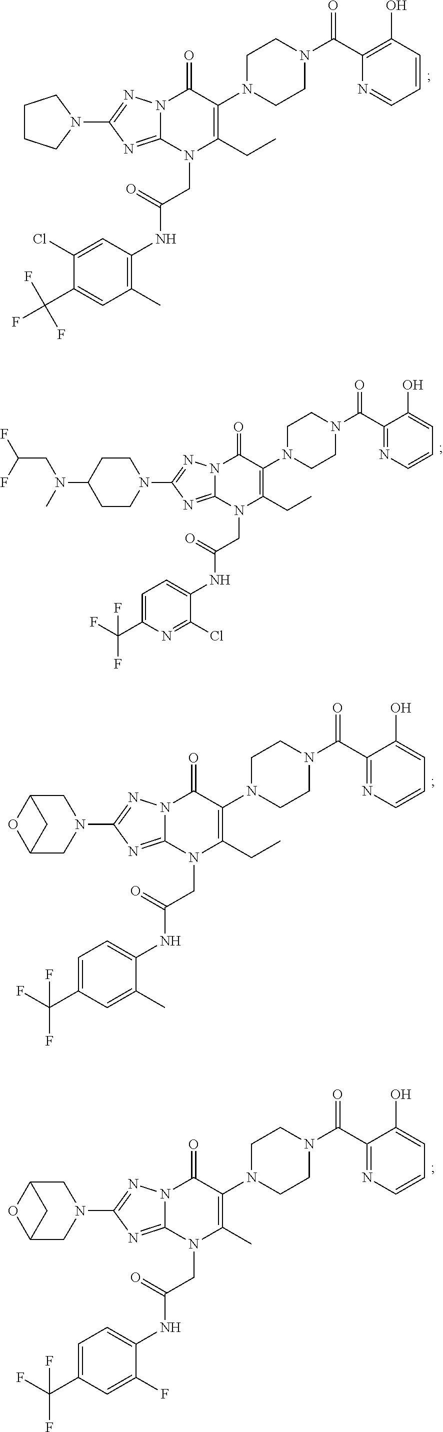 [SMILES: CCc1c(N2CCN(C(=O)c3ncccc3O)CC2)c(=O)n2nc(N3CCCC3)nc2n1CC(=O)Nc1cc(Cl)c(C(F)(F)F)cc1C ; CCc1c(N2CCN(C(=O)c3ncccc3O)CC2)c(=O)n2nc(N3CCC(N(C)CC(F)F)CC3)nc2n1CC(=O)Nc1ccc(C(F)(F)F)nc1Cl ; CCc1c(N2CCN(C(=O)c3ncccc3O)CC2)c(=O)n2nc(N3CC4CC(C3)O4)nc2n1CC(=O)Nc1ccc(C(F)(F)F)cc1C ; Cc1c(N2CCN(C(=O)c3ncccc3O)CC2)c(=O)n2nc(N3CC4CC(C3)O4)nc2n1CC(=O)Nc1ccc(C(F)(F)F)cc1F]

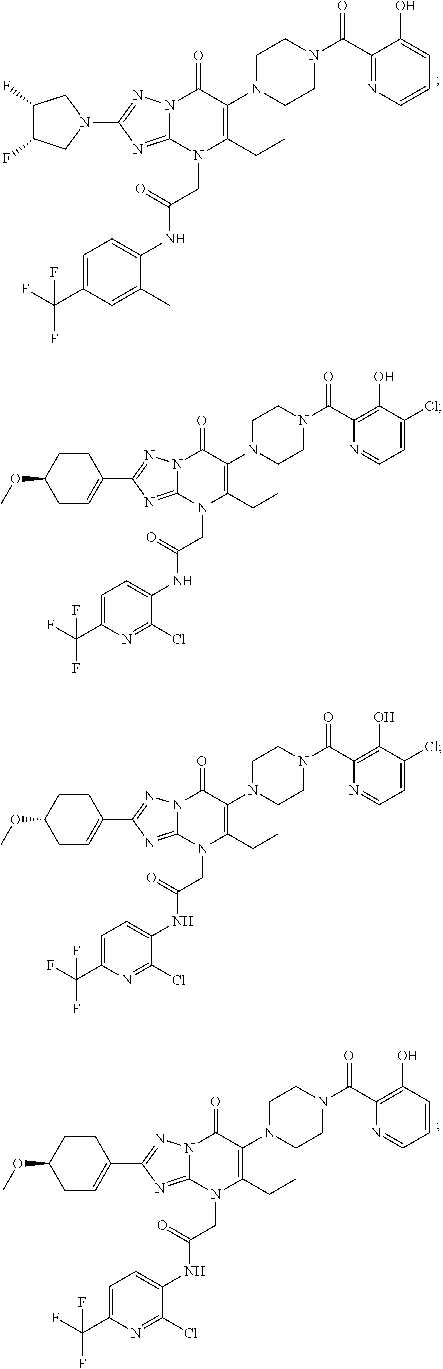 [SMILES: CCc1c(N2CCN(C(=O)c3ncccc3O)CC2)c(=O)n2nc(N3C[C@@H](F)[C@@H](F)C3)nc2n1CC(=O)Nc1ccc(C(F)(F)F)cc1C ; CCc1c(N2CCN(C(=O)c3nccc(Cl)c3O)CC2)c(=O)n2nc(C3=CC[C@@H](OC)CC3)nc2n1CC(=O)Nc1ccc(C(F)(F)F)nc1Cl ; CCc1c(N2CCN(C(=O)c3nccc(Cl)c3O)CC2)c(=O)n2nc(C3=CC[C@H](OC)CC3)nc2n1CC(=O)Nc1ccc(C(F)(F)F)nc1Cl ; CCc1c(N2CCN(C(=O)c3ncccc3O)CC2)c(=O)n2nc(C3=CC[C@@H](OC)CC3)nc2n1CC(=O)Nc1ccc(C(F)(F)F)nc1Cl]

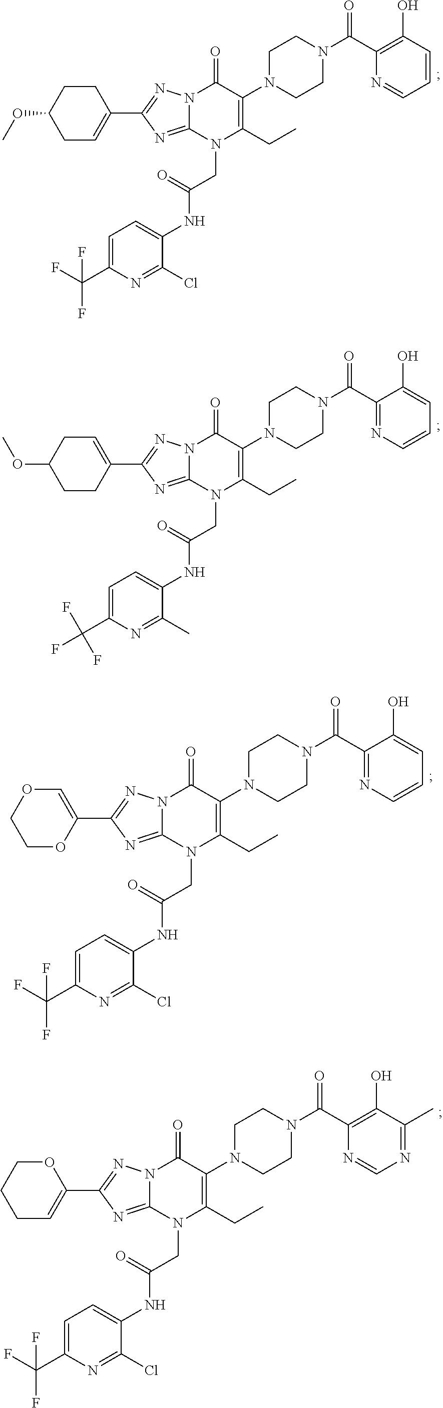 [SMILES: CCc1c(N2CCN(C(=O)c3ncccc3O)CC2)c(=O)n2nc(C3=CC[C@H](OC)CC3)nc2n1CC(=O)Nc1ccc(C(F)(F)F)nc1Cl ; CCc1c(N2CCN(C(=O)c3ncccc3O)CC2)c(=O)n2nc(C3=CCC(OC)CC3)nc2n1CC(=O)Nc1ccc(C(F)(F)F)nc1C ; CCc1c(N2CCN(C(=O)c3ncccc3O)CC2)c(=O)n2nc(C3=COCCO3)nc2n1CC(=O)Nc1ccc(C(F)(F)F)nc1Cl ; CCc1c(N2CCN(C(=O)c3ncnc(C)c3O)CC2)c(=O)n2nc(C3=CCCCO3)nc2n1CC(=O)Nc1ccc(C(F)(F)F)nc1Cl]

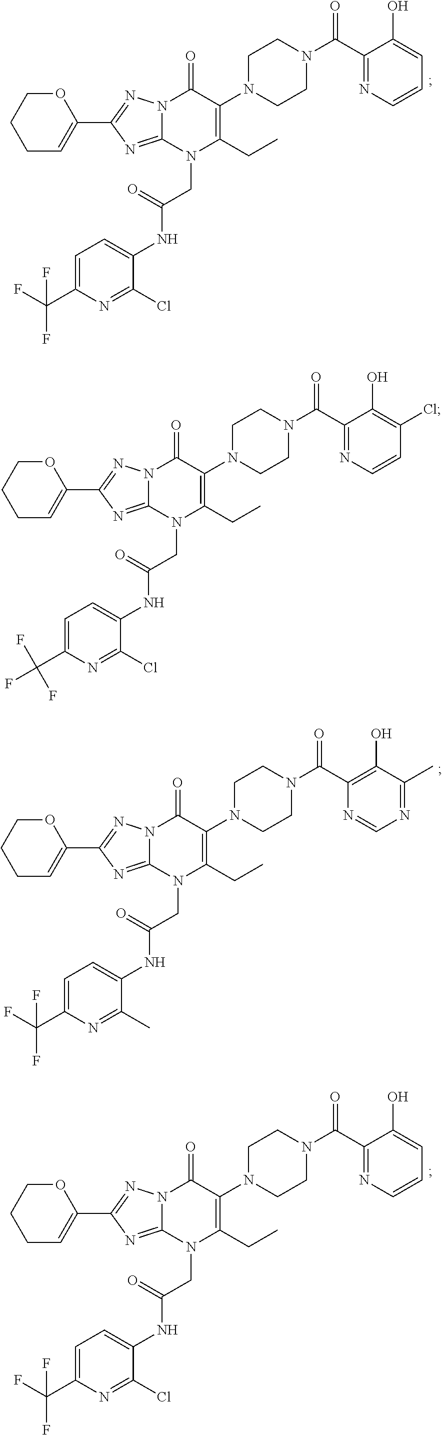 [SMILES: CCc1c(N2CCN(C(=O)c3ncccc3O)CC2)c(=O)n2nc(C3=CCCCO3)nc2n1CC(=O)Nc1ccc(C(F)(F)F)nc1Cl ; CCc1c(N2CCN(C(=O)c3nccc(Cl)c3O)CC2)c(=O)n2nc(C3=CCCCO3)nc2n1CC(=O)Nc1ccc(C(F)(F)F)nc1Cl ; CCc1c(N2CCN(C(=O)c3ncnc(C)c3O)CC2)c(=O)n2nc(C3=CCCCO3)nc2n1CC(=O)Nc1ccc(C(F)(F)F)nc1C ; CCc1c(N2CCN(C(=O)c3ncccc3O)CC2)c(=O)n2nc(C3=CCCCO3)nc2n1CC(=O)Nc1ccc(C(F)(F)F)nc1Cl]

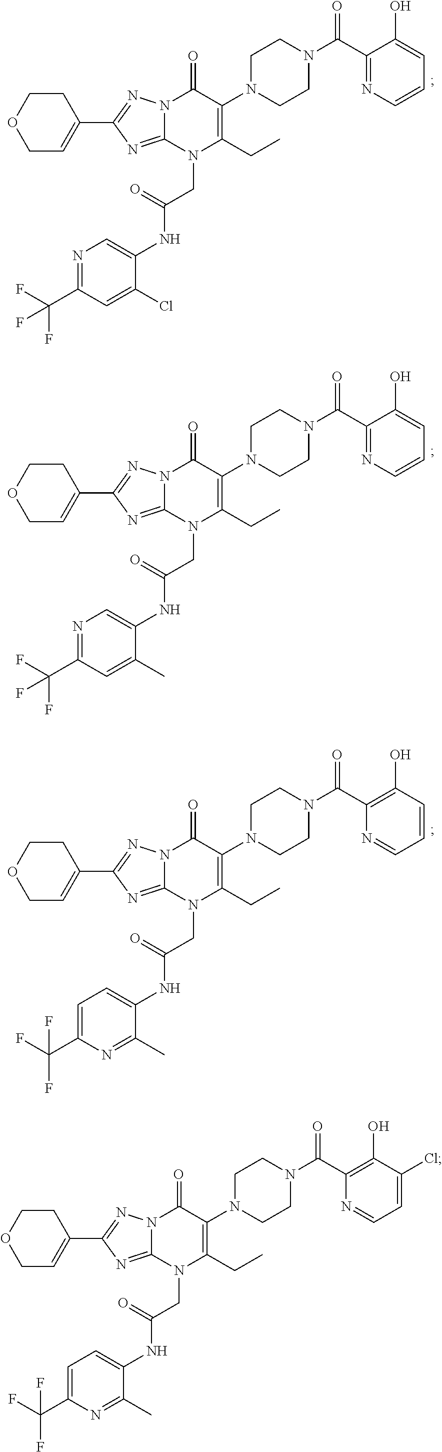 [SMILES: CCc1c(N2CCN(C(=O)c3ncccc3O)CC2)c(=O)n2nc(C3=CCOCC3)nc2n1CC(=O)Nc1cnc(C(F)(F)F)cc1Cl ; CCc1c(N2CCN(C(=O)c3ncccc3O)CC2)c(=O)n2nc(C3=CCOCC3)nc2n1CC(=O)Nc1cnc(C(F)(F)F)cc1C ; CCc1c(N2CCN(C(=O)c3ncccc3O)CC2)c(=O)n2nc(C3=CCOCC3)nc2n1CC(=O)Nc1ccc(C(F)(F)F)nc1C ; CCc1c(N2CCN(C(=O)c3nccc(Cl)c3O)CC2)c(=O)n2nc(C3=CCOCC3)nc2n1CC(=O)Nc1ccc(C(F)(F)F)nc1C]

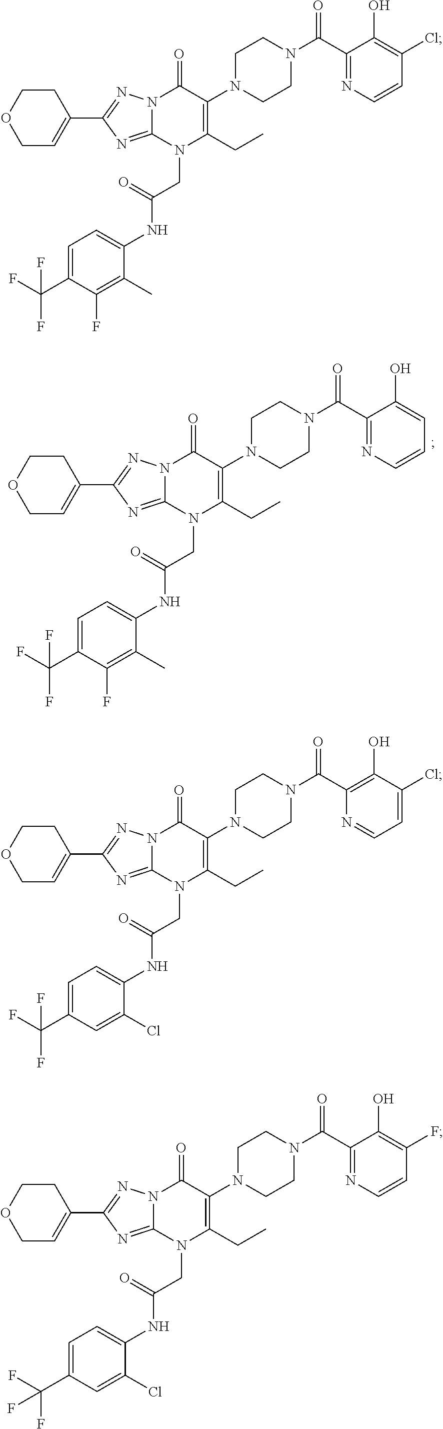 [SMILES: CCc1c(N2CCN(C(=O)c3nccc(Cl)c3O)CC2)c(=O)n2nc(C3=CCOCC3)nc2n1CC(=O)Nc1ccc(C(F)(F)F)c(F)c1C ; CCc1c(N2CCN(C(=O)c3ncccc3O)CC2)c(=O)n2nc(C3=CCOCC3)nc2n1CC(=O)Nc1ccc(C(F)(F)F)c(F)c1C ; CCc1c(N2CCN(C(=O)c3nccc(Cl)c3O)CC2)c(=O)n2nc(C3=CCOCC3)nc2n1CC(=O)Nc1ccc(C(F)(F)F)cc1Cl ; CCc1c(N2CCN(C(=O)c3nccc(F)c3O)CC2)c(=O)n2nc(C3=CCOCC3)nc2n1CC(=O)Nc1ccc(C(F)(F)F)cc1Cl]

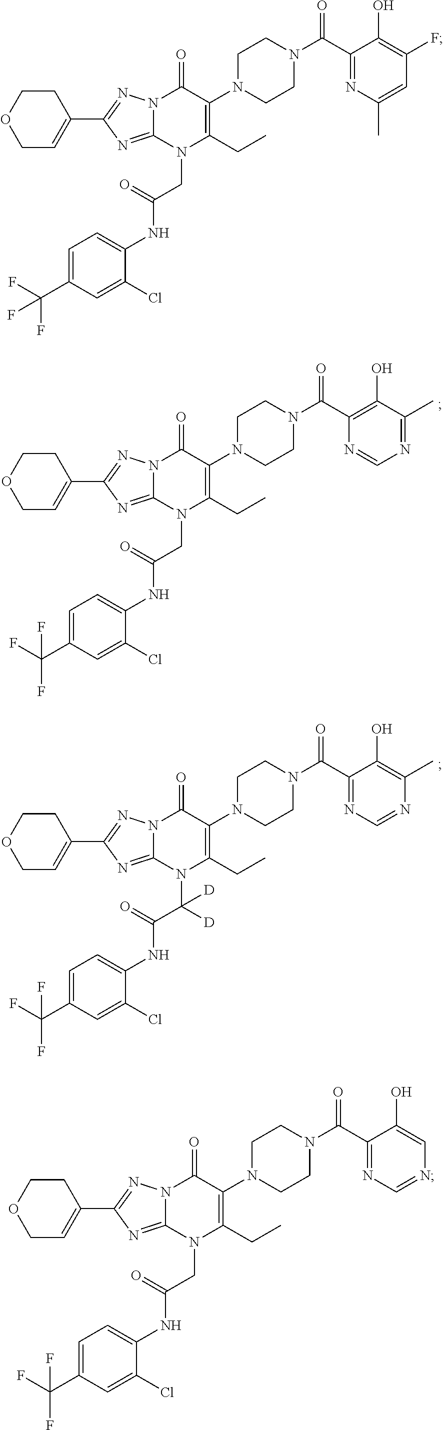 [SMILES: CCc1c(N2CCN(C(=O)c3nc(C)cc(F)c3O)CC2)c(=O)n2nc(C3=CCOCC3)nc2n1CC(=O)Nc1ccc(C(F)(F)F)cc1Cl ; CCc1c(N2CCN(C(=O)c3ncnc(C)c3O)CC2)c(=O)n2nc(C3=CCOCC3)nc2n1CC(=O)Nc1ccc(C(F)(F)F)cc1Cl ; [2H]C([2H])(C(=O)Nc1ccc(C(F)(F)F)cc1Cl)n1c(CC)c(N2CCN(C(=O)c3ncnc(C)c3O)CC2)c(=O)n2nc(C3=CCOCC3)nc12 ; CCc1c(N2CCN(C(=O)c3ncncc3O)CC2)c(=O)n2nc(C3=CCOCC3)nc2n1CC(=O)Nc1ccc(C(F)(F)F)cc1Cl]

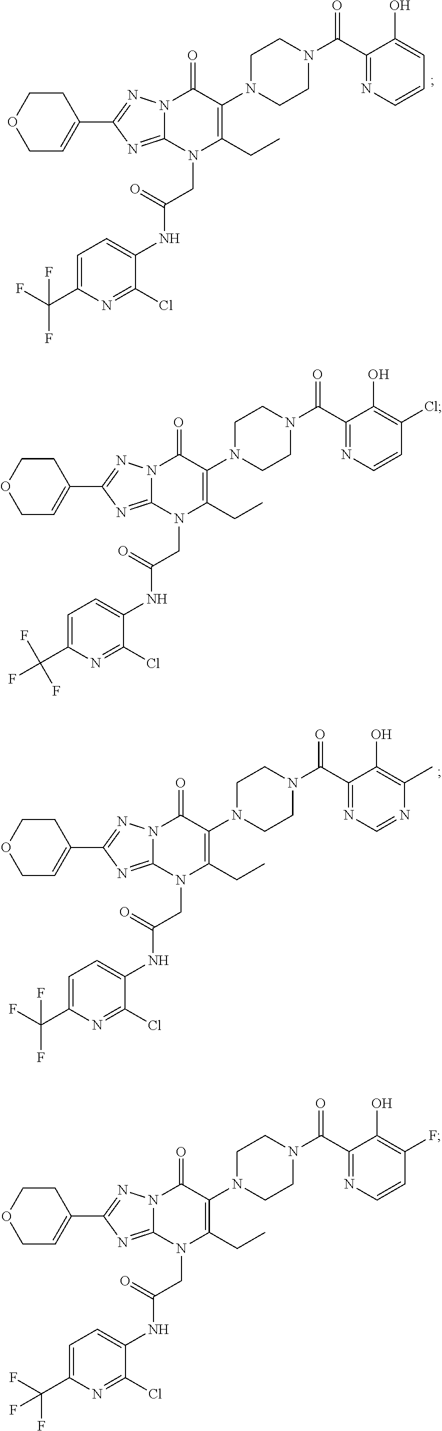 [SMILES: CCc1c(N2CCN(C(=O)c3ncccc3O)CC2)c(=O)n2nc(C3=CCOCC3)nc2n1CC(=O)Nc1ccc(C(F)(F)F)nc1Cl ; CCc1c(N2CCN(C(=O)c3nccc(Cl)c3O)CC2)c(=O)n2nc(C3=CCOCC3)nc2n1CC(=O)Nc1ccc(C(F)(F)F)nc1Cl ; CCc1c(N2CCN(C(=O)c3ncnc(C)c3O)CC2)c(=O)n2nc(C3=CCOCC3)nc2n1CC(=O)Nc1ccc(C(F)(F)F)nc1Cl ; CCc1c(N2CCN(C(=O)c3nccc(F)c3O)CC2)c(=O)n2nc(C3=CCOCC3)nc2n1CC(=O)Nc1ccc(C(F)(F)F)nc1Cl]

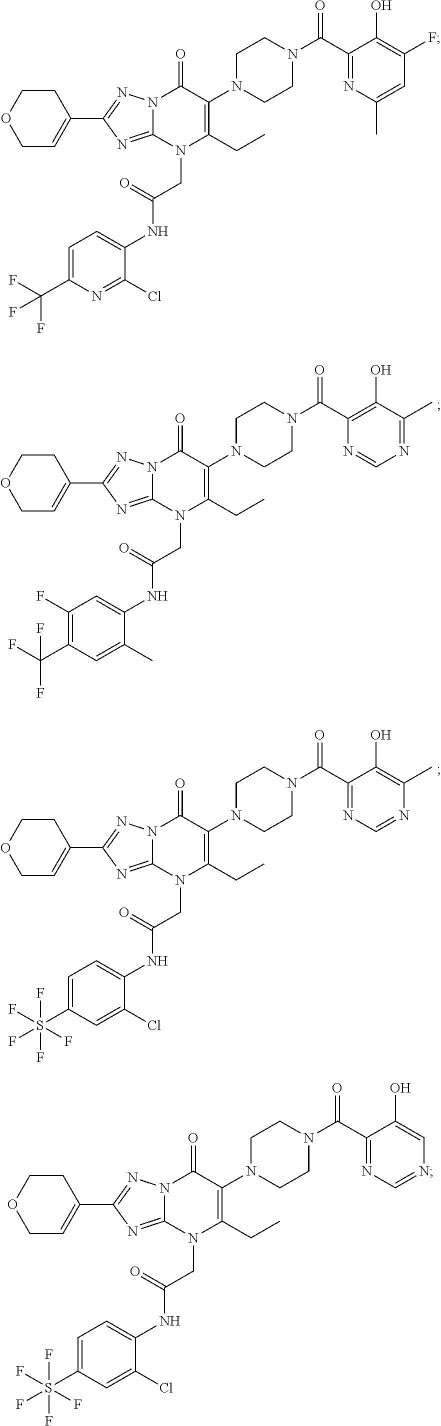 [SMILES: CCc1c(N2CCN(C(=O)c3nc(C)cc(F)c3O)CC2)c(=O)n2nc(C3=CCOCC3)nc2n1CC(=O)Nc1ccc(C(F)(F)F)nc1Cl ; CCc1c(N2CCN(C(=O)c3ncnc(C)c3O)CC2)c(=O)n2nc(C3=CCOCC3)nc2n1CC(=O)Nc1cc(F)c(C(F)(F)F)cc1C ; CCc1c(N2CCN(C(=O)c3ncnc(C)c3O)CC2)c(=O)n2nc(C3=CCOCC3)nc2n1CC(=O)Nc1ccc(S(F)(F)(F)(F)F)cc1Cl ; CCc1c(N2CCN(C(=O)c3ncncc3O)CC2)c(=O)n2nc(C3=CCOCC3)nc2n1CC(=O)Nc1ccc(S(F)(F)(F)(F)F)cc1Cl]

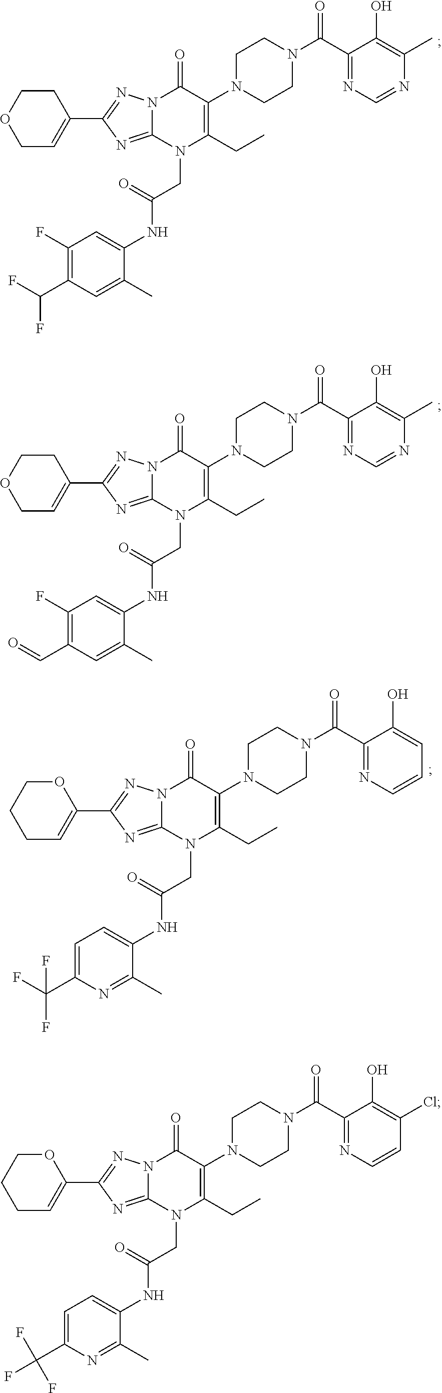 [SMILES: CCc1c(N2CCN(C(=O)c3ncnc(C)c3O)CC2)c(=O)n2nc(C3=CCOCC3)nc2n1CC(=O)Nc1cc(F)c(C(F)F)cc1C ; CCc1c(N2CCN(C(=O)c3ncnc(C)c3O)CC2)c(=O)n2nc(C3=CCOCC3)nc2n1CC(=O)Nc1cc(F)c(C=O)cc1C ; CCc1c(N2CCN(C(=O)c3ncccc3O)CC2)c(=O)n2nc(C3=CCCCO3)nc2n1CC(=O)Nc1ccc(C(F)(F)F)nc1C ; CCc1c(N2CCN(C(=O)c3nccc(Cl)c3O)CC2)c(=O)n2nc(C3=CCCCO3)nc2n1CC(=O)Nc1ccc(C(F)(F)F)nc1C]

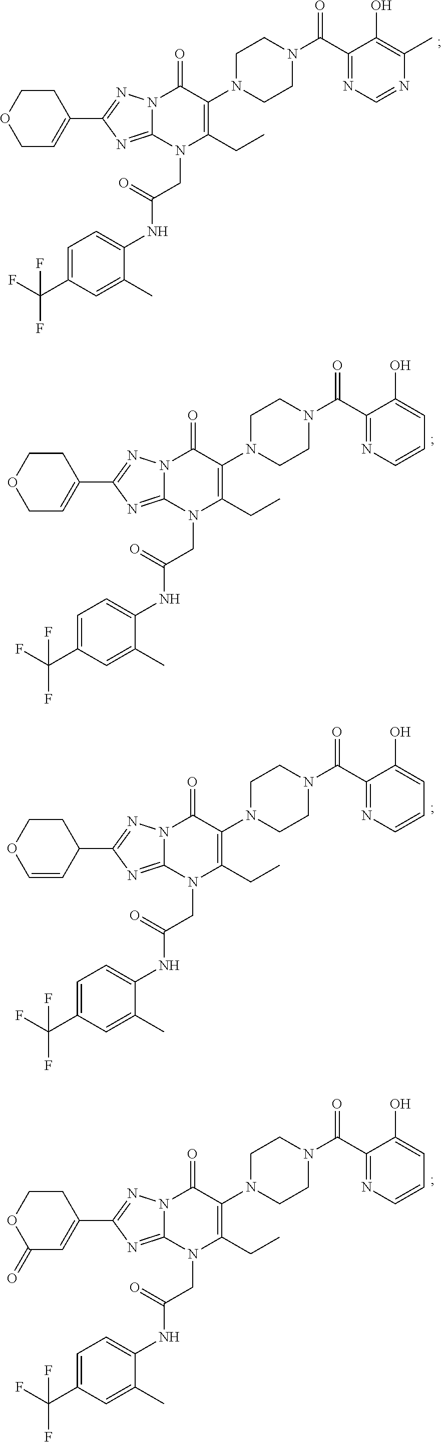 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1C ; CCc1c(N2CCN(C(=O)c3ncccc3O)CC2)c(=O)n2nc(C3=CCOCC3)nc2n1CC(=O)Nc1ccc(C(F)(F)F)cc1C ; CCc1c(N2CCN(C(=O)c3ncccc3O)CC2)c(=O)n2nc(C3C=COCC3)nc2n1CC(=O)Nc1ccc(C(F)(F)F)cc1C ; CCc1c(N2CCN(C(=O)c3ncccc3O)CC2)c(=O)n2nc(C3=CC(=O)OCC3)nc2n1CC(=O)Nc1ccc(C(F)(F)F)cc1C]

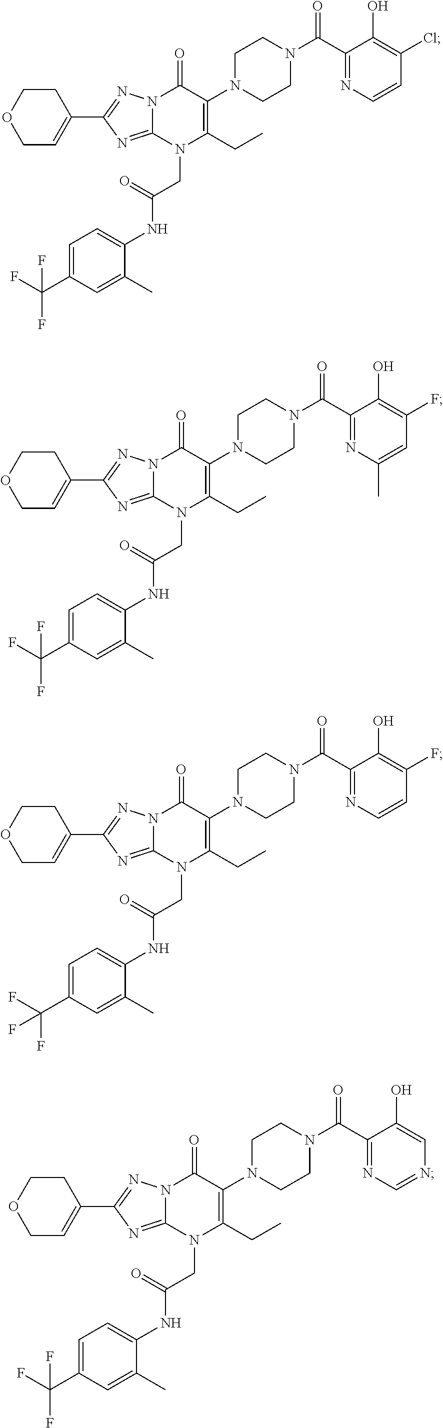 [SMILES: CCc1c(N2CCN(C(=O)c3nccc(Cl)c3O)CC2)c(=O)n2nc(C3=CCOCC3)nc2n1CC(=O)Nc1ccc(C(F)(F)F)cc1C ; CCc1c(N2CCN(C(=O)c3nc(C)cc(F)c3O)CC2)c(=O)n2nc(C3=CCOCC3)nc2n1CC(=O)Nc1ccc(C(F)(F)F)cc1C ; CCc1c(N2CCN(C(=O)c3nccc(F)c3O)CC2)c(=O)n2nc(C3=CCOCC3)nc2n1CC(=O)Nc1ccc(C(F)(F)F)cc1C ; CCc1c(N2CCN(C(=O)c3ncncc3O)CC2)c(=O)n2nc(C3=CCOCC3)nc2n1CC(=O)Nc1ccc(C(F)(F)F)cc1C]

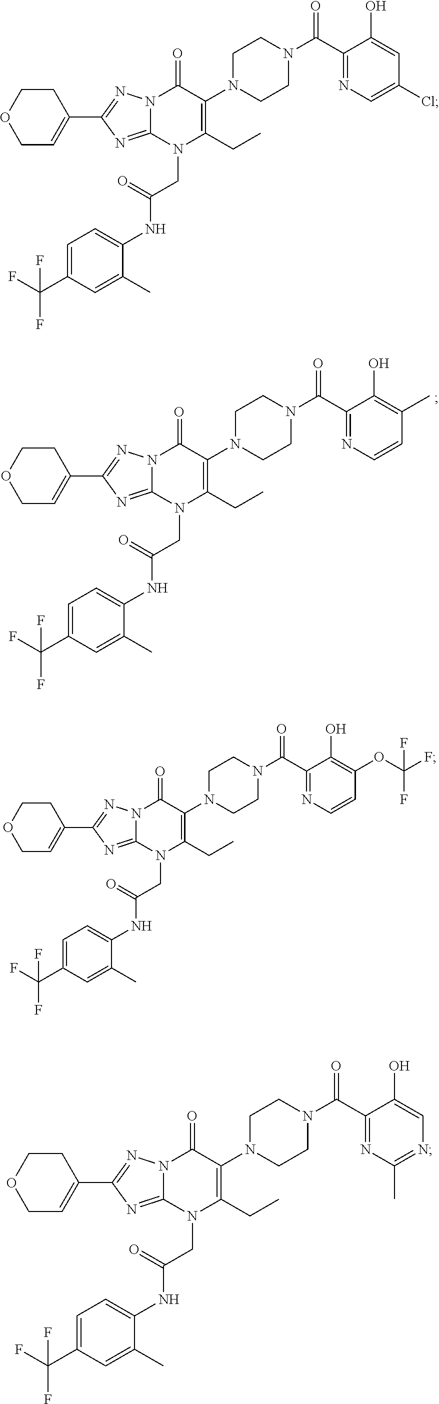 [SMILES: CCc1c(N2CCN(C(=O)c3ncc(Cl)cc3O)CC2)c(=O)n2nc(C3=CCOCC3)nc2n1CC(=O)Nc1ccc(C(F)(F)F)cc1C ; CCc1c(N2CCN(C(=O)c3nccc(C)c3O)CC2)c(=O)n2nc(C3=CCOCC3)nc2n1CC(=O)Nc1ccc(C(F)(F)F)cc1C ; CCc1c(N2CCN(C(=O)c3nccc(OC(F)(F)F)c3O)CC2)c(=O)n2nc(C3=CCOCC3)nc2n1CC(=O)Nc1ccc(C(F)(F)F)cc1C ; CCc1c(N2CCN(C(=O)c3nc(C)ncc3O)CC2)c(=O)n2nc(C3=CCOCC3)nc2n1CC(=O)Nc1ccc(C(F)(F)F)cc1C]

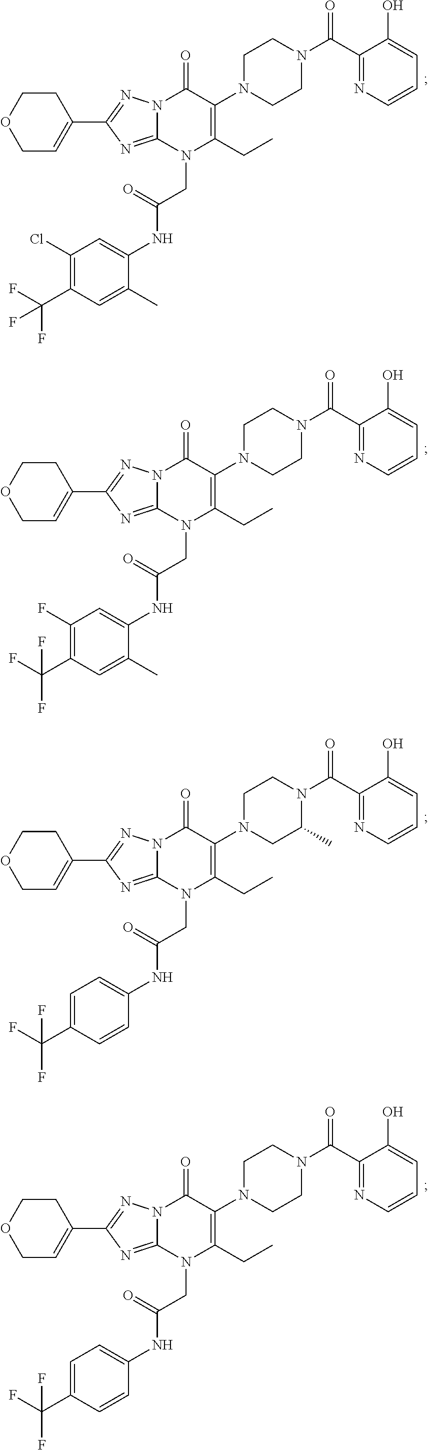 [SMILES: CCc1c(N2CCN(C(=O)c3ncccc3O)CC2)c(=O)n2nc(C3=CCOCC3)nc2n1CC(=O)Nc1cc(Cl)c(C(F)(F)F)cc1C ; CCc1c(N2CCN(C(=O)c3ncccc3O)CC2)c(=O)n2nc(C3=CCOCC3)nc2n1CC(=O)Nc1cc(F)c(C(F)(F)F)cc1C ; CCc1c(N2CCN(C(=O)c3ncccc3O)[C@H](C)C2)c(=O)n2nc(C3=CCOCC3)nc2n1CC(=O)Nc1ccc(C(F)(F)F)cc1 ; CCc1c(N2CCN(C(=O)c3ncccc3O)CC2)c(=O)n2nc(C3=CCOCC3)nc2n1CC(=O)Nc1ccc(C(F)(F)F)cc1]

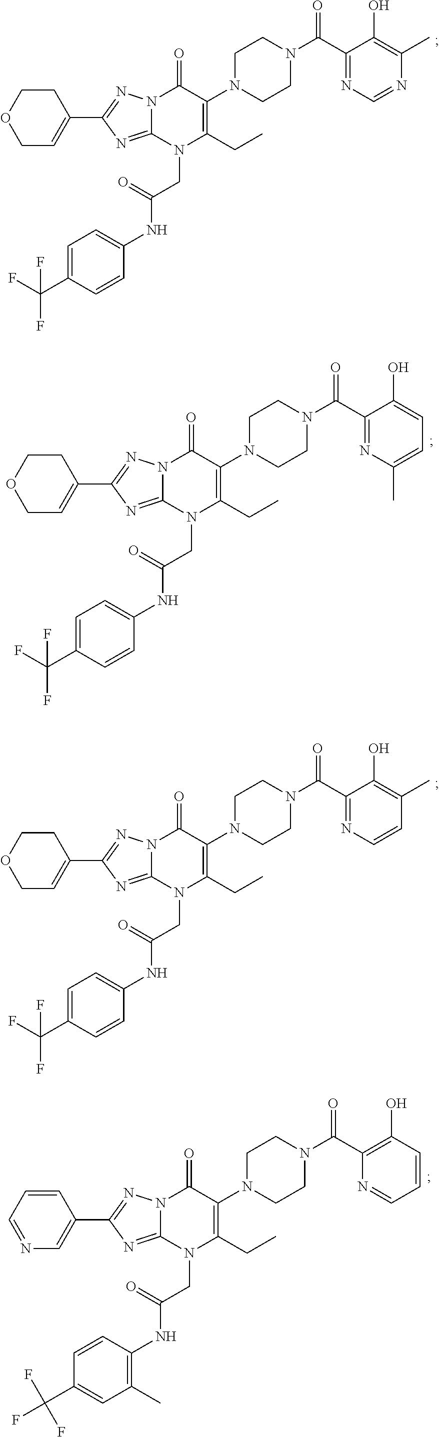 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1 ; CCc1c(N2CCN(C(=O)c3nc(C)ccc3O)CC2)c(=O)n2nc(C3=CCOCC3)nc2n1CC(=O)Nc1ccc(C(F)(F)F)cc1 ; CCc1c(N2CCN(C(=O)c3nccc(C)c3O)CC2)c(=O)n2nc(C3=CCOCC3)nc2n1CC(=O)Nc1ccc(C(F)(F)F)cc1 ; CCc1c(N2CCN(C(=O)c3ncccc3O)CC2)c(=O)n2nc(-c3cccnc3)nc2n1CC(=O)Nc1ccc(C(F)(F)F)cc1C]

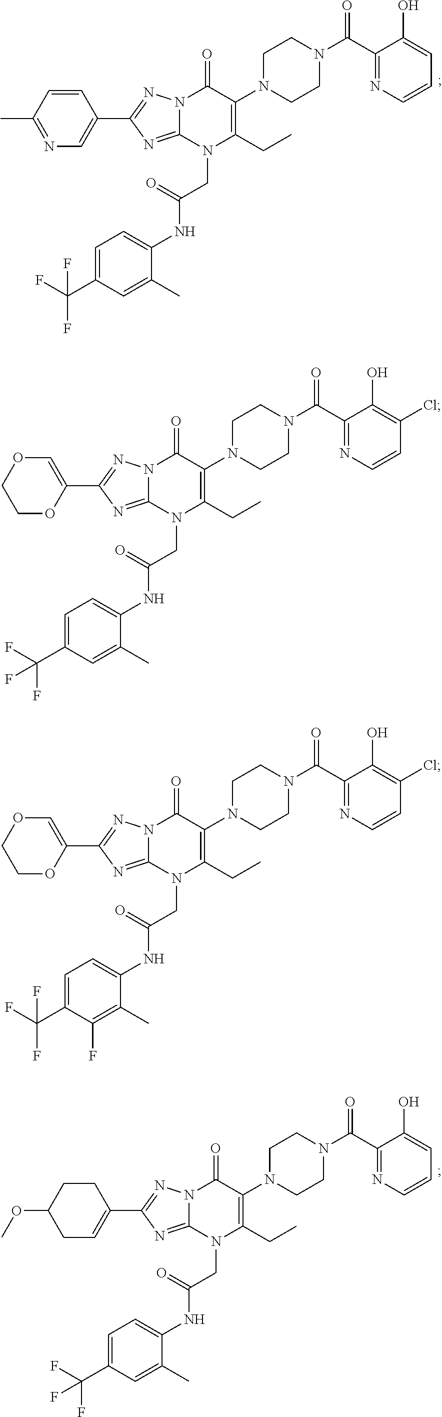 [SMILES: CCc1c(N2CCN(C(=O)c3ncccc3O)CC2)c(=O)n2nc(-c3ccc(C)nc3)nc2n1CC(=O)Nc1ccc(C(F)(F)F)cc1C ; CCc1c(N2CCN(C(=O)c3nccc(Cl)c3O)CC2)c(=O)n2nc(C3=COCCO3)nc2n1CC(=O)Nc1ccc(C(F)(F)F)cc1C ; CCc1c(N2CCN(C(=O)c3nccc(Cl)c3O)CC2)c(=O)n2nc(C3=COCCO3)nc2n1CC(=O)Nc1ccc(C(F)(F)F)c(F)c1C ; CCc1c(N2CCN(C(=O)c3ncccc3O)CC2)c(=O)n2nc(C3=CCC(OC)CC3)nc2n1CC(=O)Nc1ccc(C(F)(F)F)cc1C]

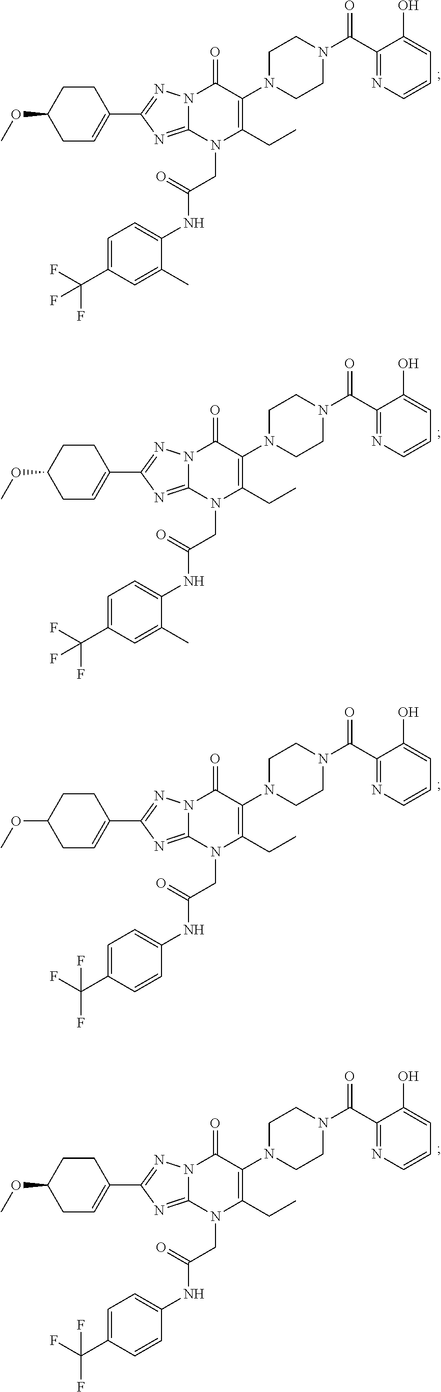 [SMILES: CCc1c(N2CCN(C(=O)c3ncccc3O)CC2)c(=O)n2nc(C3=CC[C@@H](OC)CC3)nc2n1CC(=O)Nc1ccc(C(F)(F)F)cc1C ; CCc1c(N2CCN(C(=O)c3ncccc3O)CC2)c(=O)n2nc(C3=CC[C@H](OC)CC3)nc2n1CC(=O)Nc1ccc(C(F)(F)F)cc1C ; CCc1c(N2CCN(C(=O)c3ncccc3O)CC2)c(=O)n2nc(C3=CCC(OC)CC3)nc2n1CC(=O)Nc1ccc(C(F)(F)F)cc1 ; CCc1c(N2CCN(C(=O)c3ncccc3O)CC2)c(=O)n2nc(C3=CC[C@@H](OC)CC3)nc2n1CC(=O)Nc1ccc(C(F)(F)F)cc1]

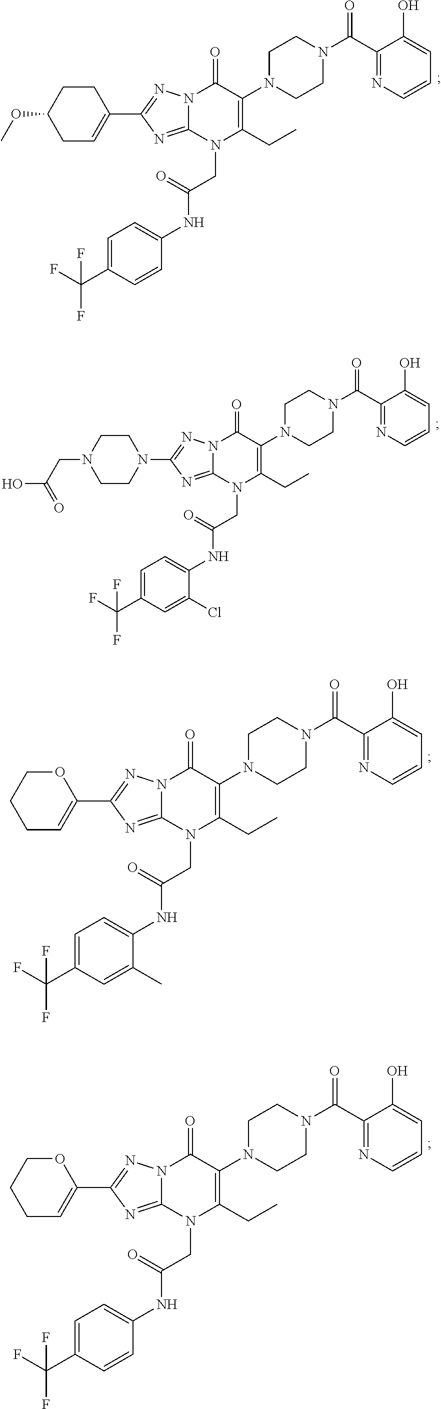 [SMILES: CCc1c(N2CCN(C(=O)c3ncccc3O)CC2)c(=O)n2nc(C3=CC[C@H](OC)CC3)nc2n1CC(=O)Nc1ccc(C(F)(F)F)cc1 ; CCc1c(N2CCN(C(=O)c3ncccc3O)CC2)c(=O)n2nc(N3CCN(CC(=O)O)CC3)nc2n1CC(=O)Nc1ccc(C(F)(F)F)cc1Cl ; CCc1c(N2CCN(C(=O)c3ncccc3O)CC2)c(=O)n2nc(C3=CCCCO3)nc2n1CC(=O)Nc1ccc(C(F)(F)F)cc1C ; CCc1c(N2CCN(C(=O)c3ncccc3O)CC2)c(=O)n2nc(C3=CCCCO3)nc2n1CC(=O)Nc1ccc(C(F)(F)F)cc1]

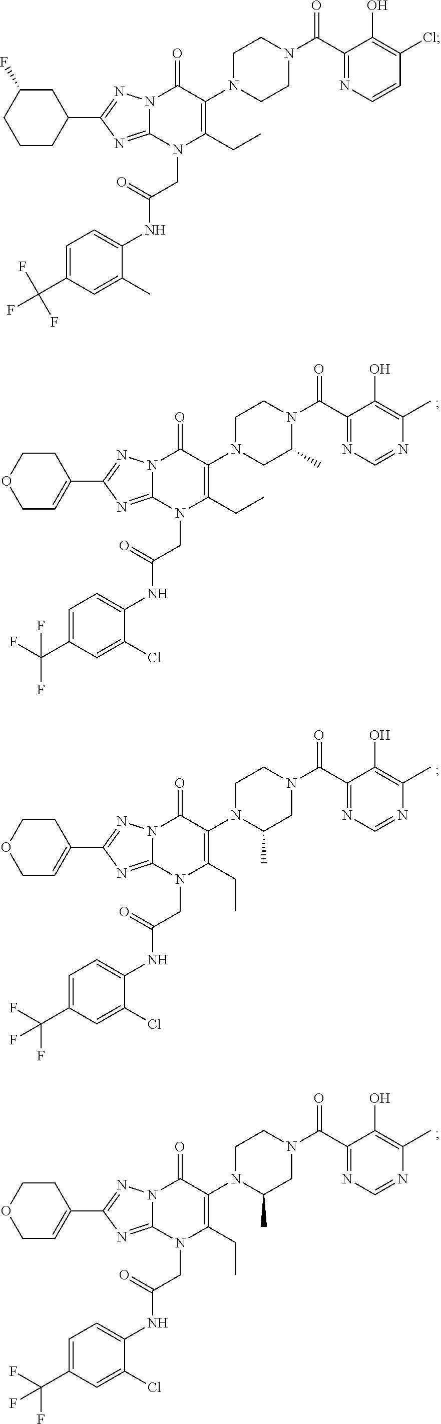 [SMILES: CCc1c(N2CCN(C(=O)c3nccc(Cl)c3O)CC2)c(=O)n2nc(C3CCC[C@H](F)C3)nc2n1CC(=O)Nc1ccc(C(F)(F)F)cc1C ; CCc1c(N2CCN(C(=O)c3ncnc(C)c3O)[C@H](C)C2)c(=O)n2nc(C3=CCOCC3)nc2n1CC(=O)Nc1ccc(C(F)(F)F)cc1Cl ; CCc1c(N2CCN(C(=O)c3ncnc(C)c3O)C[C@@H]2C)c(=O)n2nc(C3=CCOCC3)nc2n1CC(=O)Nc1ccc(C(F)(F)F)cc1Cl ; CCc1c(N2CCN(C(=O)c3ncnc(C)c3O)C[C@H]2C)c(=O)n2nc(C3=CCOCC3)nc2n1CC(=O)Nc1ccc(C(F)(F)F)cc1Cl]

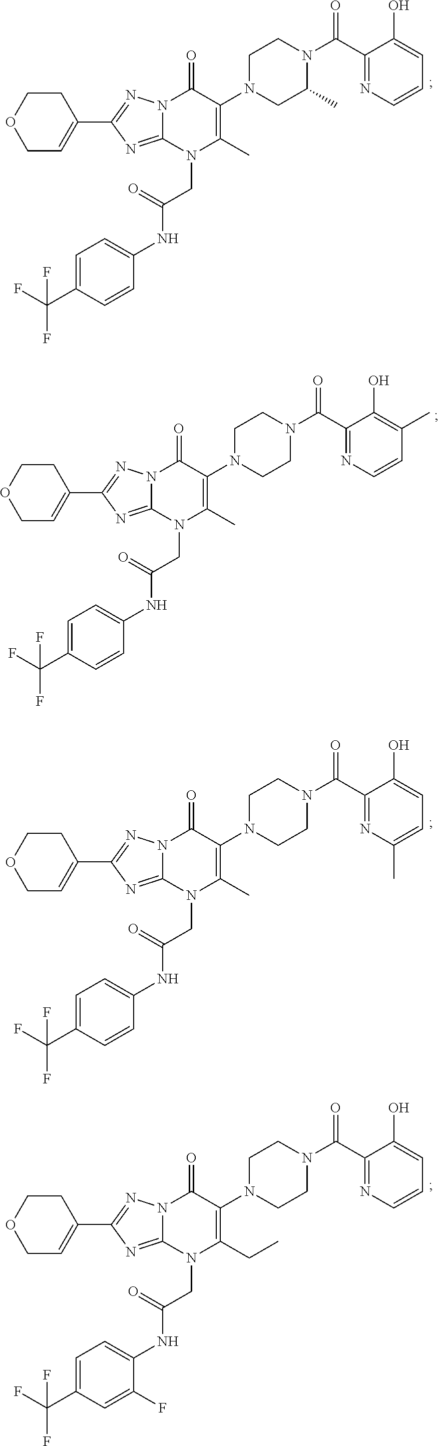 [SMILES: Cc1c(N2CCN(C(=O)c3ncccc3O)[C@H](C)C2)c(=O)n2nc(C3=CCOCC3)nc2n1CC(=O)Nc1ccc(C(F)(F)F)cc1 ; Cc1ccnc(C(=O)N2CCN(c3c(C)n(CC(=O)Nc4ccc(C(F)(F)F)cc4)c4nc(C5=CCOCC5)nn4c3=O)CC2)c1O ; Cc1ccc(O)c(C(=O)N2CCN(c3c(C)n(CC(=O)Nc4ccc(C(F)(F)F)cc4)c4nc(C5=CCOCC5)nn4c3=O)CC2)n1 ; CCc1c(N2CCN(C(=O)c3ncccc3O)CC2)c(=O)n2nc(C3=CCOCC3)nc2n1CC(=O)Nc1ccc(C(F)(F)F)cc1F]

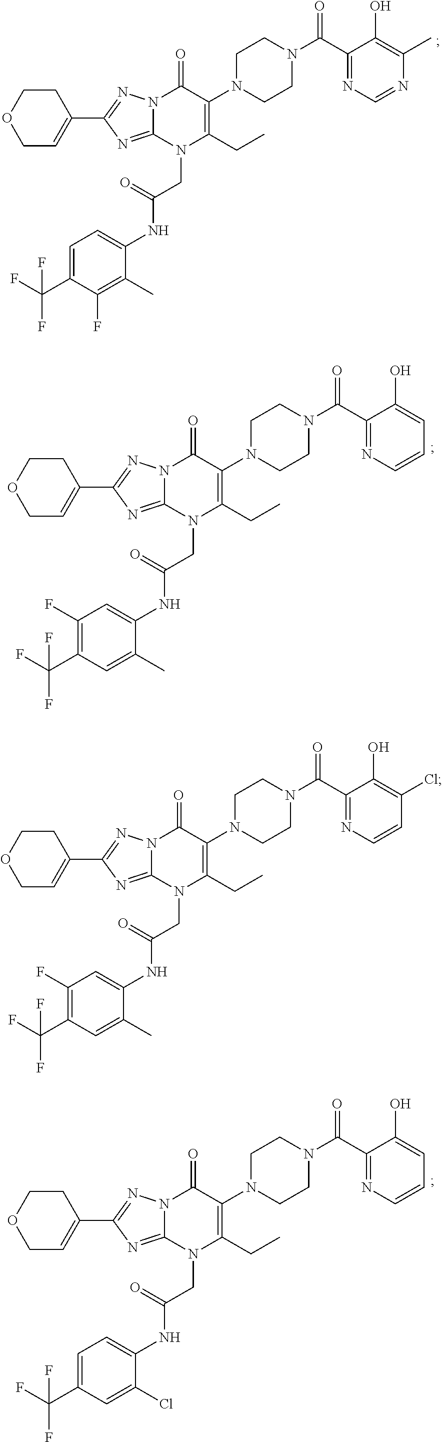 [SMILES: CCc1c(N2CCN(C(=O)c3ncnc(C)c3O)CC2)c(=O)n2nc(C3=CCOCC3)nc2n1CC(=O)Nc1ccc(C(F)(F)F)c(F)c1C ; CCc1c(N2CCN(C(=O)c3ncccc3O)CC2)c(=O)n2nc(C3=CCOCC3)nc2n1CC(=O)Nc1cc(F)c(C(F)(F)F)cc1C ; CCc1c(N2CCN(C(=O)c3nccc(Cl)c3O)CC2)c(=O)n2nc(C3=CCOCC3)nc2n1CC(=O)Nc1cc(F)c(C(F)(F)F)cc1C ; CCc1c(N2CCN(C(=O)c3ncccc3O)CC2)c(=O)n2nc(C3=CCOCC3)nc2n1CC(=O)Nc1ccc(C(F)(F)F)cc1Cl]

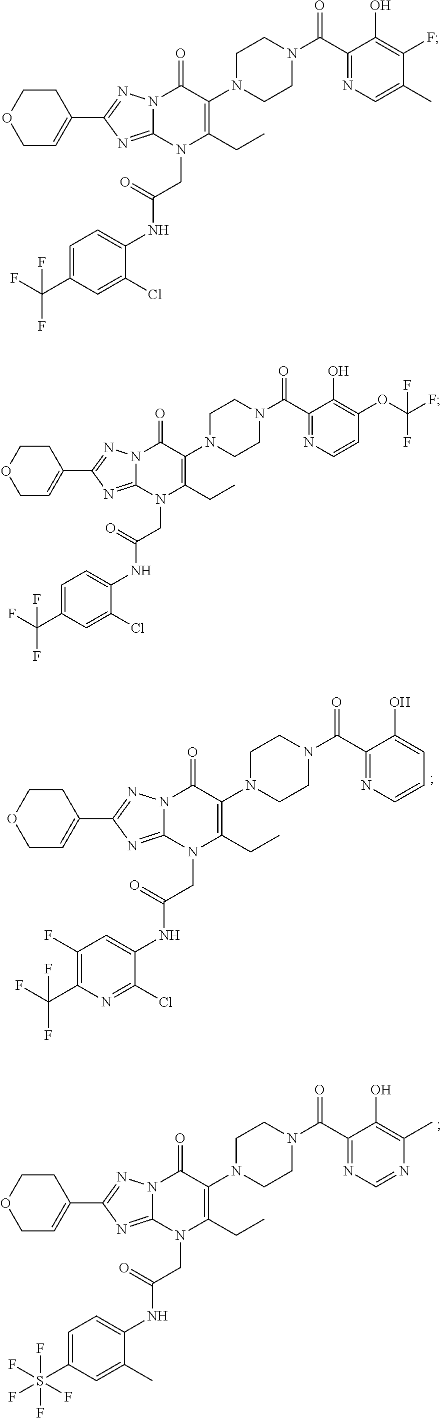 [SMILES: CCc1c(N2CCN(C(=O)c3ncc(C)c(F)c3O)CC2)c(=O)n2nc(C3=CCOCC3)nc2n1CC(=O)Nc1ccc(C(F)(F)F)cc1Cl ; CCc1c(N2CCN(C(=O)c3nccc(OC(F)(F)F)c3O)CC2)c(=O)n2nc(C3=CCOCC3)nc2n1CC(=O)Nc1ccc(C(F)(F)F)cc1Cl ; CCc1c(N2CCN(C(=O)c3ncccc3O)CC2)c(=O)n2nc(C3=CCOCC3)nc2n1CC(=O)Nc1cc(F)c(C(F)(F)F)nc1Cl ; CCc1c(N2CCN(C(=O)c3ncnc(C)c3O)CC2)c(=O)n2nc(C3=CCOCC3)nc2n1CC(=O)Nc1ccc(S(F)(F)(F)(F)F)cc1C]

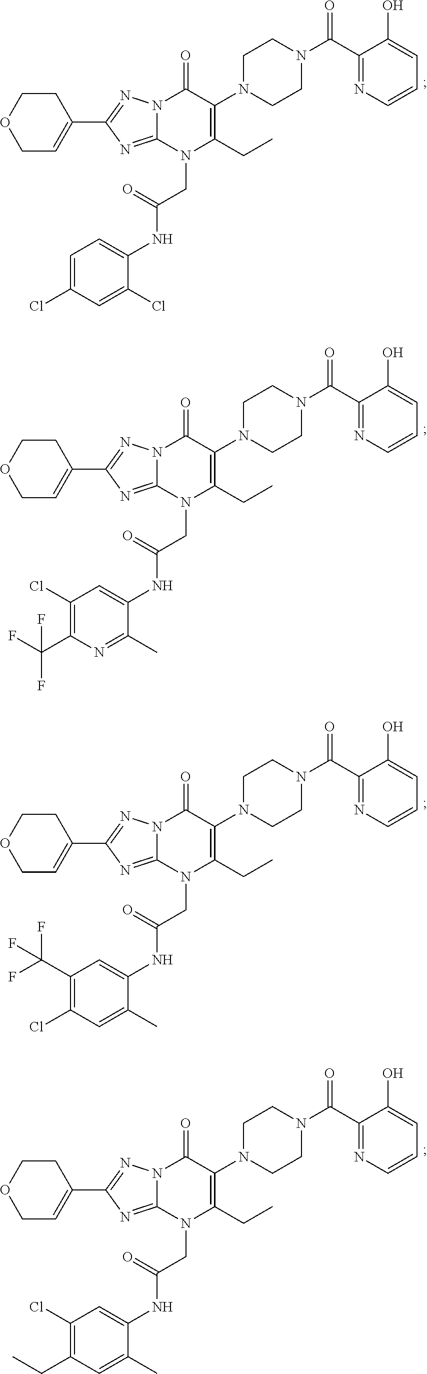 [SMILES: CCc1c(N2CCN(C(=O)c3ncccc3O)CC2)c(=O)n2nc(C3=CCOCC3)nc2n1CC(=O)Nc1ccc(Cl)cc1Cl ; CCc1c(N2CCN(C(=O)c3ncccc3O)CC2)c(=O)n2nc(C3=CCOCC3)nc2n1CC(=O)Nc1cc(Cl)c(C(F)(F)F)nc1C ; CCc1c(N2CCN(C(=O)c3ncccc3O)CC2)c(=O)n2nc(C3=CCOCC3)nc2n1CC(=O)Nc1cc(C(F)(F)F)c(Cl)cc1C ; CCc1cc(C)c(NC(=O)Cn2c(CC)c(N3CCN(C(=O)c4ncccc4O)CC3)c(=O)n3nc(C4=CCOCC4)nc23)cc1Cl]

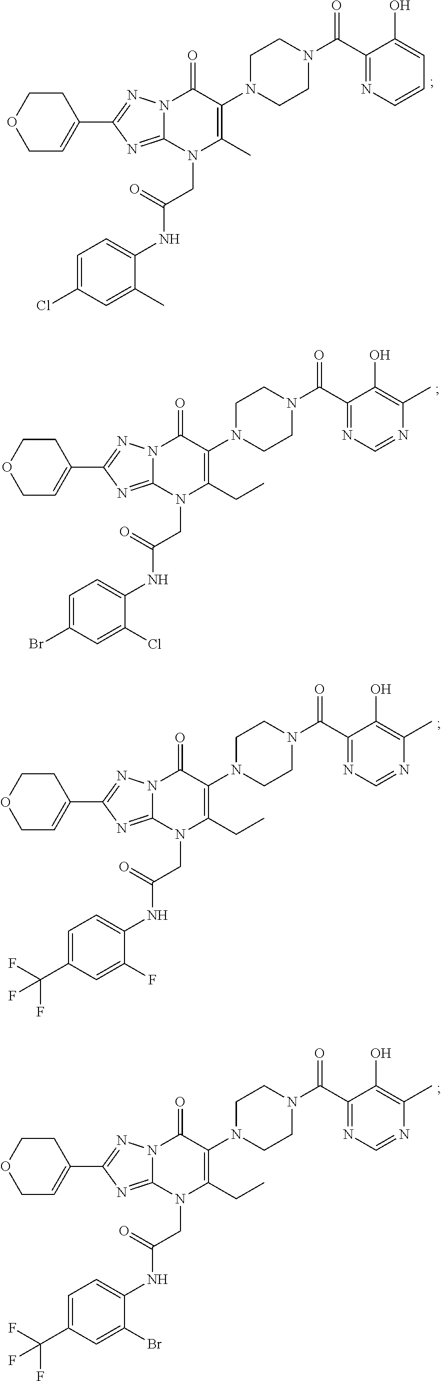 [SMILES: Cc1cc(Cl)ccc1NC(=O)Cn1c(C)c(N2CCN(C(=O)c3ncccc3O)CC2)c(=O)n2nc(C3=CCOCC3)nc12 ; CCc1c(N2CCN(C(=O)c3ncnc(C)c3O)CC2)c(=O)n2nc(C3=CCOCC3)nc2n1CC(=O)Nc1ccc(Br)cc1Cl ; CCc1c(N2CCN(C(=O)c3ncnc(C)c3O)CC2)c(=O)n2nc(C3=CCOCC3)nc2n1CC(=O)Nc1ccc(C(F)(F)F)cc1F ; CCc1c(N2CCN(C(=O)c3ncnc(C)c3O)CC2)c(=O)n2nc(C3=CCOCC3)nc2n1CC(=O)Nc1ccc(C(F)(F)F)cc1Br]

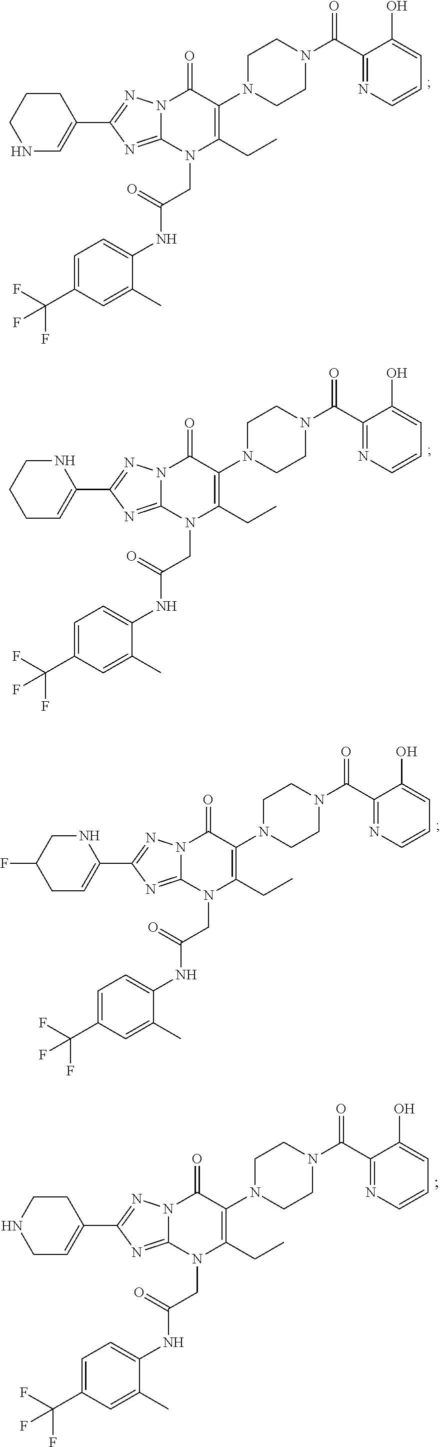 [SMILES: CCc1c(N2CCN(C(=O)c3ncccc3O)CC2)c(=O)n2nc(C3=CNCCC3)nc2n1CC(=O)Nc1ccc(C(F)(F)F)cc1C ; CCc1c(N2CCN(C(=O)c3ncccc3O)CC2)c(=O)n2nc(C3=CCCCN3)nc2n1CC(=O)Nc1ccc(C(F)(F)F)cc1C ; CCc1c(N2CCN(C(=O)c3ncccc3O)CC2)c(=O)n2nc(C3=CCC(F)CN3)nc2n1CC(=O)Nc1ccc(C(F)(F)F)cc1C ; CCc1c(N2CCN(C(=O)c3ncccc3O)CC2)c(=O)n2nc(C3=CCNCC3)nc2n1CC(=O)Nc1ccc(C(F)(F)F)cc1C]

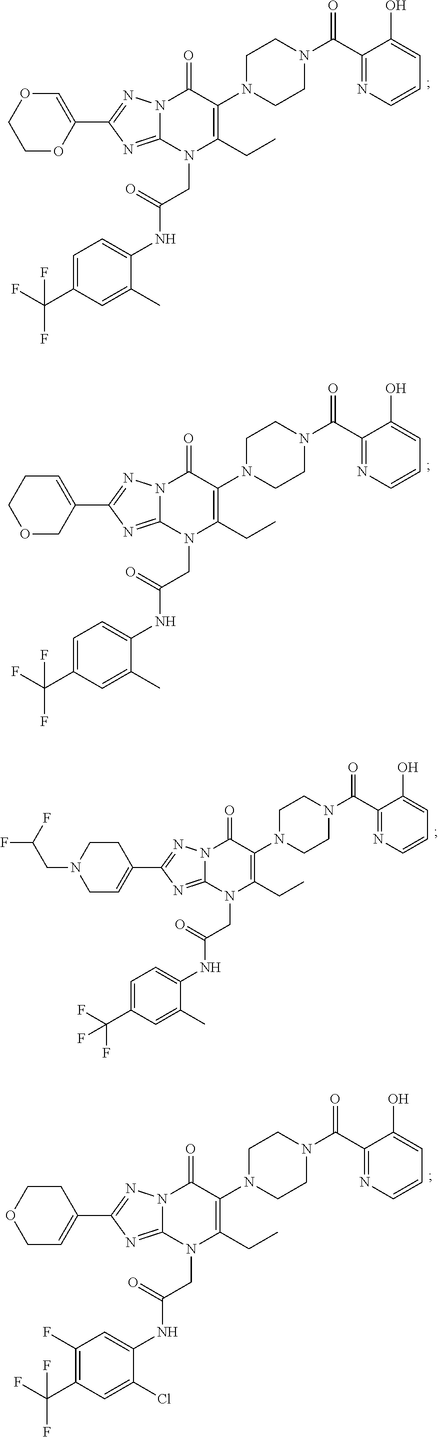 [SMILES: CCc1c(N2CCN(C(=O)c3ncccc3O)CC2)c(=O)n2nc(C3=COCCO3)nc2n1CC(=O)Nc1ccc(C(F)(F)F)cc1C ; CCc1c(N2CCN(C(=O)c3ncccc3O)CC2)c(=O)n2nc(C3=CCCOC3)nc2n1CC(=O)Nc1ccc(C(F)(F)F)cc1C ; CCc1c(N2CCN(C(=O)c3ncccc3O)CC2)c(=O)n2nc(C3=CCN(CC(F)F)CC3)nc2n1CC(=O)Nc1ccc(C(F)(F)F)cc1C ; CCc1c(N2CCN(C(=O)c3ncccc3O)CC2)c(=O)n2nc(C3=CCOCC3)nc2n1CC(=O)Nc1cc(F)c(C(F)(F)F)cc1Cl]

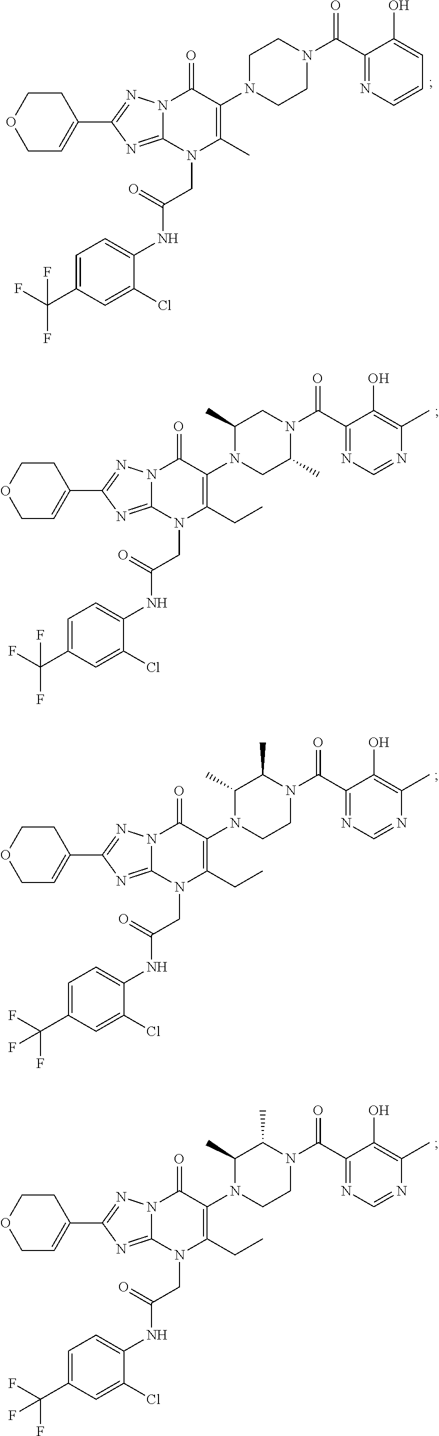 [SMILES: Cc1c(N2CCN(C(=O)c3ncccc3O)CC2)c(=O)n2nc(C3=CCOCC3)nc2n1CC(=O)Nc1ccc(C(F)(F)F)cc1Cl ; CCc1c(N2C[C@@H](C)N(C(=O)c3ncnc(C)c3O)C[C@@H]2C)c(=O)n2nc(C3=CCOCC3)nc2n1CC(=O)Nc1ccc(C(F)(F)F)cc1Cl ; CCc1c(N2CCN(C(=O)c3ncnc(C)c3O)[C@H](C)[C@H]2C)c(=O)n2nc(C3=CCOCC3)nc2n1CC(=O)Nc1ccc(C(F)(F)F)cc1Cl ; CCc1c(N2CCN(C(=O)c3ncnc(C)c3O)[C@@H](C)[C@@H]2C)c(=O)n2nc(C3=CCOCC3)nc2n1CC(=O)Nc1ccc(C(F)(F)F)cc1Cl]

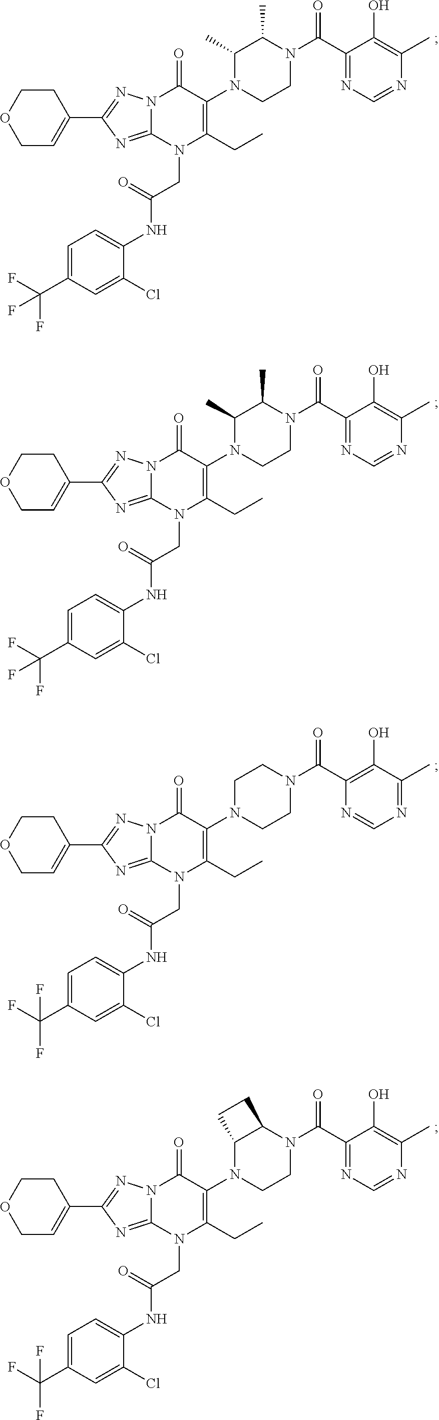 [SMILES: CCc1c(N2CCN(C(=O)c3ncnc(C)c3O)[C@@H](C)[C@H]2C)c(=O)n2nc(C3=CCOCC3)nc2n1CC(=O)Nc1ccc(C(F)(F)F)cc1Cl ; CCc1c(N2CCN(C(=O)c3ncnc(C)c3O)[C@H](C)[C@@H]2C)c(=O)n2nc(C3=CCOCC3)nc2n1CC(=O)Nc1ccc(C(F)(F)F)cc1Cl ; CCc1c(N2CCN(C(=O)c3ncnc(C)c3O)CC2)c(=O)n2nc(C3=CCOCC3)nc2n1CC(=O)Nc1ccc(C(F)(F)F)cc1Cl ; CCc1c(N2CCN(C(=O)c3ncnc(C)c3O)[C@@H]3CC[C@H]32)c(=O)n2nc(C3=CCOCC3)nc2n1CC(=O)Nc1ccc(C(F)(F)F)cc1Cl]

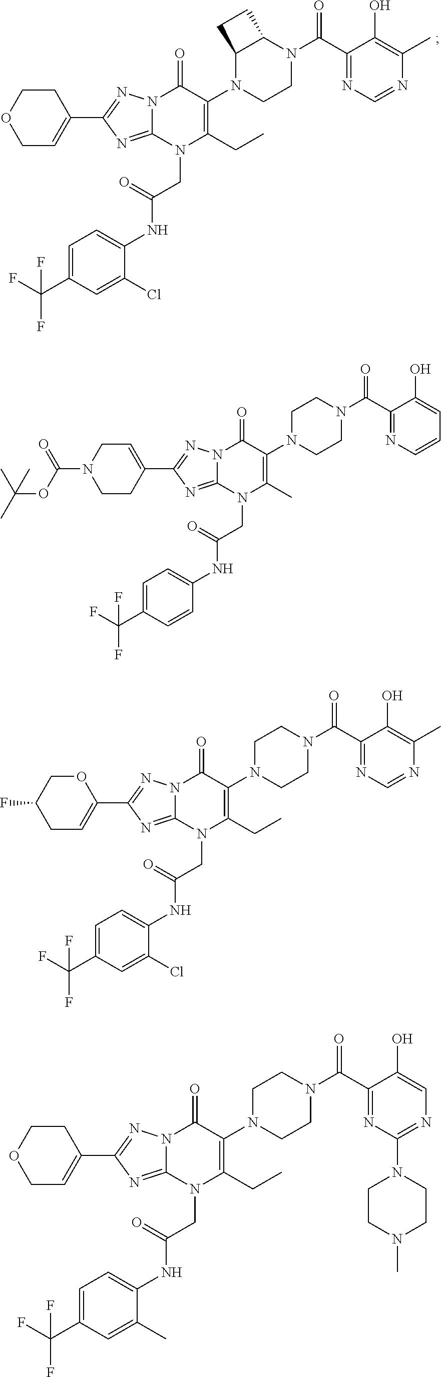

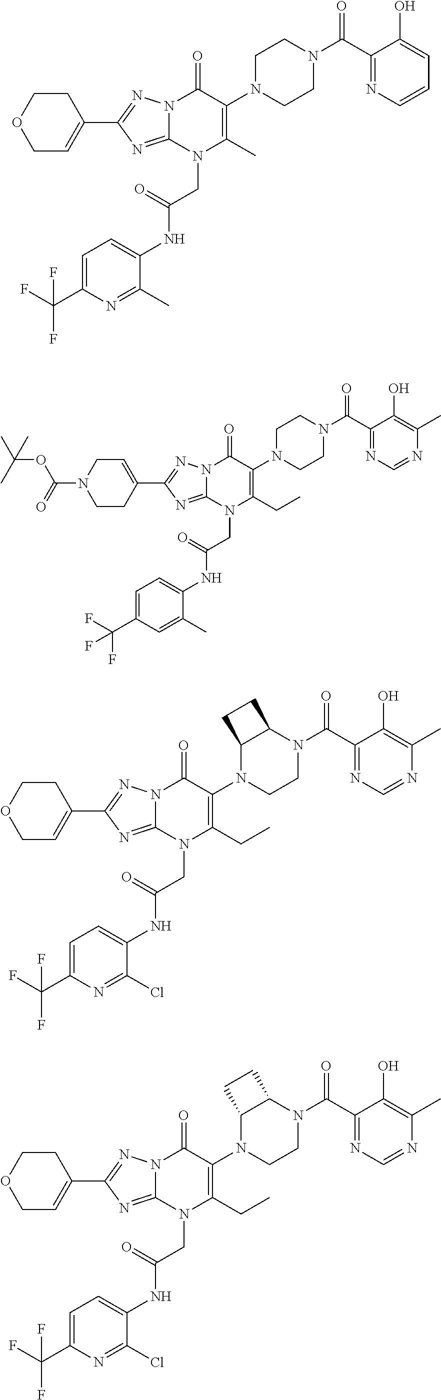 [SMILES: Cc1nc(C(F)(F)F)ccc1NC(=O)Cn1c(C)c(N2CCN(C(=O)c3ncccc3O)CC2)c(=O)n2nc(C3=CCOCC3)nc12 ; CCc1c(N2CCN(C(=O)c3ncnc(C)c3O)CC2)c(=O)n2nc(C3=CCN(C(=O)OC(C)(C)C)CC3)nc2n1CC(=O)Nc1ccc(C(F)(F)F)cc1C ; CCc1c(N2CCN(C(=O)c3ncnc(C)c3O)[C@@H]3CC[C@@H]32)c(=O)n2nc(C3=CCOCC3)nc2n1CC(=O)Nc1ccc(C(F)(F)F)nc1Cl ; CCc1c(N2CCN(C(=O)c3ncnc(C)c3O)[C@H]3CC[C@H]32)c(=O)n2nc(C3=CCOCC3)nc2n1CC(=O)Nc1ccc(C(F)(F)F)nc1Cl]

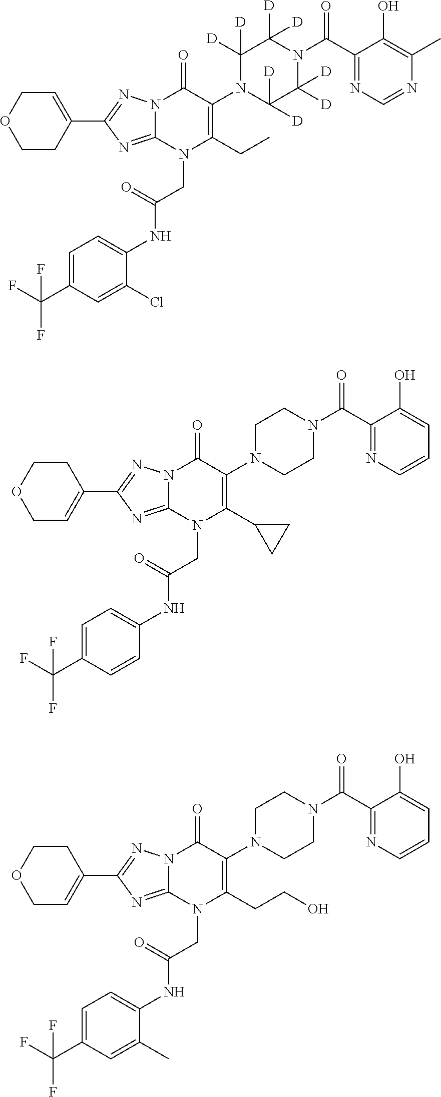

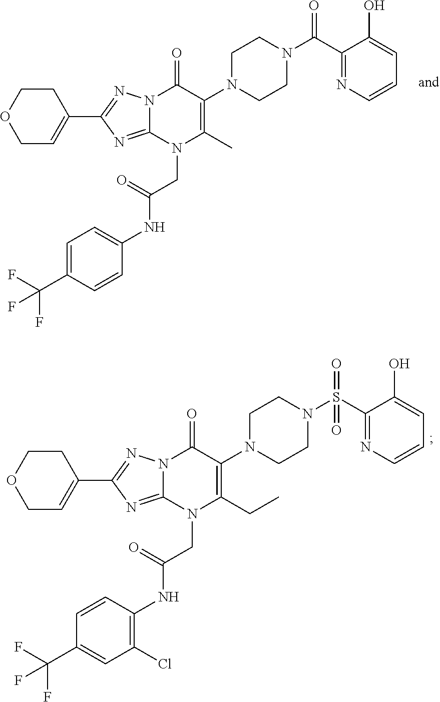 [SMILES: Cc1c(N2CCN(C(=O)c3ncccc3O)CC2)c(=O)n2nc(C3=CCOCC3)nc2n1CC(=O)Nc1ccc(C(F)(F)F)cc1 ; CCc1c(N2CCN(S(=O)(=O)c3ncccc3O)CC2)c(=O)n2nc(C3=CCOCC3)nc2n1CC(=O)Nc1ccc(C(F)(F)F)cc1Cl]

or a zwitterionic form or a salt thereof.

In an embodiment of any of the first to fourth aspects of the invention, the WRN inhibitor is selected from:

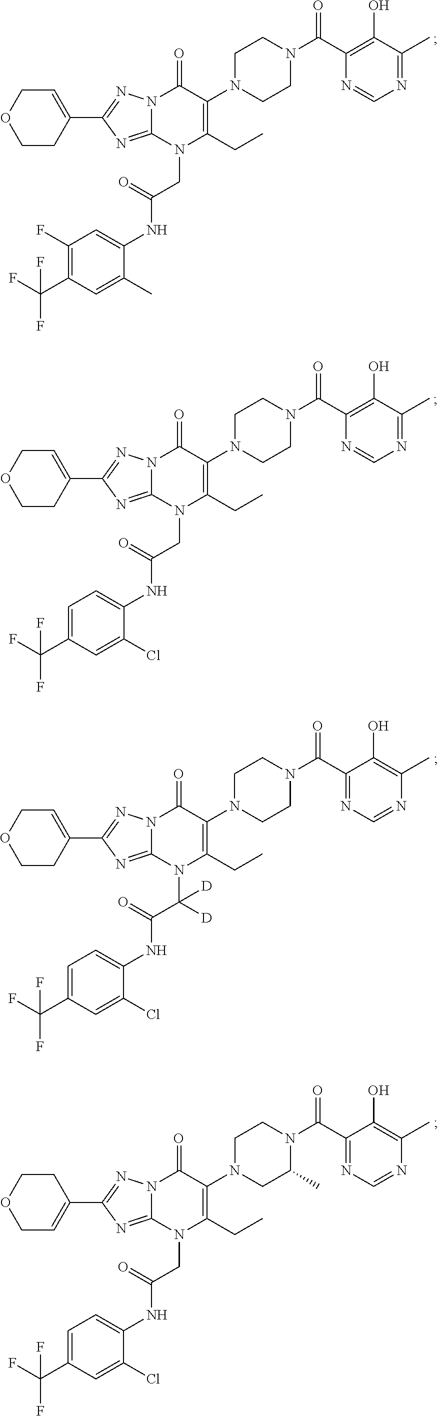 [SMILES: CCc1c(N2CCN(C(=O)c3ncnc(C)c3O)CC2)c(=O)n2nc(C3=CCOCC3)nc2n1CC(=O)Nc1cc(F)c(C(F)(F)F)cc1C ; CCc1c(N2CCN(C(=O)c3ncnc(C)c3O)CC2)c(=O)n2nc(C3=CCOCC3)nc2n1CC(=O)Nc1ccc(C(F)(F)F)cc1Cl ; [2H]C([2H])(C(=O)Nc1ccc(C(F)(F)F)cc1Cl)n1c(CC)c(N2CCN(C(=O)c3ncnc(C)c3O)CC2)c(=O)n2nc(C3=CCOCC3)nc12 ; CCc1c(N2CCN(C(=O)c3ncnc(C)c3O)[C@H](C)C2)c(=O)n2nc(C3=CCOCC3)nc2n1CC(=O)Nc1ccc(C(F)(F)F)cc1Cl]

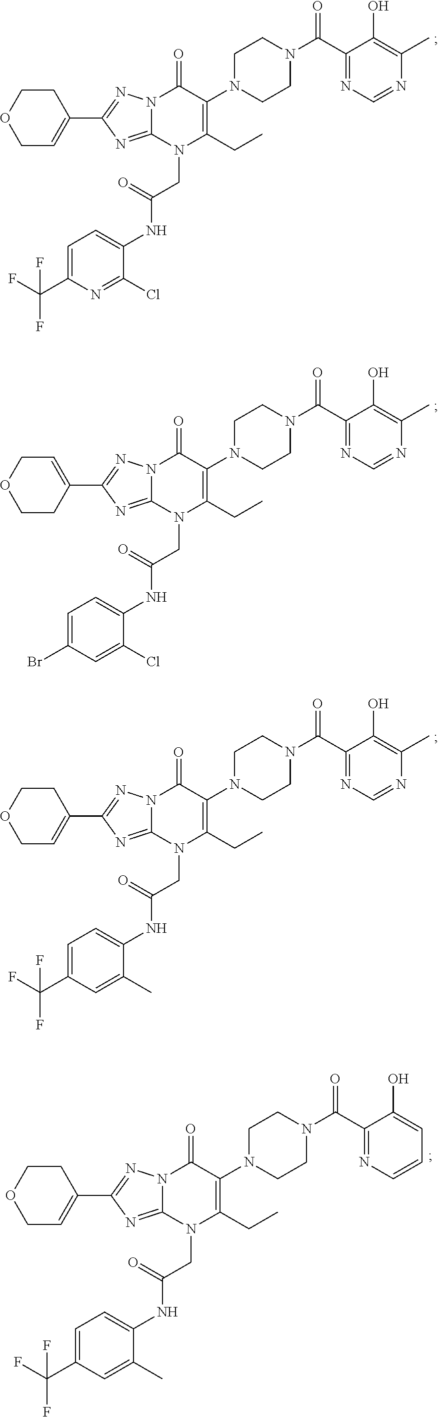 [SMILES: CCc1c(N2CCN(C(=O)c3ncnc(C)c3O)CC2)c(=O)n2nc(C3=CCOCC3)nc2n1CC(=O)Nc1ccc(C(F)(F)F)nc1Cl ; CCc1c(N2CCN(C(=O)c3ncnc(C)c3O)CC2)c(=O)n2nc(C3=CCOCC3)nc2n1CC(=O)Nc1ccc(Br)cc1Cl ; CCc1c(N2CCN(C(=O)c3ncnc(C)c3O)CC2)c(=O)n2nc(C3=CCOCC3)nc2n1CC(=O)Nc1ccc(C(F)(F)F)cc1C ; CCc1c(N2CCN(C(=O)c3ncccc3O)CC2)c(=O)n2nc(C3=CCOCC3)nc2n1CC(=O)Nc1ccc(C(F)(F)F)cc1C]

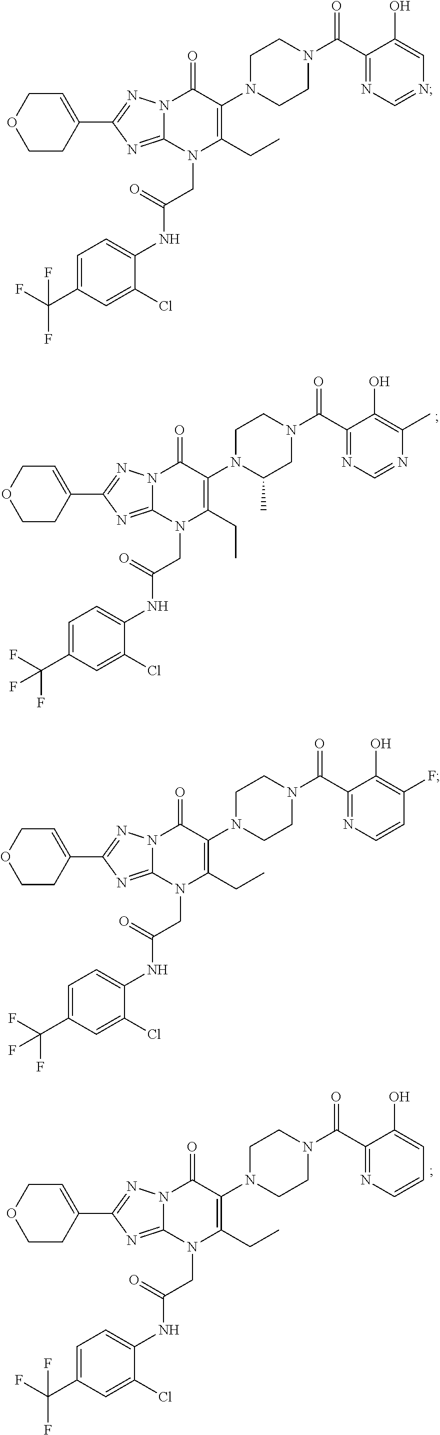 [SMILES: CCc1c(N2CCN(C(=O)c3ncncc3O)CC2)c(=O)n2nc(C3=CCOCC3)nc2n1CC(=O)Nc1ccc(C(F)(F)F)cc1Cl ; CCc1c(N2CCN(C(=O)c3ncnc(C)c3O)C[C@@H]2C)c(=O)n2nc(C3=CCOCC3)nc2n1CC(=O)Nc1ccc(C(F)(F)F)cc1Cl ; CCc1c(N2CCN(C(=O)c3nccc(F)c3O)CC2)c(=O)n2nc(C3=CCOCC3)nc2n1CC(=O)Nc1ccc(C(F)(F)F)cc1Cl ; CCc1c(N2CCN(C(=O)c3ncccc3O)CC2)c(=O)n2nc(C3=CCOCC3)nc2n1CC(=O)Nc1ccc(C(F)(F)F)cc1Cl]

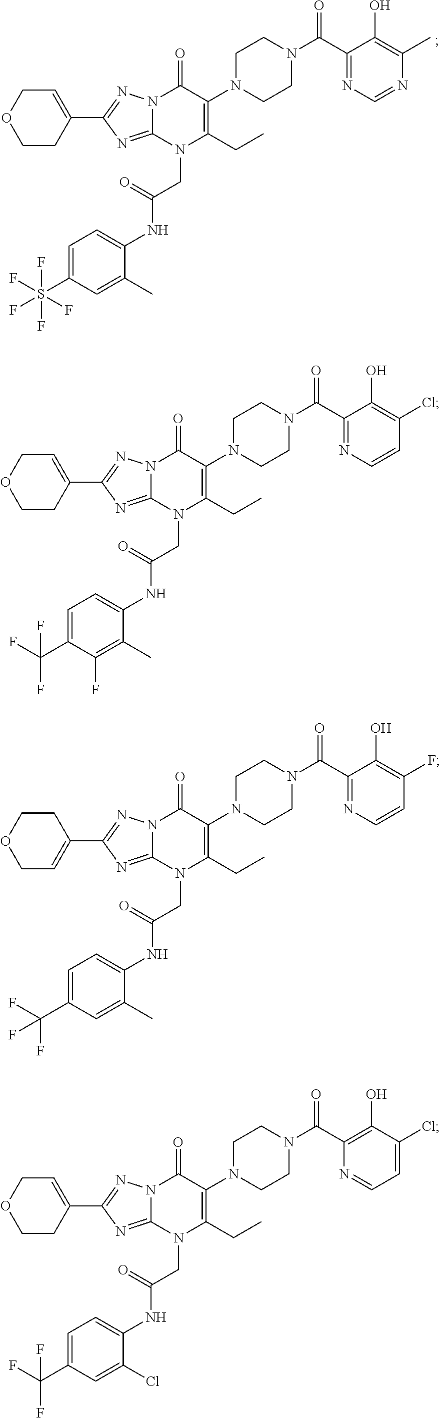 [SMILES: CCc1c(N2CCN(C(=O)c3ncnc(C)c3O)CC2)c(=O)n2nc(C3=CCOCC3)nc2n1CC(=O)Nc1ccc(S(F)(F)(F)(F)F)cc1C ; CCc1c(N2CCN(C(=O)c3nccc(Cl)c3O)CC2)c(=O)n2nc(C3=CCOCC3)nc2n1CC(=O)Nc1ccc(C(F)(F)F)c(F)c1C ; CCc1c(N2CCN(C(=O)c3nccc(F)c3O)CC2)c(=O)n2nc(C3=CCOCC3)nc2n1CC(=O)Nc1ccc(C(F)(F)F)cc1C ; CCc1c(N2CCN(C(=O)c3nccc(Cl)c3O)CC2)c(=O)n2nc(C3=CCOCC3)nc2n1CC(=O)Nc1ccc(C(F)(F)F)cc1Cl]

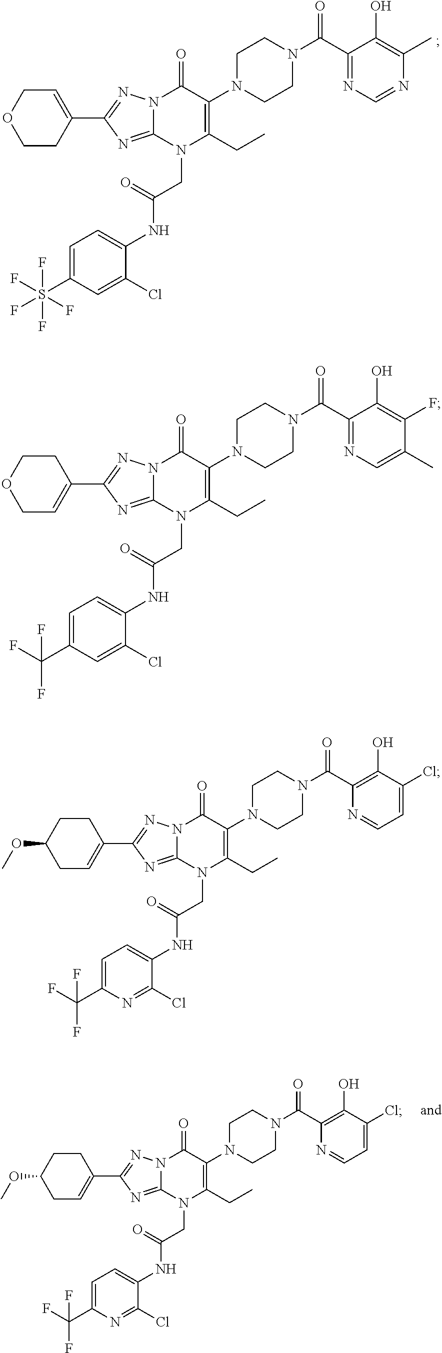

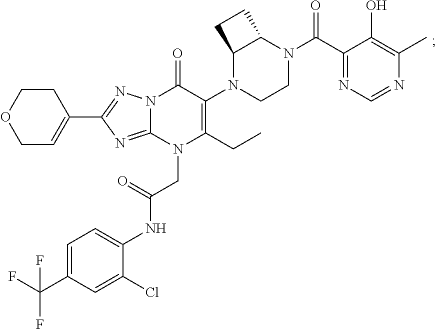 [SMILES: CCc1c(N2CCN(C(=O)c3ncnc(C)c3O)[C@H]3CC[C@@H]32)c(=O)n2nc(C3=CCOCC3)nc2n1CC(=O)Nc1ccc(C(F)(F)F)cc1Cl]

or a zwitterionic form or a salt thereof.

In an embodiment of any of the first to fourth aspects of the invention, the WRN inhibitor is selected from:

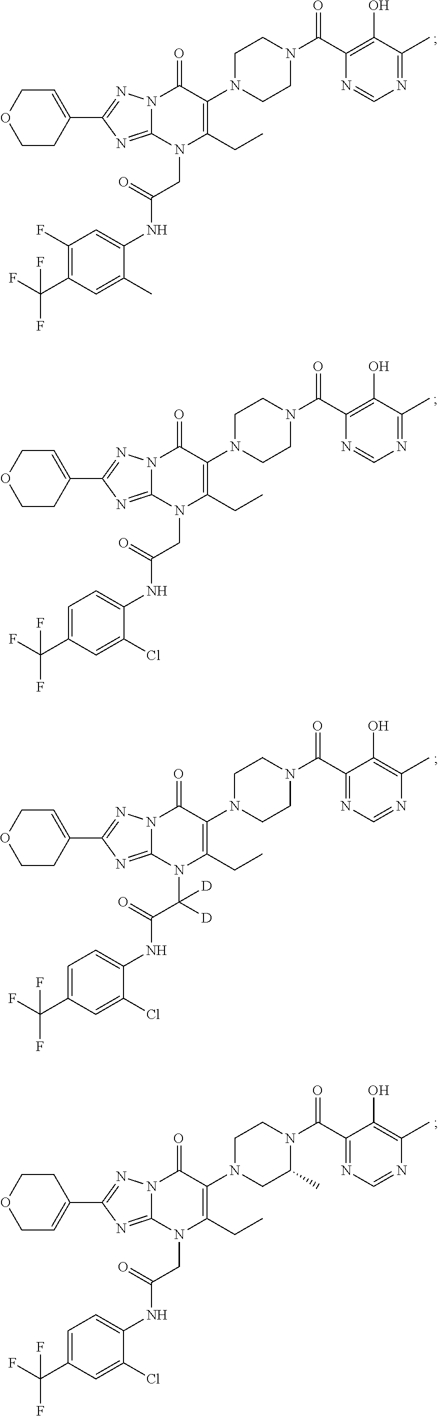 [SMILES: CCc1c(N2CCN(C(=O)c3ncnc(C)c3O)CC2)c(=O)n2nc(C3=CCOCC3)nc2n1CC(=O)Nc1cc(F)c(C(F)(F)F)cc1C ; CCc1c(N2CCN(C(=O)c3ncnc(C)c3O)CC2)c(=O)n2nc(C3=CCOCC3)nc2n1CC(=O)Nc1ccc(C(F)(F)F)cc1Cl ; [2H]C([2H])(C(=O)Nc1ccc(C(F)(F)F)cc1Cl)n1c(CC)c(N2CCN(C(=O)c3ncnc(C)c3O)CC2)c(=O)n2nc(C3=CCOCC3)nc12 ; CCc1c(N2CCN(C(=O)c3ncnc(C)c3O)[C@H](C)C2)c(=O)n2nc(C3=CCOCC3)nc2n1CC(=O)Nc1ccc(C(F)(F)F)cc1Cl]

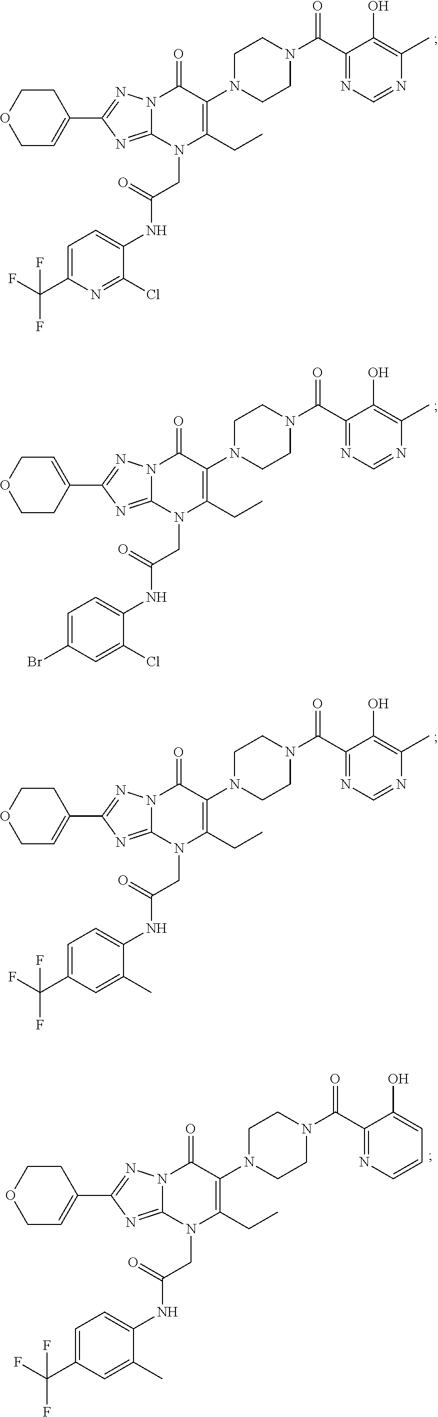 [SMILES: CCc1c(N2CCN(C(=O)c3ncnc(C)c3O)CC2)c(=O)n2nc(C3=CCOCC3)nc2n1CC(=O)Nc1ccc(C(F)(F)F)nc1Cl ; CCc1c(N2CCN(C(=O)c3ncnc(C)c3O)CC2)c(=O)n2nc(C3=CCOCC3)nc2n1CC(=O)Nc1ccc(Br)cc1Cl ; CCc1c(N2CCN(C(=O)c3ncnc(C)c3O)CC2)c(=O)n2nc(C3=CCOCC3)nc2n1CC(=O)Nc1ccc(C(F)(F)F)cc1C ; CCc1c(N2CCN(C(=O)c3ncccc3O)CC2)c(=O)n2nc(C3=CCOCC3)nc2n1CC(=O)Nc1ccc(C(F)(F)F)cc1C]

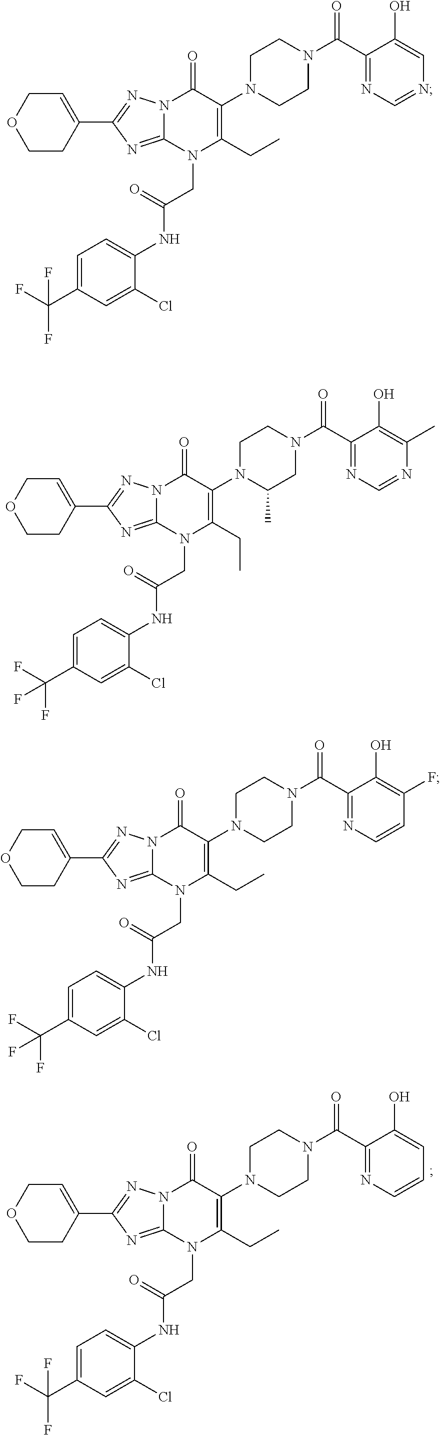 [SMILES: CCc1c(N2CCN(C(=O)c3ncncc3O)CC2)c(=O)n2nc(C3=CCOCC3)nc2n1CC(=O)Nc1ccc(C(F)(F)F)cc1Cl ; CCc1c(N2CCN(C(=O)c3ncnc(C)c3O)C[C@@H]2C)c(=O)n2nc(C3=CCOCC3)nc2n1CC(=O)Nc1ccc(C(F)(F)F)cc1Cl ; CCc1c(N2CCN(C(=O)c3nccc(F)c3O)CC2)c(=O)n2nc(C3=CCOCC3)nc2n1CC(=O)Nc1ccc(C(F)(F)F)cc1Cl ; CCc1c(N2CCN(C(=O)c3ncccc3O)CC2)c(=O)n2nc(C3=CCOCC3)nc2n1CC(=O)Nc1ccc(C(F)(F)F)cc1Cl]

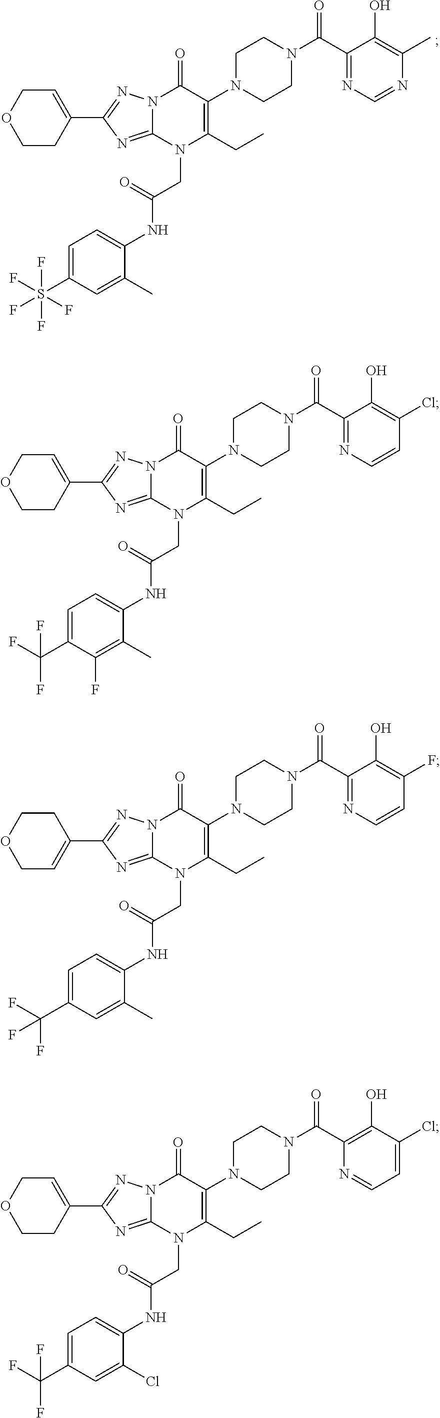 [SMILES: CCc1c(N2CCN(C(=O)c3ncnc(C)c3O)CC2)c(=O)n2nc(C3=CCOCC3)nc2n1CC(=O)Nc1ccc(S(F)(F)(F)(F)F)cc1C ; CCc1c(N2CCN(C(=O)c3nccc(Cl)c3O)CC2)c(=O)n2nc(C3=CCOCC3)nc2n1CC(=O)Nc1ccc(C(F)(F)F)c(F)c1C ; CCc1c(N2CCN(C(=O)c3nccc(F)c3O)CC2)c(=O)n2nc(C3=CCOCC3)nc2n1CC(=O)Nc1ccc(C(F)(F)F)cc1C ; CCc1c(N2CCN(C(=O)c3nccc(Cl)c3O)CC2)c(=O)n2nc(C3=CCOCC3)nc2n1CC(=O)Nc1ccc(C(F)(F)F)cc1Cl]

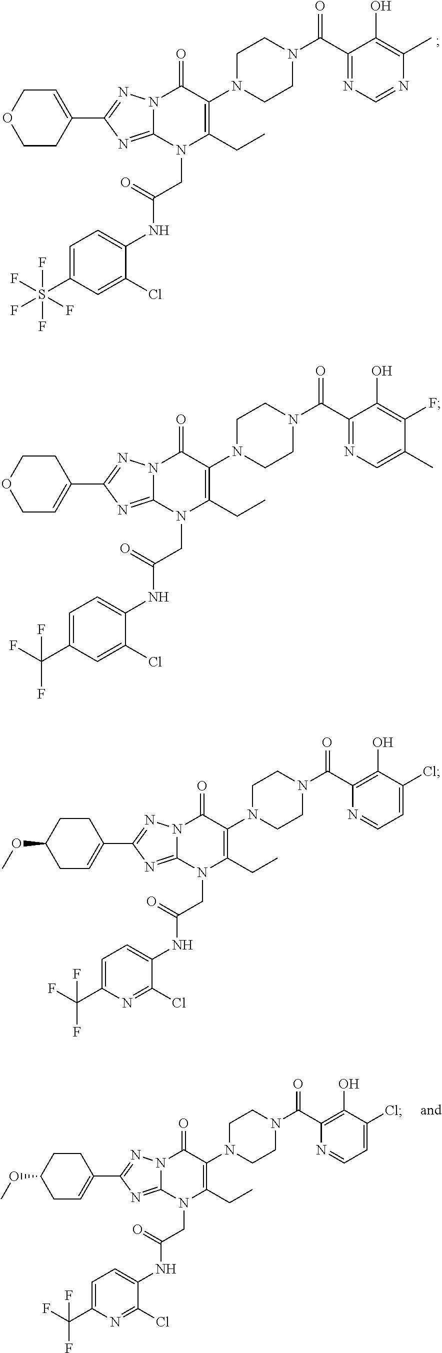

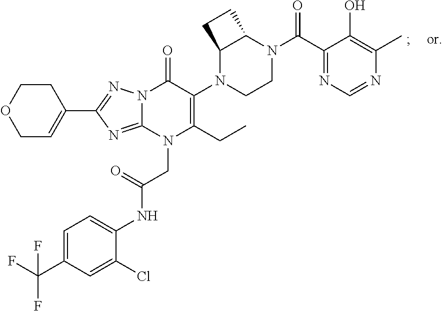 [SMILES: CCc1c(N2CCN(C(=O)c3ncnc(C)c3O)[C@H]3CC[C@@H]32)c(=O)n2nc(C3=CCOCC3)nc2n1CC(=O)Nc1ccc(C(F)(F)F)cc1Cl]

or a zwitterionic form or a salt thereof.

In an embodiment of any of the first to fourth aspects of the invention, the WRN inhibitor is

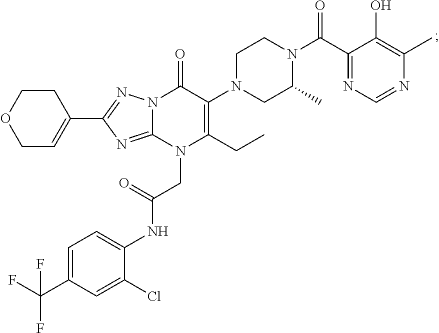 [SMILES: CCc1c(N2CCN(C(=O)c3ncnc(C)c3O)[C@H](C)C2)c(=O)n2nc(C3=CCOCC3)nc2n1CC(=O)Nc1ccc(C(F)(F)F)cc1Cl]

or a zwitterionic form or a salt thereof.

In an embodiment of any of the first to fourth aspects of the invention, the WRN inhibitor is

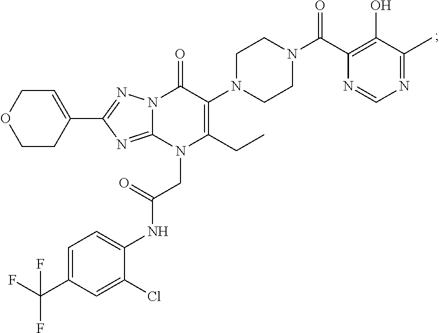 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl]

or a zwitterionic form or a salt thereof.

In an embodiment of any of the first to fourth aspects of the invention, the WRN inhibitor is

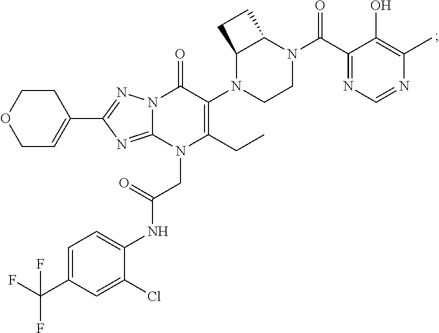 [SMILES: CCc1c(N2CCN(C(=O)c3ncnc(C)c3O)[C@H]3CC[C@@H]32)c(=O)n2nc(C3=CCOCC3)nc2n1CC(=O)Nc1ccc(C(F)(F)F)cc1Cl]

or a zwitterionic form or a salt thereof.

In an embodiment of any of the first to third aspects of the invention, the cancer is characterized as microsatellite instability-high (MSI-H) or mismatch repair deficient (dMMR).

In an embodiment of any of the first to fourth aspects of the invention, the (at least one) therapeutically active agent is a PD-1 inhibitor. In an embodiment, the PD-1 inhibitor is an anti-PD-1 antibody. In an embodiment, the PD-1 inhibitor is selected from PDR001 (Novartis), Nivolumab (Bristol-Myers Squibb), Pembrolizumab (Merck & Co), Pidilizumab (CureTech), MEDI0680 (Medimmune), Cemiplimab (REGN2810, Regeneron), Dostarlimab (TSR-042, Tesaro), PF-06801591 (Pfizer), Tislelizumab (BGB-A317, Beigene), BGB-108 (Beigene), INCSHR1210 (Incyte), Balstilimab (AGEN2035, Agenus), Sintilimab (InnoVent), Toripalimab (Shanghai Junshi Bioscience), Camrelizumab (Jiangsu Hengrui Medicine Co.), and AMP-224 (Amplimmune), in particular PDR001 or Tislelizumab. In an embodiment, the PD-1 inhibitor is Tislelizumab.

In an embodiment of any of the first to fourth aspects of the invention, the (at least one) therapeutically active agent is a chemotherapy agent. In an embodiment, the chemotherapy agent is selected from anastrozole (Arimidex®), vinblastine, vindesine, vinorelbine, vincristine, bicalutamide (Casodex®), bleomycin (e.g. bleomycin sulfate) (Blenoxane®), busulfan (Myleran®), busulfan injection (Busulfex®), calactin, capecitabine (Xeloda®), N4-pentoxycarbonyl-5-deoxy-5-fluorocytidine, carboplatin (Paraplatin®), carmustine (BICNUR), lomustine (CCNUR), chlorambucil (Leukeran®), bendamustine (Treanda®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegen), daunorubicin hydrochloride (Cerubidine®), daunorubicin citrate liposome injection (DaunoXome®), dexamethasone, docetaxel (Taxotere®), doxorubicin (e.g. doxorubicin hydrochloride) (Adriamycin®, Rubex®), etoposide (Vepesid®), fludarabine phosphate (Fludara®), 5-fluorouracil (Adrucil®, Efudex®), flutamide (Eulexin®), tezacitabine, gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), a mitomycin (e.g. mitomycin A, mitomycin B or mitomycin C, particularly mitomycin C), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), camptothecin, teniposide (Vumon®), 6-thioguanine, thiotepa, tirapazamine (Tirazone®), topotecan hydrochloride for injection (Hycamptin®), vinblastine (Velban®), vincristine (Oncovin®), oxaliplatin (Eloxatin®), epirubicin (Ellence®, Pharmorubicin®) temozolomide (Temodar®), tegafur, and vinorelbine (Navelbine®), in particular irinotecan. In an embodiment, the chemotherapy agent is selected from gemcitabine, camptothecin, irinotecan (Camptosar®), docetaxel (Taxotere®), doxorubicin (e.g. doxorubicin hydrochloride) (Adriamycin®, Rubex®), 5-fluorouracil (Adrucil®, Efudex®), capecitabine (Xeloda®), etoposide (Vepesid®), epirubicin (Ellence®, Pharmorubicin®), oxaliplatin (Eloxatin®), mitomycin (e.g. mitomycin A, mitomycin B or mitomycin C, particularly mitomycin C), cisplatin (Platinol®), carboplatin (Paraplatin®) and paclitaxel (Taxol®).

In an embodiment of any of the first to fourth aspects of the invention, the (at least one) therapeutically active agent is a WEE1 inhibitor. In an embodiment, the WEE1 inhibitor is selected from Adavosertib (also known as AZD1775 and MK-1775) and PDO166285. In an embodiment, the WEE1 inhibitor is Adavosertib (also known as AZD1775 and MK-1775).

In an embodiment of any of the first to fourth aspects of the invention, the (at least one) therapeutically active agent is an ATR inhibitor. In an embodiment, the ATR inhibitor is selected from RP-3500, ceralasertib (also known as AZD6738), berzosertib, ART-0380, gartisertib (also known as M4344), and elimusertib (also known as BAY-1895344). In an embodiment, the ATR inhibitor is elimusertib (BAY-1895344).

In an embodiment of any of the first to fourth aspects of the invention, the (at least one) therapeutically active agent is a DNA-PK inhibitor. In an embodiment, the DNA-PK inhibitor is selected from AZD-7648, NU7441 (also known as KU-57788), Omipalisib, BAY8400 and M3814. In an embodiment, the DNA-PK inhibitor is AZD-7648 or NU7441 (KU-57788), particularly AZD-7648.

In an embodiment of any of the first to fourth aspects of the invention, the (at least one) therapeutically active agent is a ionising radiation based therapy selected from i) external beam radiation, ii) brachytherapy and iii) a radiopharmaceutical. In an embodiment, the ionising radiation is external beam radiation. In an embodiment, the ionising radiation is a radiopharmaceutical. In an embodiment, the radiopharmaceutical is a radioligand agent. In an embodiment, the radioligand agent is selected from ¹⁷⁷Lu-PSMA-617, ¹⁷⁷Lu-PSMA-R2, ¹⁷⁷Lu-NeoB, ¹⁷⁷Lu-FAP-2286.

In an embodiment of any of the first to fourth aspects of the invention, the (at least one) therapeutically active agent is a MEK inhibitor. In an embodiment, the MEK inhibitor is selected from the group consisting of refametinib, pimasertib, selumetinib, trametinib, binimetinib and cobimetinib, or a pharmaceutically acceptable salt thereof. In an embodiment, the MEK inhibitor is trametinib.

In an embodiment of any of the first to fourth aspects of the invention, the (at least one) therapeutically active agent is an MDM2 inhibitor. In an embodiment, the MDM2 inhibitor is selected from the group consisting of nutlin-3a, idasanutlin (also known as RG7388), RG7112, KRT-232 (also known as AMG-232), APG-115, RAIN-32 (also known as DS-3032 and milademetan), BI-907828 and HDM201 (also known as siremadlin), or a pharmaceutically acceptable salt thereof. In an embodiment, the MDM2 inhibitor is HDM201.

In an embodiment of any of the first to fourth aspects of the invention, the (at least one) therapeutically active agent is a G4-quadruplex stabilizer. In an embodiment, the G4-quadruplex stabilizer is pyridostatin.

In an embodiment of any of the first to fourth aspects of the invention, the (at least one) therapeutically active agent is an ATM inhibitor. In an embodiment, the ATM inhibitor is selected from KU-55933, KU-60019, KU-59403, M3541, CP-466722, AZ31, AZ32, AZD0156 and AZD1390. In an embodiment, the ATM inhibitor is KU-60019.

In an embodiment of any of the first to fourth aspects of the invention, the (at least one) therapeutically active agent is a PARP inhibitor. In an embodiment, the PARP inhibitor is selected from olaparib, NMS293, niraparib, prexasertib, veliparib, rucaparib, talazoparib, AZD-5305 and KU0058948. In an embodiment, the PARP inhibitor is olaparib.

In an embodiment of any of the first to fourth aspects of the invention, the (at least one) therapeutically active agent is a chemotherapy agent, and the chemotherapy agent is a topoisomerase inhibitor. In an embodiment, the topoisomerase inhibitor is selected from QAP1, irinotecan, topotecan, camptothecin and etoposide. In an embodiment, the topoisomerase inhibitor is selected from QAP1, etoposide and irinotecan.

In an embodiment of any of the first to fourth aspects of the invention, the (at least one) therapeutically active agent is a CHK1 or CHK2 inhibitor. In an embodiment, the CHK1 or CHK2 inhibitor is selected from GDC-0575, Prexasertib (also known as LY2606368), SCH900776 (also known as MK-8776), SRA737, PF477736, LY2606368 and AZD7762.

In an embodiment of any of the first to fourth aspects of the invention, the cancer is selected from colorectal cancer (CRC), gastric cancer, prostate cancer, endometrial cancer, adrenocortical cancer, uterine cancer and cervical cancer, for example a cancer selected from uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma. In an embodiment, the cancer is colorectal cancer (CRC) or gastric cancer, particularly colorectal cancer (CRC).

Therefore, according to a fifth aspect of the invention, there is hereby provided a method of treating colorectal cancer, the method comprising administering to a subject a therapeutically effective amount of a WRN inhibitor in combination with a therapeutically effective amount of a G4-quadruplex stabilizer. In an embodiment, the G4-quaduplex stabiliser is pyridostatin.

Therefore, according to a sixth aspect of the invention, there is hereby provided a method of treating colorectal cancer, the method comprising administering to a subject a therapeutically effective amount of a WRN inhibitor in combination with a therapeutically effective amount of a MEK inhibitor. In an embodiment, the MEK inhibitor is trametinib.

Therefore, according to a seventh aspect of the invention, there is hereby provided a method of treating colorectal cancer, the method comprising administering to a subject a therapeutically effective amount of a WRN inhibitor in combination with a therapeutically effective amount of a topoisomerase inhibitor. In an embodiment, the topoisomerase inhibitor is QAP1, etoposide, irinotecan or camptothecin.

Therefore, according to an eighth aspect of the invention, there is hereby provided a method of treating colorectal or gastric cancer, the method comprising administering to a subject a therapeutically effective amount of a WRN inhibitor in combination with a therapeutically effective amount of an ATR inhibitor. In an embodiment, the ATR inhibitor is elimusertib (BAY-1895344).

Therefore, according to a ninth aspect of the invention, there is hereby provided a method of treating gastric cancer, the method comprising administering to a subject a therapeutically effective amount of a WRN inhibitor in combination with a therapeutically effective amount of ionising radiation based therapy selected from i) external beam radiation, ii) brachytherapy and ii) a radiopharmaceutical. In an embodiment, the ionising radiation is external beam radiation. In an embodiment, the ionising radiation is a radiopharmaceutical. In an embodiment, the radiopharmaceutical is a radioligand agent. In an embodiment, the radioligand agent is selected from ¹⁷⁷Lu-PSMA-617, ¹⁷⁷Lu-PSMA-R2, ¹⁷⁷Lu-NeoB, ¹⁷⁷Lu-FAP-2286.

Therefore, according to a tenth aspect of the invention, there is hereby provided a method of treating colorectal cancer, the method comprising administering to a subject a therapeutically effective amount of a WRN inhibitor in combination with a therapeutically effective amount of a chemotherapy agent. In an embodiment, the chemotherapy agent is selected from 5-fluorouracil, cisplatin, bleomycin, docetaxel, epirubicin, etoposide, camptothecin, mitomycin, oxaliplatin, mitomycin (e.g. mitomycin C) and gemcitabine.

Therefore, according to an eleventh aspect of the invention, there is hereby provided a method of treating colorectal cancer, the method comprising administering to a subject a therapeutically effective amount of a WRN inhibitor in combination with a therapeutically effective amount of an MDM2 inhibitor. In an embodiment, the MDM2 inhibitor is HDM201.

Therefore, according to a twelfth aspect of the invention, there is hereby provided a method of treating colorectal cancer, the method comprising administering to a subject a therapeutically effective amount of a WRN inhibitor in combination with a therapeutically effective amount of an ATM inhibitor. In an embodiment, the ATM inhibitor is KU-60019.

Therefore, according to a thirteenth aspect of the invention, there is hereby provided a method of treating colorectal cancer, the method comprising administering to a subject a therapeutically effective amount of a WRN inhibitor in combination with a therapeutically effective amount of a DNA-PK inhibitor. In an embodiment, the DNA-PK inhibitor is AZD-7648 or NU7441 (KU-57788).

Therefore, according to a fourteenth aspect of the invention, there is hereby provided a method of treating colorectal cancer, the method comprising administering to a subject a therapeutically effective amount of a WRN inhibitor in combination with a therapeutically effective amount of a WEE1 inhibitor. In an embodiment, the WEE1 inhibitor is Adavosertib.

Therefore, according to a fifteenth aspect of the invention, there is hereby provided a method of treating colorectal cancer, the method comprising administering to a subject a therapeutically effective amount of a WRN inhibitor in combination with a therapeutically effective amount of a CHK1 or CHK2 inhibitor. In an embodiment, the CHK1 or CHK2 inhibitor is AZD7762 or SCH900776 (also known as MK-8776).

In an embodiment of any of the fifth to fifteenth aspects of the invention, the colorectal cancer or gastric cancer is characterized as microsatellite instability-high (MSI-H) or mismatch repair deficient (dMMR). In an embodiment, the colorectal cancer or gastric cancer is characterized as microsatellite instability-high (MSI-H).

In an embodiment of the eighth or the ninth aspect of the invention, the gastric cancer is gastric adenocarcinoma.

In an embodiment of any of the aspects of the invention, the WRN inhibitor is selected from

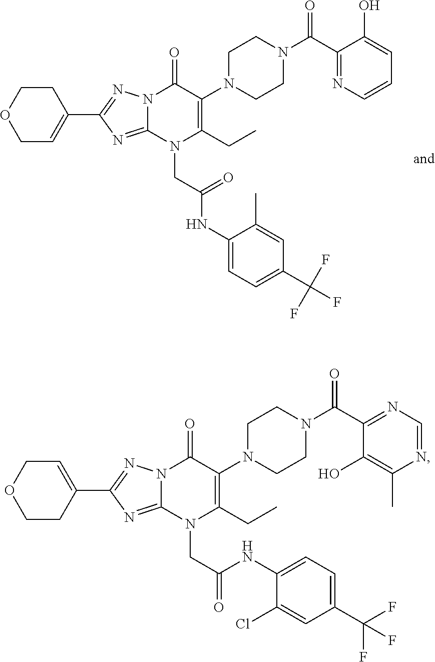 [SMILES: CCc1c(N2CCN(C(=O)c3ncccc3O)CC2)c(=O)n2nc(C3=CCOCC3)nc2n1CC(=O)Nc1ccc(C(F)(F)F)cc1C ; CCc1c(N2CCN(C(=O)c3ncnc(C)c3O)CC2)c(=O)n2nc(C3=CCOCC3)nc2n1CC(=O)Nc1ccc(C(F)(F)F)cc1Cl]

or a zwitterionic form or a pharmaceutically acceptable salt thereof.

In an embodiment, the WRN inhibitor is

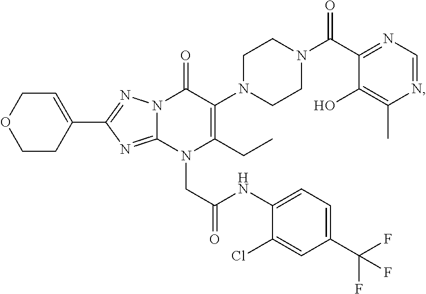 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl]

or a zwitterionic form or a pharmaceutically acceptable salt thereof.

Therefore, according to a sixteenth aspect of the invention, there is hereby provided a combination comprising i) a WRN inhibitor and ii) temozolomide, and optionally iii) irinotecan.

Therefore, according to a seventeenth aspect of the invention, there is hereby provided temozolomide for use in the treatment of cancer, wherein the treatment further comprises administration of:
a WRN inhibitor, or
a WRN inhibitor in combination with irinotecan.

Therefore, according to an eighteenth aspect of the invention, there is hereby provided a WRN inhibitor for use in the treatment of cancer, wherein the treatment further comprises administration of:
temozolomide, or
temozolomide and irinotecan.

Therefore, according to a nineteenth aspect of the invention, there is hereby provided irinotecan for use in the treatment of cancer, wherein the treatment further comprises administration of:
a WRN inhibitor, or in particular,
a WRN inhibitor and temozolomide.

Therefore, according to an embodiment of the sixteenth to nineteenth aspects of the invention, there is hereby provided temozolomide, a WRN inhibitor, or irinotecan, for use wherein the cancer is MSI-H, dMMR or MSS, in particular dMMR or MSS cancer, more particularly MSS cancer.

Therefore, according to a twentieth aspect of the invention, there is hereby provided temozolomide, a WRN inhibitor, or irinotecan, for use (in combination) according to any of the sixteenth to nineteenth aspects of the invention, wherein temozolomide is administered:
as a pre-treatment before administration of the WRN inhibitor, or before administration of irinotecan, or before administration of both the WRN inhibitor and irinotecan, without subsequent combination treatment including temozolide, or
in combination, without pre-treatment with temozolomide, or
as a pre-treatment before administration of the WRN inhibitor, or before administration of irinotecan, or before administration of both the WRN inhibitor and irinotecan, followed by combination treatment including temozolomide,

wherein said combination or combination treatment is described in the sixteenth to nineteenth aspects, and said use is for the treatment of cancer which is MSI-H, dMMR or MSS, in particular dMMR or MSS cancer, more particularly MSS cancer.

In an embodiment of the twentieth aspect, said cancer is selected from colorectal cancer (CRC), gastric cancer, prostate cancer, endometrial cancer, adrenocortical cancer, uterine cancer, small cell lung cancer and cervical cancer, for example a cancer selected from uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma, in particular colorectal cancer (CRC), gastric cancer, prostate cancer, or endometrial cancer, more particularly colorectal cancer or small cell lung cancer.

In another embodiment of the twentieth aspect, said cancer is selected from dMMR or MSS cancer. In particular, the cancer is MSS colorectal cancer (CRC) or MSS small cell lung cancer.

In another embodiment of the sixteenth to twentieth aspects, said WRN inhibitor is Compound A.

Therefore, according to a twenty-first aspect of the invention, there is hereby provided a WRN inhibitor for use in the treatment of dMMR or MSS cancer, in particular MSS cancer.

In one embodiment of the twenty-first aspect, the cancer is MSS colorectal cancer or MSS small cell lung cancer.

In an embodiment of the twenty-first aspect, the WRN inhibitor is a compound as defined in any of Embodiments 5 to 11, 83 or 84 herein. More particularly, the compound is Compound A herein.

Therefore, according to a twenty-second aspect of the invention, there is hereby provided a method of treating cancer in a subject in need thereof, the method comprising administering to the subject a therapeutically effective amount of temozolomide, wherein the treatment further comprises administration of:
a therapeutically effective amount of a WRN inhibitor, or
a therapeutically effective amount of a WRN inhibitor and a therapeutically effective amount of irinotecan.

Therefore, according to a twenty-third aspect of the invention, there is hereby provided a method of treating cancer in a subject in need thereof, the method comprising administering to the subject a therapeutically effective amount of a WRN inhibitor, wherein the treatment further comprises administration of:
a therapeutically effective amount of temozolomide, or
a therapeutically effective amount of temozolomide and a therapeutically effective amount of irinotecan.

Therefore, according to a twenty-fourth aspect of the invention, there is hereby provided a method of treating cancer in a subject in need thereof, the method comprising administering to the subject a therapeutically effective amount of irinotecan, wherein the treatment further comprises administration of:
a therapeutically effective amount of a WRN inhibitor, or in particular
a therapeutically effective amount of a WRN inhibitor and a therapeutically effective amount or temozolomide.

In an embodiment of the twenty-first to twenty-fourth aspects, the WRN inhibitor is a compound as defined in any of Embodiments 5 to 11, 83 or 84 herein. More particularly, the compound is Compound A herein.

In another embodiment of the twenty-second to twenty-fourth aspects, the cancer is MSI-H, dMMR or MSS, in particular dMMR or MSS, more particularly MSS. In particular, the cancer is MSS colorectal cancer (CRC) or MSS small cell lung cancer.

In another embodiment of the twenty-second to twenty-fourth aspects, temozolomide is administered:
as a pre-treatment before administration of the WRN inhibitor, or before administration of irinotecan, or before administration of both the WRN inhibitor and irinotecan, without subsequent combination treatment including temozolide, or
in combination, without pre-treatment with temozolomide, or
as a pre-treatment before administration of the WRN inhibitor, or before administration of irinotecan, or before administration of both the WRN inhibitor and irinotecan, followed by combination treatment including temozolide.

Therefore, according to a twenty-fifth aspect of the invention, there is hereby provided a WRN inhibitor for use in the treatment of cancer, wherein the treatment further comprises administration of an alkylating agent, in particular temozolomide, and the cancer is:
MSS cancer,
pMMR/MSS MGMT-defective, or pMMR/MSS MGMT-deficient, or pMMR/MSS MGMT-silenced, or MGMT-methylated pMMR/MSS cancer. For example, colorectal cancer,
MGMT-methylated glioblastoma,
MGMT-hypermethylated, CRC, or
MGMT-hypermethylated, RAS-mutated or RAS wild-type CRC.

Therefore, according to a twenty-sixth aspect of the invention, there is hereby provided an alkylating agent for use in the treatment of cancer, wherein the treatment further comprises administration of a WRN inhibitor, and the cancer is:
MSS cancer,
pMMR/MSS MGMT-defective, or pMMR/MSS MGMT-deficient, or pMMR/MSS MGMT-silenced, or MGMT-methylated pMMR/MSS cancer, such as colorectal cancer or small cell lung cancer.
MGMT-methylated glioblastoma,
MGMT-hypermethylated, CRC, or
MGMT-hypermethylated, RAS-mutated or RAS wild-type CRC.

Therefore according to a twenty-seventh aspect of the invention, there is provided a method of treating cancer, in particular cancer characterized as microsatellite instability-high (MSI-H) or mismatch repair deficient (dMMR), in a subject in need thereof, the method comprising administering to the subject a therapeutically effective amount of a WRN inhibitor in combination with a therapeutically effective amount of a DNA polymerase alpha inhibitor, for example aphidicolin, for example for the treatment of colorectal cancer.

Therefore, according to a twenty-eighth aspect of the invention, there is provided a method of treating cancer, in particular cancer characterized as MSS or mismatch repair deficient (dMMR), in particular MSS, in a subject in need thereof, the method comprising administering to the subject a therapeutically effective amount of a WRN inhibitor in combination with a therapeutic agent which can (or is capable of):
sensitise the cancer cells, for example for an improved response to treatment with a WRN inhibitor,
prime the cancer cells, for example, such priming may include triggering hypermutation status in cancer cells,
create or increase MMR deficiency in cancer cells,
create or increase MSI-H status in cancer cells,
increase MMR heterogeneity of cancer cells, and/or
create or increase resistance in cancer cells to temozolomide.

In particular to
create or increase MMR deficiency in cancer cells, or
create or increase MSI-H status in cancer cells.

In particular, said therapeutic agent is temozolomide.

Optionally, wherein the treatment further comprises administration of a chemotherapy, for example irinotecan.

Therefore, according to a twenty-ninth aspect of the invention, there is provided a WRN inhibitor for use in the treatment of cancer, in particular cancer characterized as MSS or mismatch repair deficient (dMMR), in particular MSS, wherein the treatment further comprises administration of a therapeutic agent which can or is capable of:
sensitise the cancer cells, for example for an improved response to treatment with a WRN inhibitor,
prime the cancer cells, for example, such priming may include triggering hypermutation status in cancer cells,
create or increase MMR deficiency in cancer cells,
create or increase MSI-H status in cancer cells,
increase MMR heterogeneity of cancer cells, and/or
create or increase resistance in cancer cells to temozolomide.

In particular to
create or increase MMR deficiency in cancer cells, or
create or increase MSI-H status in cancer cells.

In particular, said therapeutic agent is temozolomide.

Optionally, wherein the treatment further comprises administration of a chemotherapy, for example irinotecan.

The invention therefore provides the following numbered embodiments:

Embodiment 1. A method of treating cancer in a subject in need thereof, the method comprising administering to the subject a therapeutically effective amount of a WRN inhibitor in combination with a therapeutically effective amount of at least one therapeutically active agent selected from the group consisting of a PD-1 inhibitor, a chemotherapy agent, a Wee1 inhibitor, an ATR inhibitor, a DNA-PK inhibitor, ionising radiation based therapy selected from i) external beam radiation, ii) brachytherapy and iii) a radiopharmaceutical, a MEK inhibitor, a CTLA-4 inhibitor, an MDM2 inhibitor, a G4-quadruplex stabilizer, an ATM inhibitor, a CHK1 or CHK2 inhibitor and a PARP inhibitor.

Embodiment 2. A WRN inhibitor for use in the treatment of cancer, wherein the treatment further comprises administration of at least one therapeutically active agent selected from the group consisting of a PD-1 inhibitor, a chemotherapy agent, a Wee1 inhibitor, an ATR inhibitor, a DNA-PK inhibitor, ionising radiation based therapy selected from i) external beam radiation, ii) brachytherapy and iii) a radiopharmaceutical, a MEK inhibitor, a CTLA-4 inhibitor, an MDM2 inhibitor, a G4-quadruplex stabilizer, an ATM inhibitor, a CHK1 or CHK2 inhibitor and a PARP inhibitor.

Embodiment 3. A therapeutically active agent for use in the treatment of cancer, wherein the treatment further comprises administration of a WRN inhibitor, and wherein the therapeutically active agent is selected from the group consisting of a PD-1 inhibitor, a chemotherapy agent, a Wee1 inhibitor, an ATR inhibitor, a DNA-PK inhibitor, ionising radiation based therapy selected from i) external beam radiation, ii) brachytherapy and iii) a radiopharmaceutical, a MEK inhibitor, a CTLA-4 inhibitor, an MDM2 inhibitor, a G4-quadruplex stabilizer, an ATM inhibitor, a CHK1 or CHK2 inhibitor and a PARP inhibitor.

Embodiment 4. A combination comprising i) a WRN inhibitor, and ii) at least one therapeutically active agent selected from the group consisting of a Wee1 inhibitor, an ATR inhibitor, a DNA-PK inhibitor, ionising radiation selected from i) external beam radiation, ii) brachytherapy and iii) a radiopharmaceutical, a MEK inhibitor, a CTLA-4 inhibitor, an MDM2 inhibitor, a G4-quadruplex stabilizer, an ATM inhibitor, a CHK1 or CHK2 inhibitor, and a PARP inhibitor.

Embodiment 5. The method according to Embodiment 1, the WRN inhibitor for use according to Embodiment 2, the therapeutically active agent for use according to Embodiment 3, or the combination according to Embodiment 4, wherein the WRN inhibitor is a compound of formula (I), or a pharmaceutically acceptable salt thereof:

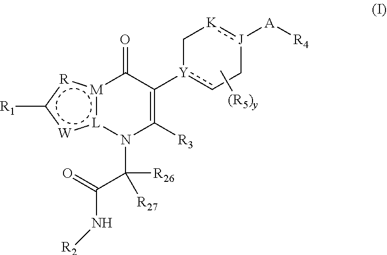

wherein
R, M, W, L, V and T are independently selected from C, CH and N, to form subformulae 1a, 1b, 1c, 1d, 1e and 1f:

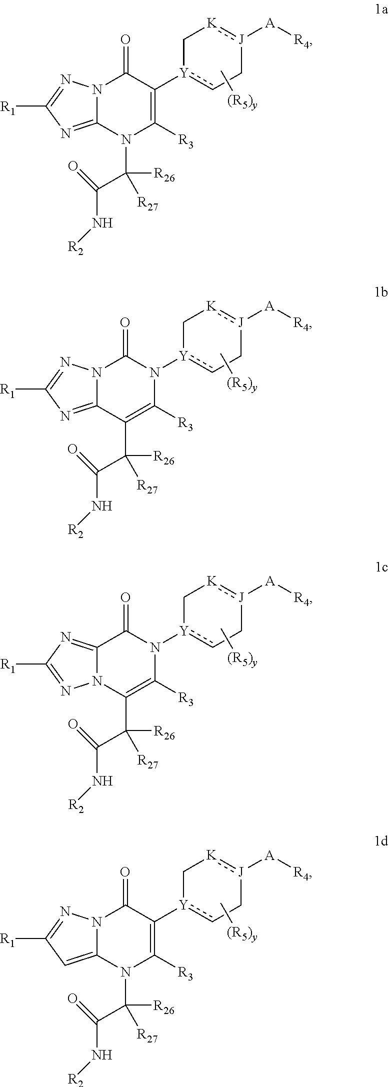

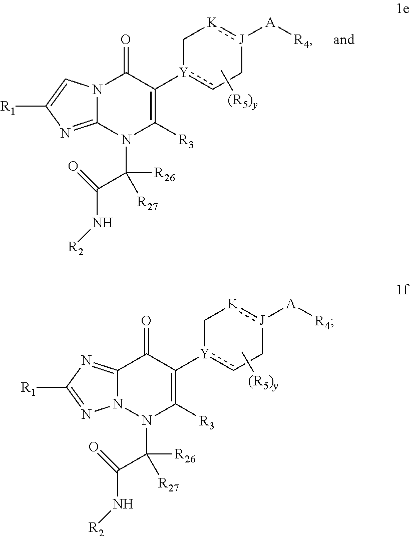

A is a linker selected from —C(O)—, —S(O)—, —S(O)₂—, and

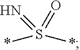

Y is N, C or CH;
y is 0, 1, 2, 3 or 4;
Y [symbol] means Y is linked via a single bond to the adjacent carbon atom when Y is CH, or Y is linked via a double bond to the adjacent atom when Y is C, and when Y [symbol] is a single bond, Y is carbon unsubstituted or substituted by OH or F;
when Y is N, Y [symbol] is a single bond;
K [symbol] means K is linked via a single or double bond to the adjacent atom;
wherein:
   when K [symbol] is a double bond, Y [symbol] is a single bond, K is CH, J is C, and A is a linker selected from —C(O)—, —S(O)—, —S(O)₂—, and

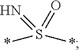

or
when K [symbol] is a single bond, K is selected from —CH₂—, —CH₂CH₂—, —NH— and a bond (to form a 5-membered ring:

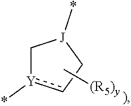

J is N, and A is a linker selected from —C(O)—, —S(O)—, —S(O)₂—, and

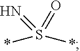

or
when K [symbol] is a single bond, K is —CH₂—, J is CH, and A is a linker selected from —S(O)—, —S(O)₂—, and

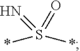

R₅ is independently selected from:
   —(C₁-C₄)alkyl,
   —(C₃-C₅)cycloalkyl,
   and wherein two R₅ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S,
   when K [symbol] J is a carbon-nitrogen single bond, a R₅ substituent on K and on the adjacent carbon atom may join to form ring C:

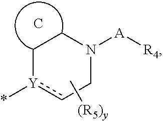

wherein ring C is a fused (C₃-C₆)cycloalkyl ring, a fused (C₃-C₆)heterocyclyl ring or a fused phenyl ring, wherein said fused (C₃-C₆)heterocyclyl ring contains ring carbon atoms and one ring heteroatom selected from O, N and S,
   when K [symbol] J is a carbon-carbon single bond, Y is N and Y [symbol] is a single bond, and A is a linker selected from —S(O)—, —S(O)₂—, and

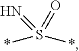

a R₅ substituent on K and on the adjacent carbon atom may join to form ring C:

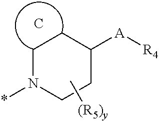

and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—;
and wherein one or more H atoms on the ring:

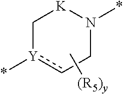

may be replaced by deuterium;
R₁ is:
cycloalkenyl, wherein said cycloalkenyl is a partially unsaturated monocyclic ring containing 5 or 6 ring carbon atoms, and said cycloalkenyl is unsubstituted or substituted by 1, 2, 3 or 4, preferably 1 or 2, R₃₃, wherein R₃₃ is halo, and wherein said cycloalkenyl or halo-substituted cycloalkenyl is substituted by 0, 1 or 2 R₁₅ substituents,
or R₁ is heterocyclyl, wherein said heterocyclyl is a 5 or 6 membered fully saturated or partially unsaturated group comprising ring carbon atoms and 1 or 2 ring heteroatoms independently selected from N, O and S, and wherein said heterocyclyl is unbridged or bridged, and said bridge is 1 or 2 carbon atoms, wherein said heterocyclyl is unsubstituted or substituted by 1, 2, 3 or 4, preferably 1 or 2, R₃₃, wherein R₃₃ is halo, and wherein said heterocyclyl or halo-substituted heterocyclyl is substituted by 0, 1 or 2 substituents independently selected from R₁₅, R₁₆, R₁₇, R₁₈, R₁₉, R₂₀, R₂₂ and R₂₃,
or said heterocyclyl or halo-substituted heterocyclyl is fused to a cyclopropyl ring, wherein said cyclopropyl ring is unsubstituted or substituted by 1, 2 or 3 F,
or said heterocyclyl or halo-substituted heterocyclyl has 2 substitutents at the same ring carbon atom which join to form a cyclopropyl spiro ring,
or said heterocyclyl or halo-substituted heterocyclyl is fused with a (C₃-C₅)heterocycloalkyl ring, wherein said (C₃-C₅)heterocycloalkyl ring contains ring carbon atoms and 1 ring O atom;
or R₁ is heteroaryl, wherein said heteroaryl is a 5 or 6 membered fully unsaturated monocyclic group comprising ring carbon atoms and 1, 2, 3 or 4 ring heteroatoms independently selected from N, O and S, preferably 1 or 2 ring heteroatoms, wherein the total number of ring S atoms does not exceed 1, and the total number of ring O atoms does not exceed 1, wherein said heteroaryl is unsubstituted or substituted by 1, 2 or 3 substituents independently selected from R₂₁ and R₃₀, wherein R₂₁ and R₃₀ are independently selected from halo and (C₁-C₄)alkyl, wherein said (C₁-C₄)alkyl is unsubstituted or substituted by 1, 2 or 3 halo,
or R₁ is phenyl, wherein said phenyl is unsubstituted or substituted by 1, 2, 3 or 4, preferably 1 or 2, R₃₃, wherein R₃₃ is halo, and wherein said phenyl or halo-substituted phenyl is substituted by 0, 1 or 2 R₁₅ substituents,
or R₁ is (C₂-C₄)alkynyl or (C₂-C₄)alkenyl, wherein said (C₂-C₄)alkynyl and (C₂-C₄)alkenyl are unsubstituted or substituted by (C₁-C₄)alkyl-O—C(O)—, or morpholinyl;
each R₁₅, R₁₆, R₁₇, R₁₈, R₁₉, R₂₀, R₂₂ and R₂₃ is independently selected from:
   halo
   (C₁-C₄)alkyl-O— unsubstituted or substituted by 1, 2 or 3 halo;
   (C₁-C₄)alkyl unsubstituted or substituted by OH, —O—(C₁-C₂)alkyl or 1, 2 or 3 halo,
   HOC(O)—(CH₂)ₙ—,
   H₃C—C(O)(CH₂)ₙ—,
   (C₁-C₄)alkyl-O—C(O)(CH₂)ₙ,
   ═O
   azetidinyl or pyrrolidinyl, wherein said azetidinyl and pyrrolidinyl are linked to the rest of the molecule via the N atom, and are each unsubstituted or substituted by 1 or 2 F,
   R₂₅(R₂₄)N—, wherein R₂₄ is H or (C₁-C₄)alkyl unsubstituted or substituted by 1, 2 or 3 halo, R₂₅ is H or (C₁-C₄)alkyl unsubstituted or substituted by 1, 2 or 3 halo,
   OH
wherein n is 0, 1 or 2,
R₂₆ is CH₃, H or deuterium;
R₂₇ is CH₃, H or deuterium;
or R₂₈ and R₂₇ join, together with the carbon atom to which they are attached, to form a cyclopropyl ring;
R₂ is the moiety:

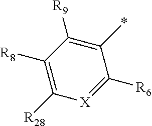

Re is selected from:
   H,
   halo,
   (C₁-C₄)alkyl unsubstituted or substituted by 1, 2 or 3 halo,
   (C₃-C₅)cycloalkyl unsubstituted or substituted by 1, 2 or 3 halo,
   —O—(C₁-C₄)alkyl unsubstituted or substituted by 1, 2 or 3 halo,
   OH, and
   CN;
R₈ is selected from H, halo, and (C₁-C₄)alkyl unsubstituted or substituted by 1, 2 or 3 halo,
R₉ is selected from H, O—CH₃, OH, CN, CH₃ and halo;
R₂₈ is selected from:
   SF₅,
   H,
   —C(O)H,
   halo,
   (C₁-C₄)alkyl unsubstituted or substituted by 1, 2 or 3 halo,
   (C₁-C₄)alkynyl,
   (C₁-C₄)alkenyl,
   (C₃-C₅)cycloalkyl unsubstituted or substituted by 1, 2 or 3 halo, and
   OCF₃;

X is selected from C—R₇ and N, wherein R₇ is H or halo, or R₇ can join, together with R₂₈ or R₆, and the atoms to which they are attached, to form a fused (C₄-C₆)cycloalkyl ring, wherein said fused (C₄-C₆)cycloalkyl ring is unsubstituted or substituted by 1, 2 or 3 halo, or
R₂ is selected from:

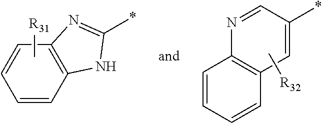

wherein
R₃₁ is selected from H, halo and CH₃,
R₃₂ is selected from H, halo and CH₃,
R₃ is:
   cyclopropyl,
   O—CH₃,
   N(CH₃)₂,
   S—CH₃,
   (C₁-C₄)alkyl unsubstituted or substituted by 1, 2 or 3 substituents independently selected from halo and OH;
R₄ is selected from:

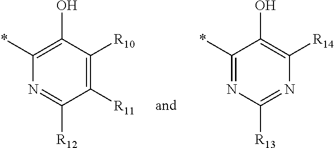

wherein
R₁₀, R₁₁, R₁₂, R₁₃ and R₁₄ are independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   S—(C₁-C₃)alkyl,
   O—(C₁-C₄)alkyl unsubstituted or substituted by 1, 2 or 3 halo substituents,
   OH,
   (C₃-C₅)cycloalkyl, wherein said (C₃-C₅)cycloalkyl is unsubstituted or substituted by 1 or 2 halo,
   O—(C₃-C₅)cycloalkyl,
   NR₃₄R₃₅ wherein R₃₄ and R₃₅ are independently selected from:
      H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
   CN,
   —(C₂-C₄)alkenyl,
   —(C₂-C₄)alkynyl,
   —C(O)H, and
   —C(O)(C₁-C₄)alkyl;
and
* indicates a point of attachment,
or
or the WRN inhibitor is a compound of formula (1g), or a pharmaceutically acceptable salt thereof:

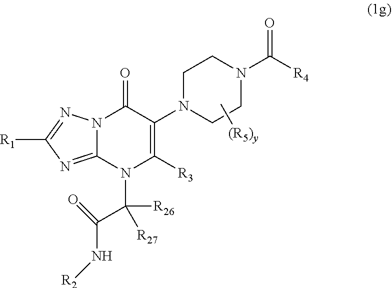

wherein
R₁ is selected from:

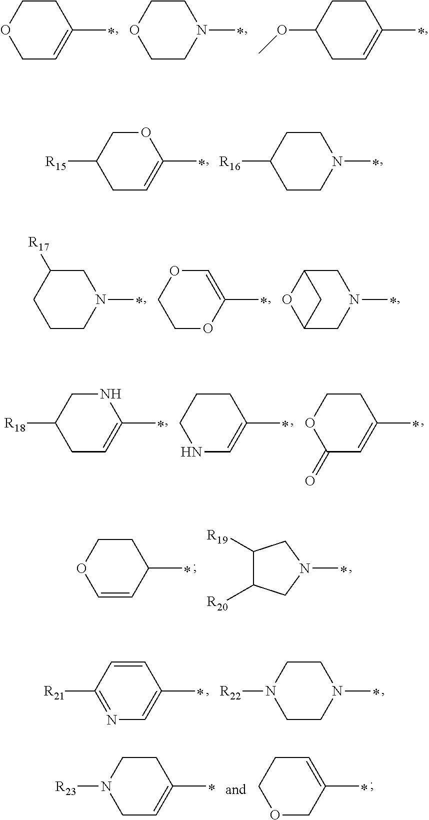

R₁₅ is H or F;
R₁₆ is H or R₂₅(R₂₄)N—;
R₁₇ is H or F;
R₁₈ is H or F;
R₁₉ is H or F;
R₂₀ is H or F;
R₂₁ is H or CH₃;
R₂₂ is H, CF₃, CHF₂CH₂, HOC(O)—CH₂—, H₃C—C(O)—, (H₃C)₃C—O—C(O)—;
R₂₃ is H, CF₃, CHF₂CH₂—, (H₃C)₃C—O—C(O)—;
R₂₄ is CH₃;
R₂₅ is CHF₂CH₂;
R₂₈ is CH₃, H or deuterium;
R₂₇ is H or deuterium;
R₂ is the moiety:

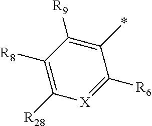

wherein Re is selected from H, Cl, CH₃, F, and Br;
R₅ is selected from H, Cl, F and CF₃;
Re is selected from H, CH₃ and Cl;
R₂₈ is selected from CF₃, CF₂H, —CH₂CH₃, Cl, SF₅, Br and —C(O)H;
X is selected from C—R₇ and N;
R₇ is selected from H and F;
R₃ is selected from CH₃, CH₂CH₃, cyclopropyl and hydroxyethyl;
R₄ is selected from:

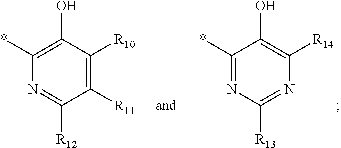

wherein
R₁₀ is selected from H, F, Cl, CH₃ and OCF₃;
R₁₁ is selected from H, Cl, F, and CH₃;
R₁₂ is selected from H, Cl and CH₃;
R₁₃ is selected from H and CH₃;
R₁₄ is selected from H, CH₃, —CH₂CH₃, cyclopropyl, —OCHF₂, OCF₃ and Cl;
y is 0, 1 or 2;
R₅ is CH₃; or alternatively, two R₅ groups on adjacent carbon atoms join, along with the carbon atoms to which they are attached, to form a fused cyclobutyl ring:

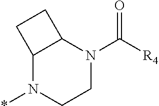

and
indicates a point of attachment.

6. The method according to Embodiment 5, the WRN inhibitor for use according to Embodiment 5, the therapeutically active agent for use according to Embodiment 5, or the combination according to Embodiment 5, wherein the WRN inhibitor is a compound selected from:

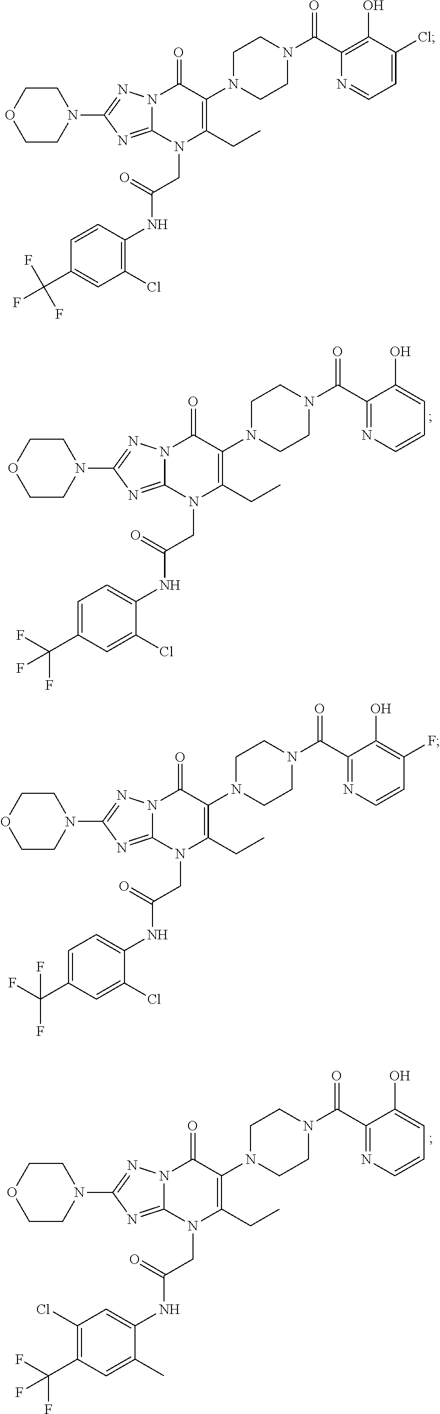 [SMILES: CCc1c(N2CCN(C(=O)c3nccc(Cl)c3O)CC2)c(=O)n2nc(N3CCOCC3)nc2n1CC(=O)Nc1ccc(C(F)(F)F)cc1Cl ; CCc1c(N2CCN(C(=O)c3ncccc3O)CC2)c(=O)n2nc(N3CCOCC3)nc2n1CC(=O)Nc1ccc(C(F)(F)F)cc1Cl ; CCc1c(N2CCN(C(=O)c3nccc(F)c3O)CC2)c(=O)n2nc(N3CCOCC3)nc2n1CC(=O)Nc1ccc(C(F)(F)F)cc1Cl ; CCc1c(N2CCN(C(=O)c3ncccc3O)CC2)c(=O)n2nc(N3CCOCC3)nc2n1CC(=O)Nc1cc(Cl)c(C(F)(F)F)cc1C]

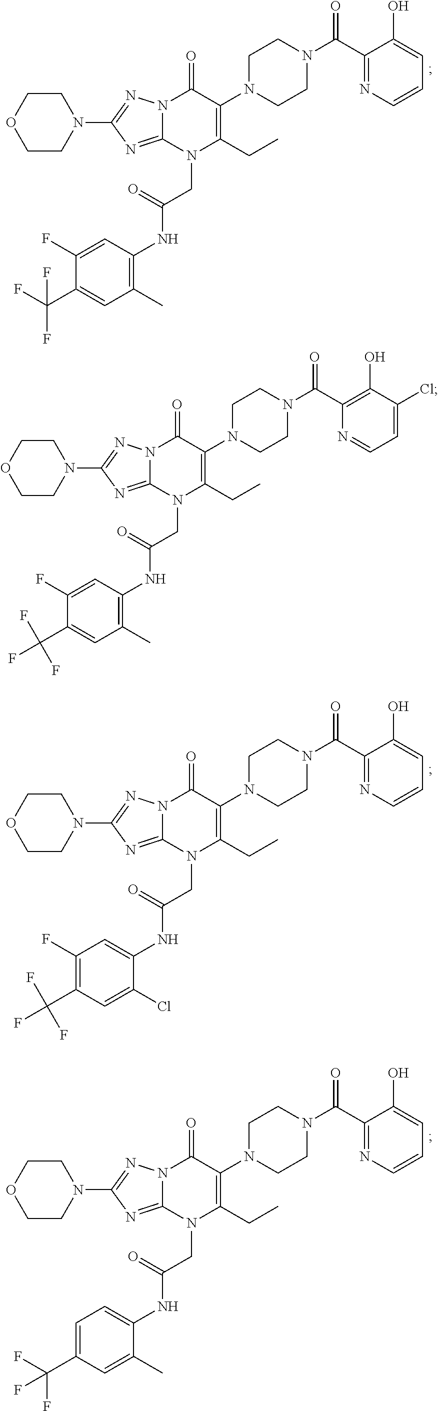 [SMILES: CCc1c(N2CCN(C(=O)c3ncccc3O)CC2)c(=O)n2nc(N3CCOCC3)nc2n1CC(=O)Nc1cc(F)c(C(F)(F)F)cc1C ; CCc1c(N2CCN(C(=O)c3nccc(Cl)c3O)CC2)c(=O)n2nc(N3CCOCC3)nc2n1CC(=O)Nc1cc(F)c(C(F)(F)F)cc1C ; CCc1c(N2CCN(C(=O)c3ncccc3O)CC2)c(=O)n2nc(N3CCOCC3)nc2n1CC(=O)Nc1cc(F)c(C(F)(F)F)cc1Cl ; CCc1c(N2CCN(C(=O)c3ncccc3O)CC2)c(=O)n2nc(N3CCOCC3)nc2n1CC(=O)Nc1ccc(C(F)(F)F)cc1C]

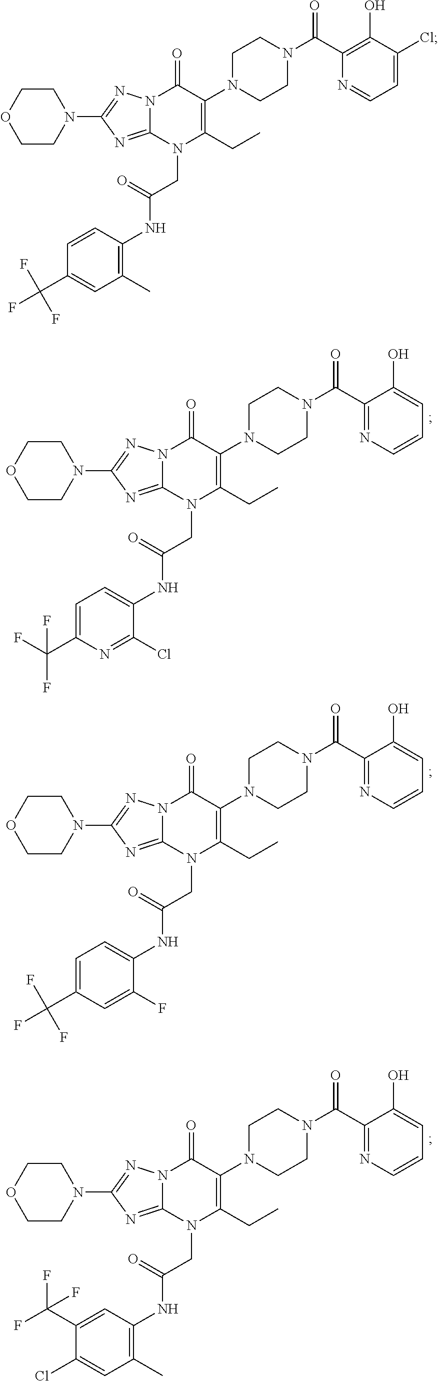 [SMILES: CCc1c(N2CCN(C(=O)c3nccc(Cl)c3O)CC2)c(=O)n2nc(N3CCOCC3)nc2n1CC(=O)Nc1ccc(C(F)(F)F)cc1C ; CCc1c(N2CCN(C(=O)c3ncccc3O)CC2)c(=O)n2nc(N3CCOCC3)nc2n1CC(=O)Nc1ccc(C(F)(F)F)nc1Cl ; CCc1c(N2CCN(C(=O)c3ncccc3O)CC2)c(=O)n2nc(N3CCOCC3)nc2n1CC(=O)Nc1ccc(C(F)(F)F)cc1F ; CCc1c(N2CCN(C(=O)c3ncccc3O)CC2)c(=O)n2nc(N3CCOCC3)nc2n1CC(=O)Nc1cc(C(F)(F)F)c(Cl)cc1C]

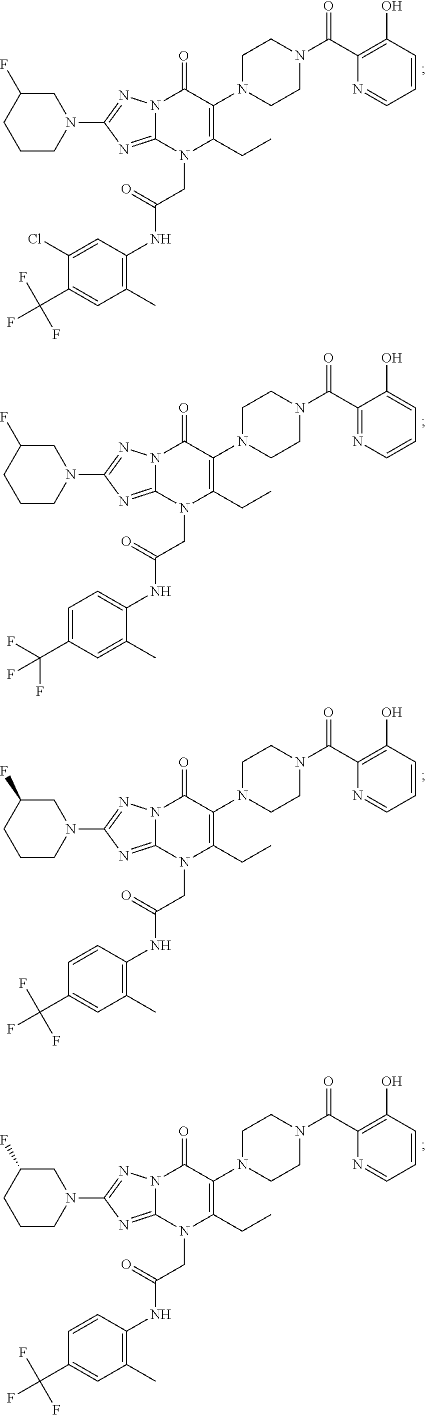 [SMILES: CCc1c(N2CCN(C(=O)c3ncccc3O)CC2)c(=O)n2nc(N3CCCC(F)C3)nc2n1CC(=O)Nc1cc(Cl)c(C(F)(F)F)cc1C ; CCc1c(N2CCN(C(=O)c3ncccc3O)CC2)c(=O)n2nc(N3CCCC(F)C3)nc2n1CC(=O)Nc1ccc(C(F)(F)F)cc1C ; CCc1c(N2CCN(C(=O)c3ncccc3O)CC2)c(=O)n2nc(N3CCC[C@@H](F)C3)nc2n1CC(=O)Nc1ccc(C(F)(F)F)cc1C ; CCc1c(N2CCN(C(=O)c3ncccc3O)CC2)c(=O)n2nc(N3CCC[C@H](F)C3)nc2n1CC(=O)Nc1ccc(C(F)(F)F)cc1C]

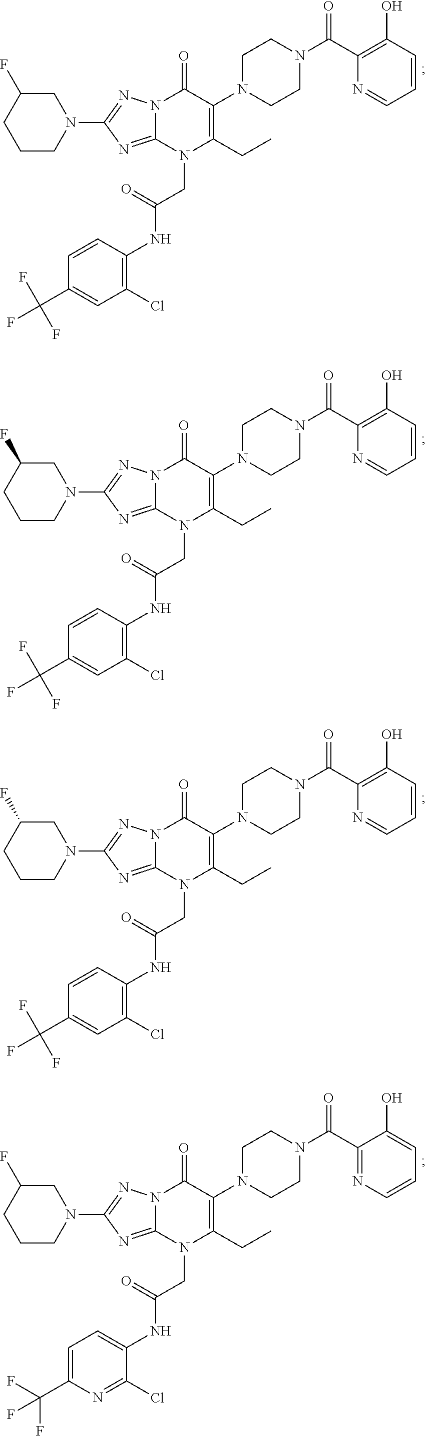 [SMILES: CCc1c(N2CCN(C(=O)c3ncccc3O)CC2)c(=O)n2nc(N3CCCC(F)C3)nc2n1CC(=O)Nc1ccc(C(F)(F)F)cc1Cl ; CCc1c(N2CCN(C(=O)c3ncccc3O)CC2)c(=O)n2nc(N3CCC[C@@H](F)C3)nc2n1CC(=O)Nc1ccc(C(F)(F)F)cc1Cl ; CCc1c(N2CCN(C(=O)c3ncccc3O)CC2)c(=O)n2nc(N3CCC[C@H](F)C3)nc2n1CC(=O)Nc1ccc(C(F)(F)F)cc1Cl ; CCc1c(N2CCN(C(=O)c3ncccc3O)CC2)c(=O)n2nc(N3CCCC(F)C3)nc2n1CC(=O)Nc1ccc(C(F)(F)F)nc1Cl]

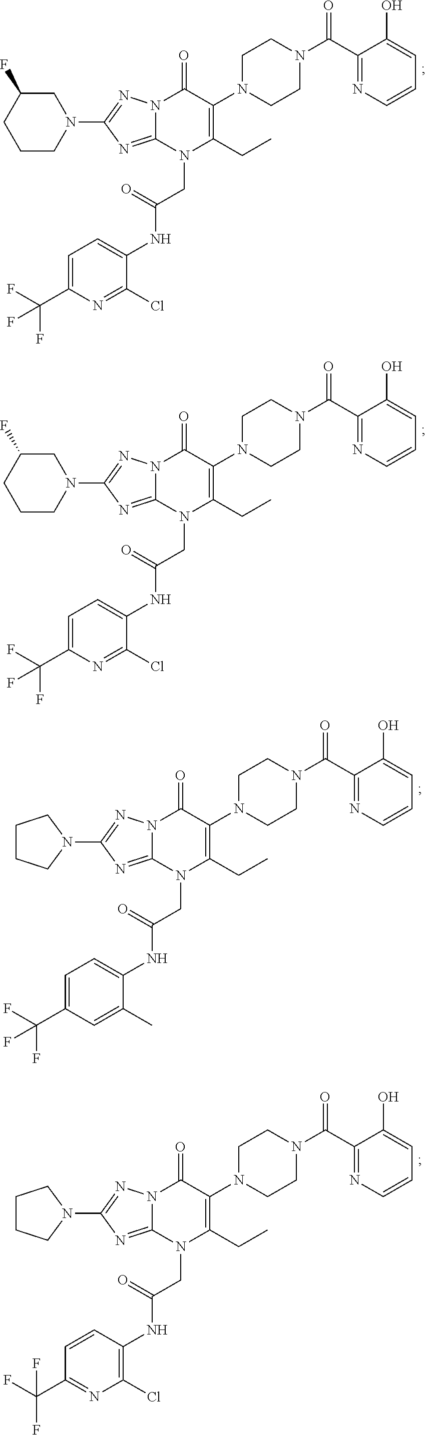 [SMILES: CCc1c(N2CCN(C(=O)c3ncccc3O)CC2)c(=O)n2nc(N3CCC[C@@H](F)C3)nc2n1CC(=O)Nc1ccc(C(F)(F)F)nc1Cl ; CCc1c(N2CCN(C(=O)c3ncccc3O)CC2)c(=O)n2nc(N3CCC[C@H](F)C3)nc2n1CC(=O)Nc1ccc(C(F)(F)F)nc1Cl ; CCc1c(N2CCN(C(=O)c3ncccc3O)CC2)c(=O)n2nc(N3CCCC3)nc2n1CC(=O)Nc1ccc(C(F)(F)F)cc1C ; CCc1c(N2CCN(C(=O)c3ncccc3O)CC2)c(=O)n2nc(N3CCCC3)nc2n1CC(=O)Nc1ccc(C(F)(F)F)nc1Cl]

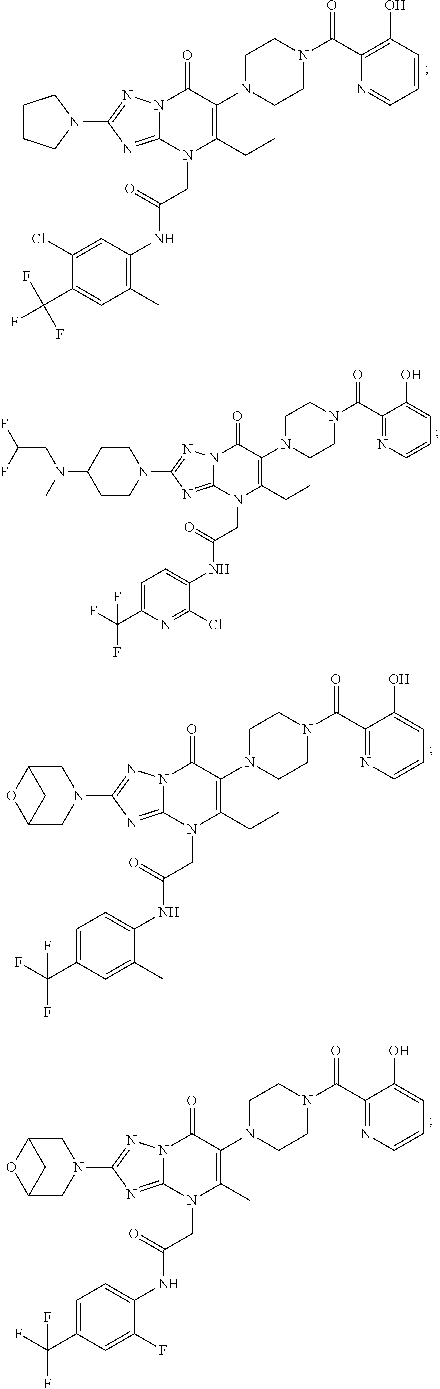 [SMILES: CCc1c(N2CCN(C(=O)c3ncccc3O)CC2)c(=O)n2nc(N3CCCC3)nc2n1CC(=O)Nc1cc(Cl)c(C(F)(F)F)cc1C ; CCc1c(N2CCN(C(=O)c3ncccc3O)CC2)c(=O)n2nc(N3CCC(N(C)CC(F)F)CC3)nc2n1CC(=O)Nc1ccc(C(F)(F)F)nc1Cl ; CCc1c(N2CCN(C(=O)c3ncccc3O)CC2)c(=O)n2nc(N3CC4CC(C3)O4)nc2n1CC(=O)Nc1ccc(C(F)(F)F)cc1C ; Cc1c(N2CCN(C(=O)c3ncccc3O)CC2)c(=O)n2nc(N3CC4CC(C3)O4)nc2n1CC(=O)Nc1ccc(C(F)(F)F)cc1F]

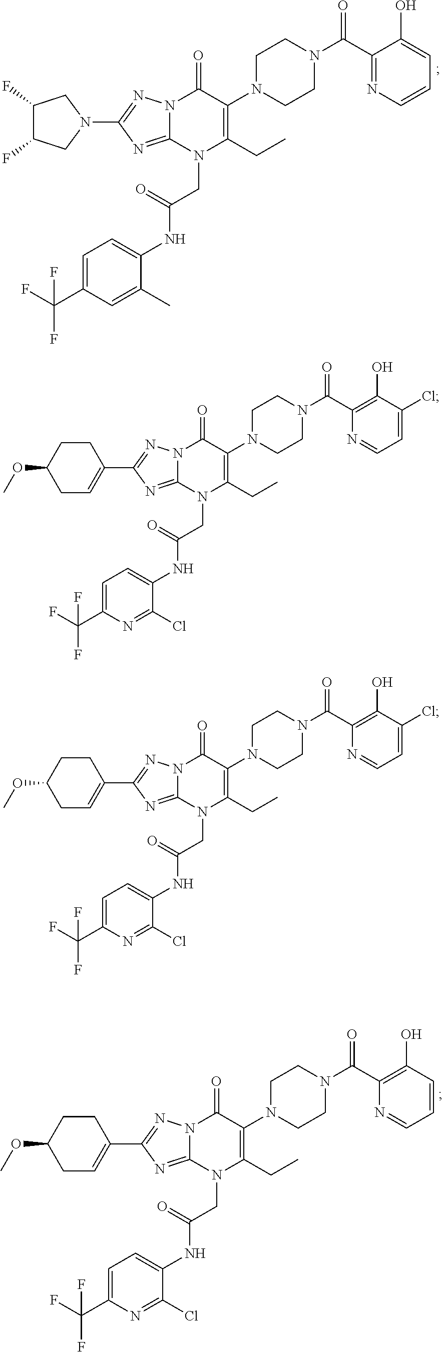 [SMILES: CCc1c(N2CCN(C(=O)c3ncccc3O)CC2)c(=O)n2nc(N3C[C@@H](F)[C@@H](F)C3)nc2n1CC(=O)Nc1ccc(C(F)(F)F)cc1C ; CCc1c(N2CCN(C(=O)c3nccc(Cl)c3O)CC2)c(=O)n2nc(C3=CC[C@@H](OC)CC3)nc2n1CC(=O)Nc1ccc(C(F)(F)F)nc1Cl ; CCc1c(N2CCN(C(=O)c3nccc(Cl)c3O)CC2)c(=O)n2nc(C3=CC[C@H](OC)CC3)nc2n1CC(=O)Nc1ccc(C(F)(F)F)nc1Cl ; CCc1c(N2CCN(C(=O)c3ncccc3O)CC2)c(=O)n2nc(C3=CC[C@@H](OC)CC3)nc2n1CC(=O)Nc1ccc(C(F)(F)F)nc1Cl]

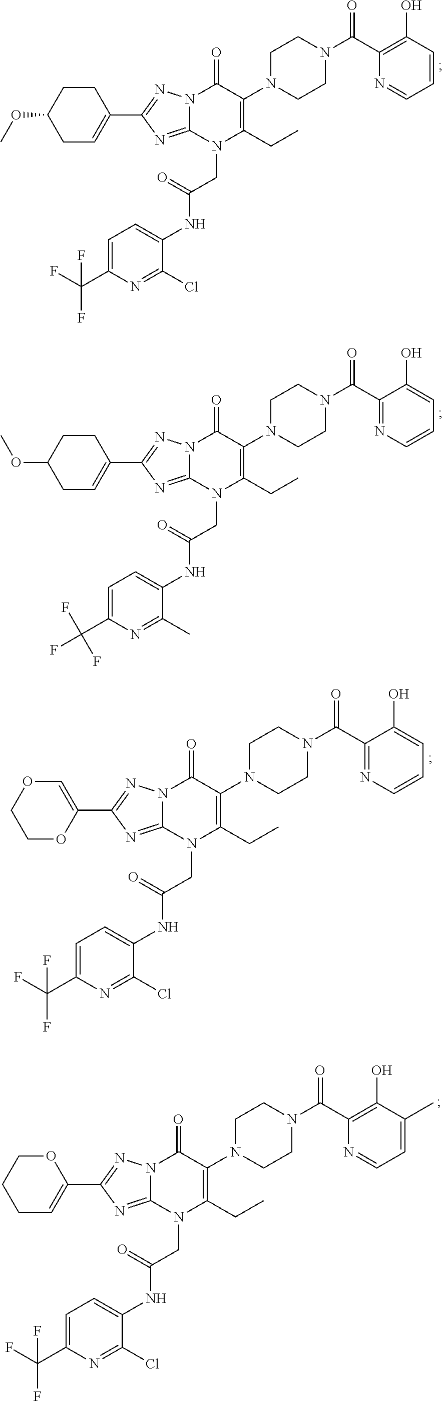 [SMILES: CCc1c(N2CCN(C(=O)c3ncccc3O)CC2)c(=O)n2nc(C3=CC[C@H](OC)CC3)nc2n1CC(=O)Nc1ccc(C(F)(F)F)nc1Cl ; CCc1c(N2CCN(C(=O)c3ncccc3O)CC2)c(=O)n2nc(C3=CCC(OC)CC3)nc2n1CC(=O)Nc1ccc(C(F)(F)F)nc1C ; CCc1c(N2CCN(C(=O)c3ncccc3O)CC2)c(=O)n2nc(C3=COCCO3)nc2n1CC(=O)Nc1ccc(C(F)(F)F)nc1Cl ; CCc1c(N2CCN(C(=O)c3nccc(C)c3O)CC2)c(=O)n2nc(C3=CCCCO3)nc2n1CC(=O)Nc1ccc(C(F)(F)F)nc1Cl]

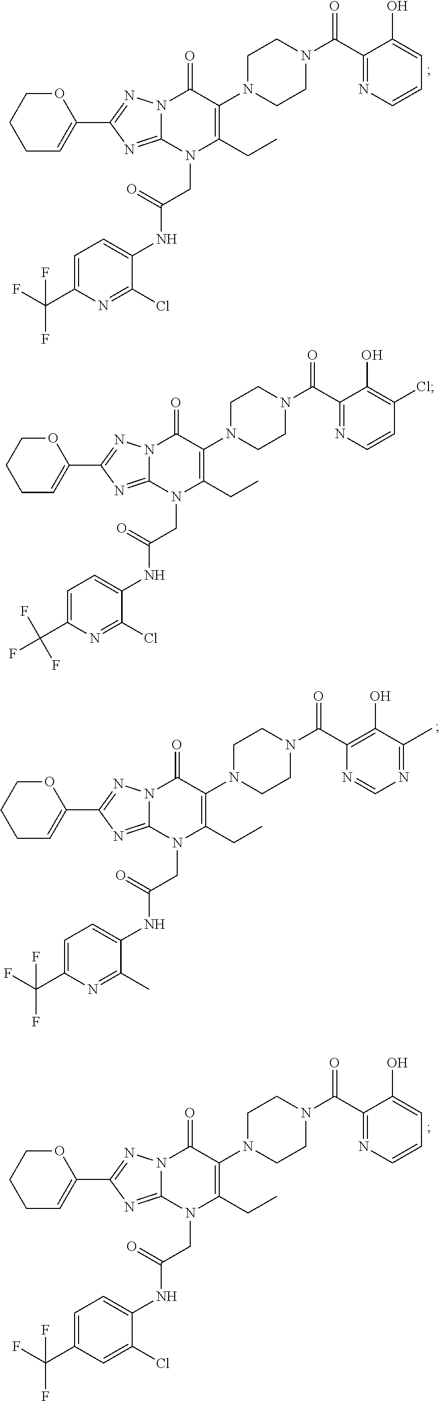 [SMILES: CCc1c(N2CCN(C(=O)c3ncccc3O)CC2)c(=O)n2nc(C3=CCCCO3)nc2n1CC(=O)Nc1ccc(C(F)(F)F)nc1Cl ; CCc1c(N2CCN(C(=O)c3nccc(Cl)c3O)CC2)c(=O)n2nc(C3=CCCCO3)nc2n1CC(=O)Nc1ccc(C(F)(F)F)nc1Cl ; CCc1c(N2CCN(C(=O)c3ncnc(C)c3O)CC2)c(=O)n2nc(C3=CCCCO3)nc2n1CC(=O)Nc1ccc(C(F)(F)F)nc1C ; CCc1c(N2CCN(C(=O)c3ncccc3O)CC2)c(=O)n2nc(C3=CCCCO3)nc2n1CC(=O)Nc1ccc(C(F)(F)F)cc1Cl]

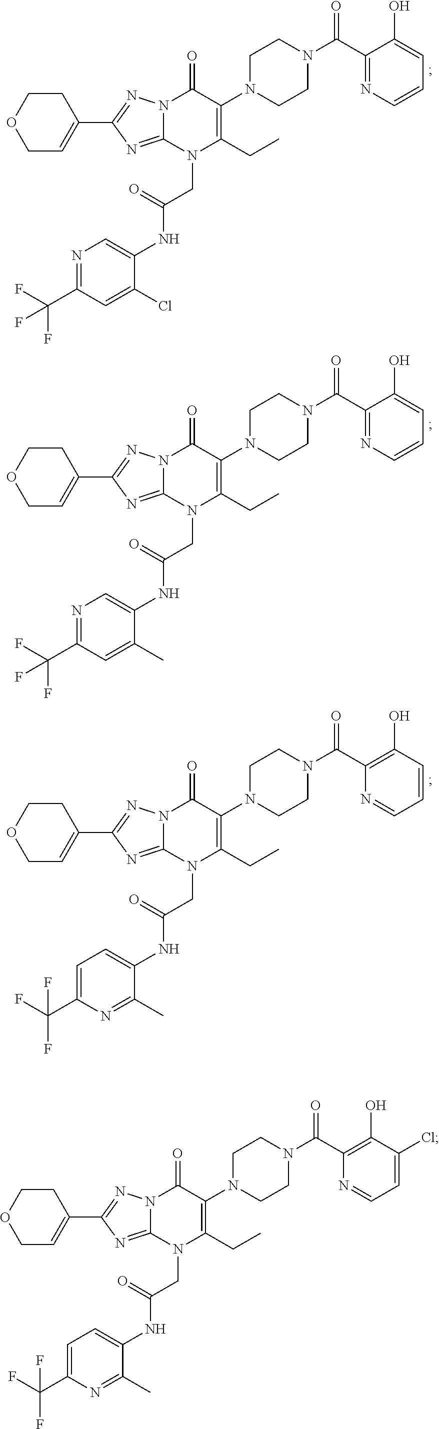 [SMILES: CCc1c(N2CCN(C(=O)c3ncccc3O)CC2)c(=O)n2nc(C3=CCOCC3)nc2n1CC(=O)Nc1cnc(C(F)(F)F)cc1Cl ; CCc1c(N2CCN(C(=O)c3ncccc3O)CC2)c(=O)n2nc(C3=CCOCC3)nc2n1CC(=O)Nc1cnc(C(F)(F)F)cc1C ; CCc1c(N2CCN(C(=O)c3ncccc3O)CC2)c(=O)n2nc(C3=CCOCC3)nc2n1CC(=O)Nc1ccc(C(F)(F)F)nc1C ; CCc1c(N2CCN(C(=O)c3nccc(Cl)c3O)CC2)c(=O)n2nc(C3=CCOCC3)nc2n1CC(=O)Nc1ccc(C(F)(F)F)nc1C]

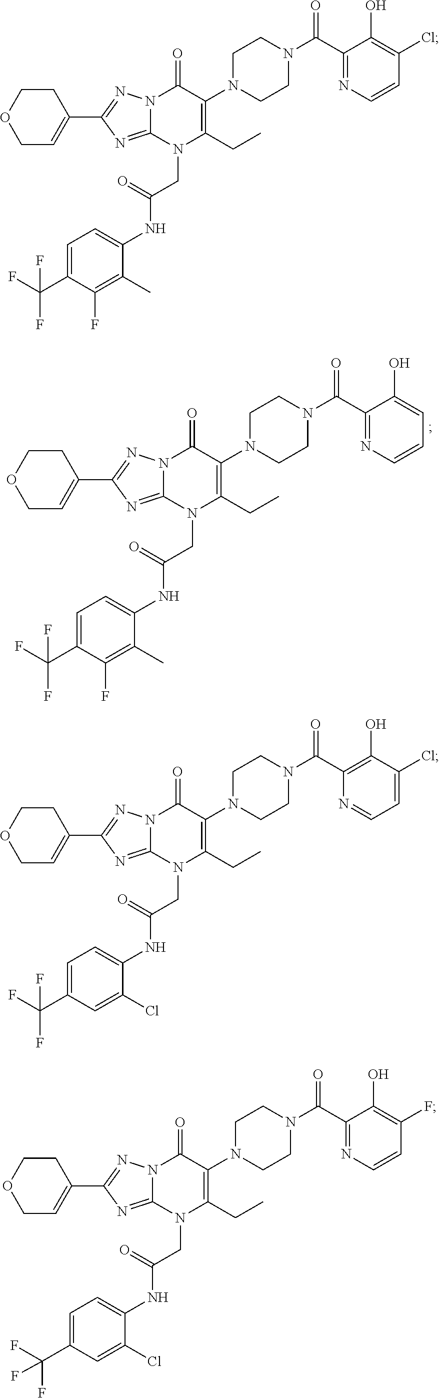 [SMILES: CCc1c(N2CCN(C(=O)c3nccc(Cl)c3O)CC2)c(=O)n2nc(C3=CCOCC3)nc2n1CC(=O)Nc1ccc(C(F)(F)F)c(F)c1C ; CCc1c(N2CCN(C(=O)c3ncccc3O)CC2)c(=O)n2nc(C3=CCOCC3)nc2n1CC(=O)Nc1ccc(C(F)(F)F)c(F)c1C ; CCc1c(N2CCN(C(=O)c3nccc(Cl)c3O)CC2)c(=O)n2nc(C3=CCOCC3)nc2n1CC(=O)Nc1ccc(C(F)(F)F)cc1Cl ; CCc1c(N2CCN(C(=O)c3nccc(F)c3O)CC2)c(=O)n2nc(C3=CCOCC3)nc2n1CC(=O)Nc1ccc(C(F)(F)F)cc1Cl]

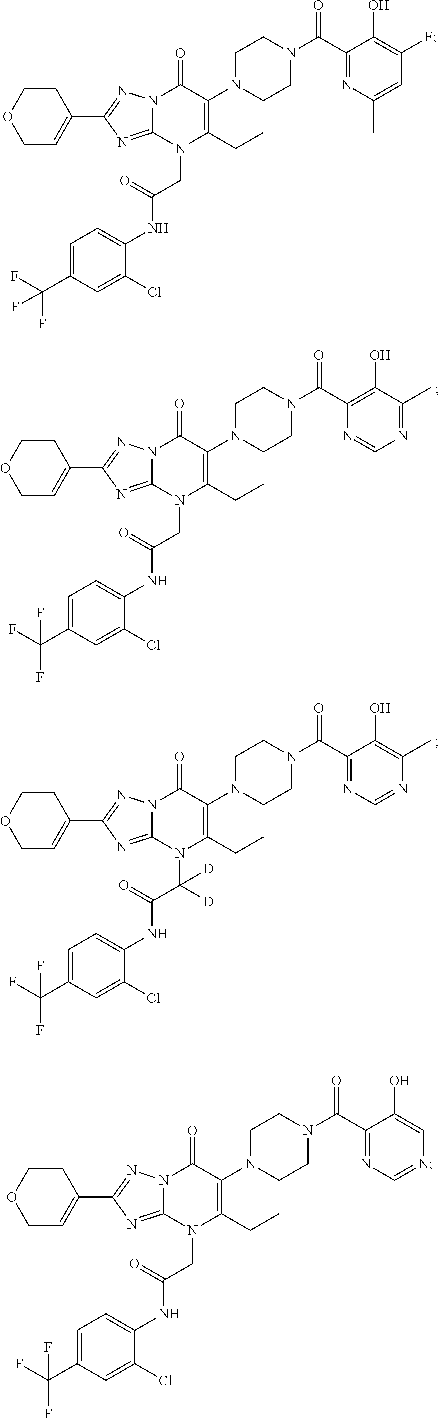 [SMILES: CCc1c(N2CCN(C(=O)c3nc(C)cc(F)c3O)CC2)c(=O)n2nc(C3=CCOCC3)nc2n1CC(=O)Nc1ccc(C(F)(F)F)cc1Cl ; CCc1c(N2CCN(C(=O)c3ncnc(C)c3O)CC2)c(=O)n2nc(C3=CCOCC3)nc2n1CC(=O)Nc1ccc(C(F)(F)F)cc1Cl ; [2H]C([2H])(C(=O)Nc1ccc(C(F)(F)F)cc1Cl)n1c(CC)c(N2CCN(C(=O)c3ncnc(C)c3O)CC2)c(=O)n2nc(C3=CCOCC3)nc12 ; CCc1c(N2CCN(C(=O)c3ncncc3O)CC2)c(=O)n2nc(C3=CCOCC3)nc2n1CC(=O)Nc1ccc(C(F)(F)F)cc1Cl]

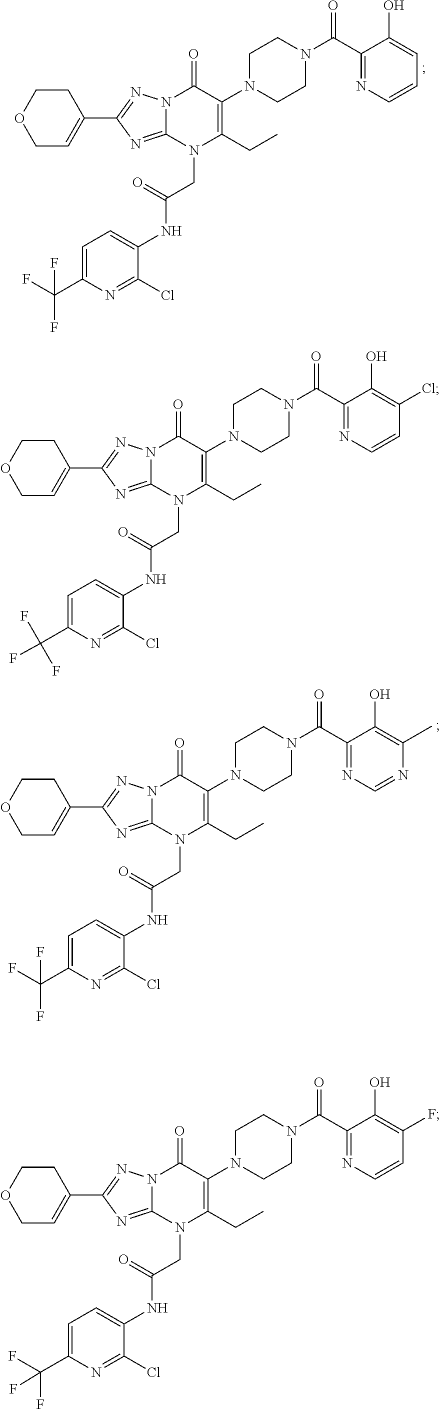 [SMILES: CCc1c(N2CCN(C(=O)c3ncccc3O)CC2)c(=O)n2nc(C3=CCOCC3)nc2n1CC(=O)Nc1ccc(C(F)(F)F)nc1Cl ; CCc1c(N2CCN(C(=O)c3nccc(Cl)c3O)CC2)c(=O)n2nc(C3=CCOCC3)nc2n1CC(=O)Nc1ccc(C(F)(F)F)nc1Cl ; CCc1c(N2CCN(C(=O)c3ncnc(C)c3O)CC2)c(=O)n2nc(C3=CCOCC3)nc2n1CC(=O)Nc1ccc(C(F)(F)F)nc1Cl ; CCc1c(N2CCN(C(=O)c3nccc(F)c3O)CC2)c(=O)n2nc(C3=CCOCC3)nc2n1CC(=O)Nc1ccc(C(F)(F)F)nc1Cl]

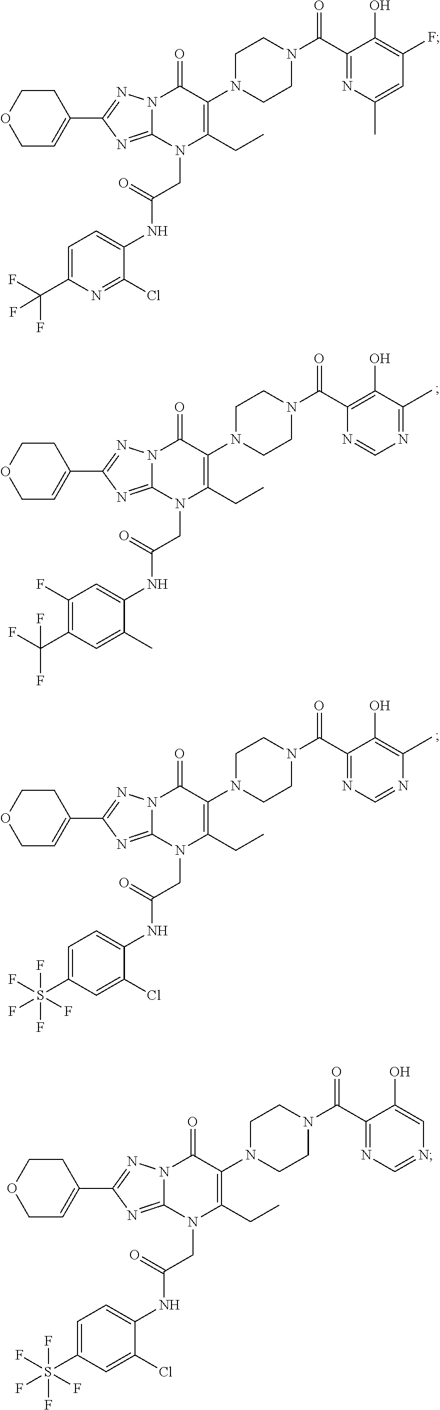 [SMILES: CCc1c(N2CCN(C(=O)c3nc(C)cc(F)c3O)CC2)c(=O)n2nc(C3=CCOCC3)nc2n1CC(=O)Nc1ccc(C(F)(F)F)nc1Cl ; CCc1c(N2CCN(C(=O)c3ncnc(C)c3O)CC2)c(=O)n2nc(C3=CCOCC3)nc2n1CC(=O)Nc1cc(F)c(C(F)(F)F)cc1C ; CCc1c(N2CCN(C(=O)c3ncnc(C)c3O)CC2)c(=O)n2nc(C3=CCOCC3)nc2n1CC(=O)Nc1ccc(S(F)(F)(F)(F)F)cc1Cl ; CCc1c(N2CCN(C(=O)c3ncncc3O)CC2)c(=O)n2nc(C3=CCOCC3)nc2n1CC(=O)Nc1ccc(S(F)(F)(F)(F)F)cc1Cl]

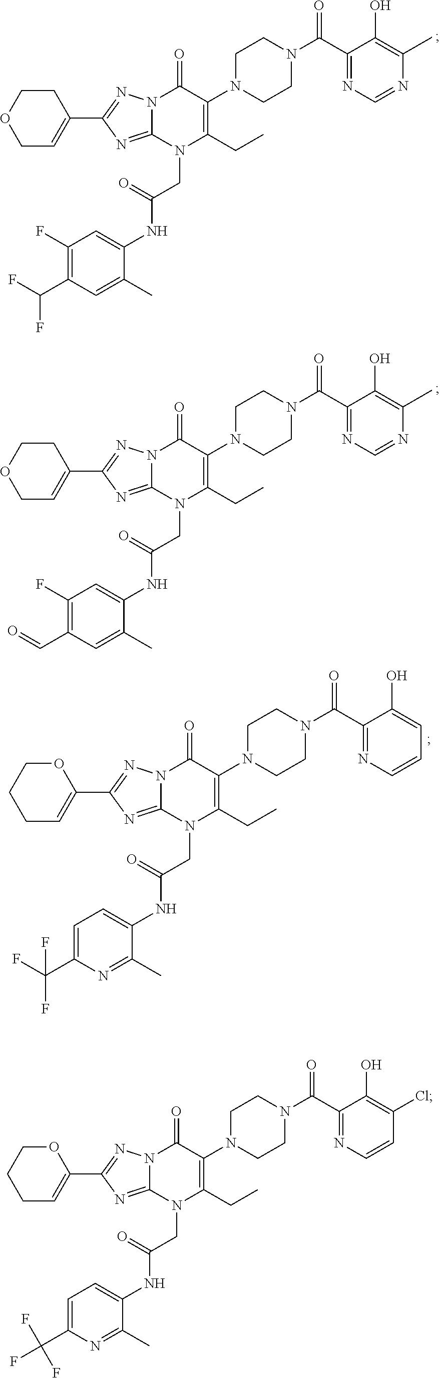 [SMILES: CCc1c(N2CCN(C(=O)c3ncnc(C)c3O)CC2)c(=O)n2nc(C3=CCOCC3)nc2n1CC(=O)Nc1cc(F)c(C(F)F)cc1C ; CCc1c(N2CCN(C(=O)c3ncnc(C)c3O)CC2)c(=O)n2nc(C3=CCOCC3)nc2n1CC(=O)Nc1cc(F)c(C=O)cc1C ; CCc1c(N2CCN(C(=O)c3ncccc3O)CC2)c(=O)n2nc(C3=CCCCO3)nc2n1CC(=O)Nc1ccc(C(F)(F)F)nc1C ; CCc1c(N2CCN(C(=O)c3nccc(Cl)c3O)CC2)c(=O)n2nc(C3=CCCCO3)nc2n1CC(=O)Nc1ccc(C(F)(F)F)nc1C]

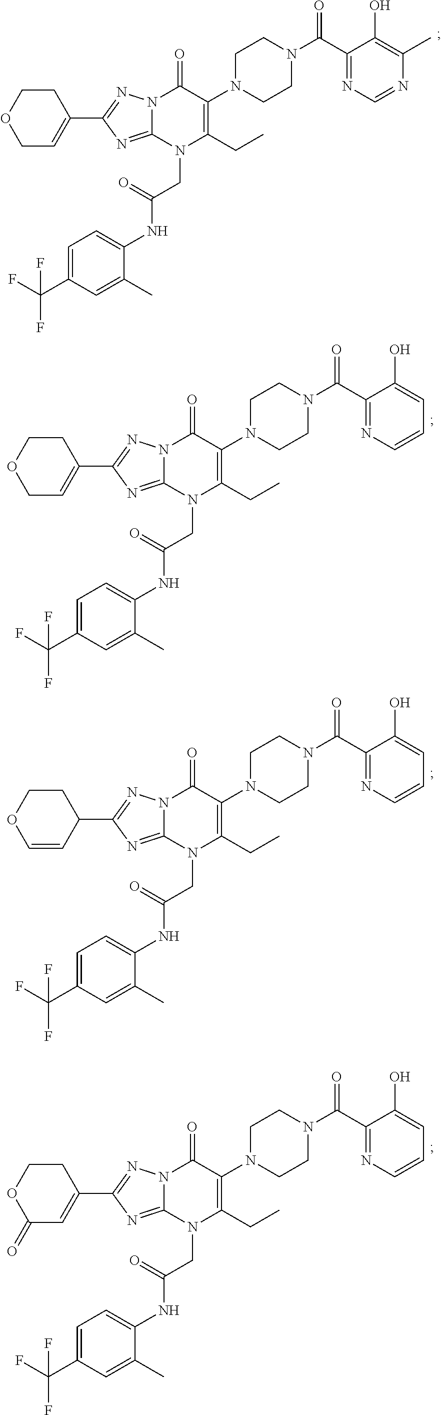 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1C ; CCc1c(N2CCN(C(=O)c3ncccc3O)CC2)c(=O)n2nc(C3=CCOCC3)nc2n1CC(=O)Nc1ccc(C(F)(F)F)cc1C ; CCc1c(N2CCN(C(=O)c3ncccc3O)CC2)c(=O)n2nc(C3C=COCC3)nc2n1CC(=O)Nc1ccc(C(F)(F)F)cc1C ; CCc1c(N2CCN(C(=O)c3ncccc3O)CC2)c(=O)n2nc(C3=CC(=O)OCC3)nc2n1CC(=O)Nc1ccc(C(F)(F)F)cc1C]

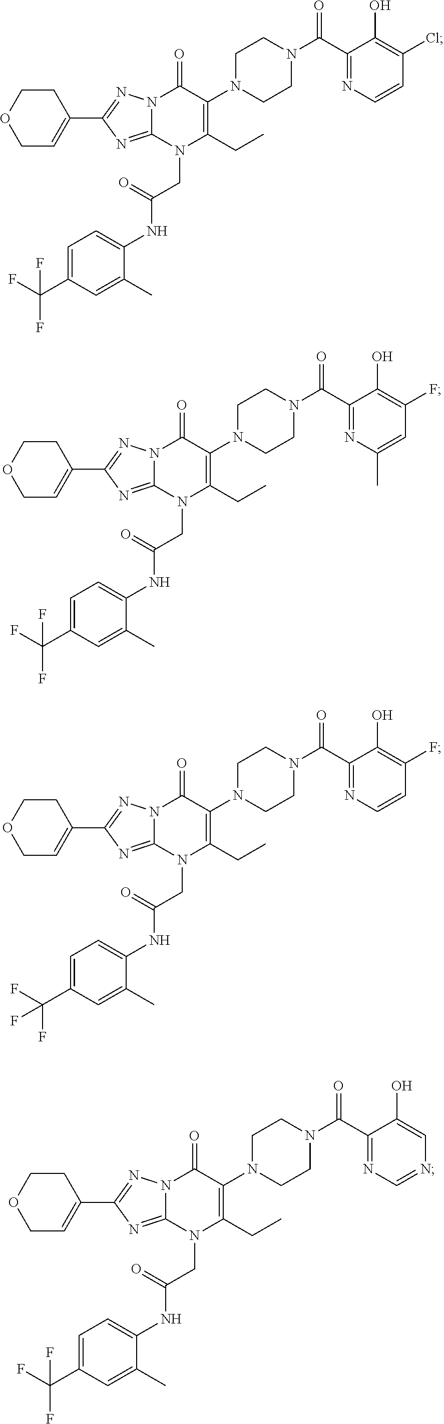 [SMILES: CCc1c(N2CCN(C(=O)c3nccc(Cl)c3O)CC2)c(=O)n2nc(C3=CCOCC3)nc2n1CC(=O)Nc1ccc(C(F)(F)F)cc1C ; CCc1c(N2CCN(C(=O)c3nc(C)cc(F)c3O)CC2)c(=O)n2nc(C3=CCOCC3)nc2n1CC(=O)Nc1ccc(C(F)(F)F)cc1C ; CCc1c(N2CCN(C(=O)c3nccc(F)c3O)CC2)c(=O)n2nc(C3=CCOCC3)nc2n1CC(=O)Nc1ccc(C(F)(F)F)cc1C ; CCc1c(N2CCN(C(=O)c3ncncc3O)CC2)c(=O)n2nc(C3=CCOCC3)nc2n1CC(=O)Nc1ccc(C(F)(F)F)cc1C]

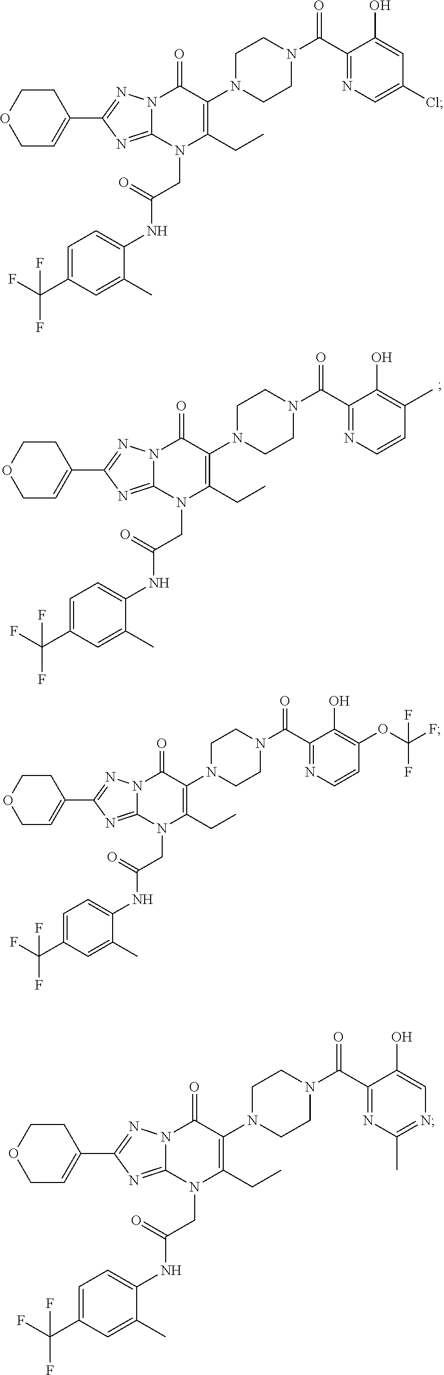 [SMILES: CCc1c(N2CCN(C(=O)c3ncc(Cl)cc3O)CC2)c(=O)n2nc(C3=CCOCC3)nc2n1CC(=O)Nc1ccc(C(F)(F)F)cc1C ; CCc1c(N2CCN(C(=O)c3nccc(C)c3O)CC2)c(=O)n2nc(C3=CCOCC3)nc2n1CC(=O)Nc1ccc(C(F)(F)F)cc1C ; CCc1c(N2CCN(C(=O)c3nccc(OC(F)(F)F)c3O)CC2)c(=O)n2nc(C3=CCOCC3)nc2n1CC(=O)Nc1ccc(C(F)(F)F)cc1C ; CCc1c(N2CCN(C(=O)c3nc(C)ncc3O)CC2)c(=O)n2nc(C3=CCOCC3)nc2n1CC(=O)Nc1ccc(C(F)(F)F)cc1C]

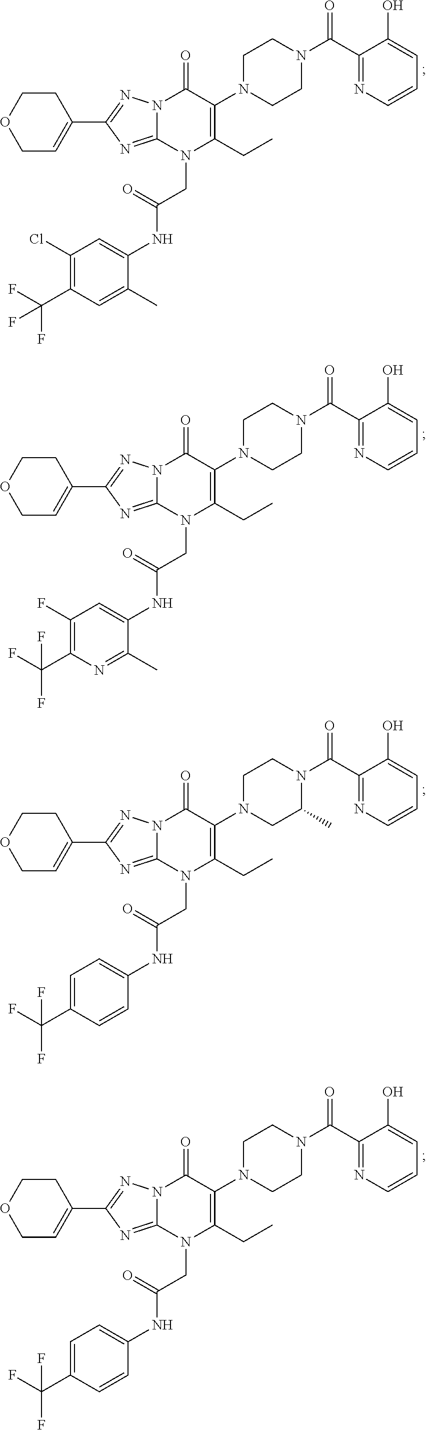 [SMILES: CCc1c(N2CCN(C(=O)c3ncccc3O)CC2)c(=O)n2nc(C3=CCOCC3)nc2n1CC(=O)Nc1cc(Cl)c(C(F)(F)F)cc1C ; CCc1c(N2CCN(C(=O)c3ncccc3O)CC2)c(=O)n2nc(C3=CCOCC3)nc2n1CC(=O)Nc1cc(F)c(C(F)(F)F)nc1C ; CCc1c(N2CCN(C(=O)c3ncccc3O)[C@H](C)C2)c(=O)n2nc(C3=CCOCC3)nc2n1CC(=O)Nc1ccc(C(F)(F)F)cc1 ; CCc1c(N2CCN(C(=O)c3ncccc3O)CC2)c(=O)n2nc(C3=CCOCC3)nc2n1CC(=O)Nc1ccc(C(F)(F)F)cc1]

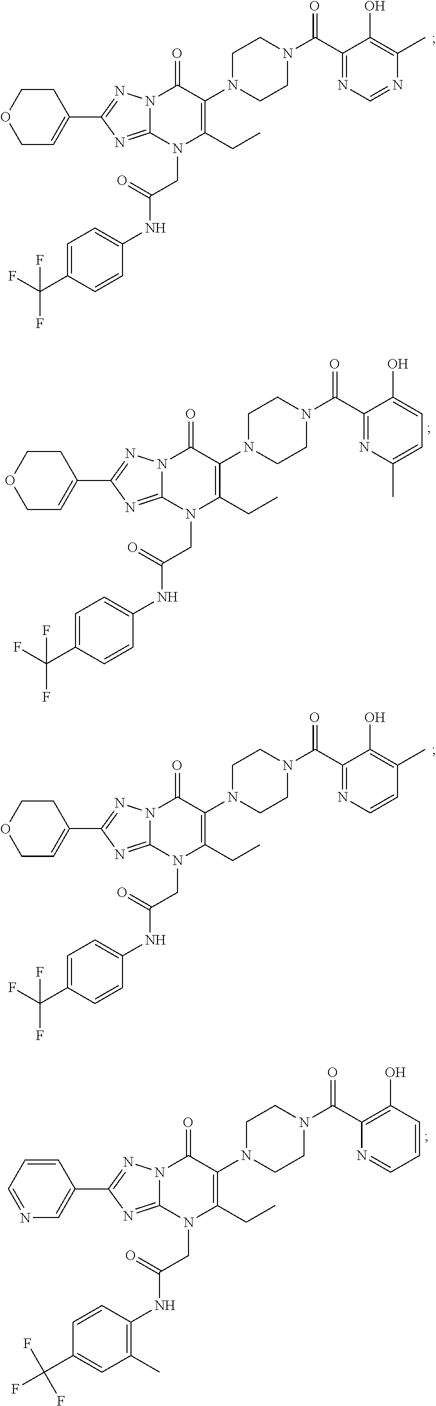 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1 ; CCc1c(N2CCN(C(=O)c3nc(C)ccc3O)CC2)c(=O)n2nc(C3=CCOCC3)nc2n1CC(=O)Nc1ccc(C(F)(F)F)cc1 ; CCc1c(N2CCN(C(=O)c3nccc(C)c3O)CC2)c(=O)n2nc(C3=CCOCC3)nc2n1CC(=O)Nc1ccc(C(F)(F)F)cc1 ; CCc1c(N2CCN(C(=O)c3ncccc3O)CC2)c(=O)n2nc(-c3cccnc3)nc2n1CC(=O)Nc1ccc(C(F)(F)F)cc1C]

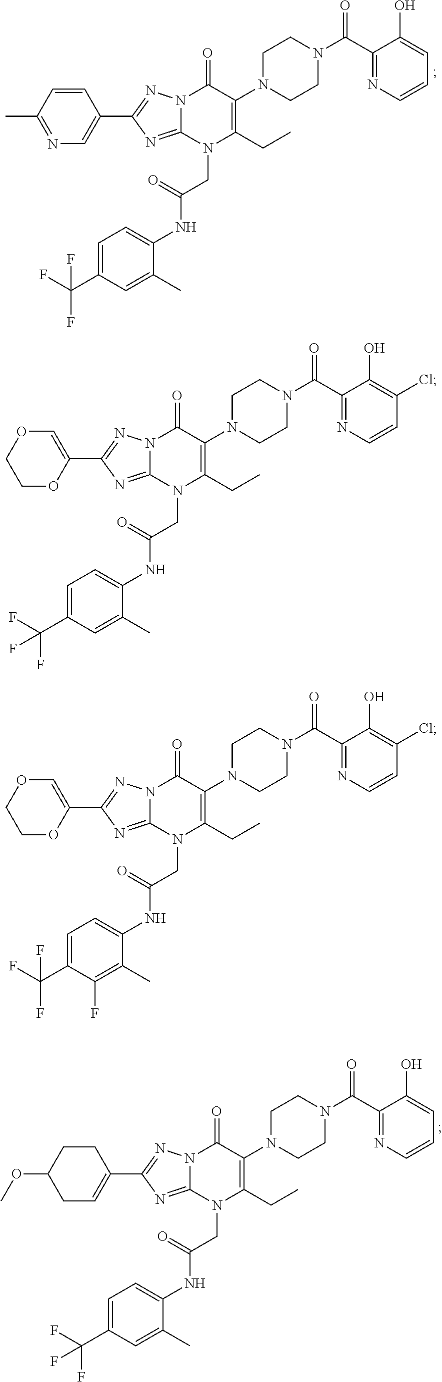 [SMILES: CCc1c(N2CCN(C(=O)c3ncccc3O)CC2)c(=O)n2nc(-c3ccc(C)nc3)nc2n1CC(=O)Nc1ccc(C(F)(F)F)cc1C ; CCc1c(N2CCN(C(=O)c3nccc(Cl)c3O)CC2)c(=O)n2nc(C3=COCCO3)nc2n1CC(=O)Nc1ccc(C(F)(F)F)cc1C ; CCc1c(N2CCN(C(=O)c3nccc(Cl)c3O)CC2)c(=O)n2nc(C3=COCCO3)nc2n1CC(=O)Nc1ccc(C(F)(F)F)c(F)c1C ; CCc1c(N2CCN(C(=O)c3ncccc3O)CC2)c(=O)n2nc(C3=CCC(OC)CC3)nc2n1CC(=O)Nc1ccc(C(F)(F)F)cc1C]

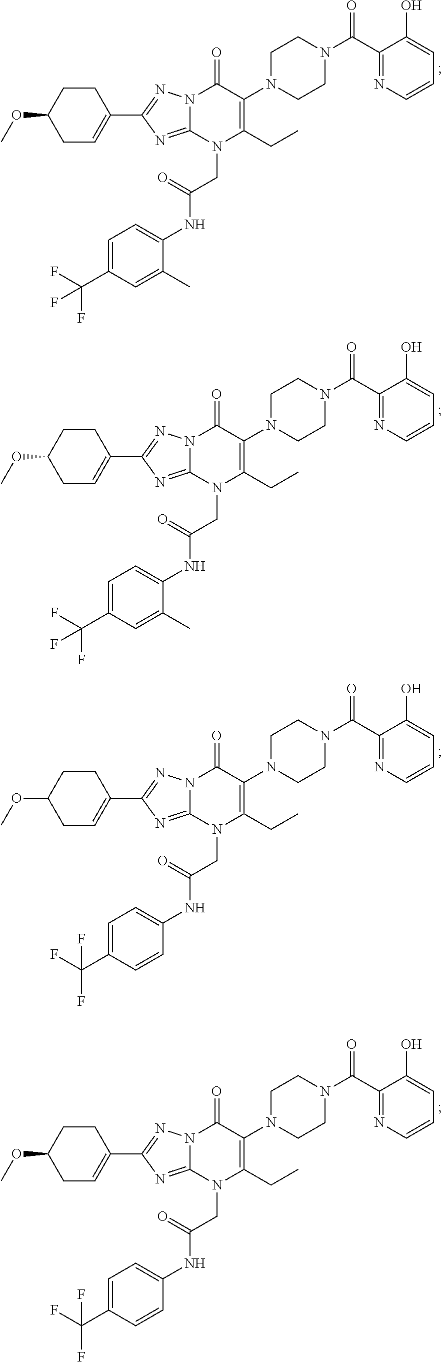 [SMILES: CCc1c(N2CCN(C(=O)c3ncccc3O)CC2)c(=O)n2nc(C3=CC[C@@H](OC)CC3)nc2n1CC(=O)Nc1ccc(C(F)(F)F)cc1C ; CCc1c(N2CCN(C(=O)c3ncccc3O)CC2)c(=O)n2nc(C3=CC[C@H](OC)CC3)nc2n1CC(=O)Nc1ccc(C(F)(F)F)cc1C ; CCc1c(N2CCN(C(=O)c3ncccc3O)CC2)c(=O)n2nc(C3=CCC(OC)CC3)nc2n1CC(=O)Nc1ccc(C(F)(F)F)cc1 ; CCc1c(N2CCN(C(=O)c3ncccc3O)CC2)c(=O)n2nc(C3=CC[C@@H](OC)CC3)nc2n1CC(=O)Nc1ccc(C(F)(F)F)cc1]

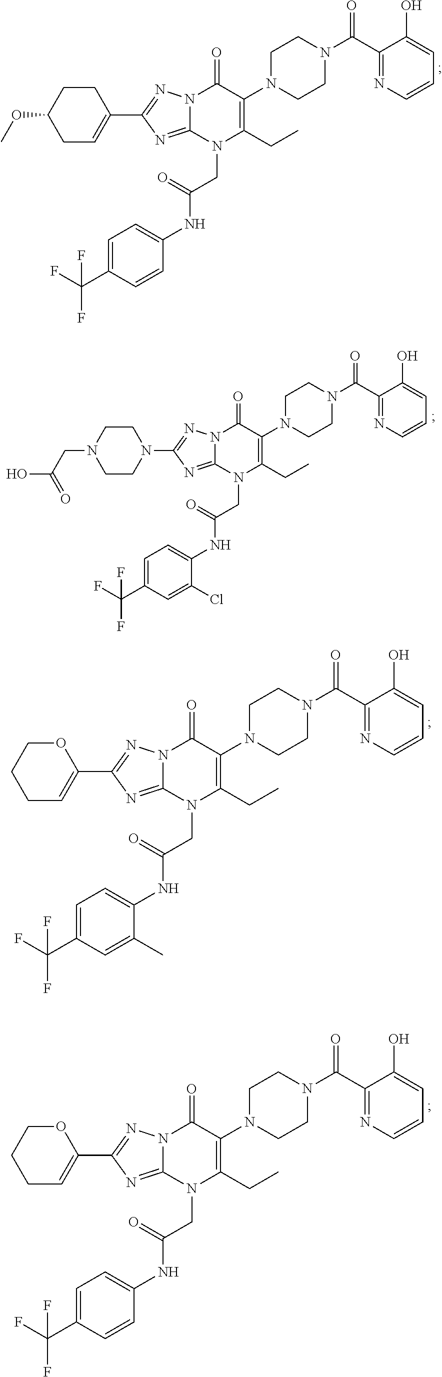 [SMILES: CCc1c(N2CCN(C(=O)c3ncccc3O)CC2)c(=O)n2nc(C3=CC[C@H](OC)CC3)nc2n1CC(=O)Nc1ccc(C(F)(F)F)cc1 ; CCc1c(N2CCN(C(=O)c3ncccc3O)CC2)c(=O)n2nc(N3CCN(CC(=O)O)CC3)nc2n1CC(=O)Nc1ccc(C(F)(F)F)cc1Cl ; CCc1c(N2CCN(C(=O)c3ncccc3O)CC2)c(=O)n2nc(C3=CCCCO3)nc2n1CC(=O)Nc1ccc(C(F)(F)F)cc1C ; CCc1c(N2CCN(C(=O)c3ncccc3O)CC2)c(=O)n2nc(C3=CCCCO3)nc2n1CC(=O)Nc1ccc(C(F)(F)F)cc1]

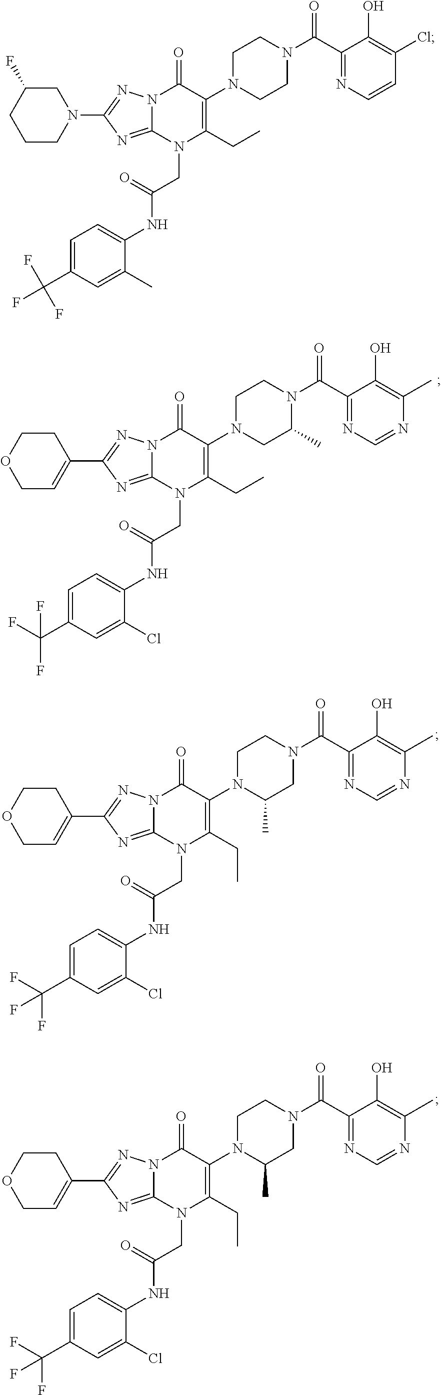 [SMILES: CCc1c(N2CCN(C(=O)c3nccc(Cl)c3O)CC2)c(=O)n2nc(N3CCC[C@H](F)C3)nc2n1CC(=O)Nc1ccc(C(F)(F)F)cc1C ; CCc1c(N2CCN(C(=O)c3ncnc(C)c3O)[C@H](C)C2)c(=O)n2nc(C3=CCOCC3)nc2n1CC(=O)Nc1ccc(C(F)(F)F)cc1Cl ; CCc1c(N2CCN(C(=O)c3ncnc(C)c3O)C[C@@H]2C)c(=O)n2nc(C3=CCOCC3)nc2n1CC(=O)Nc1ccc(C(F)(F)F)cc1Cl ; CCc1c(N2CCN(C(=O)c3ncnc(C)c3O)C[C@H]2C)c(=O)n2nc(C3=CCOCC3)nc2n1CC(=O)Nc1ccc(C(F)(F)F)cc1Cl]

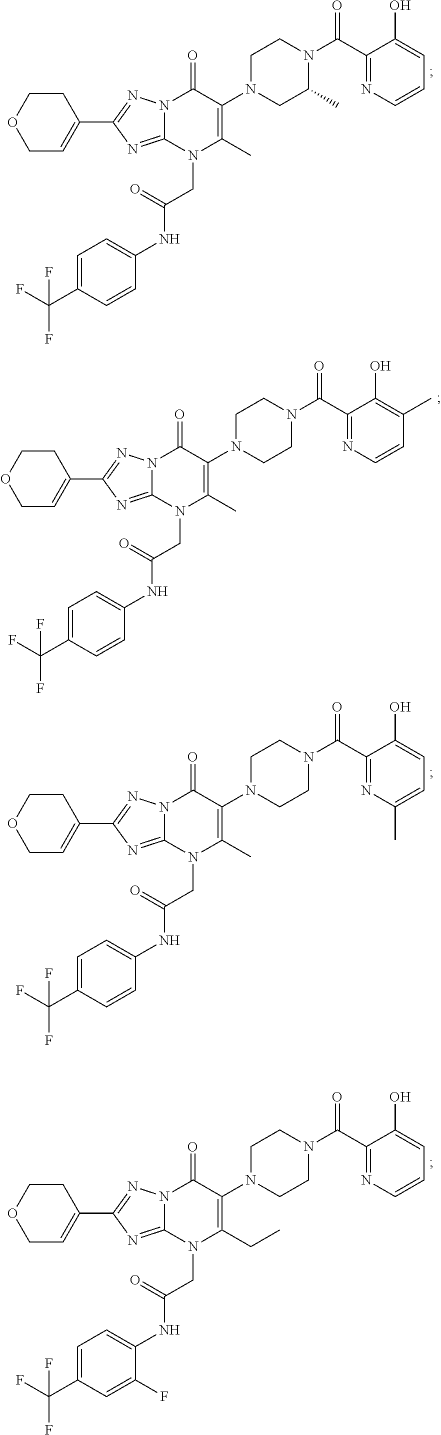 [SMILES: Cc1c(N2CCN(C(=O)c3ncccc3O)[C@H](C)C2)c(=O)n2nc(C3=CCOCC3)nc2n1CC(=O)Nc1ccc(C(F)(F)F)cc1 ; Cc1ccnc(C(=O)N2CCN(c3c(C)n(CC(=O)Nc4ccc(C(F)(F)F)cc4)c4nc(C5=CCOCC5)nn4c3=O)CC2)c1O ; Cc1ccc(O)c(C(=O)N2CCN(c3c(C)n(CC(=O)Nc4ccc(C(F)(F)F)cc4)c4nc(C5=CCOCC5)nn4c3=O)CC2)n1 ; CCc1c(N2CCN(C(=O)c3ncccc3O)CC2)c(=O)n2nc(C3=CCOCC3)nc2n1CC(=O)Nc1ccc(C(F)(F)F)cc1F]

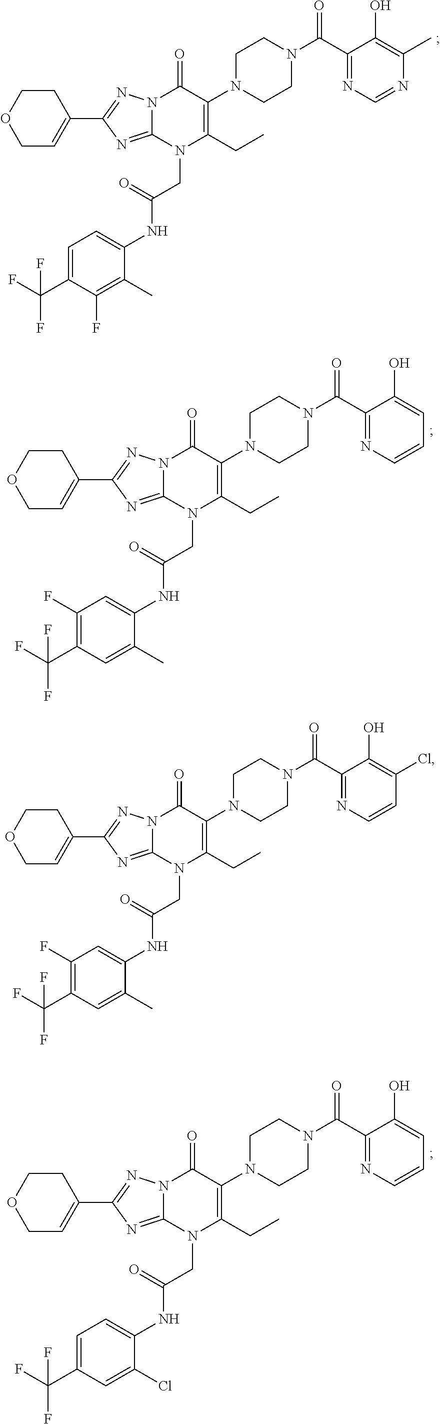 [SMILES: CCc1c(N2CCN(C(=O)c3ncnc(C)c3O)CC2)c(=O)n2nc(C3=CCOCC3)nc2n1CC(=O)Nc1ccc(C(F)(F)F)c(F)c1C ; CCc1c(N2CCN(C(=O)c3ncccc3O)CC2)c(=O)n2nc(C3=CCOCC3)nc2n1CC(=O)Nc1cc(F)c(C(F)(F)F)cc1C ; CCc1c(N2CCN(C(=O)c3nccc(Cl)c3O)CC2)c(=O)n2nc(C3=CCOCC3)nc2n1CC(=O)Nc1cc(F)c(C(F)(F)F)cc1C ; CCc1c(N2CCN(C(=O)c3ncccc3O)CC2)c(=O)n2nc(C3=CCOCC3)nc2n1CC(=O)Nc1ccc(C(F)(F)F)cc1Cl]

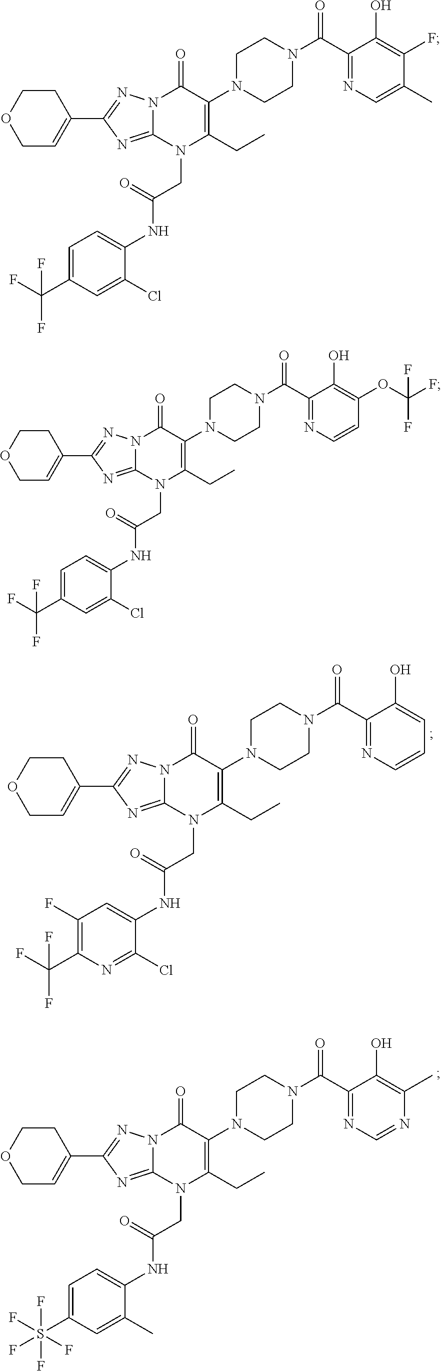 [SMILES: CCc1c(N2CCN(C(=O)c3ncc(C)c(F)c3O)CC2)c(=O)n2nc(C3=CCOCC3)nc2n1CC(=O)Nc1ccc(C(F)(F)F)cc1Cl ; CCc1c(N2CCN(C(=O)c3nccc(OC(F)(F)F)c3O)CC2)c(=O)n2nc(C3=CCOCC3)nc2n1CC(=O)Nc1ccc(C(F)(F)F)cc1Cl ; CCc1c(N2CCN(C(=O)c3ncccc3O)CC2)c(=O)n2nc(C3=CCOCC3)nc2n1CC(=O)Nc1cc(F)c(C(F)(F)F)nc1Cl ; CCc1c(N2CCN(C(=O)c3ncnc(C)c3O)CC2)c(=O)n2nc(C3=CCOCC3)nc2n1CC(=O)Nc1ccc(S(F)(F)(F)(F)F)cc1C]

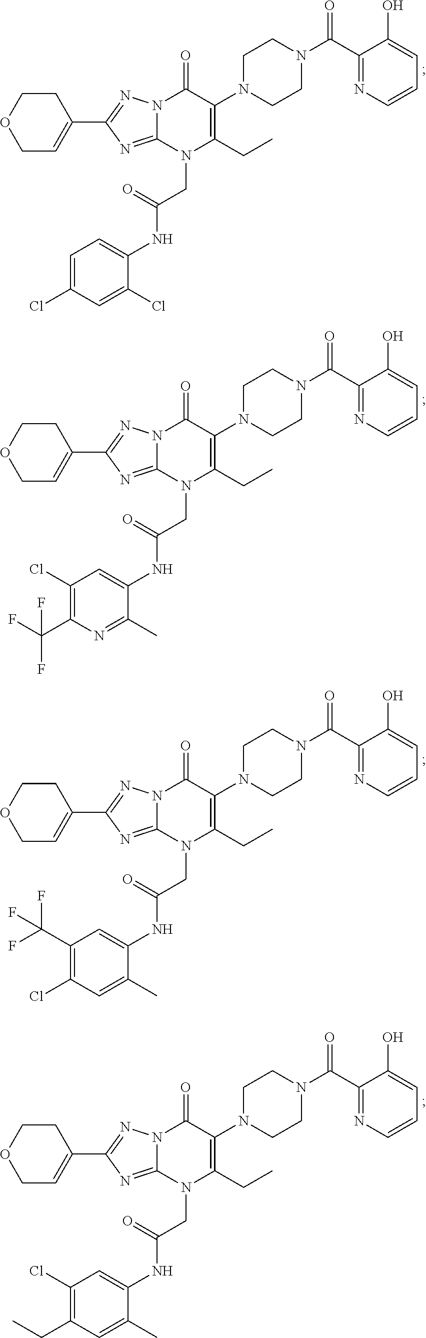 [SMILES: CCc1c(N2CCN(C(=O)c3ncccc3O)CC2)c(=O)n2nc(C3=CCOCC3)nc2n1CC(=O)Nc1ccc(Cl)cc1Cl ; CCc1c(N2CCN(C(=O)c3ncccc3O)CC2)c(=O)n2nc(C3=CCOCC3)nc2n1CC(=O)Nc1cc(Cl)c(C(F)(F)F)nc1C ; CCc1c(N2CCN(C(=O)c3ncccc3O)CC2)c(=O)n2nc(C3=CCOCC3)nc2n1CC(=O)Nc1cc(C(F)(F)F)c(Cl)cc1C ; CCc1cc(C)c(NC(=O)Cn2c(CC)c(N3CCN(C(=O)c4ncccc4O)CC3)c(=O)n3nc(C4=CCOCC4)nc23)cc1Cl]

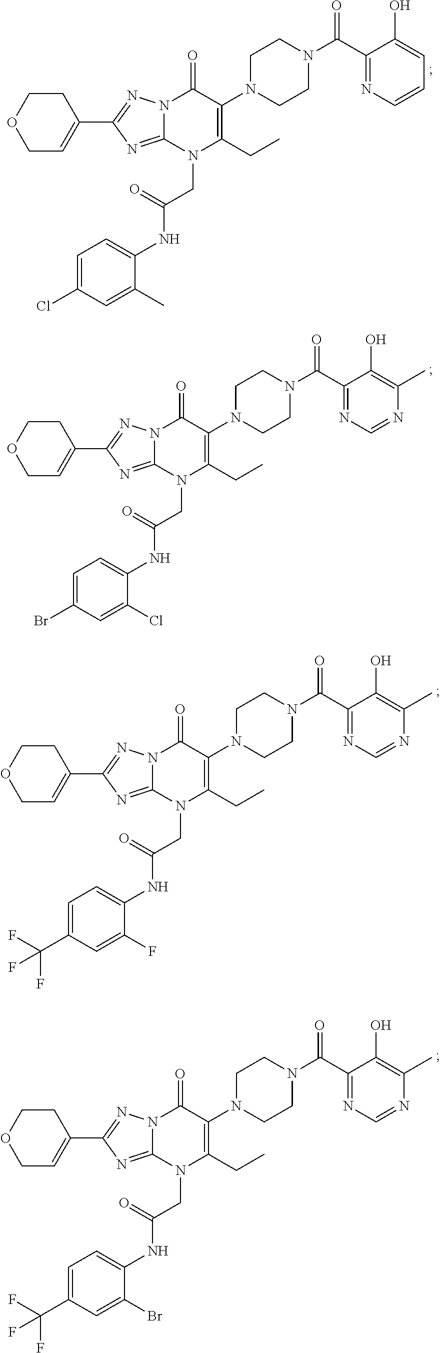 [SMILES: CCc1c(N2CCN(C(=O)c3ncccc3O)CC2)c(=O)n2nc(C3=CCOCC3)nc2n1CC(=O)Nc1ccc(Cl)cc1C ; CCc1c(N2CCN(C(=O)c3ncnc(C)c3O)CC2)c(=O)n2nc(C3=CCOCC3)nc2n1CC(=O)Nc1ccc(Br)cc1Cl ; CCc1c(N2CCN(C(=O)c3ncnc(C)c3O)CC2)c(=O)n2nc(C3=CCOCC3)nc2n1CC(=O)Nc1ccc(C(F)(F)F)cc1F ; CCc1c(N2CCN(C(=O)c3ncnc(C)c3O)CC2)c(=O)n2nc(C3=CCOCC3)nc2n1CC(=O)Nc1ccc(C(F)(F)F)cc1Br]

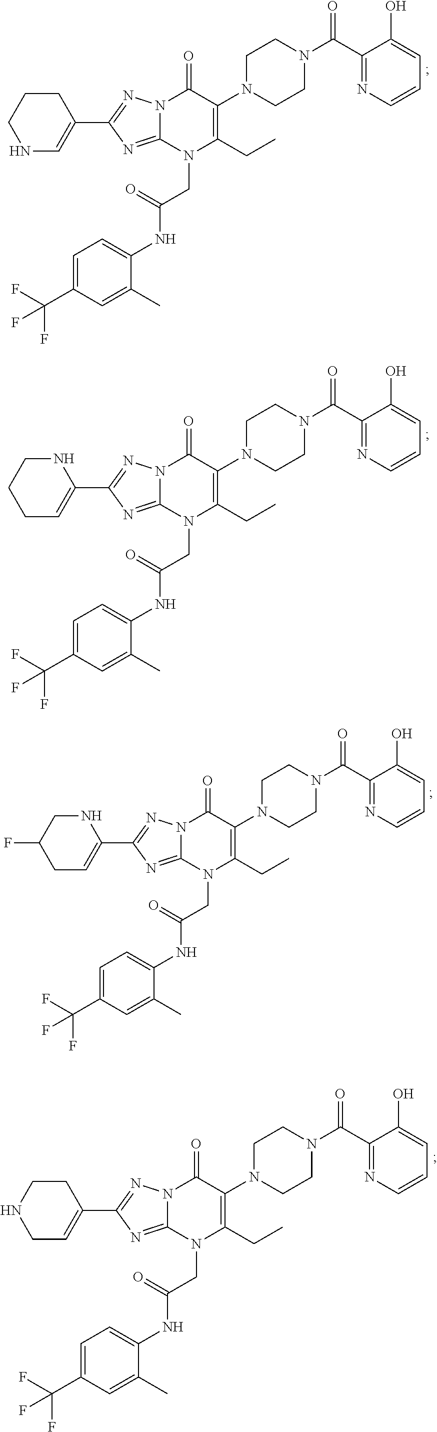 [SMILES: CCc1c(N2CCN(C(=O)c3ncccc3O)CC2)c(=O)n2nc(C3=CNCCC3)nc2n1CC(=O)Nc1ccc(C(F)(F)F)cc1C ; CCc1c(N2CCN(C(=O)c3ncccc3O)CC2)c(=O)n2nc(C3=CCCCN3)nc2n1CC(=O)Nc1ccc(C(F)(F)F)cc1C ; CCc1c(N2CCN(C(=O)c3ncccc3O)CC2)c(=O)n2nc(C3=CCC(F)CN3)nc2n1CC(=O)Nc1ccc(C(F)(F)F)cc1C ; CCc1c(N2CCN(C(=O)c3ncccc3O)CC2)c(=O)n2nc(C3=CCNCC3)nc2n1CC(=O)Nc1ccc(C(F)(F)F)cc1C]

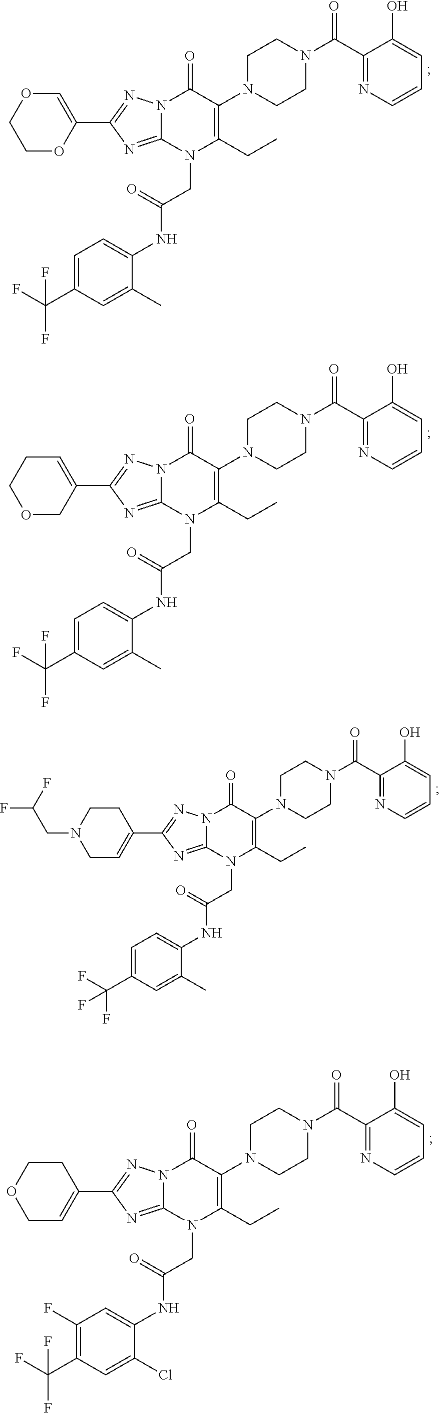 [SMILES: CCc1c(N2CCN(C(=O)c3ncccc3O)CC2)c(=O)n2nc(C3=COCCO3)nc2n1CC(=O)Nc1ccc(C(F)(F)F)cc1C ; CCc1c(N2CCN(C(=O)c3ncccc3O)CC2)c(=O)n2nc(C3=CCCOC3)nc2n1CC(=O)Nc1ccc(C(F)(F)F)cc1C ; CCc1c(N2CCN(C(=O)c3ncccc3O)CC2)c(=O)n2nc(C3=CCN(CC(F)F)CC3)nc2n1CC(=O)Nc1ccc(C(F)(F)F)cc1C ; CCc1c(N2CCN(C(=O)c3ncccc3O)CC2)c(=O)n2nc(C3=CCOCC3)nc2n1CC(=O)Nc1cc(F)c(C(F)(F)F)cc1Cl]

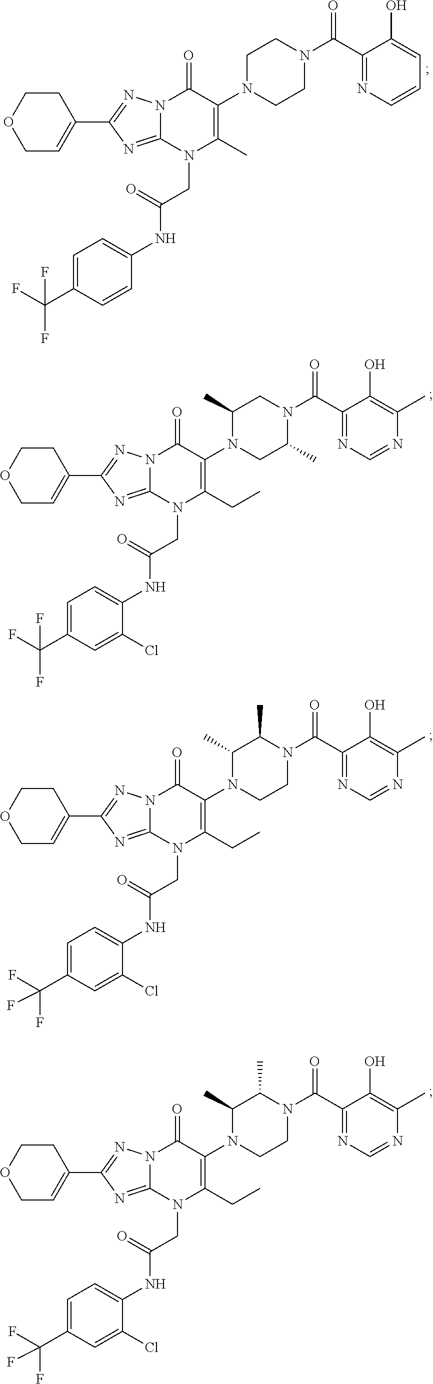 [SMILES: Cc1c(N2CCN(C(=O)c3ncccc3O)CC2)c(=O)n2nc(C3=CCOCC3)nc2n1CC(=O)Nc1ccc(C(F)(F)F)cc1 ; CCc1c(N2C[C@@H](C)N(C(=O)c3ncnc(C)c3O)C[C@@H]2C)c(=O)n2nc(C3=CCOCC3)nc2n1CC(=O)Nc1ccc(C(F)(F)F)cc1Cl ; CCc1c(N2CCN(C(=O)c3ncnc(C)c3O)[C@H](C)[C@H]2C)c(=O)n2nc(C3=CCOCC3)nc2n1CC(=O)Nc1ccc(C(F)(F)F)cc1Cl ; CCc1c(N2CCN(C(=O)c3ncnc(C)c3O)[C@@H](C)[C@@H]2C)c(=O)n2nc(C3=CCOCC3)nc2n1CC(=O)Nc1ccc(C(F)(F)F)cc1Cl]

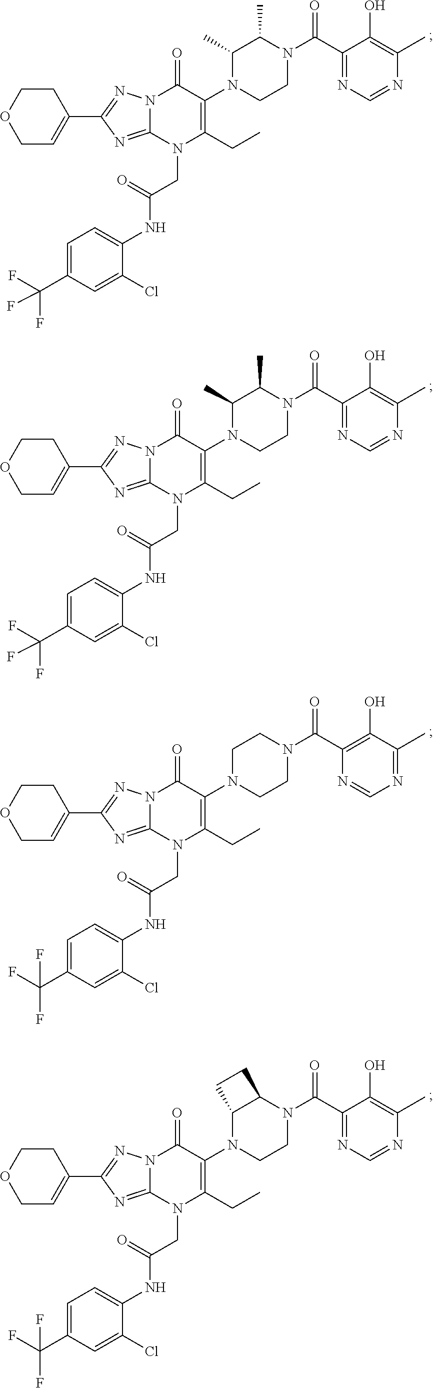 [SMILES: CCc1c(N2CCN(C(=O)c3ncnc(C)c3O)[C@@H](C)[C@H]2C)c(=O)n2nc(C3=CCOCC3)nc2n1CC(=O)Nc1ccc(C(F)(F)F)cc1Cl ; CCc1c(N2CCN(C(=O)c3ncnc(C)c3O)[C@H](C)[C@@H]2C)c(=O)n2nc(C3=CCOCC3)nc2n1CC(=O)Nc1ccc(C(F)(F)F)cc1Cl ; CCc1c(N2CCN(C(=O)c3ncnc(C)c3O)CC2)c(=O)n2nc(C3=CCOCC3)nc2n1CC(=O)Nc1ccc(C(F)(F)F)cc1Cl ; CCc1c(N2CCN(C(=O)c3ncnc(C)c3O)[C@@H]3CC[C@H]32)c(=O)n2nc(C3=CCOCC3)nc2n1CC(=O)Nc1ccc(C(F)(F)F)cc1Cl]

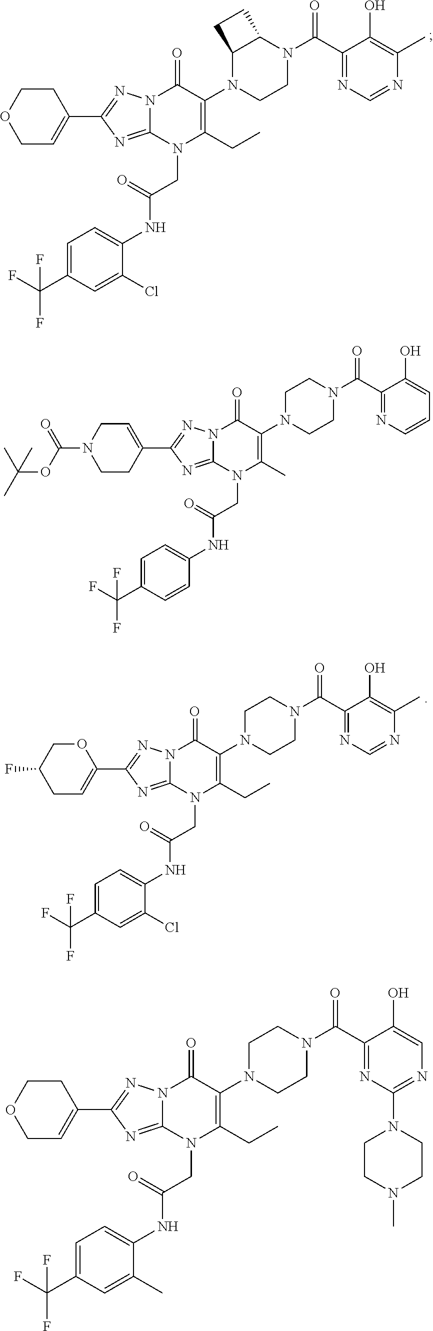

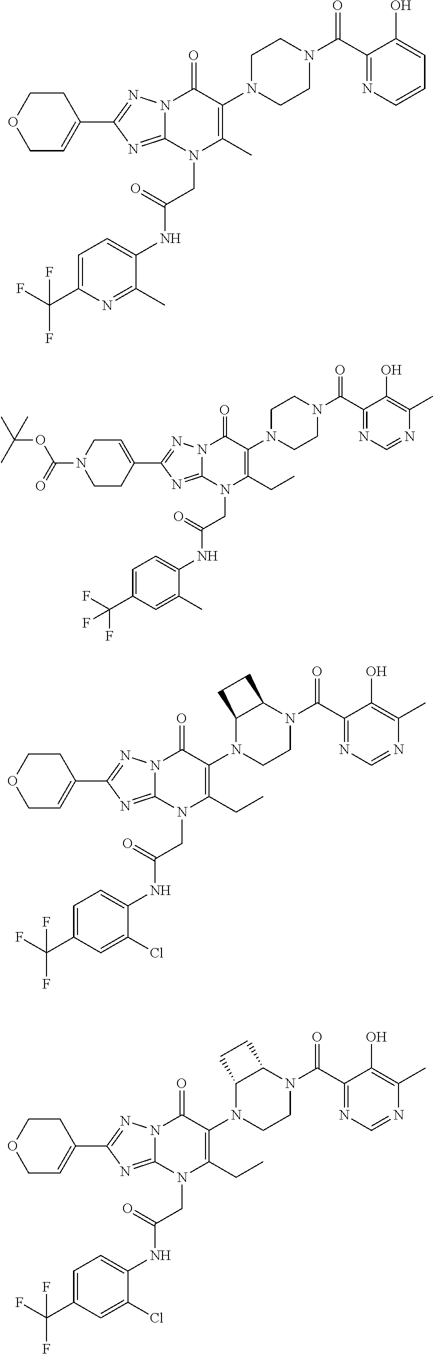 [SMILES: Cc1nc(C(F)(F)F)ccc1NC(=O)Cn1c(C)c(N2CCN(C(=O)c3ncccc3O)CC2)c(=O)n2nc(C3=CCOCC3)nc12 ; CCc1c(N2CCN(C(=O)c3ncnc(C)c3O)CC2)c(=O)n2nc(C3=CCN(C(=O)OC(C)(C)C)CC3)nc2n1CC(=O)Nc1ccc(C(F)(F)F)cc1C ; CCc1c(N2CCN(C(=O)c3ncnc(C)c3O)[C@@H]3CC[C@@H]32)c(=O)n2nc(C3=CCOCC3)nc2n1CC(=O)Nc1ccc(C(F)(F)F)cc1Cl ; CCc1c(N2CCN(C(=O)c3ncnc(C)c3O)[C@H]3CC[C@H]32)c(=O)n2nc(C3=CCOCC3)nc2n1CC(=O)Nc1ccc(C(F)(F)F)cc1Cl]

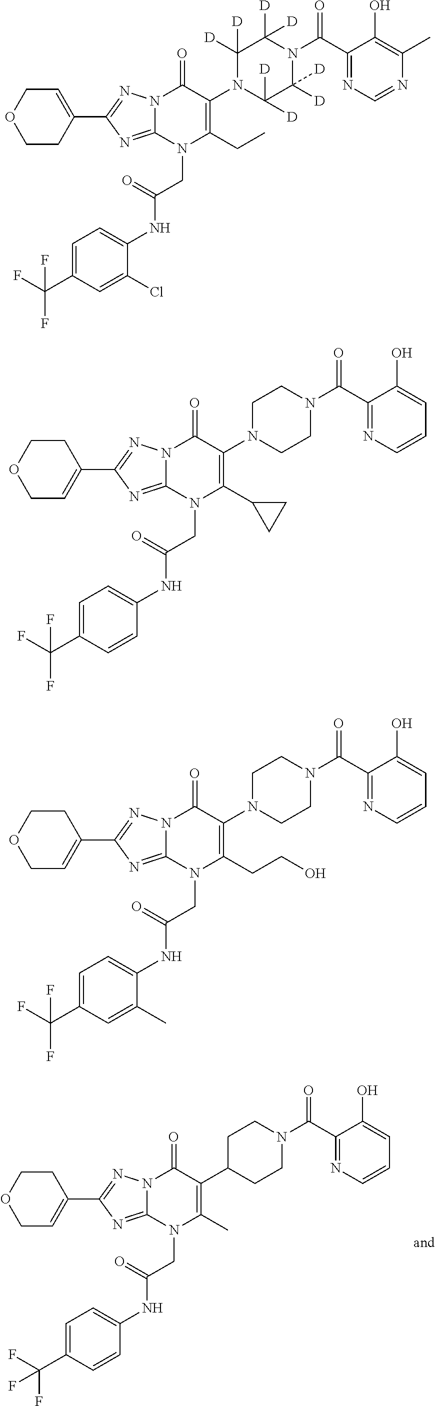

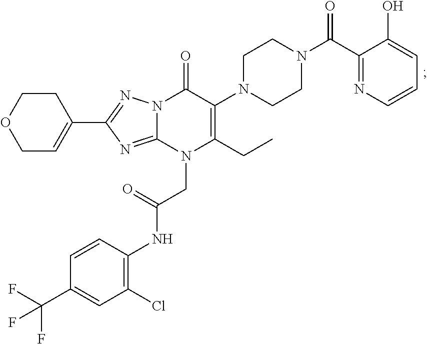 [SMILES: CCc1c(N2CCN(C(=O)c3ncccc3O)CC2)c(=O)n2nc(C3=CCOCC3)nc2n1CC(=O)Nc1ccc(C(F)(F)F)cc1Cl]

or a zwitterionic form or a salt thereof.

Embodiment 7. The method according to Embodiment 5, the WRN inhibitor for use according to Embodiment 5, the therapeutically active agent for use according to Embodiment 5, or the combination according to Embodiment 5, wherein the WRN inhibitor is selected from:

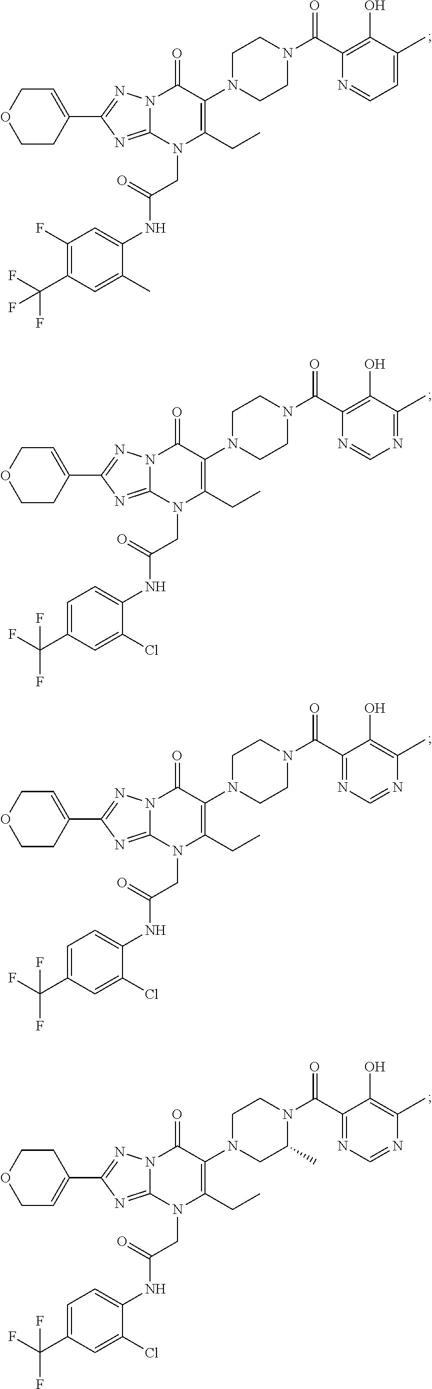 [SMILES: CCc1c(N2CCN(C(=O)c3nccc(C)c3O)CC2)c(=O)n2nc(C3=CCOCC3)nc2n1CC(=O)Nc1cc(F)c(C(F)(F)F)cc1C ; CCc1c(N2CCN(C(=O)c3ncnc(C)c3O)CC2)c(=O)n2nc(C3=CCOCC3)nc2n1CC(=O)Nc1ccc(C(F)(F)F)cc1Cl ; CCc1c(N2CCN(C(=O)c3ncnc(C)c3O)CC2)c(=O)n2nc(C3=CCOCC3)nc2n1CC(=O)Nc1ccc(C(F)(F)F)cc1Cl ; CCc1c(N2CCN(C(=O)c3ncnc(C)c3O)[C@H](C)C2)c(=O)n2nc(C3=CCOCC3)nc2n1CC(=O)Nc1ccc(C(F)(F)F)cc1Cl]

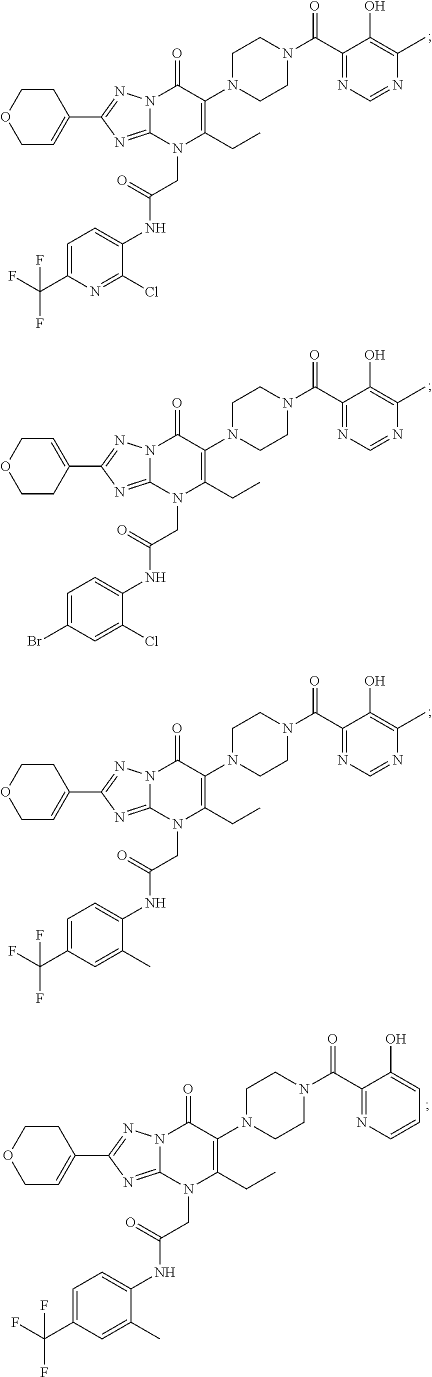 [SMILES: CCc1c(N2CCN(C(=O)c3ncnc(C)c3O)CC2)c(=O)n2nc(C3=CCOCC3)nc2n1CC(=O)Nc1ccc(C(F)(F)F)nc1Cl ; CCc1c(N2CCN(C(=O)c3ncnc(C)c3O)CC2)c(=O)n2nc(C3=CCOCC3)nc2n1CC(=O)Nc1ccc(Br)cc1Cl ; CCc1c(N2CCN(C(=O)c3ncnc(C)c3O)CC2)c(=O)n2nc(C3=CCOCC3)nc2n1CC(=O)Nc1ccc(C(F)(F)F)cc1C ; CCc1c(N2CCN(C(=O)c3ncccc3O)CC2)c(=O)n2nc(C3=CCOCC3)nc2n1CC(=O)Nc1ccc(C(F)(F)F)cc1C]

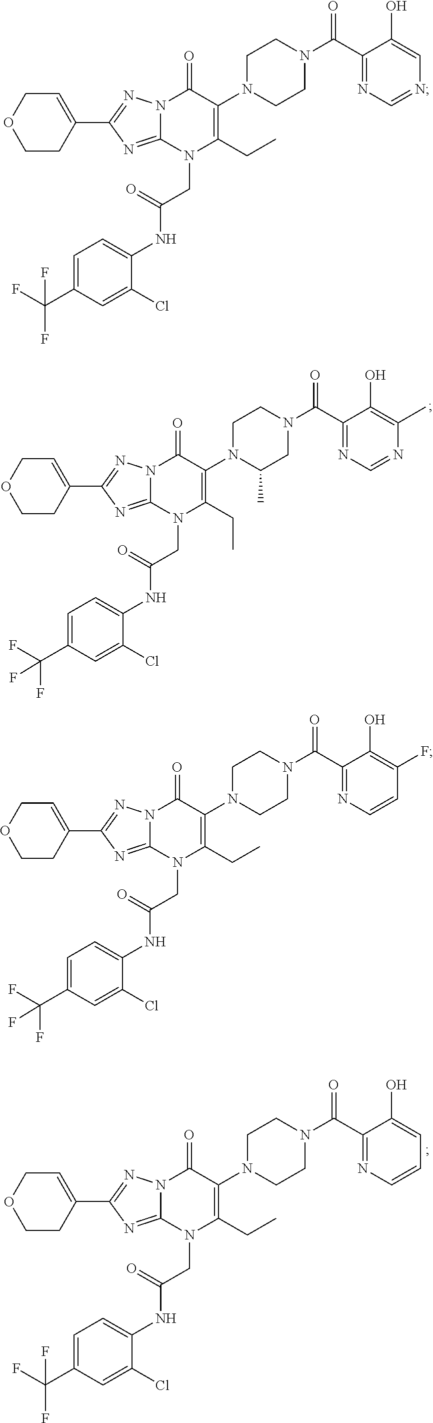 [SMILES: CCc1c(N2CCN(C(=O)c3ncncc3O)CC2)c(=O)n2nc(C3=CCOCC3)nc2n1CC(=O)Nc1ccc(C(F)(F)F)cc1Cl ; CCc1c(N2CCN(C(=O)c3ncnc(C)c3O)C[C@@H]2C)c(=O)n2nc(C3=CCOCC3)nc2n1CC(=O)Nc1ccc(C(F)(F)F)cc1Cl ; CCc1c(N2CCN(C(=O)c3nccc(F)c3O)CC2)c(=O)n2nc(C3=CCOCC3)nc2n1CC(=O)Nc1ccc(C(F)(F)F)cc1Cl ; CCc1c(N2CCN(C(=O)c3ncccc3O)CC2)c(=O)n2nc(C3=CCOCC3)nc2n1CC(=O)Nc1ccc(C(F)(F)F)cc1Cl]

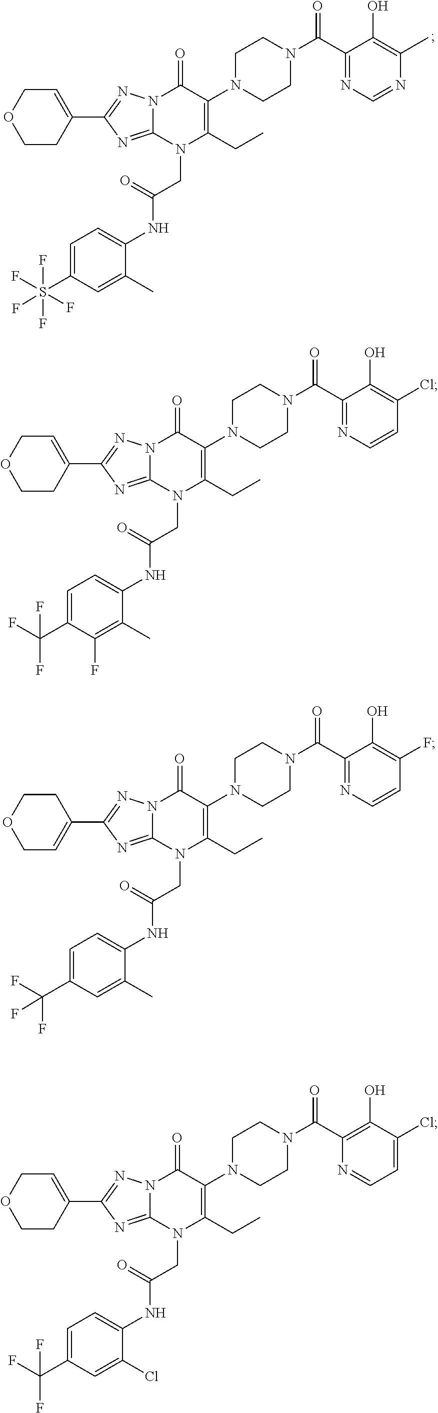 [SMILES: CCc1c(N2CCN(C(=O)c3ncnc(C)c3O)CC2)c(=O)n2nc(C3=CCOCC3)nc2n1CC(=O)Nc1ccc(S(F)(F)(F)(F)F)cc1C ; CCc1c(N2CCN(C(=O)c3nccc(Cl)c3O)CC2)c(=O)n2nc(C3=CCOCC3)nc2n1CC(=O)Nc1ccc(C(F)(F)F)c(F)c1C ; CCc1c(N2CCN(C(=O)c3nccc(F)c3O)CC2)c(=O)n2nc(C3=CCOCC3)nc2n1CC(=O)Nc1ccc(C(F)(F)F)cc1C ; CCc1c(N2CCN(C(=O)c3nccc(Cl)c3O)CC2)c(=O)n2nc(C3=CCOCC3)nc2n1CC(=O)Nc1ccc(C(F)(F)F)cc1Cl]

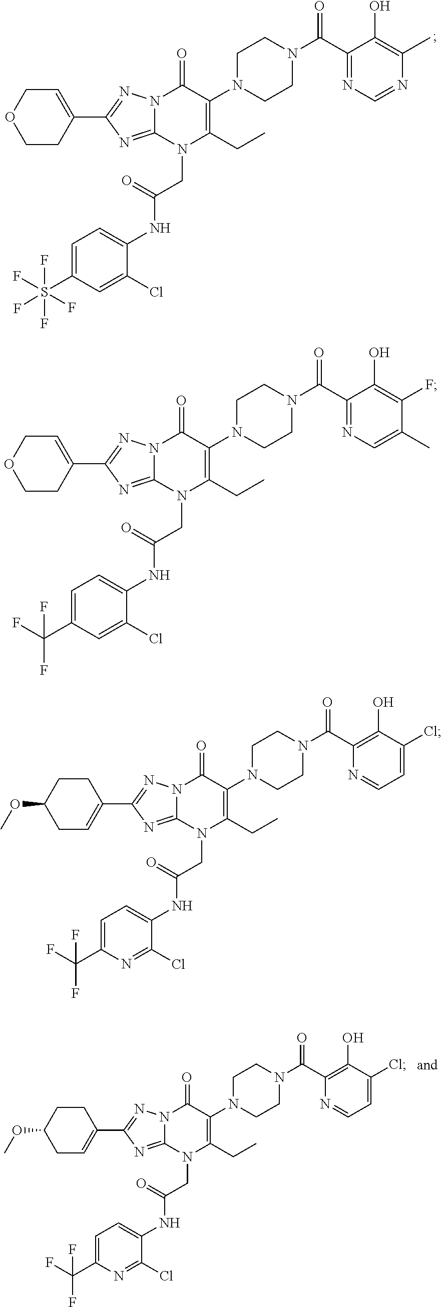

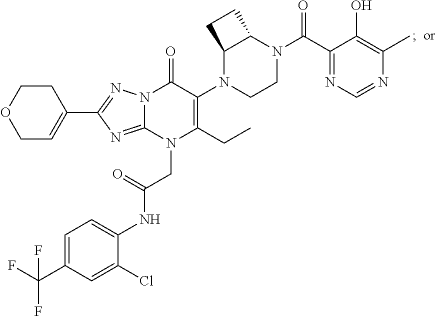 [SMILES: CCc1c(N2CCN(C(=O)c3ncnc(C)c3O)[C@H]3CC[C@@H]32)c(=O)n2nc(C3=CCOCC3)nc2n1CC(=O)Nc1ccc(C(F)(F)F)cc1Cl]

or a zwitterionic form or a salt thereof.

Embodiment 8. The method according to Embodiment 5, the WRN inhibitor for use according to Embodiment 5, the therapeutically active agent for use according to Embodiment 5, or the combination according to Embodiment 5, wherein the WRN inhibitor is selected from:

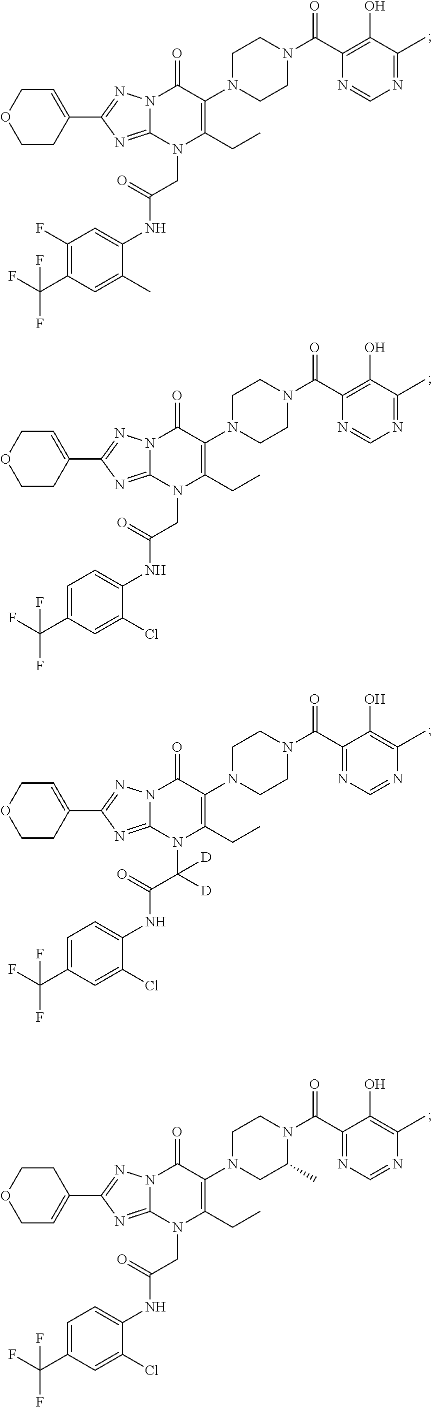 [SMILES: CCc1c(N2CCN(C(=O)c3ncnc(C)c3O)CC2)c(=O)n2nc(C3=CCOCC3)nc2n1CC(=O)Nc1cc(F)c(C(F)(F)F)cc1C ; CCc1c(N2CCN(C(=O)c3ncnc(C)c3O)CC2)c(=O)n2nc(C3=CCOCC3)nc2n1CC(=O)Nc1ccc(C(F)(F)F)cc1Cl ; [2H]C([2H])(C(=O)Nc1ccc(C(F)(F)F)cc1Cl)n1c(CC)c(N2CCN(C(=O)c3ncnc(C)c3O)CC2)c(=O)n2nc(C3=CCOCC3)nc12 ; CCc1c(N2CCN(C(=O)c3ncnc(C)c3O)[C@H](C)C2)c(=O)n2nc(C3=CCOCC3)nc2n1CC(=O)Nc1ccc(C(F)(F)F)cc1Cl]

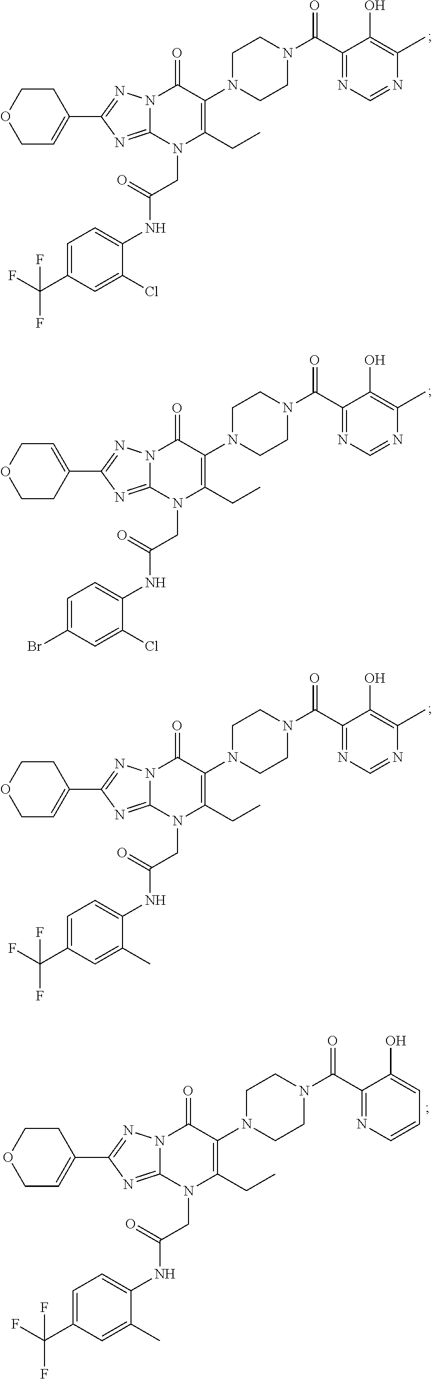 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl ; CCc1c(N2CCN(C(=O)c3ncnc(C)c3O)CC2)c(=O)n2nc(C3=CCOCC3)nc2n1CC(=O)Nc1ccc(Br)cc1Cl ; CCc1c(N2CCN(C(=O)c3ncnc(C)c3O)CC2)c(=O)n2nc(C3=CCOCC3)nc2n1CC(=O)Nc1ccc(C(F)(F)F)cc1C ; CCc1c(N2CCN(C(=O)c3ncccc3O)CC2)c(=O)n2nc(C3=CCOCC3)nc2n1CC(=O)Nc1ccc(C(F)(F)F)cc1C]

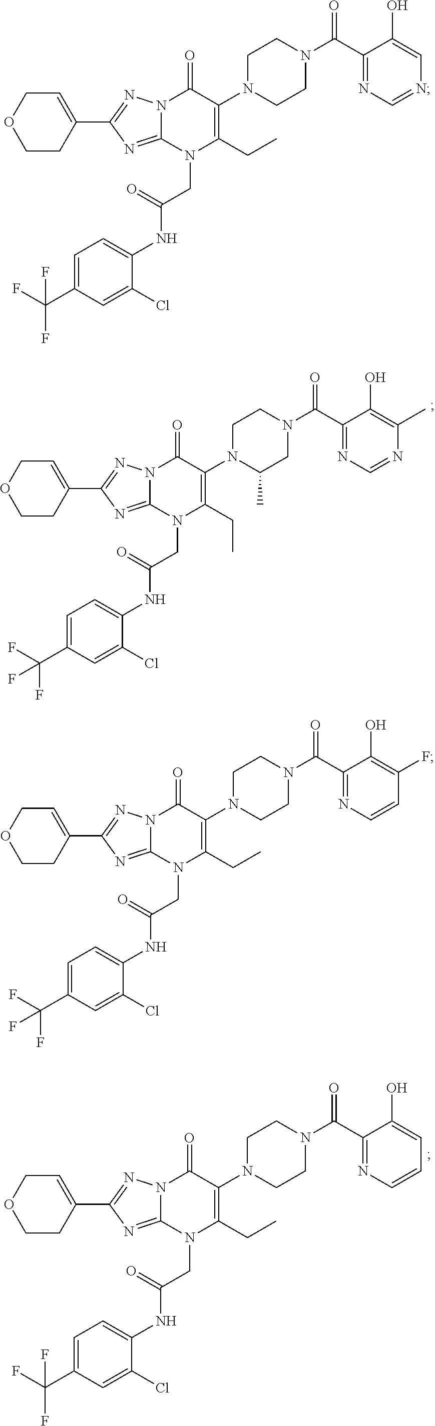 [SMILES: CCc1c(N2CCN(C(=O)c3ncncc3O)CC2)c(=O)n2nc(C3=CCOCC3)nc2n1CC(=O)Nc1ccc(C(F)(F)F)cc1Cl ; CCc1c(N2CCN(C(=O)c3ncnc(C)c3O)C[C@@H]2C)c(=O)n2nc(C3=CCOCC3)nc2n1CC(=O)Nc1ccc(C(F)(F)F)cc1Cl ; CCc1c(N2CCN(C(=O)c3nccc(F)c3O)CC2)c(=O)n2nc(C3=CCOCC3)nc2n1CC(=O)Nc1ccc(C(F)(F)F)cc1Cl ; CCc1c(N2CCN(C(=O)c3ncccc3O)CC2)c(=O)n2nc(C3=CCOCC3)nc2n1CC(=O)Nc1ccc(C(F)(F)F)cc1Cl]

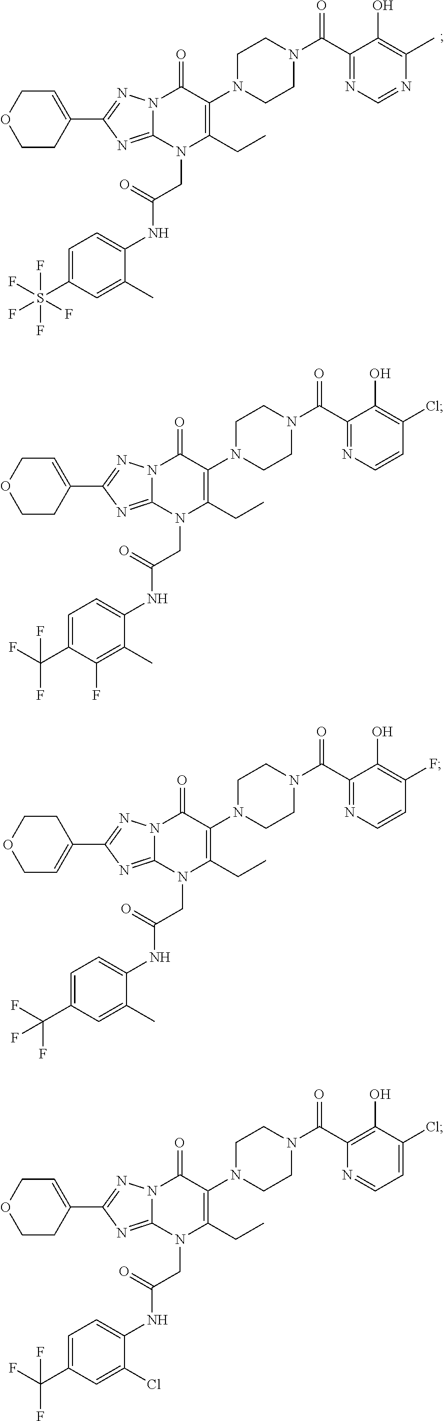 [SMILES: CCc1c(N2CCN(C(=O)c3ncnc(C)c3O)CC2)c(=O)n2nc(C3=CCOCC3)nc2n1CC(=O)Nc1ccc(S(F)(F)(F)(F)F)cc1C ; CCc1c(N2CCN(C(=O)c3nccc(Cl)c3O)CC2)c(=O)n2nc(C3=CCOCC3)nc2n1CC(=O)Nc1ccc(C(F)(F)F)c(F)c1C ; CCc1c(N2CCN(C(=O)c3nccc(F)c3O)CC2)c(=O)n2nc(C3=CCOCC3)nc2n1CC(=O)Nc1ccc(C(F)(F)F)cc1C ; CCc1c(N2CCN(C(=O)c3nccc(Cl)c3O)CC2)c(=O)n2nc(C3=CCOCC3)nc2n1CC(=O)Nc1ccc(C(F)(F)F)cc1Cl]

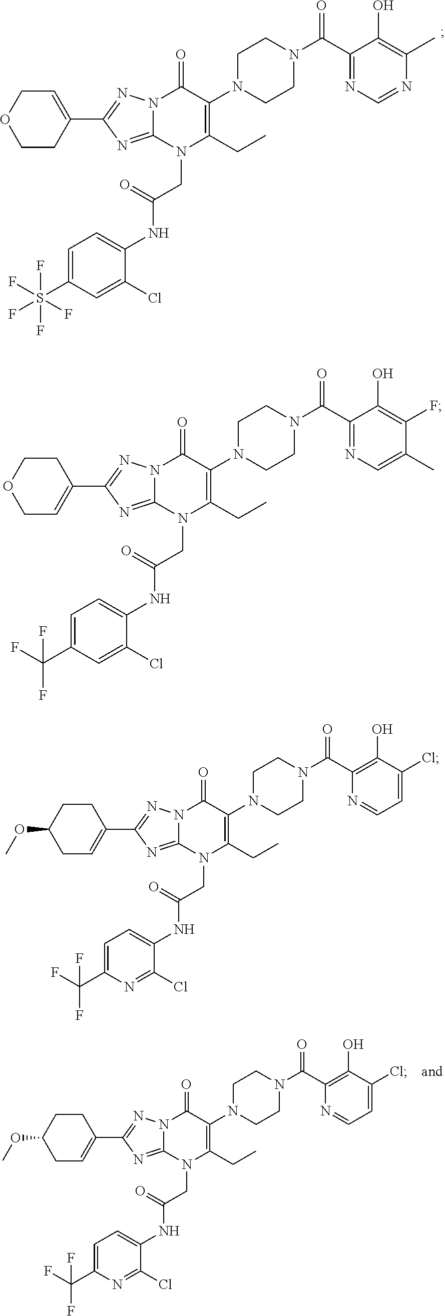

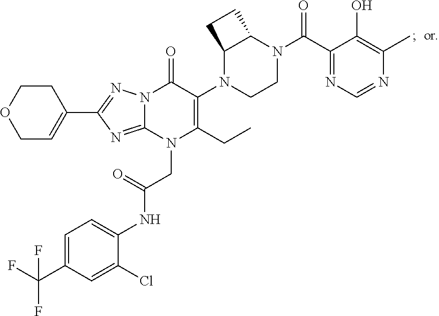 [SMILES: CCc1c(N2CCN(C(=O)c3ncnc(C)c3O)[C@H]3CC[C@@H]32)c(=O)n2nc(C3=CCOCC3)nc2n1CC(=O)Nc1ccc(C(F)(F)F)cc1Cl]

or a zwitterionic form or a salt thereof.

Embodiment 9. The method according to Embodiment 5, the WRN inhibitor for use according to Embodiment 5, the therapeutically active agent for use according to Embodiment 5, or the combination according to Embodiment 5, wherein the WRN inhibitor is

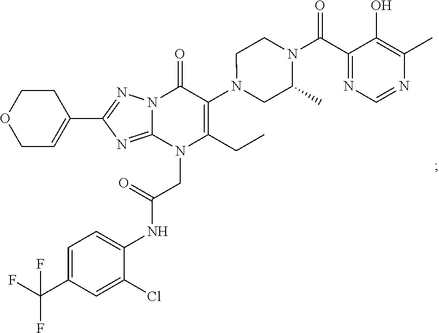 [SMILES: CCc1c(N2CCN(C(=O)c3ncnc(C)c3O)[C@H](C)C2)c(=O)n2nc(C3=CCOCC3)nc2n1CC(=O)Nc1ccc(C(F)(F)F)cc1Cl]

or a zwitterionic form or a salt thereof.

Embodiment 10. The method according to Embodiment 5, the WRN inhibitor for use according to Embodiment 5, the therapeutically active agent for use according to Embodiment 5, or the combination according to Embodiment 5, wherein the WRN inhibitor is

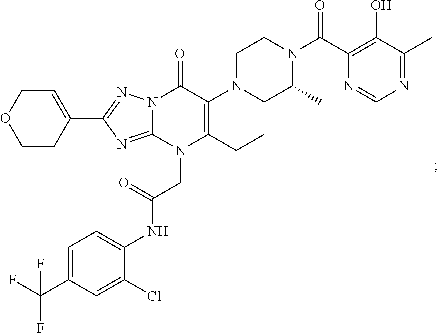 [SMILES: CCc1c(N2CCN(C(=O)c3ncnc(C)c3O)[C@H](C)C2)c(=O)n2nc(C3=CCOCC3)nc2n1CC(=O)Nc1ccc(C(F)(F)F)cc1Cl]

or a zwitterionic form or a salt thereof.

Embodiment 11. The method according to Embodiment 5, the WRN inhibitor for use according to Embodiment 5, the therapeutically active agent for use according to Embodiment 5, or the combination according to Embodiment 5, wherein the WRN inhibitor is

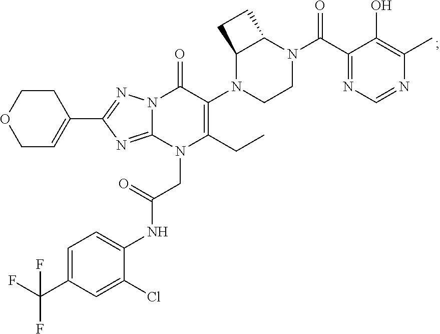 [SMILES: CCc1c(N2CCN(C(=O)c3ncnc(C)c3O)[C@H]3CC[C@@H]32)c(=O)n2nc(C3=CCOCC3)nc2n1CC(=O)Nc1ccc(C(F)(F)F)cc1Cl]

or a zwitterionic form or a salt thereof.

Embodiment 12. The method according to any one of Embodiments 1 and 5 to 11, the WRN inhibitor for use according to any one of Embodiments 2 and 5 to 11, the therapeutically active agent for use according to any one of Embodiments 3 and 5 to 11, wherein the cancer is characterized as microsatellite instability-high (MSI-H) or mismatch repair deficient (dMMR).

Embodiment 13. The method according to any one of Embodiments 1 and 5 to 12, the WRN inhibitor for use according to any one of Embodiments 2 and 5 to 12, the therapeutically active agent for use according to any one of Embodiments 3 and 5 to 12, or the combination according to any one of Embodiments 4 to 11, wherein the (at least one) therapeutically active agent is a PD-1 inhibitor.

Embodiment 14. The method according to Embodiment 13, the WRN inhibitor for use according to Embodiment 13, the therapeutically active agent for use according to Embodiment 13, or the combination according to Embodiment 13, wherein the PD-1 inhibitor is an anti-PD-1 antibody.

Embodiment 15. The method according to Embodiment 13, the WRN inhibitor for use according to Embodiment 13, the therapeutically active agent for use according to Embodiment 13, or the combination according to Embodiment 13, wherein the PD-1 inhibitor is selected from PDR001 (Novartis), Nivolumab (Bristol-Myers Squibb), Pembrolizumab (Merck & Co), Pidilizumab (CureTech), MEDI0680 (Medimmune), Cemiplimab (REGN2810, Regeneron), Dostarlimab (TSR-042, Tesaro), PF-06801591 (Pfizer), Tislelizumab (BGB-A317, Beigene), BGB-108 (Beigene), INCSHR1210 (Incyte), Balstilimab (AGEN2035, Agenus), Sintilimab (InnoVent), Toripalimab (Shanghai Junshi Bioscience), Camrelizumab (Jiangsu Hengrui Medicine Co.), and AMP-224 (Amplimmune), in particular PDR001 or Tislelizumab.

Embodiment 16. The method according to Embodiment 15, the WRN inhibitor for use according to Embodiment 15, the therapeutically active agent for use according to Embodiment 15, or the combination according to Embodiment 15, wherein the PD-1 inhibitor is Tislelizumab.

Embodiment 17. The method according to any one of Embodiments 1 and 5 to 12, the WRN inhibitor for use according to any one of Embodiments 2 and 5 to 12, the therapeutically active agent for use according to any one of Embodiments 3 and 5 to 12, or the combination according to any one of Embodiments 4 to 11, wherein the (at least one) therapeutically active agent is a chemotherapy agent.

Embodiment 18. The method according to Embodiment 17, the WRN inhibitor for use according to Embodiment 17, the therapeutically active agent for use according to Embodiment 17 or the combination according to Embodiment 17, wherein the chemotherapy agent is selected from anastrozole (Arimidex®), vinblastine, vindesine, vinorelbine, vincristine, bicalutamide (Casodex®), bleomycin (e.g. bleuomycin sulfate) (Blenoxane®), busulfan (Myleran®), busulfan injection (Busulfex®), calactin, capecitabine (Xeloda®), N4-pentoxycarbonyl-5-deoxy-5-fluorocytidine, carboplatin (Paraplatin®), carmustine (BICNUR), lomustine (CCNUR), chlorambucil (Leukeran®), bendamustine (Treanda®), cisplatin (Platinol®), cladribine (Leustatin®), cyclophosphamide (Cytoxan® or Neosar®), cytarabine, cytosine arabinoside (Cytosar-U®), cytarabine liposome injection (DTIC-Dome®), dactinomycin (Actinomycin D, Cosmegen), daunorubicin hydrochloride (Cerubidine®), daunorubicin citrate liposome injection (DaunoXome®), dexamethasone, docetaxel (Taxotere®), doxorubicin (e.g. doxorubicin hydrochloride) (Adriamycin®, Rubex®), etoposide (Vepesid®), fludarabine phosphate (Fludara®), 5-fluorouracil (Adrucil®, Efudex®), flutamide (Eulexin®), tezacitabine, gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), a mitomycin (e.g. mitomycin A, mitomycin B or mitomycin C, particularly mitomycin C), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), camptothecin, teniposide (Vumon®), 6-thioguanine, thiotepa, tirapazamine (Tirazone®), topotecan hydrochloride for injection (Hycamptin®), vinblastine (Velban®), vincristine (Oncovin®), oxaliplatin (Eloxatin®), epirubicin (Ellence®, Pharmorubicin®) temozolomide (Temodar®), tegafur, and vinorelbine (Navelbine®), in particular irinotecan.

Embodiment 19. The method according to Embodiment 17 or Embodiment 18, the WRN inhibitor for use according to Embodiment 17 or Embodiment 18, the therapeutically active agent for use according to Embodiment 17 or Embodiment 18 or the combination according to Embodiment 17 or Embodiment 18, wherein the chemotherapy agent is selected from gemcitabine, camptothecin, irinotecan (Camptosar®), docetaxel (Taxotere®), doxorubicin (e.g. doxorubicin hydrochloride) (Adriamycin®, Rubex®), 5-fluorouracil (Adrucil®, Efudex®), capecitabine (Xeloda®), etoposide (Vepesid®), epirubicin (Ellence®, Pharmorubicin®), oxaliplatin (Eloxatin®), mitomycin (e.g. mitomycin A, mitomycin B or mitomycin C, particularly mitomycin C), cisplatin (Platinol®), carboplatin (Paraplatin®) and paclitaxel (Taxol®).

Embodiment 20. The method according to any one of Embodiments 1 and 5 to 12, the WRN inhibitor for use according to any one of Embodiments 2 and 5 to 12, the therapeutically active agent for use according to any one of Embodiments 3 and 5 to 12, or the combination according to any one of Embodiments 4 to 11, wherein the (at least one) therapeutically active agent is a WEE1 inhibitor.

Embodiment 21. The method according to Embodiment 20, the WRN inhibitor for use according to Embodiment 20, the therapeutically active agent for use according to Embodiment 20, or the combination according to Embodiment 20, wherein the WEE1 inhibitor is selected from Adavosertib (also known as AZD1775 and MK-1775) and PDO166285.

Embodiment 22. The method according to Embodiment 21, the WRN inhibitor for use according to Embodiment 21, the therapeutically active agent for use according to Embodiment 21, or the combination according to Embodiment 21, wherein the WEE1 inhibitor is Adavosertib (also known as AZD1775 and MK-1775).

Embodiment 23. The method according to any one of Embodiments 1 and 5 to 12, the WRN inhibitor for use according to any one of Embodiments 2 and 5 to 12, the therapeutically active agent for use according to any one of Embodiments 3 and 5 to 12, or the combination according to any one of Embodiments 4 to 11, wherein the (at least one) therapeutically active agent is an ATR inhibitor.

Embodiment 24. The method according to Embodiment 23, the WRN inhibitor for use according to Embodiment 23, the therapeutically active agent for use according to Embodiment 23, or the combination according to Embodiment 23, wherein the ATR inhibitor is selected from RP-3500, ceralasertib (also known as AZD6738), berzosertib, ART-0380, gartisertib (also known as M4344), and elimusertib (also known as BAY-1895344).

Embodiment 25. The method according to Embodiment 24, the WRN inhibitor for use according to Embodiment 24, the therapeutically active agent for use according to Embodiment 24, or the combination according to Embodiment 24, wherein the ATR inhibitor is elimusertib (BAY-1895344).

Embodiment 26. The method according to any one of Embodiments 1 and 5 to 12, the WRN inhibitor for use according to any one of Embodiments 2 and 5 to 12, the therapeutically active agent for use according to any one of Embodiments 3 and 5 to 12, or the combination according to any one of Embodiments 4 to 11, wherein the (at least one) therapeutically active agent is a DNA-PK inhibitor.

Embodiment 27. The method according to Embodiment 26, the WRN inhibitor for use according to Embodiment 26, the therapeutically active agent for use according to Embodiment 26, or the combination according to Embodiment 26, wherein the DNA-PK inhibitor is selected from AZD-7648, NU7441 (also known as KU-57788), Omipalisib, BAY8400 and M3814.

Embodiment 28. The method according to Embodiment 27, the WRN inhibitor for use according to Embodiment 27, the therapeutically active agent for use according to Embodiment 27, or the combination according to Embodiment 27, wherein the DNA-PK inhibitor is AZD-7648 or NU7441 (KU-57788), particularly AZD-7648.

Embodiment 29. The method according to any one of Embodiments 1 and 6 to 12, the WRN inhibitor for use according to any one of Embodiments 2 and 5 to 12, the therapeutically active agent for use according to any one of Embodiments 3 and 5 to 12, or the combination according to any one of Embodiments 4 to 11, wherein the (at least one) therapeutically active agent is a ionising radiation based therapy selected from i) external beam radiation, ii) brachytherapy and iii) a radiopharmaceutical.

Embodiment 30. The method according to Embodiment 29, the WRN inhibitor for use according to Embodiment 29, the therapeutically active agent according to Embodiment 29, or the combination according to Embodiment 29, wherein the ionising radiation is external beam radiation.

Embodiment 31. The method according to Embodiment 29, the WRN inhibitor for use according to Embodiment 29, the therapeutically active agent for use according to Embodiment 29, or the combination according to Embodiment 29, wherein the ionising radiation is a radiopharmaceutical.

Embodiment 32. The method according to Embodiment 31, the WRN inhibitor for use according to Embodiment 31, the therapeutically active agent for use according to Embodiment 31, or the combination according to Embodiment 31, wherein the radiopharmaceutical is a radioligand agent.

Embodiment 33. The method according to Embodiment 32, the WRN inhibitor for use according to Embodiment 32, the therapeutically active agent for use according to Embodiment 32, or the combination according to Embodiment 32, wherein the radioligand agent is selected from ¹⁷⁷Lu-PSMA-617, ¹⁷⁷Lu-PSMA-R2, ¹⁷⁷Lu-NeoB, ¹⁷⁷Lu-FAP-2286.

Embodiment 34. The method according to any one of Embodiments 1 and 5 to 12, the WRN inhibitor for use according to any one of Embodiments 2 and 5 to 12, the therapeutically active agent for use according to any one of Embodiments 3 and 5 to 12, or the combination according to any one of Embodiments 4 to 11, wherein the (at least one) therapeutically active agent is a MEK inhibitor.

Embodiment 35. The method according to Embodiment 34, the WRN inhibitor for use according to Embodiment 34, the therapeutically active agent for use according to Embodiment 34, or the combination according to Embodiment 34, wherein the MEK inhibitor is selected from the group consisting of refametinib, pimasertib, selumetinib, trametinib, binimetinib and cobimetinib, or a pharmaceutically acceptable salt thereof.

Embodiment 36. The method according to Embodiment 35, the WRN inhibitor for use according to Embodiment 35, the therapeutically active agent for use according to Embodiment 35, or the combination according to Embodiment 35, wherein the MEK inhibitor is trametinib.

Embodiment 37. The method according to any one of Embodiments 1 and 5 to 12, the WRN inhibitor for use according to any one of Embodiments 2 and 5 to 12, the therapeutically active agent for use according to any one of Embodiments 3 and 5 to 12, or the combination according to any one of Embodiments 4 to 11, wherein the (at least one) therapeutically active agent is an MDM2 inhibitor.

Embodiment 38. The method according to Embodiment 37, the WRN inhibitor for use according to Embodiment 37, the therapeutically active agent for use according to Embodiment 37, or the combination according to Embodiment 37, wherein the MDM2 inhibitor is selected from the group consisting of nutlin-3a, idasanutlin (also known as RG7388), RG7112, KRT-232 (also known as AMG-232), APG-115, RAIN-32 (also known as DS-3032 and milademetan), BI-907828 and HDM201 (also known as siremadlin), or a pharmaceutically acceptable salt thereof.

Embodiment 39. The method according to Embodiment 38, the WRN inhibitor for use according to Embodiment 38, the therapeutically active agent for use according to Embodiment 38, or the combination according to Embodiment 38, wherein the MDM2 inhibitor is HDM201.

Embodiment 40. The method according to any one of Embodiments 1 and 5 to 12, the WRN inhibitor for use according to any one of Embodiments 2 and 5 to 12, the therapeutically active agent for use according to any one of Embodiments 3 and 5 to 12, or the combination according to any one of Embodiments 4 to 11, wherein the (at least one) therapeutically active agent is a G4-quadruplex stabilizer.

Embodiment 41. The method according to Embodiment 40, the WRN inhibitor for use according to Embodiment 40, the therapeutically active agent for use according to Embodiment 40, or the combination according to Embodiment 40, wherein the G4-quadruplex stabilizer is pyridostatin.

Embodiment 42. The method according to any one of Embodiments 1 and 5 to 12, the WRN inhibitor for use according to any one of Embodiments 2 and 5 to 12, the therapeutically active agent for use according to any one of Embodiments 3 and 5 to 12, or the combination according to any one of Embodiments 4 to 11, wherein the (at least one) therapeutically active agent is an ATM inhibitor.

Embodiment 43. The method according to Embodiment 42, the WRN inhibitor for use according to Embodiment 42, the therapeutically active agent for use according to Embodiment 42, or the combination according to Embodiment 42, wherein the ATM inhibitor is selected from KU-55933, KU-60019, KU-59403, M3541, CP-466722, AZ31, AZ32, AZD0156 and AZD1390.

Embodiment 44. The method according to Embodiment 43, the WRN inhibitor for use according to Embodiment 43, the therapeutically active agent for use according to Embodiment 43, or the combination according to Embodiment 43, wherein the ATM inhibitor is KU-60019.

Embodiment 45. The method according to any one of Embodiments 1 and 5 to 12, the WRN inhibitor for use according to any one of Embodiments 2 and 5 to 12, the therapeutically active agent for use according to any one of Embodiments 3 and 5 to 12, or the combination according to any one of Embodiments 4 to 11, wherein the (at least one) therapeutically active agent is a PARP inhibitor.

Embodiment 46. The method according to Embodiment 45, the WRN inhibitor for use according to Embodiment 45, the therapeutically active agent for use according to Embodiment 45, or the combination according to Embodiment 45, wherein the PARP inhibitor is selected from olaparib, NMS293, niraparib, prexasertib, veliparib, rucaparib, talazoparib, AZD-5305 and KU0058948.

Embodiment 47. The method according to Embodiment 46, the WRN inhibitor for use according to Embodiment 46, the therapeutically active agent for use according to Embodiment 46, or the combination according to Embodiment 46, wherein the PARP inhibitor is olaparib.

Embodiment 48. The method according to any one of Embodiments 1 and 5 to 12, the WRN inhibitor for use according to any one of Embodiments 2 and 5 to 12, the therapeutically active agent for use according to any one of Embodiments 3 and 5 to 12 or the combination according to any one of Embodiments 4 to 11, wherein the (at least one) therapeutically active agent is a chemotherapy agent, and the chemotherapy agent is a topoisomerase inhibitor.

Embodiment 49. The method according to Embodiment 48, the WRN inhibitor for use according to Embodiment 48, the therapeutically active agent for use according to Embodiment 48 or the combination according to Embodiment 48, wherein the topoisomerase inhibitor is selected from QAP1, irinotecan, topotecan, camptothecin and etoposide.

Embodiment 50. The method according to Embodiment 49, the WRN inhibitor for use according to Embodiment 49, the therapeutically active agent for use according to Embodiment 49 or the combination according to Embodiment 49, wherein the topoisomerase inhibitor is selected from QAP1, etoposide and irinotecan.

Embodiment 51. The method according to any one of Embodiments 1 and 5 to 12, the WRN inhibitor for use according to any one of Embodiments 2 and 5 to 12, the therapeutically active agent for use according to any one of Embodiments 3 and 5 to 12, or the combination according to any one of Embodiments 4 to 11, wherein the (at least one) therapeutically active agent is a CHK1 or CHK2 inhibitor.

Embodiment 52. The method according to Embodiment 51, the WRN inhibitor for use according to Embodiment 51, the therapeutically active agent for use according to Embodiment 51, or the combination according to Embodiment 51, wherein the CHK1 or CHK2 inhibitor, is selected from GDC-0575, Prexasertib (also known as LY2606368), SCH900776 (also known as MK-8776), SRA737, PF477736, LY2606368 and AZD7762, in particular AZD7762.

Embodiment 53. The method according to any one of Embodiments 1 and 5 to 52, the WRN inhibitor for use according to any one of Embodiments 2 and 5 to 52 or the therapeutically active agent for use according to any one of Embodiments 3 and 5 to 52, wherein the cancer is selected from colorectal cancer (CRC), gastric cancer, prostate cancer, endometrial cancer, adrenocortical cancer, uterine cancer and cervical cancer, for example a cancer selected from uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma.

Embodiment 54. The method according to Embodiment 53, the WRN inhibitor for use according to Embodiment 53 or the therapeutically active agent for use according to Embodiment 53, wherein the cancer is colorectal cancer (CRC) or gastric cancer, particularly colorectal cancer (CRC).

Embodiment 55. A method of treating colorectal cancer, the method comprising administering to a subject a therapeutically effective amount of a WRN inhibitor in combination with a therapeutically effective amount of a G4-quadruplex stabilizer.

Embodiment 56. The method according to Embodiment 55, wherein the G4-quaduplex stabiliser is pyridostatin.

Embodiment 57. A method of treating colorectal cancer, the method comprising administering to a subject a therapeutically effective amount of a WRN inhibitor in combination with a therapeutically effective amount of a MEK inhibitor.

Embodiment 58. The method according to Embodiment 57, wherein the MEK inhibitor is trametinib.

Embodiment 59. A method of treating colorectal cancer, the method comprising administering to a subject a therapeutically effective amount of a WRN inhibitor in combination with a therapeutically effective amount of a topoisomerase inhibitor.

Embodiment 60. The method according to Embodiment 59, wherein the topoisomerase inhibitor is QAP1, etoposide, irinotecan or camptothecin.

Embodiment 61. A method of treating colorectal or gastric cancer, the method comprising administering to a subject a therapeutically effective amount of a WRN inhibitor in combination with a therapeutically effective amount of an ATR inhibitor.

Embodiment 62. The method according to Embodiment 61, wherein the ATR inhibitor is elimusertib (BAY-1895344).

Embodiment 63. A method of treating gastric cancer or colorectal cancer, the method comprising administering to a subject a therapeutically effective amount of a WRN inhibitor in combination with a therapeutically effective amount of ionising radiation based therapy selected from i) external beam radiation, ii) brachytherapy and ii) a radiopharmaceutical.

Embodiment 64. The method according to Embodiment 63, wherein the ionising radiation is external beam radiation.

Embodiment 65. The method according to Embodiment 63, wherein the ionising radiation is a radiopharmaceutical.

Embodiment 66. The method according to Embodiment 65, wherein the radiopharmaceutical is a radioligand agent.

Embodiment 67. The method according to Embodiment 66, wherein the radioligand agent is selected from ¹⁷⁷Lu-PSMA-617, ¹⁷⁷Lu-PSMA-R2, ¹⁷⁷Lu-NeoB, ¹⁷⁷Lu-FAP-2286.

Embodiment 68. A method of treating colorectal cancer, the method comprising administering to a subject a therapeutically effective amount of a WRN inhibitor in combination with a therapeutically effective amount of a chemotherapy agent.

Embodiment 69. The method according to Embodiment 68, wherein the chemotherapy agent is selected from 5-fluorouracil, cisplatin, bleomycin, docetaxel, doxorubicin, epirubicin, etoposide, camptothecin, mitomycin, oxaliplatin, mitomycin (e.g. mitomycin C) and gemcitabine.

Embodiment 70. A method of treating colorectal cancer, the method comprising administering to a subject a therapeutically effective amount of a WRN inhibitor in combination with a therapeutically effective amount of an MDM2 inhibitor.

Embodiment 71. The method according to Embodiment 70, wherein the MDM2 inhibitor is HDM201.

Embodiment 72. A method of treating colorectal cancer, the method comprising administering to a subject a therapeutically effective amount of a WRN inhibitor in combination with a therapeutically effective amount of an ATM inhibitor.

Embodiment 73. The method according to Embodiment 72, wherein the ATM inhibitor is KU-60019.

Embodiment 74. A method of treating colorectal cancer, the method comprising administering to a subject a therapeutically effective amount of a WRN inhibitor in combination with a therapeutically effective amount of a DNA-PK inhibitor.

Embodiment 75. The method according to Embodiment 74, wherein the DNA-PK inhibitor is AZD-7648 or NU7441 (KU-57788).

Embodiment 76. A method of treating colorectal cancer, the method comprising administering to a subject a therapeutically effective amount of a WRN inhibitor in combination with a therapeutically effective amount of a WEE1 inhibitor.

Embodiment 77. The method according to Embodiment 76, wherein the WEE1 inhibitor is Adavosertib.

Embodiment 78. A method of treating colorectal cancer, the method comprising administering to a subject a therapeutically effective amount of a WRN inhibitor in combination with a therapeutically effective amount of a CHK1 or CHK2 inhibitor.

Embodiment 79. The method according to Embodiment 78, wherein the CHK1 or CHK2 inhibitor is AZD7762 or SCH900776 (also known as MK-8776).

Embodiment 80. A method according to any one of Embodiments 55 to 79, wherein the colorectal cancer or gastric cancer is characterized as microsatellite instability-high (MSI-H) or mismatch repair deficient (dMMR).

Embodiment 81. The method according to Embodiment 80, wherein the colorectal cancer or gastric cancer is characterized as microsatellite instability-high (MSI-H).

Embodiment 82. The method according to according to any one of Embodiments 54, 61 to 67, 80 and 81, wherein the gastric cancer is gastric adenocarcinoma.

Embodiment 83. The method according to any one of the Embodiments 55 to 82, wherein the WRN inhibitor is selected from

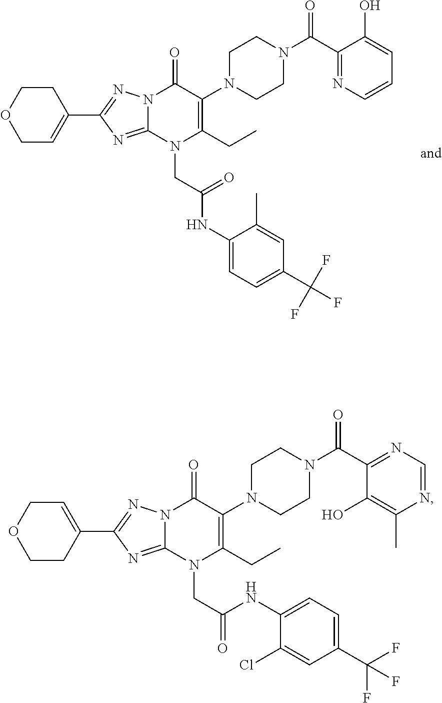 [SMILES: CCc1c(N2CCN(C(=O)c3ncccc3O)CC2)c(=O)n2nc(C3=CCOCC3)nc2n1CC(=O)Nc1ccc(C(F)(F)F)cc1C ; CCc1c(N2CCN(C(=O)c3ncnc(C)c3O)CC2)c(=O)n2nc(C3=CCOCC3)nc2n1CC(=O)Nc1ccc(C(F)(F)F)cc1Cl]

or a zwitterionic form or a pharmaceutically acceptable salt thereof.

Embodiment 84. The method according to Embodiment 83, wherein the WRN inhibitor is

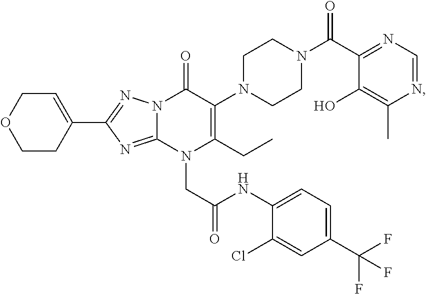 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl]

or a zwitterionic form or a pharmaceutically acceptable salt thereof.

Embodiment 85: A combination comprising i) a WRN inhibitor and ii) temozolomide, and optionally iii) irinotecan.

Embodiment 86: A combination according to Embodiment 85, as a non-fixed dose combination.

Embodiment 87: Temozolomide for use in the treatment of cancer, wherein the treatment further comprises administration of:
a WRN inhibitor, or
a WRN inhibitor in combination with irinotecan.

Embodiment 88. A WRN inhibitor for use in the treatment of cancer, wherein the treatment further comprises administration of:
temozolomide, or
temozolomide and irinotecan.

Embodiment 89. Irinotecan for use in the treatment of cancer, wherein the treatment further comprises administration of:
a WRN inhibitor, or in particular,
a WRN inhibitor and temozolomide.

Embodiment 90. Temozolomide for use according to Embodiment 87, or a WRN inhibitor for use according to Embodiment 88, or irinotecan for use according to Embodiment 89, wherein the cancer is MSI-H, dMMR or MSS, in particular MSS. For example, the cancer is pMMR/MSS MGMT-defective, or pMMR/MSS MGMT-deficient, or pMMR/MSS MGMT-silenced, or MGMT-methylated pMMR/MSS cancer. For example, the cancer is selected from a cancer as described herein. In another example, the cancer is pMMR/MSS MGMT-defective CRC (colorectal cancer), or pMMR/MSS MGMT-deficient CRC or pMMR/MSS MGMT-silenced CRC, or pMMR/MSS MGMT methylated colorectal cancer. Alternatively, the cancer is MSI-H MGMT-defective, or MSI-H MGMT-deficient, or MSI-H MGMT-silenced, or MSI-H MGMT-methylated cancer. In particular, colorectal cancer. In another example, the pMMR/MSS or MSH-H cancer is RAS-mutated or RAS wild-type CRC.

Embodiment 91. Temozolomide for use, or a WRN inhibitor for use, or irinotecan for use, according to Embodiment 90, wherein the cancer is dMMR or MSS cancer, in particular MSS. In one example, the cancer is pMMR/MSS MGMT-defective, or pMMR/MSS MGMT-deficient, or pMMR/MSS MGMT-silenced, or MGMT-methylated pMMR/MSS cancer. For example, the cancer is selected from a cancer as described herein. In another example, the tumor is pMMR/MSS MGMT-defective CRC, or pMMR/MSS MGMT-deficient CRC, or pMMR/MSS MGMT-silenced CRC, or MGMT-methylated pMMR/MSS CRC (colorectal cancer).

Embodiment 92. Temozolomide for use according to Embodiment 87, 90 or 91, or a WRN inhibitor for use according to Embodiment 88, 90 or 91, or irinotecan according to Embodiment 89, 90 or 91, wherein temozolomide is administered:
as a pre-treatment before administration of the WRN inhibitor, or before administration of irinotecan, or before administration of both the WRN inhibitor and irinotecan, without subsequent combination treatment including temozolide, or
in combination, without pre-treatment with temozolomide, or
as a pre-treatment before administration of the WRN inhibitor, or before administration of irinotecan, or before administration of both the WRN inhibitor and irinotecan, followed by combination treatment including temozolomide,

wherein said combination or combination treatment is described in embodiments 87, 88, 89, 90 or 91.

Embodiment 93. Temozolomide for use according to any of Embodiments 87, 90 or 91, or a WRN inhibitor for use according to any of Embodiments 88, 90 or 91, or irinotecan for use according to any of Embodiments 89, 90 or 91, wherein said cancer, in particular said MSI-H, dMMR or MSS cancer is selected from colorectal cancer (CRC), gastric cancer, prostate cancer, endometrial cancer, adrenocortical cancer, uterine cancer and cervical cancer, for example a cancer selected from uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma, or glioma, glioblastoma, neuroendocrine tumors, melanoma, colorectal cancer, small cell lung cancer, triple negative breast cancer, and sarcoma.

Embodiment 94. Temozolomide for use, or a WRN inhibitor for use, or irinotecan for use according to Embodiment 93, wherein said cancer, in particular said MSI-H, dMMR or MSS cancer is colorectal cancer (CRC), gastric cancer, prostate cancer, small cell lung cancer or endometrial cancer, in particular colorectal cancer.

Embodiment 95. Temozolomide for use, or a WRN inhibitor for use, or irinotecan for use, according to Embodiment 94, wherein the cancer is colorectal cancer (CRC), in particular MSS colorectal cancer or dMMR colorectal cancer, or MSS small cell lung cancer or dMMR small cell lung cancer.

Embodiment 96: A WRN inhibitor for use in the treatment of dMMR or MSS cancer, in particular MSS cancer. In particular, the WRN inhibitor is a compound as defined in any of embodiments 5 to 11, 83 or 84 herein. More particularly, the compound is Compound A herein.

Embodiment 97: A combination according to Embodiment 85, wherein the WRN inhibitor is a compound as defined in any of embodiments 5 to 11, 83 or 84 herein. In particular, the WRN inhibitor is compound A herein.

Embodiment 98: A method of treating cancer in a subject in need thereof, the method comprising administering to the subject a therapeutically effective amount of temozolomide, wherein the treatment further comprises administration of:
a therapeutically effective amount of a WRN inhibitor, or
a therapeutically effective amount of a WRN inhibitor in combination with a therapeutically effective amount of irinotecan.

Embodiment 99: A method of treating cancer in a subject in need thereof, the method comprising administering to the subject a therapeutically effective amount of a WRN inhibitor, wherein the treatment further comprises administration of:
a therapeutically effective amount of temozolomide, or
a therapeutically effective amount of temozolomide and a therapeutically effective amount of irinotecan.

Embodiment 100: A method of treating cancer in a subject in need thereof, the method comprising administering to the subject a therapeutically effective amount of irinotecan, wherein the treatment further comprises administration of:
a therapeutically effective amount of a WRN inhibitor, or in particular
a therapeutically effective amount of a WRN inhibitor and a therapeutically effective amount or temozolomide.

Embodiment 101: A method according to any of Embodiments 98 to 100, wherein the cancer is MSI-H, dMMR or MSS, in particular dMMR or MSS, for example MSS. In one example, the cancer is pMMR/MSS MGMT-defective, or pMMR/MSS MGMT-deficient, or pMMR/MSS MGMT-silenced, or MGMT-methylated pMMR/MSS cancer. In another example, the tumor is pMMR/MSS MGMT-defective, or pMMR/MSS MGMT-deficient, or pMMR/MSS MGMT-silenced, or pMMR/MSS MGMT-methylated colorectal cancer. In another example, the cancer is MSI-H MGMT-defective, or MSI-H MGMT-deficient, or MSI-H MGMT-silenced, or MSI-H MGMT-methylated cancer. In particular, colorectal cancer (CRC). More particularly, RAS-mutated or RAS wild-type CRC.

Embodiment 102: A method according to any of Embodiments 98 to 101, wherein temozolomide is administered:
as a pre-treatment before administration of the WRN inhibitor, or before administration of irinotecan, or before administration of both the WRN inhibitor and irinotecan, without subsequent combination treatment including temozolide, or
in combination, without pre-treatment with temozolomide, or
as a pre-treatment before administration of the WRN inhibitor, or before administration of irinotecan, or before administration of both the WRN inhibitor and irinotecan, followed by combination treatment including temozolide,

wherein said combination or combination treatment is described in embodiments 98 to 101.

Embodiment 103: Temozolomide for use, or a WRN inhibitor for use, or irinotecan for use, or a method according to any of Embodiments 87 to 95 or 98 to 102, wherein temozolomide is used as:
a combination agent with at least one combination partner including a WRN inhibitor, or
as a pre-treatment agent prior to combination treatment wherein said combination treatment includes at least one combination partner which is a WRN inhibitor, or
as both a pre-treatment agent and combination agent with a WRN inhibitor,

in order to:
sensitise the cancer cells, for example for an improved response to treatment with a WRN inhibitor,
prime the cancer cells, for example, such priming may include triggering hypermutation status in cancer cells,
create or increase MMR deficiency in cancer cells,
create or increase MSI-H status in cancer cells,
increase MMR heterogeneity of cancer cells, and/or
create or increase resistance in cancer cells to temozolomide.

Embodiment 104: A WRN inhibitor for use in the treatment of cancer, wherein the treatment further comprises administration of an alkylating agent, in particular temozolomide, and the cancer is:
MSS cancer,
pMMR/MSS MGMT-defective, or pMMR/MSS MGMT-deficient, or pMMR/MSS MGMT-silenced, or MGMT-methylated pMMR/MSS cancer, such as colorectal cancer
MGMT-methylated glioblastoma,
MGMT-hypermethylated, CRC, or
MGMT-hypermethylated, RAS-mutated or RAS wild-type CRC.

Optionally, said use further comprises administration of irinotecan.

Embodiment 105: A WRN inhibitor for use in the treatment of cancer, according to embodiment 104, wherein the treatment further comprises administration of an alkylating agent, and the cancer is MSS. For example, the cancer is pMMR/MSS MGMT-defective, or pMMR/MSS MGMT-deficient, or pMMR/MSS MGMT-silenced, or MGMT-methylated pMMR/MSS cancer. For example, the alkylating agent is temozolomide.

Embodiment 106: A WRN inhibitor for use in the treatment of cancer, wherein the treatment further comprises administration of an alkylating agent, for example temozolomide, and the cancer is MGMT-defective, or MGMT-deficient, or MGMT-silenced, or MGMT-methylated pMMR/MSS CRC.

Embodiment 107: Temozolomide for use, or a WRN inhibitor for use, or irinotecan for use, or a method according to any of Embodiments 87 to 95 or 98 to 105, wherein the WRN inhibitor is as described in any of embodiments 5 to 8, 83 or 84. In particular, the WRN inhibitor is compound A herein.

Embodiment 108: An alkylating agent for use in the treatment of cancer, wherein the treatment further comprises administration of a WRN inhibitor, and the cancer is:
MSS cancer,
pMMR/MSS MGMT-defective, or pMMR/MSS MGMT-deficient, or pMMR/MSS MGMT-silenced, or MGMT-methylated pMMR/MSS cancer, such as colorectal cancer
MGMT-methylated glioblastoma,
MGMT-hypermethylated, CRC, or
MGMT-hypermethylated, RAS-mutated or RAS wild-type CRC.

For example, the cancer is pMMR/MSS MGMT-defective, or pMMR/MSS MGMT-deficient, or pMMR/MSS MGMT-silenced, or MGMT-methylated pMMR/MSS cancer. For example, the WRN inhibitor has a structure according to a formula described herein, in particular compound A. For example, the alkylating agent is temozolomide. Optionally, said use further comprises administration of irinotecan.

Embodiment 109. A method of treating cancer, in particular cancer characterized as microsatellite instability-high (MSI-H) or mismatch repair deficient (dMMR), in a subject in need thereof, the method comprising administering to the subject a therapeutically effective amount of a WRN inhibitor in combination with a therapeutically effective amount of a DNA polymerase alpha inhibitor, for example aphidicolin, for example for the treatment of colorectal cancer.

Embodiment 110. A method of treating cancer, in particular cancer characterized as microsatellite instability-high (MSI-H) or mismatch repair deficient (dMMR), in a subject in need thereof, the method comprising administering to the subject a therapeutically effective amount of a WRN inhibitor in combination with a therapeutically effective amount of a DNA polymerase alpha inhibitor, for example aphidicolin, for example for the treatment of colorectal cancer.

Embodiment 111: A method of treating cancer, in particular cancer characterized as MSS or mismatch repair deficient (dMMR), in particular MSS, in a subject in need thereof, the method comprising administering to the subject a therapeutically effective amount of a WRN inhibitor in combination with a therapeutic agent which can:
create or increase MMR deficiency in cancer cells, or
create or increase MSI-H status in cancer cells.

In particular, said therapeutic agent is temozolomide.

Embodiment 112: A WRN inhibitor for use in the treatment of cancer, in particular cancer characterized as MSS or mismatch repair deficient (dMMR), in particular MSS, wherein the treatment further comprises administration of a therapeutic agent which can:
create or increase MMR deficiency in cancer cells, or
create or increase MSI-H status in cancer cells.

In particular, said therapeutic agent is temozolomide.

Embodiment 113: Temozolomide for use, or a WRN inhibitor for use, or irinotecan for use, according to any of the Embodiments herein, wherein temozolomide is administered in repeated doses, for example 2, 3 or more repeated doses.

There are also provided the following aspects and embodiments of the invention:

In another aspect there is provided a WRN inhibitor for use in the treatment of cancer, wherein the treatment further comprises administration of:
i. an agent which can
   sensitise the cancer cells, for example for an improved response to treatment with a WRN inhibitor,
   prime the cancer cells, for example, such priming may include triggering hypermutation status in cancer cells,
   create or increase MMR deficiency in cancer cells,
   create or increase MSI-H status in cancer cells, or
   increase MMR heterogeneity of cancer cells,

and optionally wherein the treatment further comprises administration of:
ii. a chemotherapy, for example irinotecan.

In one embodiment, there is provided a WRN inhibitor for use in the treatment of MSS (microsatellite stable) cancer. In another embodiment, the agent i. is selected from temozolomide, cisplatin and 6-thioguanine, or the agent i. is an ionising radiation based therapy selected from i) external beam radiation, ii) brachytherapy and ii) a radiopharmaceutical. In particular, the agent i. is temozolomide. In another embodiment, the treatment further comprises administration of both i. temozolomide and ii. irinotecan.

In another embodiment, the WRN inhibitor is as described herein. In particular, the WRN inhibitor is a compound of Formula (I), or Formula (1g) as described herein. More particularly, the WRN inhibitor is Compound A:

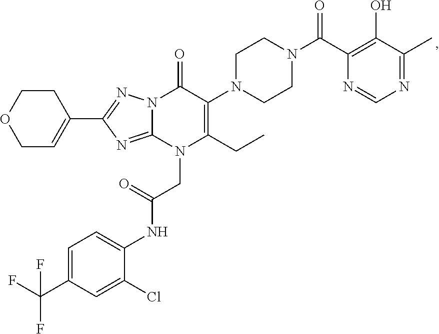 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl]

or a pharmaceutically acceptable salt thereof.

In particular, the WRN inhibitor is for use in the treatment of cancer, wherein the cancer is selected from colorectal cancer (CRC), such as colon adenocarcinoma or rectal adenocarcinoma, gastric cancer, such as stomach adenocarcinoma, prostate cancer, endometrial cancer, adrenocortical cancer, such as adrenocortical carcinoma, cervical cancer, such as cervical squamous cell carcinoma or endocervical adenocarcinoma, uterine cancer, such as uterine corpus endometrial carcinoma and uterine carcinosarcoma, esophageal cancer, such as esophageal carcinoma, breast cancer, such as breast carcinoma or triple negative breast cancer, kidney cancer, such as kidney renal clear cell carcinoma, ovarian cancer, such as ovarian serous cystadenocarcinoma, glioma, glioblastoma, neuroendocrine tumors, melanoma, small cell lung cancer and sarcoma. In particular, colorectal cancer or small cell lung cancer.

More particularly, the cancer is selected from MSS (microsatellite stable) MGMT-defective, or MSS (microsatellite stable) MGMT-deficient, or MSS (microsatellite stable) MGMT-silenced, or MGMT-methylated MSS (microsatellite stable) cancer.

In one embodiment, the agent i. is administered as:
a pre-treatment agent prior to treatment with the WRN inhibitor,
a combination agent with the WRN inhibitor, without pre-treatment,
both a pre-treatment agent and combination agent with a WRN inhibitor.

More particularly, the agent i. is temozolomide, and temozolomide is administered:
as a pre-treatment before administration of the WRN inhibitor, or
in combination with the WRN inhibitor, without pre-treatment with temozolomide, or
as a pre-treatment before administration of the WRN inhibitor, followed by combination treatment including temozolomide and the WRN inhibitor,

and the treatment preferably further comprises administration of irinotecan.

In yet another aspect, there is provided a combination comprising i) a WRN inhibitor and ii) temozolomide, and optionally iii) irinotecan. More particularly, the cancer is MSI-H, dMMR or MSS, in particular MSS. In one embodiment, the cancer is selected from colorectal cancer (CRC), such as colon adenocarcinoma or rectal adenocarcinoma, gastric cancer, such as stomach adenocarcinoma, prostate cancer, endometrial cancer, adrenocortical cancer, such as adrenocortical carcinoma, cervical cancer, such as cervical squamous cell carcinoma or endocervical adenocarcinoma, uterine cancer, such as uterine corpus endometrial carcinoma and uterine carcinosarcoma, esophageal cancer, such as esophageal carcinoma, breast cancer, such as breast carcinoma or triple negative breast cancer, kidney cancer, such as kidney renal clear cell carcinoma, ovarian cancer, such as ovarian serous cystadenocarcinoma, glioma, glioblastoma, neuroendocrine tumors, melanoma, small cell lung cancer and sarcoma. In particular, colorectal cancer or small cell lung cancer, especially MSS colorectal cancer or MSS small cell lung cancer. In another embodiment, the cancer is selected from MSS (microsatellite stable) MGMT-defective, or MSS (microsatellite stable) MGMT-deficient, or MSS (microsatellite stable) MGMT-silenced, or MGMT-methylated MSS (microsatellite stable) cancer.

In another embodiment, the WRN inhibitor is as described herein. In particular, the WRN inhibitor is a compound of Formula (I), or Formula (1g) as described herein. More particularly, the WRN inhibitor is Compound A:

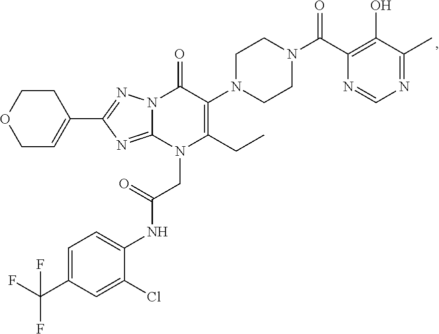 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl]

or a pharmaceutically acceptable salt thereof.

In one embodiment, temozolomide is administered:
as a pre-treatment before administration of the WRN inhibitor, or before administration of irinotecan, or before administration of both the WRN inhibitor and irinotecan, without subsequent combination treatment including temozolide, or
in combination, without pre-treatment with temozolomide, or
as a pre-treatment before administration of the WRN inhibitor, or before administration of irinotecan, or before administration of both the WRN inhibitor and irinotecan, followed by combination treatment including temozolomide.

In yet another aspect there is provided a combination of a WRN inhibitor, and:
i. an agent which can
   sensitise the cancer cells, for example for an improved response to treatment with a WRN inhibitor,
   prime the cancer cells, for example, such priming may include triggering hypermutation status in cancer cells,
   create or increase MMR deficiency in cancer cells,
   create or increase MSI-H status in cancer cells, or
   increase MMR heterogeneity of cancer cells,

and optionally:
ii. a chemotherapy, for example irinotecan.

In yet another aspect there is provided a method of treating cancer in a subject in need thereof, in particular microsatellite stable cancer (MSS), the method comprising administering to the subject a therapeutically effective amount of a WRN inhibitor in combination with a therapeutically effective amount of:
i. an agent which can
   sensitise the cancer cells, for example for an improved response to treatment with a WRN inhibitor,
   prime the cancer cells, for example, such priming may include triggering hypermutation status in cancer cells,
   create or increase MMR deficiency in cancer cells,
   create or increase MSI-H status in cancer cells, or
   increase MMR heterogeneity of cancer cells,

and optionally:
ii. a chemotherapy, for example irinotecan.

In another embodiment there is provided temozolomide for use, or a WRN inhibitor for use, or irinotecan for use, according to any of the embodiments herein, wherein temozolomide is administered in repeated doses, for example 2, 3 or more repeated doses.

In yet another aspect there is provided a pharmaceutical composition comprising a combination of a WRN inhibitor, such as a compound of formula (I), (1g), Compound A or Compound B, or a pharmaceutically acceptable salt thereof, and agent i. as defined herein, and optionally ii. a chemotherapy, for example irinotecan, according to the combinations described herein, and one or more pharmaceutically acceptable carriers.

In particular there is provided a pharmaceutical composition comprising a combination of a WRN inhibitor, such as a compound of formula (I), (1g), Compound A or Compound B, or a pharmaceutically acceptable salt thereof, and agent i. which is temozolomide, and optionally ii. a chemotherapy, in particular irinotecan.

In an aspect there is provided a method of treating cancer in a subject in need thereof, wherein the subject has microsatellite stable cancer (MSS), the method comprising administering to the subject a therapeutically effective amount of
a) temozolomide; and

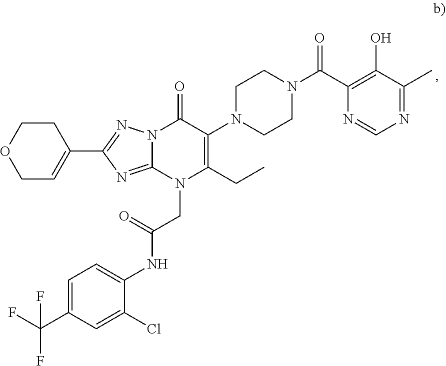 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl]

or a pharmaceutically acceptable salt thereof,

and optionally,
c) irinotecan.

In an embodiment, the temozolomide is administered in an amount sufficient to change the status of the MSS cancer, for example to dMMR or MSI-H, in particular MSI-H cancer in the subject. In a further embodiment, the status of MSS and MSI-H cancer in the subject is determined by an FDA-approved test.

In another embodiment, the temozolomide is administered in an amount sufficient to:
sensitise the cancer cells, for example for an improved response to treatment with a WRN inhibitor,
prime the cancer cells, for example, such priming may include triggering hypermutation status in cancer cells,
create or increase MMR deficiency in cancer cells,
create or increase MSI-H status in cancer cells, or
increase MMR heterogeneity of cancer cells.

In another aspect there is provided a method of treating cancer in a subject in need thereof, wherein the subject has microsatellite stable cancer (MSS), and wherein the patient is administered:
a) an agent which:
   sensitises the cancer cells, for example for an improved response to treatment with a WRN inhibitor,
   primes the cancer cells, for example, such priming may include triggering hypermutation status in cancer cells,
   creates or increases MMR deficiency in cancer cells,
   creates or increases MSI-H status in cancer cells, or
   increases MMR heterogeneity of cancer cells,

(for example, as determined according to tests taught in the art, or tests commercially available, or an FDA-approved test, for example the agent may be selected from temozolomide, cisplatin and 6-thioguanine, or the agent is an ionising radiation based therapy selected from i) external beam radiation, ii) brachytherapy and ii) a radiopharmaceutical) and

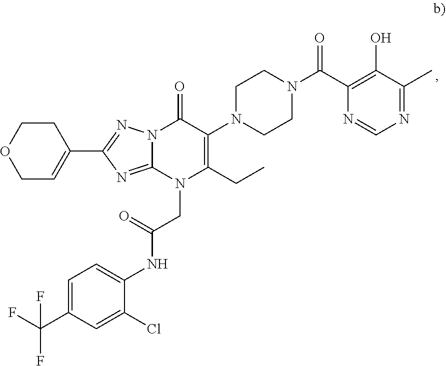 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl]

or a pharmaceutically acceptable salt thereof,

and optionally,
c) irinotecan.

In an embodiment, the status of MSS and MSI-H cancer in the subject is determined by an FDA-approved test.

In another embodiment, the agent in a) is administered in an amount sufficient to cause the effect described in a).

In another embodiment, the WRN inhibitor is administered to a patient already treated with an agent a), and the effects described in a) are confirmed according to tests described above.

### Definitions

“MGMT” means O⁶-methylguanine DNA methyltransferase.

“MGMT” refers to the gene encoding MGMT. The MGMT gene encodes a repair protein (MGMT; formerly also termed alkyl guanine alkyltransferase) that removes DNA alkylation modifications from DNA. Alkylating chemotherapeutic agents such as TMZ (temozolomide) induce cytotoxic cell death in tumor cells by alkylating DNA at multiple sites. Repair of the most toxic event, alkylation of the O⁶ group of guanine, is dependent on MGMT.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, patients, cancers and the like, this is taken to mean also a single compound, patient, or the like.

References in this specification to “the invention” are intended to reflect embodiments of the several inventions disclosed in this specification and should not be taken as unnecessarily limiting of the claimed subject matter.

The term “Compound A” as used herein refers to the compound of Example 42 and Example 123 as described in WO 2022/249060:

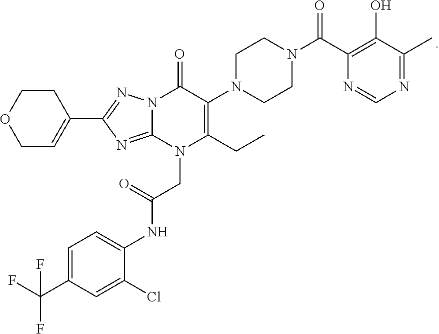 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl]

The term “Compound B” as used herein refers to the compound of Example 58 as described in WO 2022/249060:

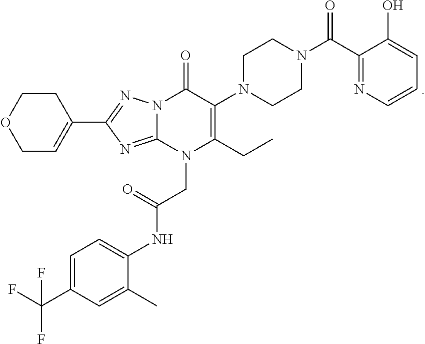 [SMILES: CCc1c(N2CCN(C(=O)c3ncccc3O)CC2)c(=O)n2nc(C3=CCOCC3)nc2n1CC(=O)Nc1ccc(C(F)(F)F)cc1C]

The WRN inhibitor for use according to the invention described herein may alternatively be selected from a compound disclosed in WO2023/062575 or WO2019/241802.

“Combination” refers to either a fixed combination in one dosage unit form, or a combined administration, for example where a WRN inhibitor, such as a compound of formula (I), or (1g), or a pharmaceutically acceptable salt thereof, and a combination partner (e.g. another drug as explained herein, also referred to as “therapeutic agent” or “co-agent”) may be administered independently at the same time or separately within time intervals, especially where these time intervals allow that the combination partners show a cooperative, e.g. synergistic effect. The combination partner for the WRN inhibitor such as the compound of formula (I) or (1g), may also for example be an agent that is capable of sensitising or priming the cancer cells to treatment, for example for an improved response to treatment with a WRN inhibitor. The combination partner may therefore be used for example to:
sensitise the cancer cells, for example for an improved response to treatment with a WRN inhibitor,
prime the cancer cells, for example, such priming may include triggering hypermutation status in cancer cells,
create or increase MMR deficiency in cancer cells,
create or increase MSI-H status in cancer cells,
increase MMR heterogeneity of cancer cells, and/or
create or increase resistance in cancer cells to temozolomide.

The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. combination partners of the present invention, are both administered to a patient simultaneously in the form of a single entity or dosage. The term “non-fixed combination” means that the therapeutic agents, e.g. combination partners of the present invention, are both administered to a patient as separate entities either simultaneously, concurrently or sequentially with no specific time limits, wherein such administration provides therapeutically effective levels of the two compounds in the body of the patient, or one agent provides a sensitizing or priming effect before or during treatment with a combination partner. This also applies to cocktail therapy, e.g. the administration of three or more therapeutic agents.

In the combination therapies of the invention, the therapeutic agents may be manufactured and/or formulated by the same or different manufacturers. Moreover, the therapeutic agents may be brought together into a combination therapy: (i) prior to release of the combination product to physicians (e.g. in the case of a kit comprising the therapeutic agents); (ii) by the physician themselves (or under the guidance of the physician) shortly before administration; (iii) in the patient themselves, e.g. during sequential administration of the therapeutic agents.

‘Pre-treatment’ means administration separately and prior to administration of certain other active compounds, in particular the WRN inhibitor. For example, pre-treatment may sensitise or prime the cancer cells to be more responsive or sensitive to the active compound(s) subsequently administered. Pre-treatment may be used for example to increase MMR deficiency, or may be used to increase resistance of cancer cells to the pre-treatment agent. Pre-treatment may be used to create or increase MSI-H status in cancer cells, create or increase MMR deficiency in cancer cells, or increase MMR heterogeneity of cancer cells. Temozolomide can be used herein as a pre-treatment agent to sensitise or prime the cancer cells to subsequent treatment, in particular to subsequent treatment with a WRN inhibitor. It is recognized that such effects may also occur when the agent such as temozolomide is used directly in combination with a WRN inhibitor, without pre-treatment administration in advance. The effects of such-pre-treatment may be determined according to tests taught in the art, or tests commercially available, or an FDA-approved test. The pre-treatment may be for example using an agent selected from temozolomide, cisplatin and 6-thioguanine, or the agent may be an ionising radiation based therapy selected from i) external beam radiation, ii) brachytherapy and ii) a radiopharmaceutical).

The term “synergistic effect” as used herein refers to action of two or three therapeutic agents producing an effect, for example, slowing the progression of a proliferative disease, particularly cancer, or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median effect equation (Chou, T. C. and Talalay, P., Adv. Enzyme Regul. 22: 27-55 (1984)). Each equation referred to above can be applied to experimental data to generate a corresponding graph to aid in assessing the effects of a drug combination. The corresponding graphs associated with the equations referred to above are the concentration-effect curve, isobologram curve and combination index curve, respectively.

The term “pharmaceutically acceptable salts” refers to salts that retain the biological effectiveness and properties of the compound and which typically are not biologically or otherwise undesirable. The compound may be capable of forming acid addition salts by virtue of the presence of an amino group.

Unless otherwise specified, or clearly indicated by the text, reference to therapeutic agents useful in the pharmaceutical combination of the present invention includes both the free base of the compounds, and all pharmaceutically acceptable salts of the compounds.

The term “combination” or “pharmaceutical combination” is defined herein to refer to either a fixed combination in one dosage unit form, a non-fixed combination or a kit of parts for the combined administration where the therapeutic agents may be administered together, independently at the same time or separately within time intervals, which preferably allows that the combination partners show a cooperative, e.g. synergistic effect. Thus, the single compounds of the pharmaceutical combination of the present invention could be administered simultaneously or sequentially. Furthermore, the pharmaceutical combination of the present invention may be in the form of a fixed combination or in the form of a non-fixed combination.

The term “fixed combination” means that the therapeutic agents, e.g., the single compounds of the combination, are in the form of a single entity or dosage form.

The term “non-fixed combination” means that the therapeutic agents, e.g., the single compounds of the combination, are administered to a patient as separate entities or dosage forms either simultaneously or sequentially with no specific time limits, wherein preferably such administration provides therapeutically effective levels of the two therapeutic agents in the body of the subject, e.g., a mammal or human in need thereof.

The pharmaceutical combinations can further comprise at least one pharmaceutically acceptable carrier. Thus, the present invention relates to a pharmaceutical composition comprising the pharmaceutical combination of the present invention and at least one pharmaceutically acceptable carrier.

As used herein, the term “carrier” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Generally, the term “pharmaceutical composition” is defined herein to refer to a mixture or solution containing at least one therapeutic agent to be administered to a subject, e.g., a mammal or human. The present pharmaceutical combinations can be formulated in a suitable pharmaceutical composition for enteral or parenteral administration are, for example, those in unit dosage forms, such as sugar-coated tablets, tablets, capsules or suppositories, or ampoules. If not indicated otherwise, these are prepared in a manner known per se, for example by means of various conventional mixing, comminution, direct compression, granulating, sugar-coating, dissolving, lyophilizing processes, or fabrication techniques readily apparent to those skilled in the art. It will be appreciated that the unit content of a combination partner contained in an individual dose of each dosage form need not in itself constitute an effective amount since the necessary effective amount may be reached by administration of a plurality of dosage units. The pharmaceutical composition may contain, from about 0.1% to about 99.9%, preferably from about 1% to about 60%, of the therapeutic agent(s). One of ordinary skill in the art may select one or more of the aforementioned carriers with respect to the particular desired properties of the dosage form by routine experimentation and without any undue burden. The amount of each carriers used may vary within ranges conventional in the art. The following references disclose techniques and excipients used to formulate oral dosage forms. See The Handbook of Pharmaceutical Excipients, 4th edition, Rowe et al., Eds., American Pharmaceuticals Association (2003); and Remington: the Science and Practice of Pharmacy, 20th edition, Gennaro, Ed., Lippincott Williams & Wilkins (2003). These optional additional conventional carriers may be incorporated into the oral dosage form either by incorporating the one or more conventional carriers into the initial mixture before or during granulation or by combining the one or more conventional carriers with granules comprising the combination of agents or individual agents of the combination of agents in the oral dosage form. In the latter embodiment, the combined mixture may be further blended, e.g., through a V-blender, and subsequently compressed or molded into a tablet, for example a monolithic tablet, encapsulated by a capsule, or filled into a sachet. Clearly, the pharmaceutical combinations of the present invention can be used to manufacture a medicine.

The present invention relates to such pharmaceutical combinations or pharmaceutical compositions that are particularly useful as a medicine.

Specifically, the combinations or compositions of the present invention can be applied in the treatment of cancer.

The present invention also relates to use of pharmaceutical combinations or pharmaceutical compositions of the present invention for the preparation of a medicament for the treatment of a cancer, and to a method for treating cancer in a subject in need thereof comprising administering to the subject a therapeutically effective amount of a pharmaceutical combination according to the present invention, or the pharmaceutical composition according to the present invention.

The term “treatment” as used herein comprises a treatment relieving, reducing or alleviating at least one symptom in a subject, increasing progression-free survival, overall survival, extending duration of response or delaying progression of a disease. For example, treatment can be the diminishment of one or several symptoms of a disorder or complete eradication of a disorder, such as cancer. Within the meaning of the present invention, the term “treatment” also denotes to arrest, delay the onset (i.e., the period prior to clinical manifestation of a disease) and/or reduce the risk of developing or worsening a disease in a patient, e.g., a mammal, particularly the patient is a human. The term “treatment” as used herein comprises an inhibition of the growth of a tumor incorporating a direct inhibition of a primary tumor growth and/or the systemic inhibition of metastatic cancer cells.

A “subject,” “individual” or “patient” is used interchangeably herein, which refers to a vertebrate, preferably a mammal, more preferably a human. Mammals include, but are not limited to, mice, simians, humans, farm animals, sport animals, and pets.

The term “a therapeutically effective amount” of a compound (e.g. chemical entity or biologic agent) of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one embodiment a therapeutically effective amount in vivo may range depending on the route of administration, between about 0.1-500 mg/kg, or between about 1-100 mg/kg.

As used herein, the term “inhibit”, “inhibition” or “inhibiting” refers to the reduction or suppression of a given condition, symptom, or disorder, or disease, or a significant decrease in the baseline activity of a biological activity or process.

The term “therapeutically effective agent” as used herein is intended to be construed broadly and includes both drug molecules and ionising radiation based therapies. The ionizing radiation based therapy may be provided in any suitable form known in the art, for example in the form of an external beam radiation therapy, brachytherapy or via a radiopharmaceutical (such as a radioligand agent). External beam radiation therapy refers to wherein radiation is directed at the tumor from a source outside of the body. The term “brachytherapy” refers to a form of radiation therapy whereby a radiation source is positioned at the tumor site, enabling a high dose of localized radiation to be administration to that tumor site. Radiopharmaceuticals are discussed in more detail below.

The optimal dosage of each combination partner for treatment of a cancer can be determined empirically for each individual using known methods and will depend upon a variety of factors, including, though not limited to, the degree of advancement of the disease; the age, body weight, general health, gender and diet of the individual; the time and route of administration; and other medications the individual is taking. Optimal dosages may be established using routine testing and procedures that are well known in the art. The amount of each combination partner that may be combined with the carrier materials to produce a single dosage form will vary depending upon the individual treated and the particular mode of administration. In some embodiments the unit dosage forms containing the combination of agents as described herein will contain the amounts of each agent of the combination that are typically administered when the agents are administered alone.

Frequency of dosage may vary depending on the compound used and the particular condition to be treated or prevented. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

The combination of the present invention may, for example, be in unit dosage of about 1-1000 mg of each active ingredient for a subject of about 50-70 kg.

‘Zwitterion’ or ‘zwitterionic form’ means a compound containing both positive and negatively charged functional groups.

For example, the compound of formula (I) described herein can include the following forms, wherein R₄ is the zwitterionic form (c) or non-zwitterionic form (d),

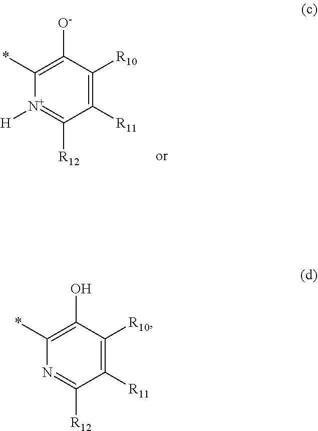

or a mixture thereof.

The compound of formula (I) described herein can also include the following forms, wherein R₄ is the zwitterionic form (a) or (b) or the non-zwitterionic form (e),

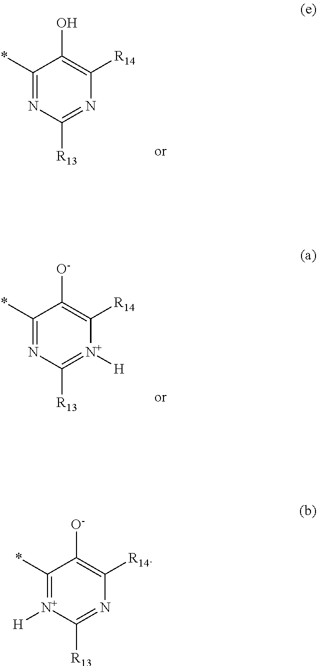

or a mixture of two thereof, or a mixture of all three thereof.

A ‘compound of formula (I)’ includes zwitterionic and non-zwitterionic forms, and mixtures thereof.

halo means fluoro, chloro or bromo, particularly fluoro or chloro.

Alkyl, and alkoxy groups, containing the requisite number of carbon atoms, can be unbranched or branched. Examples of alkyl include, but are not limited to, methyl, ethyl, n-propyl, i-propyl, n-butyl, i-butyl, sec-butyl and t-butyl. Examples of alkoxy include methoxy, ethoxy, n-propoxy, i-propoxy, n-butoxy, i-butoxy, sec-butoxy and t-butoxy.

‘═O’ means an oxo substituent.

When R₁ is substituted or unsubstituted cycloalkenyl, said cycloalkenyl includes, but is not limited to, groups such as cyclohexenyl, in particular cyclohex-1-en-1-yl.

When R₁ is substituted or unsubstituted heterocyclyl, said heterocyclyl includes, but is not limited to, groups such as morpholinyl, piperidinyl, pyrrolidinyl, 6-oxa-3-azabicyclo[3.1.1]heptan-3-yl, 5,6-dihydro-1,4-dioxin-2-yl, dihydropyranyl, in particular 3,4-dihydro-2H-pyran-6-yl, 5,6-dihydro-2H-pyran-3-yl and 3,6-dihydro-2H-pyran-4-yl, piperazinyl, tetrahydropyridinyl, such as 1,4,5,6-tetrahydropyridin-3-yl and 1,2,3,6-tetrahydropyridin-4-yl and dihydropyridinyl, such as 3,6-dihydropyridinyl.

When R₁ is substituted or unsubstituted heteroaryl, said heteroaryl includes, but is not limited to, groups such as pyridinyl, in particular pyridin-3-yl.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to colorectal, gastric, endometrial, prostate, adrenocortical, uterine, cervical, esophageal, breast, kidney, ovarian cancer and the like.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

‘WRN inhibitor’ or ‘WRN helicase inhibitor’ as used herein means a compound or therapeutic agent that inhibits Werner Syndrome RecQ DNA helicase (WRN). The term “WRN” as used herein refers to the protein of Werner Syndrome RecQ DNA helicase. The term “WRN” includes mutants, fragments, variants, isoforms, and homologs of full-length wild-type WRN. In one embodiment, the protein is encoded by the WRN gene (Entrez gene ID 7486; Ensembl ID ENSG00000165392). Exemplary WRN sequences are available at the Uniprot database under accession number Q14191.

‘Disease or condition mediated by WRN’ includes a disease or condition, such as cancer, which is treated by WRN inhibition. In particular this can include cancers characterized as microsatellite instability-high (MSI-H) or mismatch repair deficient (dMMR).

‘Microsatellite unstable cancer’, microsatellite instability-high cancer’, ‘microsatellite high cancer’ and ‘MSI-high cancer’ ‘MSI^{hi}’ and ‘MSI-H’ when used herein, are used interchangeably, and describe cancers that have a high number of alterations in the length of simple repetitive genomic sequences within microsatellites.

The determination of MSI-H or dMMR tumor status for patients can be performed using, e.g., polymerase chain reaction (PCR) tests for MSI-H status or immunohistochemistry (IHC) tests for dMMR. Methods for identification of MSI-H or dMMR tumor status are described, e.g., in Ryan et al. Crit Rev Oncol Hematol. 2017; 116:38-57; Dietmaier and Hofstadter. Lab Invest 2001, 81:1453-1456; and Kawakami et al. Curr Treat Options Oncol. 2015; 16(7): 30).

“MSS” means microsatellite stable. A cancer is deemed to have MSS status following a negative result in the MSI-H status test. Accordingly, in an embodiment, the methods or uses disclosed herein may further comprise administering an MSI-H status test to the patient. In some embodiments, the MSI-H status test is an FDA-approved test, e.g., FoundationOne CDx.

“pMMR” means proficient mismatch repair.

“pMMR/MSS” means “MSS cancer” or “pMMR cancer”. MSS cancer cells are proficient for MMR (pMMR), thus the terms “MSS cancer” or “pMMR cancer” can be used separately and interchangeably.

Microsatellite instability is present in various cancers, including but not limited to colorectal cancer, gastric cancer and endometrial cancer in particular, but also in adrenocortical, uterine, cervical, esophageal, breast, kidney, prostate and ovarian cancers. Examples of microsatellite high cancers include uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

A cancer that has “defective mismatch repair” (dMMR) or “dMMR character” includes cancer types associated with documented MLH1, PMS2, MSH2, MSH3, MSH6, MLH3, and PMS1 mutations or epigenetic silencing, microsatellite fragile sites, or other gene inactivation mechanisms, including but not limited to cancers of the lung, breast, kidney, large intestine, ovary, prostate, upper aerodigestive tract, stomach, endometrium, liver, pancreas, haematopoietic and lymphoid tissue, skin, thyroid, pleura, autonomic ganglia, central nervous system, soft tissue, pediatric rhabdoid sarcomas, melanomas and other cancers. A cell or cancer with “defective” mismatch repair has a significantly reduced (e.g., at least about 25%, 30%, 40%, 50%, 60%, 70%, 80% or 90% decrease) amount of mismatch repair. In some cases, a cell or cancer which is defective in mismatch repair will perform no mismatch repair.

As described herein, the WRN inhibitor may be used in combination with at least one therapeutic agent which can be used in one or more of the following ways, to:
sensitise the cancer cells, for example for an improved response to treatment with a WRN inhibitor,
prime the cancer cells, for example, such priming may include triggering hypermutation status in cancer cells,
create or increase MMR deficiency in cancer cells,
create or increase MSI-H status in cancer cells,
increase MMR heterogeneity of cancer cells, and/or
create or increase resistance in cancer cells to temozolomide.

These effects may be determined according to tests taught in the art, or tests commercially available, or an FDA-approved test. Such agents are known in the art. Said therapeutic agent may be an alkylating agent, for example temozolomide. Said therapeutic agent may also be cisplatin or 6-thioguanine. Said therapeutic agent may also be an ionising radiation based therapy selected from i) external beam radiation, ii) brachytherapy and ii) a radiopharmaceutical, for example as described herein.

### REFERENCES

Cell 177, 821-836, May 2, 2019,
Journal of Clinical Oncology 40, no. 14 (May 10, 2022) 1562-1573 DOI: 10.1200/JCO.21.02583,
Biochemical Pharmacology, Vol 54, pp. 419-424, 1997, Nat Genet. 2021 July; 53(7): 1088-1096. doi: 10.1038/s41588-021-00874-3.
Germano, G., Lamba, S., Rospo, G. et al. Inactivation of DNA repair triggers neoantigen generation and impairs tumour growth. Nature 552, 116-120 (2017), https://doi.org/10.1038/nature24673
Cancer Discov 2022; 12:1656-75 doi: 10.1158/2159-8290.CD-21-1434

Priming with temozolomide may be performed in patients with MSS cancer, or in patients with MGMT-defective, or MGMT-deficient, or MGMT-silenced tumors. Such priming may take place in patients with pMMR/MSS MGMT-defective, or pMMR/MSS MGMT-deficient, or pMMR/MSS MGMT-silenced.tumors. In one example, the tumor is MGMT-defective, or MGMT-deficient, or MGMT-silenced CRC. In particular, the tumor is pMMR/MSS and MGMT-silenced mCRC. In another aspect, the tumor is MGMT-methylated glioblastoma.

Assessment of MGMT tumor status can be for example by protein expression promoter methylation, as described in the references below which are hereby incorporated by reference in their entirety, or also by MGMT gene mutations. Methods to evaluate MGMT status are summarized in this reference: Mansouri A, Hachem L D, Mansouri S, et al. MGMT promoter methylation status testing to guide therapy for glioblastoma: refining the approach based on emerging evidence and current challenges. Neuro Oncol. 2019; 21(2): 167-178. doi: 10.1093/neuonc/noy132.

### REFERENCES

Cancer Discov 2022; 12:1656-75, doi: 10.1158/2159-8290.CD-21-1434,
Amodio et al., 2023, Cancer Cell 41, 196-209, https://doi.org/10.1016/j.ccell.2022.12.003,
J Clin Oncol 40: 1562-1573, http://ascopubs.org/doi/full/10.1200/JCO.21.02583,
118 Nature Vol 552 7 Dec. 2017, http://www.nature.com/doifinder/10.1038/nature24673.

In an alternative aspect, other alkylating agents can be used instead of temozolide, for example as a pre-treatment agent or priming agent, or to:
create or increase MMR deficiency in cancer cells,
create or increase MSI-H status in cancer cells.

Thus, there is also provided an alkylating agent for use in the embodiments described herein, in place of temozolomide. Said treatment is optionally in combination with irinotecan.

In another aspect, the WRN inhibitor is used in combination with cisplatin, instead of temozolide, for example as a pre-treatment agent or priming agent, or to:
create or increase MMR deficiency in cancer cells,
create or increase MSI-H status in cancer cells.

Said treatment is optionally in further combination with irinotecan.

In another aspect, the WRN inhibitor is used in combination with an ionising radiation based therapy, for example as a pre-treatment agent or priming agent, or to:
create or increase MMR deficiency in cancer cells,
create or increase MSI-H status in cancer cells.

Said treatment is optionally in further combination with irinotecan.

In another aspect, the WRN inhibitor is used in combination with 6-thioguanine, instead of temozolide, in the context as described herein, for example as a pre-treatment agent or priming agent, or to:
create or increase MMR deficiency in cancer cells,
create or increase MSI-H status in cancer cells.

Said treatment is optionally in further combination with irinotecan.

‘May join’ means joins or does not join.

‘May be replaced by deuterium’ means is replaced by deuterium, or is not replaced by deuterium.

The term “chemotherapy agent” or interchangeably “chemotherapeutic agent” as used herein refers in a specific embodiment to a cytotoxic drug. Examples include alkylating agents, anthracyclines, antimetabolites, intercalating agents (e.g. doxorubicin or epirubicin) and topoisomerase inhibitors. In an embodiment, the chemotherapy agent is selected from vinblastine, vindesine, vinorelbine, vincristine, anastrozole (Arimidex®), bicalutamide (Casodex®), bleomycin (e.g. bleuomycin sulfate) (Blenoxane®), busulfan (Myleran®), busulfan injection (Busulfex®), calactin, capecitabine (Xeloda®), N4-pentoxycarbonyl-5-deoxy-5-fluorocytidine, carboplatin (Paraplatin®), carmustine (BICNUR), lomustine (CCNUR), chlorambucil (Leukeran®), bendamustine (Treanda®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegen), daunorubicin n hydrochloride (Cerubidine®), daunorubicin citrate liposome injection (DaunoXome®), dexamethasone, docetaxel (Taxotere®), doxorubicin (e.g. doxorubicin hydrochloride) (Adriamycin®, Rubex®), etoposide (Vepesid®), fludarabine phosphate (Fludara®), 5-fluorouracil (Adrucil®, Efudex®), flutamide (Eulexin®), tezacitabine, gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), a mitomycin (e.g. mitomycin A, mitomycin B or mitomycin C, particularly mitomycin C), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), camptothecin, teniposide (Vumon®), 6-thioguanine, thiotepa, tirapazamine (Tirazone®), topotecan hydrochloride for injection (Hycamptin®), vinblastine (Velban®), vincristine (Oncovin®), oxaliplatin (Eloxatin®), epirubicin (Ellence®, Pharmorubicin®), temozolomide (Temodar®), tegafur, gimeracil and vinorelbine (Navelbine®), in particular irinotecan.

In an embodiment, the chemotherapy agent is selected from gemcitabine, camptothecin, irinotecan (Camptosar®), docetaxel (Taxotere®), doxorubicin (e.g. doxorubicin hydrochloride) (Adriamycin®, Rubex®), 5-fluorouracil (Adrucil®, Efudex®), capecitabine (Xeloda®), etoposide (Vepesid®), epirubicin (Ellence®, Pharmorubicin®), oxaliplatin (Eloxatin®), mitomycin (e.g. mitomycin A, mitomycin B or mitomycin C, particularly mitomycin C), cisplatin (Platinol®), carboplatin (Paraplatin®) and paclitaxel (Taxol®).

In an embodiment, the chemotherapy agent is an alkylating agent, e.g. an alkylating agent selected from cyclophosphamide, ifosfamide, melphalan, chlorambucil and bendamustine.

In an embodiment, the chemotherapy agent is a topoisomerase inhibitor such as from QAP1, irinotecan, topotecan, camptothecin and etoposide

In an embodiment, the chemotherapy agent is a DNA alkylating agent such as cisplatin, carboplatin or oxaliplatin.

In an embodiment, the chemotherapy agent is an antimetabolite such as 5-fluorouracil or tegafur (which is a prodrug of 5-fluorouracil).

In an embodiment, the chemotherapy agent is a microtubule polymer stabilizer such as docetaxel or paclitaxel.

In an embodiment, the chemotherapy agent is a antineoplastic agent such as mitomycin (e.g. mitomycin C).

In an embodiment, the chemotherapy agent is an intercalating agent (e.g. doxorubicin or epirubicin).

In an embodiment, the chemotherapy agent is a vinca alkaloid, such as vinblastine, vindesine, vinorelbine or vincristine.

In an embodiment, where the chemotherapy agent is tegafur, the tegafur is administered in the form of TS-1 (also known as teysuno and S-1), which is a combination of Tegafur, gimeracil and oteracil.

In an embodiment, the PD-1 inhibitor is an anti-PD-1 antibody. In an embodiment, the PD-1 inhibitor may be selected from PDR001 (Novartis), Nivolumab (Bristol-Myers Squibb), Pembrolizumab (Merck & Co), Pidilizumab (CureTech), MEDI0680 (Medimmune), Cemiplimab (REGN2810, Regeneron), Dostarlimab (TSR-042, Tesaro), PF-06801591 (Pfizer), Tislelizumab (BGB-A317, Beigene), BGB-108 (Beigene), INCSHR1210 (Incyte), Balstilimab (AGEN2035, Agenus), Sintilimab (InnoVent), Toripalimab (Shanghai Junshi Bioscience), Camrelizumab (Jiangsu Hengrui Medicine Co.), and AMP-224 (Amplimmune), in particular PDR001 or Tislelizumab.

In an embodiment of any one of the aspects of the invention, where an MDM2 inhibitor is present, the MDM2 inhibitor may be selected from the group consisting of nutlin-3a, idasanutlin (also known as RG7388), RG7112, AMG-232 (also known as KRT-232), APG-115, BI-907828, milademetan and HDM201 (also known as siremadlin), or a pharmaceutically acceptable salt thereof.

In an embodiment of any one of the aspects of the invention, where a MEK inhibitor is present, the MEK inhibitor may be selected from the group consisting of refametinib, pimasertib, selumetinib, trametinib, binimetinib and cobimetinib, or a pharmaceutically acceptable salt thereof.

In an embodiment of any one of the aspects of the invention, where a MEK inhibitor is present, the MEK inhibitor may be trametinib, or a pharmaceutically acceptable salt thereof.

In an embodiment of any one of the aspects of the invention, where a WEE1 inhibitor is present, the WEE1 inhibitor may be selected from Adavosertib (also known as AZD1775 and MK-1775) and PDO166285. In an embodiment, the WEE1 inhibitor is Adavosertib.

In an embodiment of any one of the aspects of the invention, where an ATR inhibitor is present, the ATR inhibitor may be selected from RP-3500, ceralasertib (also known as AZD6738), berzosertib, ART-0380, gartisertib (also known as M4344), and elimusertib (BAY-1895344). In an embodiment, the ATR inhibitor is elimusertib (BAY-1895344).

In an embodiment of any one of the aspects of the invention, where a DNA-PK inhibitor is present, the DNA-PK inhibitor may be selected from AZD-7648, NU7441 (also known as KU-57788), Omipalisib, BAY8400 and M3814. In an embodiment, the DNA-PK inhibitor is AZD-7648 or NU7441 (KU-57788), particularly AZD-7648.

In an embodiment of any one of the aspects of the invention, where a G4-quadruplex stabilizer is present, the G4-quadruplex stabilizer may be selected from 5ME, Ant1,5, BRAC019, C8, C14, c-exNDIs, CORON, CX-3543 (also known as Quarfloxin), EMICORON, IZCZ-0, IZCZ-3, IZTC-1, N,N′-bis(3,4-dihydroxybenzylidene)-1,2-diaminobenzene, PhenDC3, Phenyl 1,2,3-triazole-thymidine ligands, Pyridostatin, RHPS4, TMPyP4 and trans-resveratrol (tRES). In an embodiment, the G4-quadruplex stabilizer is preferably pyridostatin.

In an embodiment of any one of the aspects of the invention, where an ATM inhibitor is present, the ATM inhibitor may be selected from KU-55933, KU-60019, KU-59403, M3541, CP-466722, AZ31, AZ32, AZD0156 and AZD1390. In an embodiment, the ATM inhibitor is KU-60019.

In an embodiment of any one of the aspects of the invention, where a PARP inhibitor is present, the PARP inhibitor may be selected from olaparib, NMS293, niraparib veliparib, rucaparib, prexasertib, talazoparib, AZD-5305 and KU0058948. In an embodiment, the PARP inhibitor is olaparib.

In an embodiment of any one of the aspects of the invention, where a topoisomerase inhibitor is present, the topoisomerase inhibitor may be selected from QAP1, irinotecan, topotecan, camptothecin and etoposide. In an embodiment, the topoisomerase inhibitor is selected from QAP1, etoposide and irinotecan. In an embodiment, the topoisomerase inhibitor is a topoisomerase I inhibitor. In another embodiment, the topoisomerase inhibitor is a topoisomerase II inhibitor.

In an embodiment of any one of the aspects of the invention, where a CHK1 or CHK2 inhibitor is present, the CHK1 or CHK2 inhibitor is selected from GDC-0575, Prexasertib (LY2606368), SCH900776 (also known as MK-8776), SRA737, PF477736, LY2606368 and AZD7762. The CHK1 or CHK2 inhibitor may be a dual CHK1/2 inhibitor (such as AZD7762). As used herein, ‘CHK1 or CHK2 inhibitor’ means an agent which is a selective inhibitor of CHK1 over CHK2, or which is a selective inhibitor of CHK2 over CHK1, or an agent which is an inhibitor of both CHK2 and CHK1 (‘dual CHK1/2 inhibitor’).

In an embodiment of any one of the aspects of the invention, the therapeutically active agent is a PI3K inhibitor, e.g. a PI3K-alpha inhibitor. In an embodiment, the PI3K inhibitor is selected from AMG511, buparlisib, Idelalisib, Copanlisib, Duvelisib, Alpelisib, and Umbralisib. In an embodiment, the PI3K inhibitor is Alpelisib. In an embodiment, the therapeutically active agent is a PI3K inhibitor, e.g. a PI3K-alpha inhibitor, e.g. Alpelisib, and the cancer is MSI-H. In an embodiment, the PI3K inhibitor is a PI3K-alpha inhibitor selected from RLY-2608, BPI-21668, PF-06843195, LX-086, HS-10352, HH-CYH33, JS-105, MEN-1611, LOX-22783, TOS-358, STX-478, Alpelisib, Serabelisib and Inavolisib. In an embodiment, the PI3K-alpha inhibitor is selected from Alpelisib, Serabelisib and Inavolisib.

According to another aspect of the invention, there is hereby provided a method of treating cancer in a subject in need thereof, the method comprising administering to the subject a therapeutically effective amount of a WRN inhibitor in combination with a polymerase theta inhibitor. In an embodiment, the polymerase theta inhibitor is ART812. In an alternative embodiment the polymerase theta inhibitor is RP-2119 or Pol Theta Helicase Inhibitor (Ideaya/GSK). In an embodiment, the therapeutically active agent is a polymerase theta inhibitor (e.g. ART812) and the cancer is MSI-H.

According to another aspect of the invention, there is hereby provided a method of treating cancer in a subject in need thereof, the method comprising administering to the subject a therapeutically effective amount of a WRN inhibitor in combination with IAP inhibitor/an SMAC mimetic. In an embodiment, the IAP inhibitor is selected from LCL161, Birinapant and Xevinapant.

According to another aspect of the invention, there is hereby provided a method of treating cancer in a subject in need thereof, the method comprising administering to the subject a therapeutically effective amount of a WRN inhibitor in combination with a CTLA-4 inhibitor. In an embodiment, the CTLA-4 inhibitor is ipilimumab or tremelimumab, for example ipilimumab.

According to another aspect of the invention, there is hereby provided a method of treating cancer in a subject in need thereof, the method comprising administering to the subject a therapeutically effective amount of a WRN inhibitor in combination with a KRAS G12C inhibitor.

KRAS G12C inhibitors useful in combinations and methods of the invention include a compound selected from sotorasib, adagrasib, GDC6036, D-1553, and in particular, JDQ443. JDQ443 is described in Example 1 of PCT application WO2021/124222, published 24 Jun. 2021. WO2021/124222 is hereby incorporated by reference in its entirety.

According to another aspect of the invention, there is hereby provided a method of treating cancer in a subject in need thereof, the method comprising administering to the subject a therapeutically effective amount of a WRN inhibitor in combination with a SHP2 inhibitor. Examples of SHP2 inhibitors useful in combinations and methods of the present invention include TNO155, JAB3312 or JAB-3068 (Jacobio), RLY1971 (Roche), SAR442720/RMC-4630 (Sanofi/Revolution Medicines), RMC4450 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), HS-10381 (Hansoh Pharma/Jiangsu Hansoh), BPI-442096 (Betta Pharmaceuticals), I-0436650 (IRBM), PCC-0208023 (Binzhou Medical University), IACS-15414 (Navire) and X-37-SHP2 (X-37). A particularly preferred SHP2 inhibitor for use according to the invention is (3S,4S)-8-(6-amino-5-((2-amino-3-chloropyridin-4-yl)thio)pyrazin-2-yl)-3-methyl-2-oxa-8-azaspiro[4.5]decan-4-amine (TNO155), or a pharmaceutically acceptable salt thereof. TNO155 is synthesized according to example 69 of WO2015/107495, which is hereby incorporated by reference in its entirety. A preferred salt of TNO155 is the succinate salt.

According to another aspect of the invention, there is hereby provided a method of treating cancer in a subject in need thereof, the method comprising administering to the subject a therapeutically effective amount of a WRN inhibitor in combination with a KRAS G12D inhibitor. Examples of KRAS G12D inhibitors useful in combinations and methods of the present invention include siG12D LODER, HRS-4642 and ASP-3082.

According to another aspect of the invention, there is hereby provided a method of treating cancer in a subject in need thereof, the method comprising administering to the subject a therapeutically effective amount of a WRN inhibitor in combination with a YAP/TEAD inhibitor. Examples of YAP/TEAD inhibitors useful in combinations and methods of the present invention include IAG933 (Novartis), NSC-682769 (University of California), MSC-4106 (Merck), GNE-7883 (Genentech), TED-347 (Indiana University), K-975 (Kyowa Kirin) and the compounds in WO 2021/186324, WO2022/087008; WO2021/102204; WO2020/214734; WO2020/097389; WO2019/222431; WO2019/113236; WO2019/040380; WO2018/204532; WO2017/058716; WO2022/159986; WO2022/120354; WO2022/120355; WO2022/120353; WO2020/243423 or WO2020/243415.

According to another aspect of the invention, there is hereby provided a method of treating cancer in a subject in need thereof, the method comprising administering to the subject a therapeutically effective amount of a WRN inhibitor in combination with a BCL2 inhibitor, or a BCL2/BCLxl dual inhibitor. Examples of such inhibitors useful in combinations and methods of the present invention include venetoclax, APG-2575 (lisaftoclax), obatoclax meylate, BGB-11417 (Beigene), pelcitoclax, Zn-d5 (Zentalis), AZD-0466 (Astra Zeneca), ABBV-453, ABBV-167 (AbbVie), LP-118, LP-108, (Guangzhou Lupeng), FCN-338 (Fochon Pharmaceuticals) and navitoclax.

According to another aspect of the invention, there is hereby provided a method of treating cancer in a subject in need thereof, the method comprising administering to the subject a therapeutically effective amount of a WRN inhibitor in combination with a MCL1 inhibitor. Examples of MCL1 inhibitors useful in combinations and methods of the present invention include AMG176 (tapotoclax), GS-9716, ABBV-467, Murizatoclax, AZD-5991, JNJ-1245, JNJ-4355, and PRT-1419 (Prelude Therapeutics).

According to another aspect of the invention, there is hereby provided a method of treating cancer in a subject in need thereof, the method comprising administering to the subject a therapeutically effective amount of a WRN inhibitor in combination with a CDK2 inhibitor. Examples of CDK2 inhibitors useful in combinations and methods of the present invention include BLU222 (Blueprint Medicines), PF-07104091 (Pfizer) and INCB-0123667 (Incyte). Examples of CDK2/CDK9 or CDK2/CDK9/CDK7 inhibitors useful in combinations and methods of the present invention include fadraciclib and seliciclib.

According to another aspect of the invention, there is hereby provided a method of treating cancer in a subject in need thereof, the method comprising administering to the subject a therapeutically effective amount of a WRN inhibitor in combination with a CDK4 or CDK4/6 inhibitor. Examples of CDK4/6 inhibitors useful in combinations and methods of the present invention include ribociclib, palbociclib, trilaciclib, birociclib and lerociclib.

According to another aspect of the invention, there is hereby provided a method of treating cancer in a subject in need thereof, the method comprising administering to the subject a therapeutically effective amount of a WRN inhibitor in combination with a HIF2 alpha inhibitor. Examples of HIF2 alpha inhibitors useful in combinations and methods of the present invention include belzutifan, MK-6482, PT2385 and DFF332.

According to another aspect of the invention, there is hereby provided a method of treating cancer in a subject in need thereof, the method comprising administering to the subject a therapeutically effective amount of a WRN inhibitor in combination with:
a PLK1 inhibitor, such as volasertib,
a PRMT5 inhibitor, such as SCR-6277, AMG-193, SKL-27969, MRTX-1719, onametostat/JNJ-64619178, TNG-90 and PF-0693999,
a STING agonist, such as CDK-002, TAK-500, ONO-7914, VB-85247, KL-340399, TAK-676, SNX-281, SB-11285 and IMSA-101,
a TRAIL receptor agonist, such as drozitumab, PRO-95780, CS-1008, IGM-8444, and lexatumumab.
a TAK1 inhibitor, such as takinib,
a MK2 inhibitor, such as CC-99677,
a HDAC inhibitor such as panobinostat, vorinostat, romidepsin, or belinostat,
an androgen biosynthesis inhibitor, such as abiraterone, or
an androgen receptor modulator, such as enzalutamide.

Radiopharmaceutical

The term “Radiopharmaceutical” as used herein refers to a pharmaceutical drug comprising one or more radioactive isotopes configured such that the radioactive isotopes are preferentially delivered to the tumor site. In some cases, the radiopharmaceutical can simply be a radioactive isotope or a pharmaceutically acceptable salt thereof. For example, ²²³Ra-Radium Dichloride (BAY88-8223, Xofigo® previously known as ²²³Ra-Alpharadin) (available from Bayer Pharma) is an approved medication for the treatment of cancer types that commonly metastasize to the bone. The radium is preferentially delivered to the bone as a result of its chemical similarity to calcium. Other examples are iodine salts based on ¹³¹I (usually in the form of Sodium Iodide), and ³²P-Sodium Phosphate used in the palliation of bone pain, ¹⁵³Sm-Lexidronam pentasodium (¹⁵³Sm-Samarium Ethylene Diamine Tetramethylene Phosphoric Acid, ¹⁵³Sm-Samarium EDTMP, Quadramet®) used for the relief of bone pain in patients with multiple osteoblastic skeletal metastases, ¹⁵³Sm-DOTMP (CycloSAM™), ¹⁵³Sm-Oxabiphor (¹⁵³Sm-Samarium oxa-bis(ethylenedithio) tetramethylphosphonium acid, ¹⁵³Sm-OXB, ¹⁵³Sm-Oxabifor, ¹⁵³Sm-ETMP) used for the relief of bone pain in patients with multiple osteoblastic skeletal metastases, ¹⁶⁶Ho-Phytate used for the potential treatment of chronic synovitis, ¹⁷⁷Lu-ST2210 (IART—¹⁷⁷Lu-DOTA-Biotin) (available from Sigma-Tau), ¹⁸⁶Re-Rhenium Etidronate (¹⁸⁶Re-HEDP, hydroxyethylidene diphosphonate) used in relieving the pain associated with metastatic bone cancer, ¹⁸⁶Re-Rhenium Sulfide used for isotopic radiation synovectomy of medium size joints, ¹⁸⁸Re-Rhenium Etidronate (HEDP) is indicated for bone pain palliation in cancer metastases (prostate, breast).

In other embodiments, the radioactive isotope is combined with a targeting vector intended to drive the radionuclide to the target. The combination of such a radioactive isotope and a targeting vector is referred to herein as a “radioligand agent”. The radionuclide is selected on the basis of the application of the drug (diagnostic or therapeutic agent), of the type of radiation and of its energy. The targeting vector is intended to drive the radionuclide preferentially to the target tissue, target organ or target cell and can be chemical molecules, peptides, polypeptide, proteins (such as antibodies, antigen binding fragments, Bispecific Antibodies, affibodies, or Fibronectin type III domains), peptidomimetics, fusion proteins/polypeptides, Aptamers, Antisense oligonucleotides, siRNA, microparticles or nanoparticles. In order to link the radionuclide to the vector, chemists may have to develop special chemical structures called linkers. The linkers may be inert moieties used to increase the distance of binding moieties from chelators in order to prevent steric influence and loss of activity on the cell receptors upon functionalization. The length and composition of the linker may influence the binding affinity of the radiopharmaceutical to the receptor, the accumulation of radionuclides in tumor cells and the pharmacokinetic.

Direct binding through so-called ‘covalent’ bonds is possible, e.g. with radionuclides such as the radiohalogens ¹³¹I or ²¹¹At. Radiometals may need a so-called ‘chelator’, or “chelating agent”, a molecule moiety in form of a cage that can trap the radiometal. Examples of chelating agents include but not limited to DOTA, DTPA, AAZTA, TCMC, DAT, DFO, DOTAGA, DOTAM, EDTA, HBED/HBED-CC, HYNIC, NODAGA, NODA, NODASA, NOPO, NOTA and PCTA and their derivates.

In one embodiment the radionuclides can be administrated in gel, micelle, sphere, particle, microparticle or nanoparticle forms and including therapeutics such as: ¹⁶⁶Ho-Chitosan used for hepatocellular carcinoma treatment (available from Dong Wha.), ⁹⁰Y—SIR-Spheres a resin-based microspheres used for hepatocellular carcinoma treatment (available from Sirtex), ⁹⁰Y-TheraSpheres a suspension of insoluble glass microspheres used for transarterial radioembolization in hepatic neoplasia including hepatocellular carcinoma (available from BTG/Boston Scientific), ⁹⁰Y-RadioGel a hydrogel liquid made of water-based biodegradable polymer that delivers ⁹⁰Y microspheres directly into tumor tissues (Vivos Inc.), ¹³¹I-SapC-DOPS (¹³¹I-Saposin; ¹³¹I-BXD-350) a nanovesicle composed of Saposin C (SapC) coupled to dioleoylphosphatidylserine (DOPS) (Molecular Targeting Technologies Inc.), ¹³¹I-Lipiodol (¹³¹I-Ethiodized Oil, ¹³¹I-IOM-40) a mixture of iodinated ethyl esters of fatty acids of poppy seed oil used for the treatment of hepatocarcinoma (HCC), ¹⁶⁶Ho-QuiremSpheres a poly-L-lactic acid (PLLA) based particles (available from Quirem Medical BV/Terumo Corp.), ¹⁶⁶Ho-TheraneaM an holmium based microparticles (Novartis), ¹⁷⁷Lu-DOTA-αMSH-PEG-C′ dots a dual modality product (Therapy/fluorescence) developed on the basis of ultrasmall fluorescent (Cy5) silica nanoparticles (C′ dots), conjugated with MC1-R targeting alpha melanocyte stimulating hormone (αMSH) peptides on the polyethylene glycol (PEG) coated surface (Elucida Oncology Inc.), ¹⁸⁸Re-ImDendrim a delivery system made from dendrimer (G5) diffusible probes for targeting hypoxic tumoral cells combined with the beta emitter Rhenium-188 (Nano Gun Technology), 224Ra-RadSpherin a calcium carbonate microspheres based for the treatment of ovarian cancer (Oncoinvent), ²²⁵Ac-Au@TADOTAGA (²²⁵AC-Au-2,2′,2″-(10-(4-((2-(5-(1,2-dithiolan-3-yl)pentanamido)ethyl)amino)-1-carboxy-4-oxobutyl)-1,4,7,10-tetraazacyclododecane-1,4,7-triyl)triacetic acid) a gold nanoparticle as an injectable radiopharmaceutical form of brachytherapy for local radiation treatment of cancer (NCSR Demokritos).

In one embodiment the radionuclides can be conjugated with small molecules (with molecular weight from a hundred to several thousand daltons). These molecules are generally but not limited designed on the basis of natural ligands that have a specific affinity for some receptors that are expressed on the surface of tumors. Natural molecules already used as drugs are often starting points for the development of such tracers and drugs. As a consequence, small molecules have the highest potential to cover all types of indications and including therapeutics such as: ⁴⁷Sc-cm10 (⁴⁷Sc-DOTA-Folate, ⁴⁷Sc-Folate) a folate analogue cm-10 is a combination of three entities: the folic acid, a DOTA-chelator and an albumin binding. Mechanism of action: Folate (Paul Scherrer Institute), ⁹⁰Y/¹⁷⁷Lu-FAPI-04 and ⁹⁰Y/¹⁷⁷Lu-FAPI-46 a DOTA-coupled quinolone analogue based on a Fibroblast Activation Protein (FAP)-specific enzyme inhibitor (FAPI). Mechanism of action: Fibroblasts (Novartis), ⁹⁰Y/¹⁷⁷Lu-NM600 (DOTA-18-(p-aminophenyl)octadecyl phosphocholine) a tumor-targeting alkylphosphocholine. Mechanism of action: Alkyl PhosphoCholine (University of Wisconsin-Madison), ^{117m}Sn-RAGE a targeting agent to the receptor against glycation end-products (RAGE) for the treatment of Alzheimer's disease. Mechanism of action: Receptor Against Glycation End-products (NeuroSn, Inc.), ¹³¹I-CLR-131 (¹³¹I-CLR-1404, ¹³¹I-NM404, ¹³¹I-18-p-iodophenyl-octadecyl phosphocholine) an alkyl phosphocholine (APC) from the family of phospholipid ether (PLE) analogs. Mechanism of action: (PI3K)/Akt (Cellectar Biosciences), ¹⁴⁹Tb-DOTA-Folate a folate derivate. Mechanism of action: Folate receptor (Paul Scherrer Institute), ¹³¹I-IITM (¹³¹I-Iodo-N-[4-(6-(isopropylamino)pyridine-4-yl)-1,3-thiazol-2-yl]-N-methyl benzamide) a benzamide targeting the ectopic metabotropic glutamate receptor 1 (mGluR1) used in melanomas. Mechanism of action: mGluR1 (NIQRST), ¹³¹I-BA52 (¹³¹I-benzo(1,3)dioxolo-5-carboxylic acid (4-(2-diethylamino-ethylcarbamoyl)-2-iodo-5-methoxy-phenyl)-amide) a melanin-binding benzamide for the therapy of malignant melanomas. Mechanism of action: Melamine (Bayer Pharmaceuticals), ¹³¹I-Iobenguane (¹³¹I-Metaiodobenzylguanidine, ¹³¹I-MIBG, ¹²³I-Azedra) a small molecule similar to norepinephrine used in the detection and therapy of primary or metastatic pheochromocytoma and paraganglioma (Progenics-Lantheus), ¹⁷⁷Lu-DOTAZOL (¹⁷⁷Lu-DOTAZOL, ¹⁷⁷Lu-Zoledronic acid, ¹⁷⁷Lu-DOTA-ZOL, ¹⁷⁷Lu-DOTA-Zoledronate, ¹⁷⁷Lu-Zoledronate, ¹⁷⁷Lu-DOTA-BP DOTAZOL, ¹⁷⁷Lu-ZLD, ¹⁷⁷Lu-DOTAMZOL, ¹⁷⁷Lu-DP-4411) a zoledronic acid derivative (¹⁷⁷Lu-(2,2′,2″-(10-(2-(2-(1-(2-hydroxy-2,2-diphosphonoethyl)-1H-imidazol-4-yl)ethylamino)-2-oxoethyl)-1,4,7,10-tetraazacyclo dodecane-1,4,7-triyl)triacetic acid) used for the therapy metastasized prostate cancer. Mechanism of action: bisphosphonate (Mainz University/ITM), ¹⁷⁷Lu-FF-10158 an antagonist targeting integrin αvβ3 and αvβ5 receptors. Mechanism of action: Integrin (AAA/Novartis/FUJIFILM), ⁹⁰Y-DOTA-EB-MCG a MCG ((I-1-carboxy-2-mercaptoethyl)carbamoyl)-L-glutamic acid) conjugated with the albumin-binding Evans Blue (EB) derivative and of a DOTA group. Mechanism of action: GRPR (Johns Hopkins Medical Institutions/NIH), ¹⁷⁷Lu-DO3A-VS-Cys40-Exendin-4 (¹⁷⁷Lu-Exendin-4; ¹⁷⁷Lu-DO3A-Exendin-4) a molecule targeting the Glucagon-like peptide-1. Mechanism of action: GLP-1 (Sanofi), ¹⁷⁷Lu-DOTA-LLP2A (¹⁷⁷Lu-LLP2A; ¹⁷⁷Lu-DOTA-PEG4-LLP2A) a molecule with high affinity for VLA-4. Mechanism of action: VLA-4 (University of Pittsburgh), ¹⁷⁷Lu-EBRGD (¹⁷⁷Lu-EB-RGD; ¹⁷⁷Lu-DOTA-EBRGD) a molecule conjugated with Evans Blue (EB) structure to bind albumin, targeting integrin αvβ3 receptor. Mechanism of action: Integrin (Molecular Targeting Technologies Inc.), ¹⁷⁷Lu-NM600 (¹⁷⁷Lu-DOTA-18-(p-aminophenyl)octadecyl phosphocholine) a tumor-targeting alkylphosphocholine. Mechanism of action: alkylphosphocholine (APC). (University of Wisconsin-Madison), ¹⁷⁷Lu-CTT1403 a phosphoramide based molecule for the therapy of prostate cancer developed by Mechanism of action: PSMA (Cancer Targeted Technology), ²¹¹At-AITM (²¹¹At-Astatino-N-[4-(6-(isopropylamino)pyridine-4-yl)-1,3-thiazol-2-yl]-N-methyl benzamide) a benzamide targeting the ectopic metabotropic glutamate receptor 1 (mGluR1) used in melanomas Mechanism of action: mGluR1 (NIQRST), ¹⁷⁷Lu-CTT1403 a phosphoramidate-based molecule (Cancer Targeted Technology), ²²⁵Ac-DOTA-MC1RL a melanocortin 1 receptor ligand. Mechanism of action: MCR1 (Moffit Cancer Center and Research Institute), ²²⁵Ac-DOTAZOL (²²⁵Ac-DOTAZOL, ²²⁵Ac-DOTA-ZOL, ²²⁵Ac-DOTA-Zoledronate) a Zoledronic acid derivative. Mechanism of action: bisphosphonate (University of Mainz).

In one embodiment the radionuclides can be conjugated with proteins (such as antibodies, antigen binding fragments, Bispecific Antibodies, affibodies, or Fibronectin type III domains) including therapeutics such as: ⁶⁷Cu-CTPA-mAB35 an anti CEA monoclonal antibody, ⁶⁷Ga-THP-Trastuzumab a mAb targeting HER2 (St. Thomas' Hospital, London), ⁹⁰Y-DOTA-FF-21101 (⁹⁰Y-FF-21101, FF-21101) a chimeric monoclonal anti-P-Cadherin (CDH3) mAb (FUJIFILM Pharmaceuticals), ⁹⁰Y-OTSA 101-DTPA (⁹⁰Y-OTSA 101, ⁹⁰Y-Tabituximab barzuxetan, TT641 pAb, FZD10 mAb) an anti-FZD10 (Frizzled Homolog 10) antibody (OncoTherapy Science Inc.), ⁹⁰Y-Clivatuzumab tetraxetan (hPAM4-Cide™) a fully human IgG1 monoclonal antibody directed against the human insulin-like growth factor (Immunomedics Inc.), ²²⁵Ac-Codrituzumab a full length humanized monoclonal antibody directed at Glypican 3 (NIH/MSKCC), ⁹⁰Y-Daclizumab a humanized murine monoclonal antibody that binds to CD25 (Interleukin-2), ⁹⁰Y-Epratuzumab tetraxetan (IMMU-102, ⁹⁰Y-Lymphocide, ⁹⁰Y-DOTA-hLL2) a humanized IgG1 antibody targeting CD22 for treatment of non-Hodgkin's lymphoma (NHL) and diffuse large B-cell lymphoma (DLBCL) (Immunomedics Inc.), ⁹⁰Y-Ibritumomab tiuxetan (Zevalin®) an IgG1 kappa-monoclonal murine antibody directed against the CD20 antigen. (available from Biogen Idec, Acrotech Biopharma), ⁹⁰Y-Ferritarg a rabbit polyclonal antibody which targets Ferritin (Alissa Pharma), ⁹⁰Y-IDEC-159 a monoclonal antibody which targets TAG-72 (Biogen-IDEC), ⁹⁰Y-IDEC-159 a monoclonal antibody reactive to tumor-associated glycoprotein (TAG-72). (Biogen Idec), ^{117m}Sn-DOTA-Annexin-V a natural protein that can be used to both image and treat vulnerable plaque, cancers or rheumatoid arthritis (Serene LLC), ¹³¹I-Tenatumomab a Tenascin-C targeting monoclonal antibody (ST2146) (Sigma-tau), ¹³¹I-chTNT (¹³¹I-chTNT-1/B; ¹³¹I-TNT, Cotara®, Vivatuxin®, ¹³¹I-derlotuximab biotin) a DNA/histone-targeting monoclonal antibody used to treat brain cancer through in situ infusion directly into the tumor. (Avid Bioservices, Shanghai Medipharm Biotech), ¹³¹I-Metuximab an antibody fragment targeting the hepatocellular cancer (HCC)-associated antigen HAb18G/CD147. (Chengdu Taihe Health Technology), ¹³¹I-Tositumomab (Bexxar®) an anti-CD20 antibody (GlaxoSmithKline), ¹³¹I-Weimeisheng a chimeric antibody (CIRC—Shanghai Meien Biotechnology), ¹³¹I-CAM-H₂ (¹³¹I-SGMIB anti-HER2-VHH1; ¹³¹I-SGMIB) a single domain antibody fragment (sdAb) targeting HER2. (Camel-IDS), ¹³¹I-CR3022 an antibody targeting specifically the SARS-CoV-2 RBD (host cell receptor binding domain). (MSKCC), ¹³¹I-81C6 (¹³¹I-Monoclonal Antibody 81C6, ¹³¹I-MoAB 81C6, Neuradiab™) a murine IgG2 anti-tenascin monoclonal antibody (Bradmer Pharmaceuticals), ¹³¹I-Naxitamab (¹³¹I-3F8, ¹³¹I-MoAb-3F8) a murine IgG3 monoclonal antibody which binds to the cell-surface GD2, a disialoganglioside antigen (Y-Mabs Therapeutics), ¹³¹I-Omburtamab (¹³¹I-Burtomab, ¹³¹I-8H9; ¹³¹I-8H9 (B7-H3), ¹³¹I-MoAb-8H9) a murine monoclonal antibody IgG1 recognizing cell surface antigen 4Ig-B7H₃, (Y-Mabs Therapeutics), ¹³¹I-chTNT (Tumor Necrosis Therapy-1, ¹³¹I-chTNT-1/B; ¹³¹I-TNT, Cotara®, Vivatuxin®, ¹³¹I-derlotuximab biotin) a DNA/histone-targeting monoclonal antibody (Avid Bioservices/Shanghai MediPharm Biotech), ¹³¹I-81C6 mAb (¹³¹I-Monoclonal Antibody 81C6, ¹³¹I-MoAB 81C6, Neuradiab™) a murine IgG2 anti-tenascin monoclonal antibody (Bradmer Pharmaceuticals), ¹³¹I-BC8 (Iomab-B™, ¹³¹I-apamistamab) a murine monoclonal antibody that targets CD45. (Actinium Pharmaceuticals), ¹³¹I Radretumab (¹³¹I-L19-SIP, ¹³¹I-L19SIP) a human recombinant antibody fragment consisting of the variable regions of the L19 mAb directed against the extra-domain B (ED-B) of fibronectin that is overexpressed in tumoral vasculature. (Philogen), ¹⁷⁷Lu-Lilotomab (Betalutin®) an anti-CD37 antibody (Nordic Nanovector), ¹⁷⁷Lu-Lilotomab satetraxetan (Betalutin™) an anti-CD37 antibody (Nordic Nanovector), ¹⁷⁷Lu-MVT-1075 (¹⁷⁷Lu-DFO-HuMab-5B1) an antibody binds to the carbohydrate antigen sialyl-Lewis a (sLea) (CA19-9) (BioNTech SE), ¹⁷⁷Lu/²²⁷Th APOMAB a murine monoclonal antibody DAB4, (AusHealth Corp Pty Ltd), ¹⁷⁷Lu-IMP-288 a Dock-and-Lock bispecific antibody anti-CEA (Nantes University—Radboud University), ¹⁷⁷Lu-TLX591 (¹⁷⁷Lu-Rosapatumab, ¹⁷⁷Lu-MLN591, ¹⁷⁷Lu-huJ591, ¹⁷⁷Lu-J591, ¹⁷⁷Lu-ATL-101, ¹⁷⁷Lu-TLX591t) an anti-PSMA (prostate-specific membrane antigen) antibody with high specificity for prostate tumor cells. (Telix Pharmaceuticals Ltd), ¹⁷⁷Lu-DTPA-TRC105 an anti-CD105 antibody (Tracon Pharmaceuticals), ¹⁷⁷Lu-²²⁵Ac-hu11B6 (¹⁷⁷Lu-DTPA-hu11B6; ¹⁷⁷Lu-h11B6) a PSMA targeting antibody (Lund University), ¹⁷⁷Lu-Humalutin (¹⁷⁷Lu-NNV003) an anti-CD37 antibody (Nordic Nanovector), ¹⁷⁷Lu/²²⁵Ac-Rosapatumab (J591, TLX591) an anti-PSMA (prostate-specific membrane antigen) an antibody with high specificity for prostate tumor cells (Telix Pharmaceuticals), ¹⁷⁷Lu-TLX250 (¹⁷⁷Lu-cG250, TLX250, ¹⁷⁷Lu-TLX250t, ¹⁷⁷Lu-Lutarex®, ¹⁷⁷Lu-DOTA-Girentuximab, ¹⁷⁷Lu-Girentuximab) a chimeric murine human monoclonal antibody which targets the Carbonic Anhydrase IX (CA-IX) molecule/G250 antigen. (Telix Pharmaceuticals), ¹⁸⁸Re/²¹³Bi-8C3 a murine antibody to melanin of the IgG isotype (Radimmune Inc.), ²¹¹At-81C6 a chimeric monoclonal antibody anti tenascin (Duke University), ²¹¹At-MX35-F(ab′)₂ (²¹¹At-labeled-MX35), ²¹¹At-MX35, anti-human SLC34A2) anti body fragment of the murine IgG1-class monoclonal antibody directed toward a cell-surface glycoprotein of 95 kDa on OVCAR-3 cells (MSKCC), ²¹²Pb-TCMC-Trastuzumab a Tetrakis Carbamoyl Methyl tetraaza Cyclododecane (TCMC) conjugated with a monoclonal antibody interfering with the HER2/neu receptor (an epidermal growth factor receptor EGFR) (Orano Med), ²¹²Pb-Daratumumab (²¹²Pb-anti-CD38, ²¹²Pb-Dara, ²¹²Pb-TCMC-Daratumumab) a human immunoglobulin G1 kappa (IgG1k) monoclonal antibody directed against the cell surface glycoprotein CD38 which is expressed on various hematopoietic cells and is overexpressed on multiple myeloma (MM) cells (Orano Med), ²¹²Pb-376.96 (²¹²Pb-TCMC-376.96) a monoclonal antibody which recognizes a B7-H₃ (CD276) epitope expressed on ovarian cancer cells, pancreas tumor cells and cancer initiating cells (CICs) (University of Alabama), ²¹²Pb-NNV003 an anti-CD37 humanized antibody (Orano Med/Nordic Nanovector), ²¹³Bi-DTPA-PAN-622 (²¹³Bi-PAN-622) a radiolabeled fully human monoclonal antibody (PAN-622) to human aspartyl (asparaginyl)-hydroxylase (HAAH) (Sensei Biotherapeutics Inc.), ²¹³Bi-Lintuzumab (²¹³Bi-Bismab-A™, ²¹³Bi-DTPA-Lintuzumab, ²¹³Bi-CHX-A″-DTPA-huM195) an anti-CD33 humanized antibody huM195 (Actinium Pharmaceuticals), ²²⁵Ac-Cixutumumab (²²⁵Ac-IMC-A12, ²²⁵Ac-DOTA-Cixutumumab) a fully human IgG1 monoclonal antibody directed against the human insulin-like growth factor-1 receptor (IGF-1R). (University of Saskatchewan), ²²⁵Ac-Girentuximab (TLX251) a chimeric murine human monoclonal antibody which targets the Carbonic Anhydrase IX (CA-IX) molecule/G250 antigen, expressed on over 90% of clear cell renal cell carcinomas. (Telix Pharmaceuticals), ²²⁵Ac-J591 (²²⁵Ac-ATL-101, ²²⁵Ac-TLX591) an anti-PSMA (prostate-specific membrane antigen) antibody with high specificity for prostate tumor cells (Telix Pharmaceuticals), ²²⁵Ac-TLX251 (²²⁵Ac-cG250, ²²⁵Ac-DOTA-Girentuximab, ²²⁵Ac-Girentuximab) a chimeric murine human monoclonal antibody which targets the Carbonic Anhydrase IX (CA-IX) molecule/G250 antigen, expressed on over 90% of clear cell renal cell carcinomas. (Telix Pharmaceuticals), ²²⁵Ac-TLX591 (²²⁵Ac-J591) anti-PSMA (prostate-specific membrane antigen) antibody with high specificity for prostate tumor cells (Telix Pharma/Weill Medical College of Cornell University), ²²⁵Ac-TLX592 (²²⁵Ac-J591, improved ²²⁵Ac-TLX591) a re-engineered antibody hu591 targeting PSMA (Telix Pharma), ²²⁵Ac-FPI-1434 an humanized monoclonal antibody which targets the insulin-like growth factor-1 receptor (IGF-1R) (Fusion Pharmaceuticals), ²²⁵Ac-Lintuzumab (²²⁵Ac-Actimab-A™, ²²⁵Ac-DOTA-huM195, ²²⁵Ac-DOTA-Lintuzumab, ²²⁵Ac-CHX-A″-DOTA-huM195, Lin-Ac225, ²²⁵Ac-huM195) an anti-CD33 humanized antibody huM195 (Actinium Pharmaceuticals), ²²⁵Ac Daratumumab (²²⁵Ac-anti-CD38, ²²⁵Ac-Dara, ²²⁵Ac-DOTA-Daratumumab) a human immunoglobulin G1 kappa (IgG1k) monoclonal antibody directed against the cell surface glycoprotein CD38 which is expressed on various hematopoietic cells and is overexpressed on multiple myeloma (MM) cells (Actinium Pharmaceuticals), ²²⁷Th-Anetumab (²²⁷Th-BAY2287411; BAY2287411; BAY 2287411) a human immunoglobulin G1 (IgG1) monoclonal antibody which targets mesothelin (Bayer Pharma), ²²⁷Th-Epratuzumab a humanized IgG1 antibody targeting CD22 (Bayer), ²²⁷Th-Trastuzumab a humanized HER2 monoclonal antibody (Bayer Pharma), ²²⁷Th-CD33-TTC (²²⁷Th-Lintuzumab; ²²⁷Th-Anti-CD33-Conjugate) a monoclonal antibody which binds to the sialoadhesin receptor CD33 (Siglec-3), a 67-kDa protein, expressed on leukemic blasts of AML patients (Bayer Pharma), ²²⁷Th-CD70-TTC (²²⁷Th-CD70-Thorium Targeted Conjugate) a CD70 targeting antibody (IgG1) (Bayer Pharma).

In one embodiment the radionuclides can be conjugated with a peptide or polypeptide including therapeutics such as:

somatostatin analogues targeting somatostatin (SST) receptors with for e.g. 90Y-DOTATATE (⁹⁰Y-DOTA0-Phe1-Tyr3-octreotate, ⁹⁰Y-Octreotate, and ⁹⁰Y-DOTA-Octreotate), Lutetium (¹⁷⁷Lu)Oxodotreotide [INN] (Lutathera®, ¹⁷⁷Lu-DOTATATE, ¹⁷⁷Lu-DOTA0-Tyr3-octreotate, ¹⁷⁷Lu-Octreotate, ¹⁷⁷Lu-Lutate, ¹⁷⁷Lu-Edotreotate officially USAN: lutetium Lu-177 dotatate and INN: lutetium (¹⁷⁷Lu)oxodotreotide) (AAA/Novartis), ²¹³Bi-DOTATATE (²¹³Bi-[DOTA0, Tyr3]-octreotate), ²²⁵Ac-DOTATATE (²²⁵Ac-[DOTA0, Tyr3]octreotate) (Bad Berka), ⁶⁷Cu-SARTATE (⁶⁷Cu-MeCOSAR-Octreotate; ⁶⁷Cu-SAR-[Tyr3]-Octreotate) (Clarity Pharmaceuticals), ¹⁷⁷Lu-DOTA-EB-TATE (¹⁷⁷Lu-DOTA-EBTATE; EBTATE; lutetium-177-1,4,7,10-tetra-azacyclododecane-1,4,7,10-tetraacetic acid-Evans blue-Tyr3-octreotate) a molecule based on octreotate that uses an Evans blue structure to bind albumin (Molecular Targeting Technologies, Inc.), ¹⁷⁷Lu-HA-DOTATATE (¹⁷⁷Lu-high affinity-DOTATATE, ¹⁷⁷Lu-DOTA-3-iodo-Tyr3-octreotate) (Scintomics), ⁹⁰Y-DOTATOC (⁹⁰Y-DOTATOC, ⁹⁰Y-SMT487, ⁹⁰Y-Onalta®, ⁹⁰Y-OctreoTher®, ⁹⁰Y-DOTA0-Phe1-Tyr3-Octreotide), ¹⁷⁷Lu-Edotreotide (¹⁷⁷Lu-DOTA0-Phe1-Tyr3-Octreotide, ¹⁷⁷Lu-Octreotide, ¹⁷⁷Lu-SMT-487, ¹⁷⁷Lu-DOTATOC, ¹⁷⁷Lu-Solucin®, ¹⁷⁷Lu-Edo, ¹⁷⁷Lu-Edotreotide PRRT) a somatostatin analogue (ITM Solucin GmbH), ¹⁴⁹Tb-DOTANOC (Paul Scherrer Institute), ¹⁵²Tb-DOTATOC (Paul Scherrer Institute), ²¹³Bi-DOTATOC, ²²⁵Ac-DOTATOC (LANL), ¹⁷⁷Lu-DOTANOC (¹⁷⁷Lu-DOTA-1-Nal3-octreotide), ¹⁷⁷Lu-IPN-01072 (¹⁷⁷Lu-OPS201, ¹⁷⁷Lu-DOTA-JR11637, ¹⁷⁷Lu-OPSC001, ¹⁷⁷Lu-Satoreotide tetraxetan) a somatostatin analogue binding sst2 receptor (Octreopharm/Ipsen Pharma), ²¹²Pb-DOTAMTATE (²¹²Pb-AR-RMX; ²¹²Pb-AlphaMedix™, ORM2110) (Orano Med), ⁹⁰Y-OPS201 (⁹⁰Y-DOTA-JR11, ⁹⁰Y-SOMTher®) ⁹⁰Y-DOTALAN (⁹⁰Y-DOTA-Lanreotide, ⁹⁰Y-Lanreotide, or ⁹⁰Y-Somatulin), ¹⁷⁷Lu-DOTA-LM3 (¹⁷⁷Lu-1,4,7,10-tetraazacyclododecane, 1,4,7-triacetic acid, 10-acetamide N-p-Cl-Phe-cyclo(d-Cys-Tyr-d-4-amino-Phe(carbamoyl)-Lys-Thr-Cys)-d-Tyr-NH2) (Zentralklinik Bad Berka), Peptides targeting Prostate-specific membrane antigen (PSMA) with for e.g. ⁴⁴Sc-PSMA-617, ⁹⁰Y-PSMA-617 (Paul Scherrer Institute), ¹⁵²Tb-PSMA-617 (Paul Scherrer Institute—Bad Berka Hospital), ¹⁷⁷Lu-PSMA-617 (¹⁷⁷Lu-DKFZ-PSMA-617, ¹⁷⁷Lu-PSMA617, lutetium (¹⁷⁷Lu)vipivotide tetraxetan [INN]) (Novartis), ²²⁵Ac-PSMA-617 (Novartis), ¹⁶¹Tb-PSMA-617 (Paul Scherrer Institute/ETH/ILL), ²¹³Bi-PSMA-617 (²¹³Bi-Vipivotide tetraxetan) (University Hospital of Heidelberg), ¹⁷⁷Lu-EB-PSMA-617 a PSMA-617 analogue conjugated with a truncated Evans Blue (Peking Union Medical College), ¹⁷⁷Lu-FC705 (FutureChem), 203Pb-CA012 (University Hospital of Heidelberg), ¹⁷⁷Lu-PSMA-I&T (¹⁷⁷Lu-PSMAI&T, ¹⁷⁷Lu-PSMA-TUM1, ¹⁷⁷Lu-DOTAGA-(I-y)fk(Sub-KuE) (University of Munich—Scintomics), ¹⁷⁷Lu-PSMA-R₂ ((Nε-[¹⁷⁷Lu-(4,7,10-Tricarboxymethyl-1,4,7,10-tetrazacyclododec-1-yl)acetyl]-6-aminohexanoic)-(Nε′-4-bromobenzyl) lysine-CO-glutamic-acid) (Novartis)

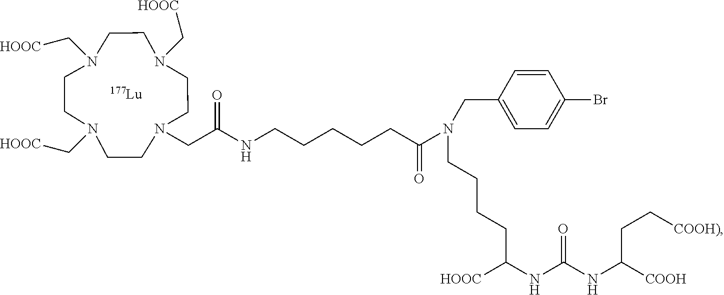

¹⁷⁷Lu-PSMA-CC-34 (¹⁷⁷Lu-NODAGA-PSMA-CC-34; ¹⁷⁷Lu-PSMA-CC34), ¹⁷⁷Lu-L1 (¹⁷⁷Lu-DOTAMA-L1) (Johns Hopkins University), ¹⁷⁷Lu-PSMA-ALB-56 (¹⁷⁷Lu-DOTA-PSMA-ALB-56) a PSMA ligand combining with an albumin binder based on the p-(tolyl)-moiety (ETH Zurich/PSI), ¹⁷⁷Lu-RPS-063 a molecule with a high-affinity PSMA-binding domain, linked to an albumin-binding group (ABG) (Weill Cornell Medicine), ¹⁷⁷Lu-iPSMA (¹⁷⁷Lu-DOTA-HYNIC-Lys(Nal)-Urea-Glu) (ININ), ²¹²Pb-NG001 (²¹²Pb-p-SCN-Bn-TCMC-PSMA; ²¹²Pb-TCMC-PSMA) (Nucligen AS), ¹³¹I-RPS-027 molecule with a high-affinity PSMA-binding domain, linked to an albumin-binding group (ABG) (Weill Cornell Medicine), ²²⁵Ac-RPS-074 (Weill Cornell Medical), ¹⁸⁸Re-P2045 (BAY 86-5284, Tozaride®) a 11-amino acid somatostatin peptide (Andarix Pharmaceuticals), ¹³¹I-RPS-001 (¹³¹I-MIP-1095; ¹³¹I—(S)-2-(3-((S)-1-carboxy-5-(3-(4-iodophenyl) ureido) pentyl) ureido) pentanedioic acid) a glutamate-urea-lysine analogue for therapeutic application in prostate cancer (Progenics/Lantheus), ¹⁷⁷Lu-DOTA-Peptide-2 (¹⁷⁷Lu-DOTA-p-Cl-Phe-Cyclo(d-Cys-I-BzThi-d-Aph-Lys-Thr-Cys)-d-Tyr-NH2; ¹⁷⁷Lu-[(1,4,7,10-Tricarboxymethyl-1,4,7,10-tetrazacyclododec-1-yl) acetyl]-(L) pChlorophenylalanyl-(D) Cysteinyl-(L)-3-BenzoThienylalanyl (L-BzThi)-(D)-4-Amino carbamoyl phenylalanyl(D-Aph)-(L)-Lysyl-(L)-Threoninyl-(L)-Cysteinyl-(D)-Tyrosine-NH2-cyclic disulfide) (Tehran Faculty of Pharmacy).

In another embodiment, the radioligand agent is selected from any of the agents disclosed in 1) Theranostics 2016; 7(7): 1928-1939, 2) The Journal of Nuclear Medicine, Vol. 60, No. 7, 910-916, 3) Mol Imaging Biol 2020 April; 22(2): 274-284, and related electronic supplementary material, all of which are hereby incorporated by reference. In a specific embodiment, the radioligand agent is selected from ¹⁷⁷Lu-CTT1401, ¹⁷⁷Lu-CTT1403, CTT1057 (which incorporates 18F) and ¹⁷⁷Lu-CTT1751.

Bombesin analogues targeting Gastrin Releasing Peptide Receptor (GRPR) with for e.g. ¹⁷⁷Lu-NeoB (¹⁷⁷Lu-NeoBomb1, ¹⁷⁷Lu-DOTA-(p-aminobenzylamine-diglycolic acid)-[D-Phe6-His-NHCH-[(CH2CH(CH3)2]212-des-Leu13-des-Met14] BBN) (Novartis), ¹⁷⁷Lu-ProBOMB1 (68 Ga-DOTA-pABZA-DIG-d-Phe-Gln-Trp-Ala-Val-Gly-His-Leu-ψ(CH₂N)-Pro-NH2) (University of British Columbia), ²¹²Pb-RM2 (²¹²Pb-DOTA-4-amino-1-carboxymethyl-piperidine-D-Phe-Gln-Trp-Ala-Val-Gly-His-Sta-Leu-NH2) (US Department of Veterans Affairs), ¹⁷⁷Lu-RM2 (¹⁷⁷Lu-DOTA-4-amino-1-carboxymethyl-piperidine-D-Phe-Gln-Trp-Ala-Val-Gly-His-Sta-Leu-NH2, 177 Lu-BAY-1017858) (Rostock University),

Fibroplast Activation Proteins (FAP) Inhibitor targeting FAP with for e.g ¹⁷⁷Lu-FAP-2286 (¹⁷⁷Lu-3BP-3554, ¹⁷⁷Lu-3B-201) (Clovis Oncology/3B Pharma),

Peptides targeting L-Amino Acid Transporter-1 (LAT-1) with for e.g ¹³¹I-TLX101 (4-[131|]-4-L-iodophenylalanine, ¹³¹I-|PA, ¹³¹I-TLX101t, TLX101, previously developed under the name ¹³¹I-ACD-101) (Telix Pharmaceutical), ²¹¹At-TLX102 (4-[211At]-astatinophenylalanine) an analogue of IPA (iodophenylalanine) (Telix Pharmaceuticals Ltd), Minigastrin analogues targeting Cholecystokinin-2 (CCK-2) receptors with for e.g ¹⁷⁷Lu-DOTA-MGS5 (¹⁷⁷Lu-MGS5; ¹⁷⁷Lu-DOTA-D-Glu-Ala-Tyr-Gly-Trp-(N-Me)Nle-Asp-1-Nal-NH2) (Innsbruck University), ¹⁷⁷Lu-Debio 1124 (¹⁷⁷Lu-DOTA-(DGlu)₆-Ala-Tyr-Gly-Trp-Nle-Asp-Phe-NH2, Debio 1124, ¹⁷⁷Lu-PSIG-2, 177Lu—PP-F11N) (Debiopharm International SA/PSI/University Hospital of Basel),

Modified substance P targeting GPCR neurokinin type 1 receptor (NK-1R) with for e.g ²¹³Bi-DOTA-SP (²¹³Bi-Substance-P, ²¹³Bi-DOTA-Substance P, ²¹³Bi-[Thi8,Met(O2)11]-substance-P) (Novacurie), ²²⁵Ac-DOTA-SP (²²⁵Ac-Substance-P, ²²⁵Ac-DOTA-Substance P) (Novacurie),

Peptide targeting CXCR4 receptor with for e.g

¹⁷⁷Lu-Pentixather (3-iodo-D-Tyr1-pentixafor; cyclo(D-3-iodo-Tyr1-[NMe]-D-Orn2(AMBS-DOTA)-Arg3-2-Nal4-Gly5) (Scintomics GmbH), ¹⁷⁷Lu-BL01 (¹⁷⁷Lu-cyclo[Phe-Tyr-Lys(iPr)-d-Arg-2-Nal-Gly-d-Glu]-Lys(iPr)-NH2), (University of British Columbia), ²¹²Pb-DOTA-VMT-MCR1 a peptides that bind with high affinity and specificity to melanocortin receptor subtype I (MCR1) (Viewpoint Molecular Targeting LLC/RadioMedix), ¹⁷⁷Lu-IPN-01087 (¹⁷⁷Lu-3BP-227) a neurotensin antagonist peptide targeting NTR-1 (or NTSR1—neurotensin receptor-1) (3B Pharmaceuticals/Ipsen Pharma), ¹⁷⁷Lu-DOTA-Exendin-4 (¹⁷⁷Lu-Exendin-4; ¹⁷⁷Lu-DOTA-Ahx-Lys40-Exendin-4) a peptide targeting the Glucagon-like peptide-1 receptor (GLP-1) (Bhabha Atomic Research Center).

Integrin Radioligands

⁶⁸Ga-FF58 (⁶⁸Ga-2,2′,2″-(10-(2-(((R)-1-((2-(4-(4-(N—((S)-1-carboxy-2-(5-(5,6,7,8-tetrahydro-1,8-naphthyridin-2-yl)pentanamide)ethyl)sulfamoyl)-3,5-dimethylphenoxy)butanamide)ethyl)amino)-1-oxo-3-sulfopropan-2-yl)amino)-2-oxoethyl)-1,4,7,10-tetraazacyclododecane-1,4,7-triyl)triacetic acid) and ¹⁷⁷Lu-FF58 (¹⁷⁷Lu-2,2′,2″-(10-(2-(((R)-1-((2-(4-(4-(N—((S)-1-carboxy-2-(5-(5,6,7,8-tetrahydro-1,8-naphthyridin-2-yl)pentanamide)ethyl)sulfamoyl)-3,5-dimethylphenoxy)butanamide)ethyl)amino)-1-oxo-3-sulfopropan-2-yl)amino)-2-oxoethyl)-1,4,7,10-tetraazacyclododecane-1,4,7-triyl)triacetic acid).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Isomeric Forms

Any asymmetric atom (e.g., carbon or the like) of the compound(s) that can be used in the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound that can be used in the present invention can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds that can be used in the present invention or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds that can be used in the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds that can be used in the present or racemic intermediates can also be resolved by chiral chromatography, e.g., high pressure liquid chromatography (HPLC) using a chiral adsorbent.

Compounds that can be used in the present, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163.

Furthermore, the compounds that can be used in the present invention, including their salts, can also be obtained in the form of their hydrates, or include other solvents used for their crystallization. The compounds of the present invention may inherently or by design form solvates with pharmaceutically acceptable solvents (including water). The term “solvate” refers to a molecular complex of a compound (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Bifunctional Degrader Compounds

Compounds of Formula (I) as described herein may also be used to form bifunctional degrader compounds, using the methods described herein and known synthetic routes.

In an embodiment, the WRN bifunctional degrader molecule is a compound of Formula 1a:

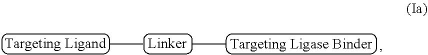

or a pharmaceutically acceptable salt thereof, wherein:
the Targeting Ligand is a group that is capable of binding to Werner Syndrome RecQ DNA helicase (WRN), such as a compound of Formula (I) or 1g, or Compound A, or compound B herein;
the Linker is a group that covalently links the Targeting Ligand to the Targeting Ligase Binder; and
the Targeting Ligase Binder is a group that is capable of binding to a ligase (e.g., VHL, IAP or Cereblon E3 Ubiquitin ligase, for example using the binder moieties of compounds X, Y and Z below). Bifunctional degrader compounds X, Y and Z were made and tested for WRN activity.

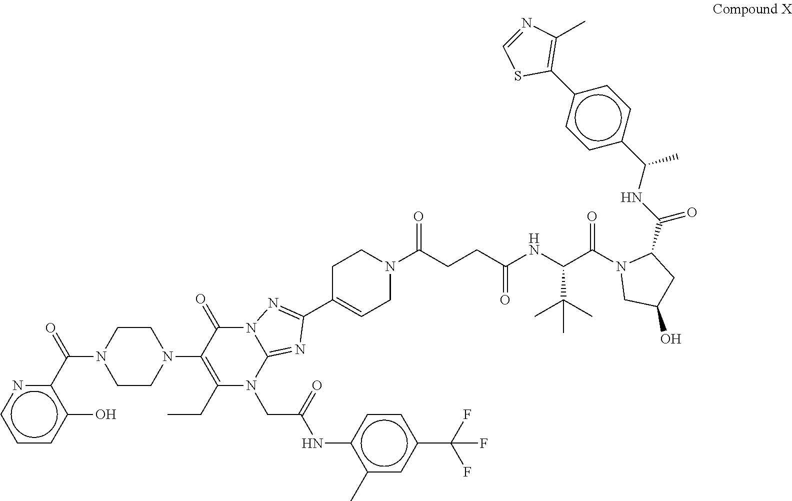

(2S,4R)-1-((S)-2-(4-(4-(5-ethyl-6-(4-(3-hydroxypicolinoyl)piperazin-1-yl)-4-(2-((2-methyl-4-(trifluoromethyl)phenyl)amino)-2-oxoethyl)-7-oxo-4,7-dihydro-[1,2,4]triazolo[1,5-a]pyrimidin-2-yl)-3,6-dihydropyridin-1(2H)-yl)-4-oxobutanamido)-3,3-dimethylbutanoyl)-4-hydroxy-N—((S)-1-(4-(4-methylthiazol-5-yl)phenyl)ethyl)pyrrolidine-2-carboxamide

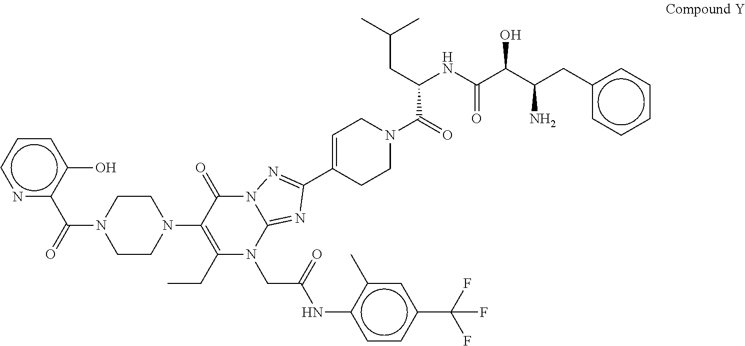

(2S,3R)-3-amino-N—((S)-1-(4-(5-ethyl-6-(4-(3-hydroxypicolinoyl)piperazin-1-yl)-4-(2-((2-methyl-4-(trifluoromethyl)phenyl)amino)-2-oxoethyl)-7-oxo-4,7-dihydro-[1,2,4]triazolo[1,5-a]pyrimidin-2-yl)-3,6-dihydropyridin-1(2H)-yl)-4-methyl-1-oxopentan-2-yl)-2-hydroxy-4-phenylbutanamide

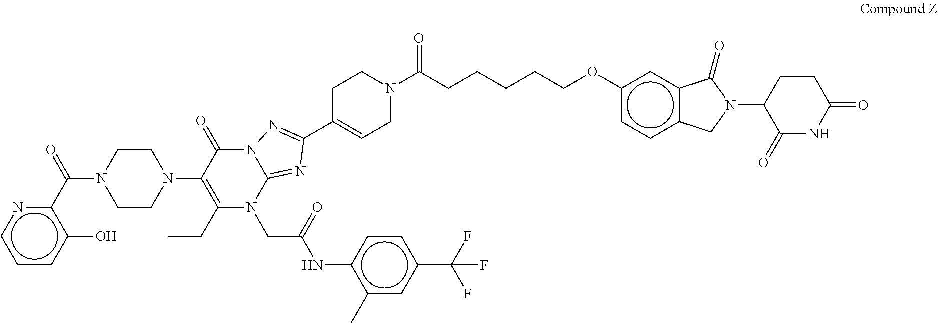

2-(2-(1-(6-((2-(2,6-dioxopiperidin-3-yl)-3-oxoisoindolin-5-yl)oxy)hexanoyl)-1,2,3,6-tetrahydropyridin-4-yl)-5-ethyl-6-(4-(3-hydroxypicolinoyl)piperazin-1-yl)-7-oxo-[1,2,4]triazolo[1,5-a]pyrimidin-4(7H)-yl)-N-(2-methyl-4-(trifluoromethyl)phenyl)acetamide

According to the assays disclosed in the application PCT/IB2022/054850 published as WO2022/249060, which is hereby incorporated by reference, the compounds were active as shown in Table X

TABLE X

| | WRN | Proliferation | Proliferation |
|---|---|---|---|
| | ATPase | Assay | Assay |
| | Activity | SW48 | DLD1-WRN-KO |
| | (IC₅₀) | (GI₅₀) | (GI₅₀) |
| Compound | μM | μM | μM |
| X | 0.16 | 3.84 | >10 |
| Y | 0.14 | 1.19 | >10 |
| Z | 0.09 | 1.31 | >10 |

TABLE Y
LCMS

| Compound | Rt, min | MS m/z |
|---|---|---|
| X | 1.14 | M + Na 1214.5 |
| Y | 1.07 | [M + H]⁺ 956.4 |
| Z | 1.11 | [M − H]⁻ 1020.4 |

LCMS method compounds X, Y and Z:
Instrumentation: Thermo Exactive Plus HRMS, Thermo Vanquish LC Instrument
Column: Agilent Zorbax SB-AQ 1.8 μM
Column dimension: 2.1*30 mm
Column temperature: 40° C.
Eluents: A: water and 0.04% formic acid
   B: acetonitrile and 0.04% formic acid
Flow Rate: 0.5 mL/min
Gradient: from 5% to 95% B in 0.5 min, 95% B for 1 min, 95% to 5% B in 0.1 min, 5% B for 0.4 min
MS settings: polarity positive ion mode, resolution 35,000, Scan range 150 to 1800 m/z, AGC target 3 e6, Max IT 200 ms

In another aspect of the invention there is provided an antibody-drug conjugate comprising a WRN inhibitor compound as described herein. Antibodies and linker components can be selected according to those known in the art.

### Synthesis of WRN Inhibitors and Assays Thereof

The synthesis of WRN inhibitors, biological assays and data, according to specific embodiments of the present invention, are described in PCT/IB2022/054850 (WO2022/249060), which is hereby incorporated by reference in its entirety.

### EXAMPLES

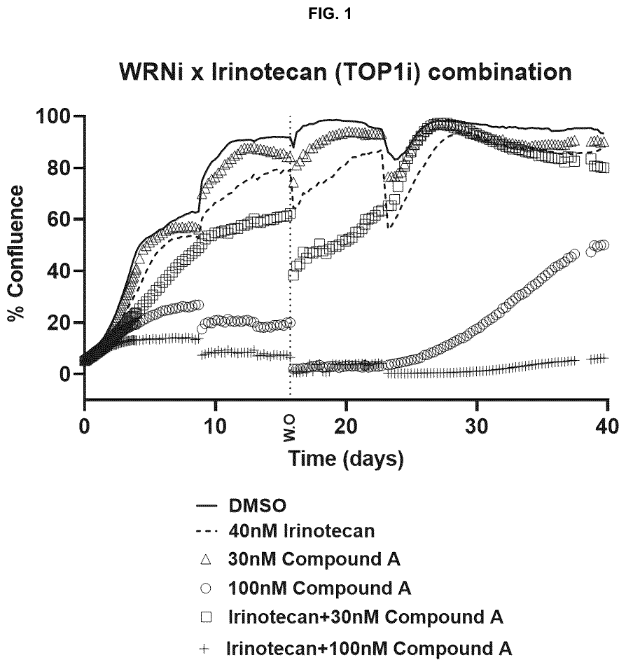

### Examples 1 to 11, 13, 14, 21 and 34 (FIGS. 1 to 11, 13, 14, 21 and 34 Respectively)

### 1. Methods

### 1.1. Cell Culture

The cell lines below were obtained from ATCC. Media and culture conditions used are as recommended by ATCC. All cells were maintained at 37° C. in a humidified 5% CO₂ incubator. SW48 (CCL-231, ATCC) were cultured in RPMI 1640 (#1-41F01-1, AMIMED) supplemented with 10% FCS (#2-O-1F30-I, Bio concept), 2 mM L-glutamine (#5-10K50-H, BioConcept), 1 mM sodium pyruvate (#5-60F00-H, BioConcept) and 10 mM HEPES (#5-31F00-H, Bio Concept). HCT116 (CCL-247, ATCC) were cultured in McCoy's 5A (#16600082, Gibco) supplemented with 10% FCS (#2-O-1F30-I, Bio concept), 2 mM L-glutamine (#5-10K50-H, BioConcept).

Cells were passaged by washing first with Dulbecco's PBS without Ca²⁺/Mg²⁺ (#3-05F29-1, AMIMED), trypsinizing cells with Trypsin 0.05% in PBS with EDTA (#5-51F00-H, AMIMED), centrifuging in the respective culture media, and splitting cells into fresh media at a ratio of 1:8, 2 times per week.

### 1.2. In Vitro Experiments

### 1.2.1. Long Term Proliferation Assays

On day 1, cells were trypsinized, resuspended in the respective media and counted using TC20 cell counter from Bio-Rad. Cells were seeded in 200 microliters growth medium at 3,000 to 4,000 cells/well into white, clear-bottom 96-well plates (Corning Cat #3903).

On day 2, cells were incubated overnight at 37° C. in a humidified 5% CO₂ atmosphere, and then treated in triplicate, with the indicated concentrations, using a HP 300D non-contact Digital Dispenser (TECAN). The final concentration of DMSO was normalized to 0.1% in all wells. On Day 7 of the experiment, compound treatment was refreshed, by carefully removing media by aspiration and adding fresh medium, followed by compound dosing as on Day 2. Compound treatment was removed around Day 15, by carefully aspirating the media, washing once with fresh media and adding 200 ul of fresh medium. Around day 21, media was refreshed by carefully aspirating the media and adding 200 ul of fresh medium. Experiment was monitored, and images acquired using Incucyte® S3 live-cell analysis instrument (Sartorius). Images were captured every 6 h from day 2 up to 40 days. Data was analyzed and represented using Prism-GraphPad.

### 1.2.2. Irradiation (Examples 6 and 7)

On day 1, cells were trypsinized, resuspended in the respective media and counted using TC20 cell counter from Bio-Rad. Cells were seeded in 200 microliters growth medium at 3,000 to 4,000 cells/well into white, clear-bottom 96-well plates (Corning Cat #3903) and incubated overnight at 37° C. in a humidified 5% CO2 atmosphere.

On day 2, cells were exposed to Ionizing Radiation (IR) using external beam radiation device (RS 1800 Q X-ray cell irradiator) at a dose of 3 Gy/min. Irradiated cells were treated in triplicate with compound A with the indicated concentrations, using a HP 300D non-contact Digital Dispenser (TECAN).

### 2. List of Abbreviations

| Abbreviation | Description |
|---|---|
| WRN | Werner Syndrome RecQ helicase |
| CRC | Colorectal carcinoma |
| MSS | Microsatellite stability |
| MSI | Microsatellite instability |
| MSI^{high} | Microsatellite instability high |
| W.O | Wash Out |
| ATRi | Ataxia Telangiectasia and Rad3-related protein inhibitor |
| DNA-PKi | DNA-dependent protein kinase inhibitor |
| TOP1i | Topoisomerase I inhibitor |
| WEE1i | Wee1-like protein kinase inhibitor |
| IR | Ionizing Radiation |
| 5FU | 5-Fluorouracil |
| CHK1/2i | Checkpoint kinase 1 and 2 |

Results

### Examples 1, 8-10 and 13-14: In Vitro Proliferation Effect of Compound A in Combination with Chemotherapy Drugs in MSI^{high} Colorectal Cancer (CRC) Cell Models

### Example 1

In vitro combination of Compound A with irinotecan, in SW48 MSI^{high} CRC cell model potentiates WRN inhibition and prevent/delay the relapse observed after Compound A monotherapy at 100 nM, on a long-term proliferation assay up to 40 days (FIG. 1).

### Example 8

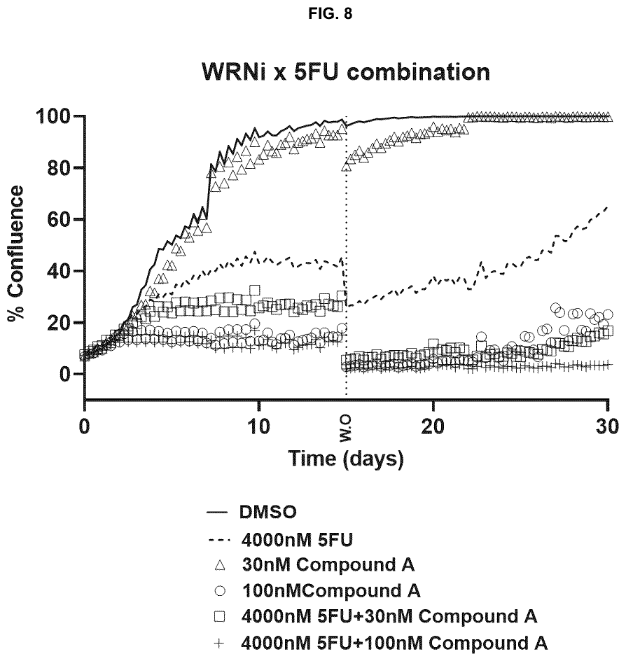

We could also observe a longer-lasting response in the combination of Compound A with 5FU, in the SW48 cell model. (FIG. 8).

### Examples 9 and 10

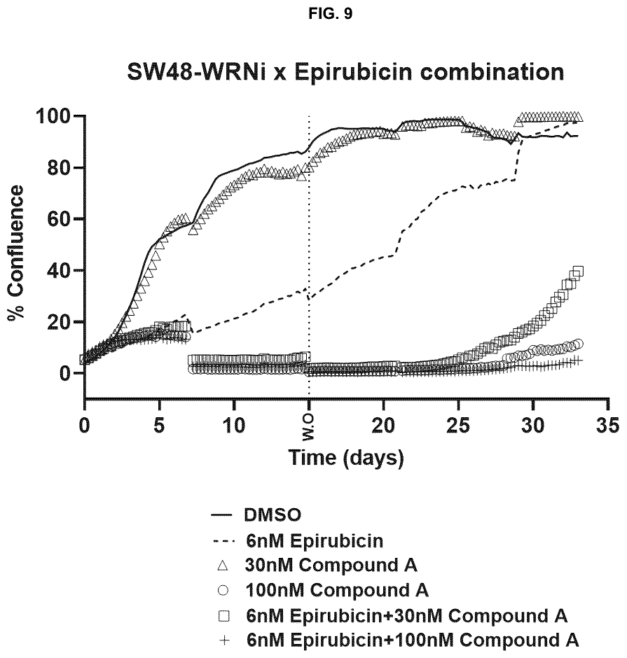

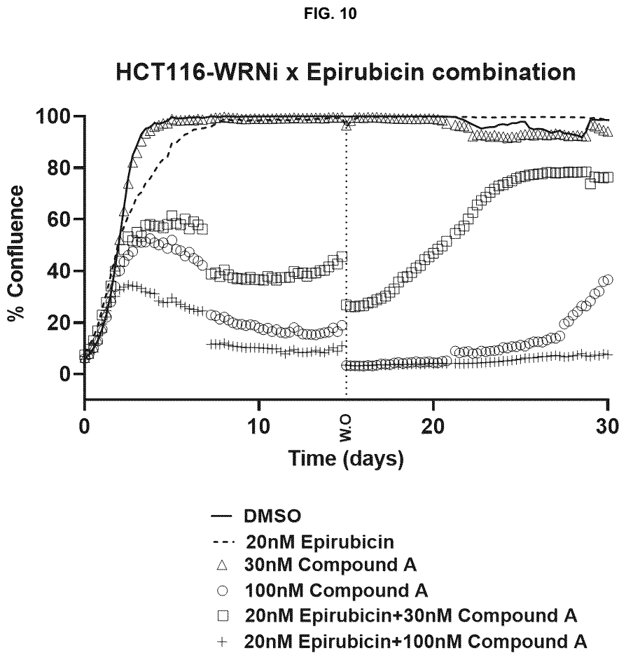

Further data indicates that combination of Epirubicin with Compound A achieves strong synergism with anti-proliferative effect even using a sub-optimal concentration of Compound A at 30 nM in SW48 model. Deeper response is obtained when combining Compound A (100 nM) with Epirubicin in both CRC MSI^{high} models (HCT116 and SW48) on a long-term proliferation assay up to 35 days (FIGS. 9 and 10)

### Example 13

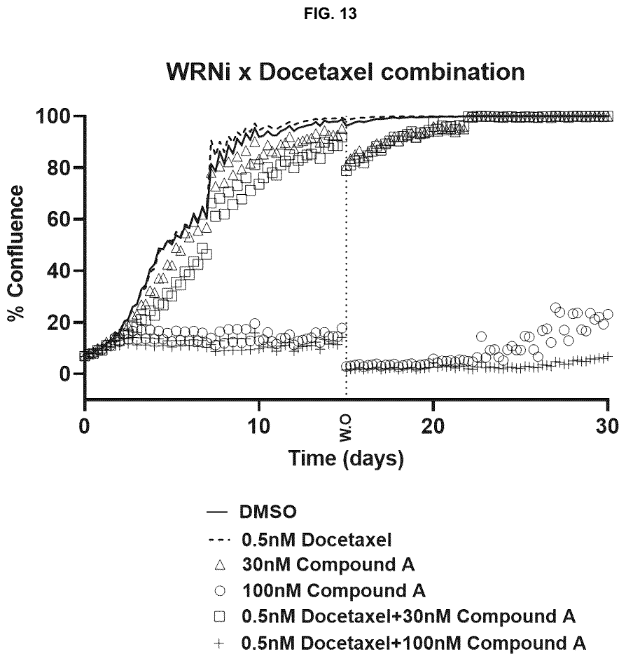

Docetaxel combination with Compound A shows a modest effect preventing/delaying the relapse observed after Compound A monotherapy at 100 nM, on a long-term proliferation assay up to 30 days delay in SW48. (FIG. 13)

### Example 14

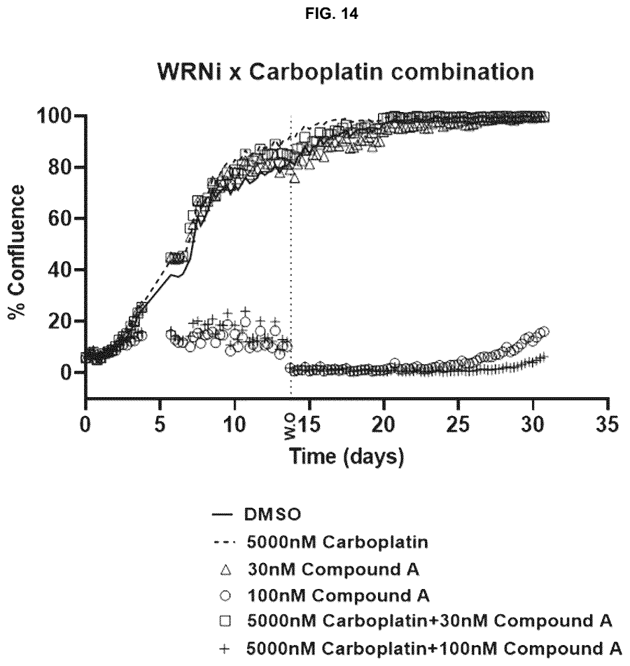

Combination of Compound A with Carboplatin shows a modest beneficial combo effect in SW48 model (FIG. 14).

### Examples 2-5, 11, 21 and 34: In Vitro Proliferation Effect of Compound A in Combination with DNA Damage Response (DDR) Inhibitors and Other Targeted Therapies in MSI^{high} Colorectal Cancer (CRC) Cell Models

### Example 2

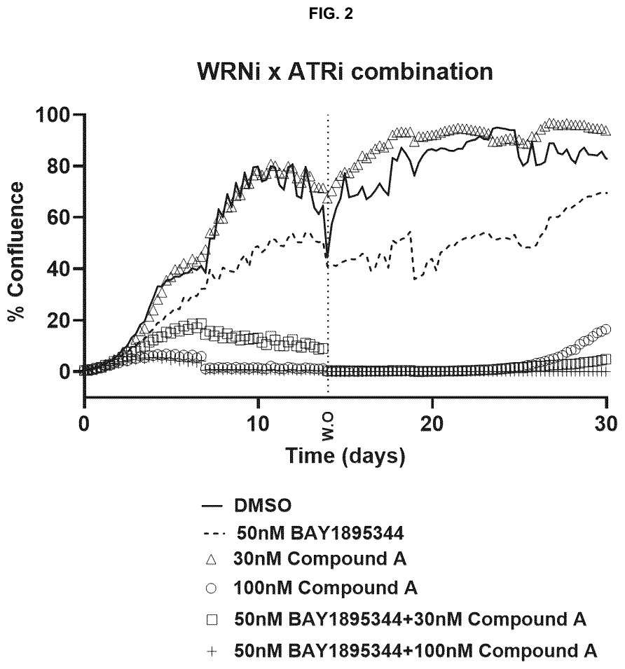

In vitro combination of Compound A with ATRi (BAY1895344) in SW48 model demonstrates a robust inhibition of cellular proliferation and deeper response compared to Compound A monotherapy, even using a sub-efficacious concentration, at 30 nM on a long-term proliferation assay up to 30 days (FIG. 2).

### Example 3

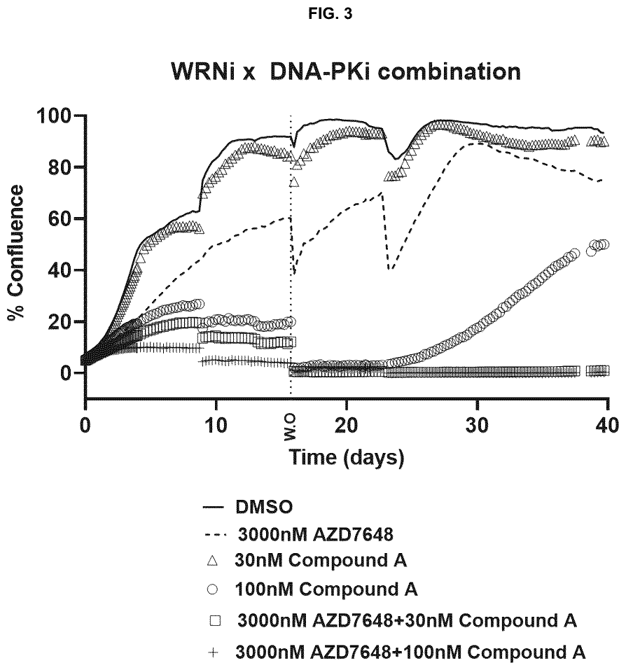

Further data indicates that combination of DNA-PKi with Compound A could prevent/delay the relapse observed after Compound A monotherapy (30-100 nM), on a long-term proliferation assay up to 40 days in SW48 model (FIG. 3).

### Example 4

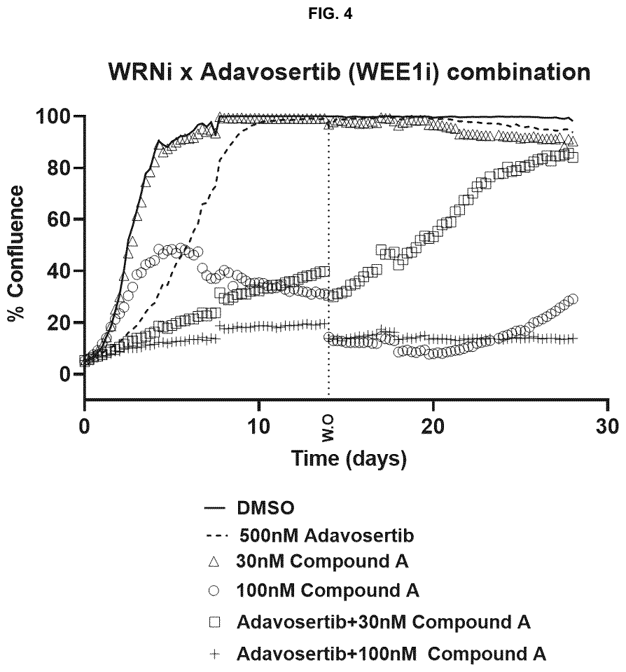

Combination of Compound A (30-100 nM) with WEE1i adavosertib (AZD1775) demonstrates a longer-lasting response in HCT116 (FIG. 4) and SW48 (Data not shown).

### Example 5

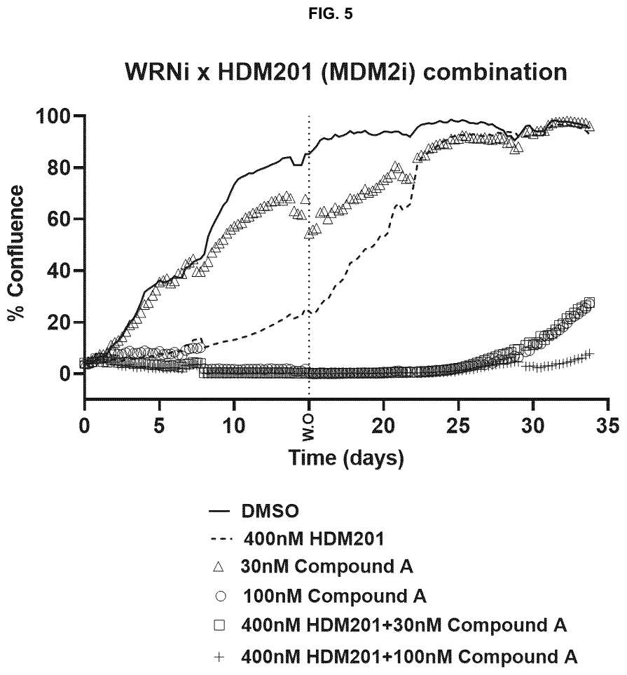

Combination of the selective inhibitor of the p53-MDM2 interaction, HDM201, with Compound A revealed a strong inhibition of cell proliferation, in SW48, even when using a sub-efficatious concentration of Compound A (30 nM) (FIG. 5).

### Example 11

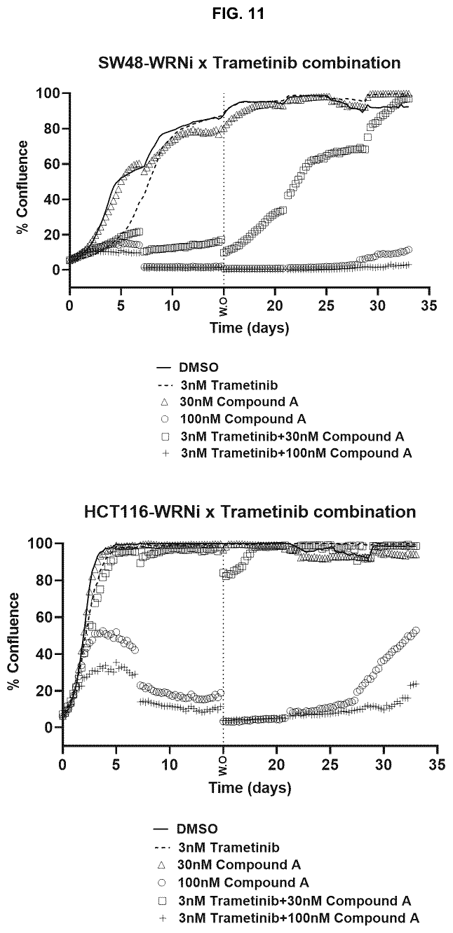

Combination of Compound A and Trametinib induces stronger inhibition of cell proliferation when comparing with compound A monotherapy (30 nM), in HCT116 and SW48 models. A longer-lasting response was observed when comparing with Compound A monotherapy at 100 nM on a long-term proliferation assay up to 35 days (FIG. 11).

### Example 21

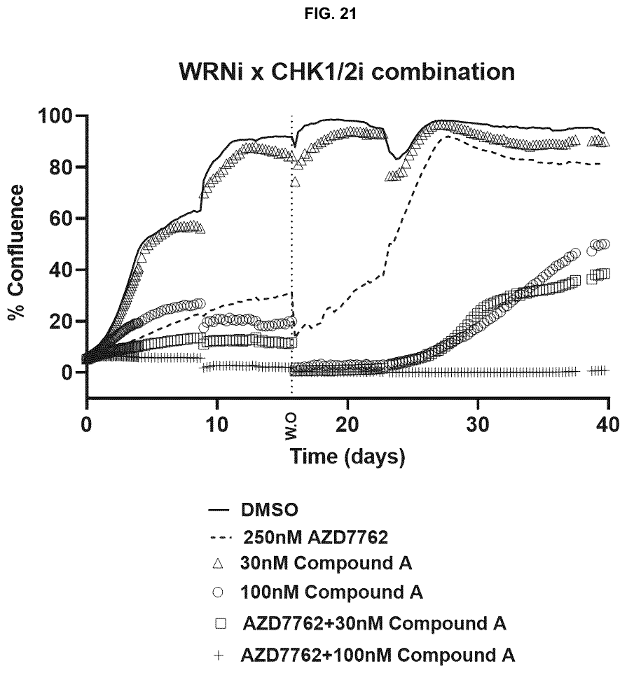

Compound A and dual CHK1/2i (AZD7762) combination leads to enhanced anti-proliferative effect in SW48 model even using a sub-efficacious concentration of Compound A at 30 nM and longer-lasting response is achieved using 100 nM Compound A on a long-term proliferation assay up to 40 days (FIG. 21).

### Example 34

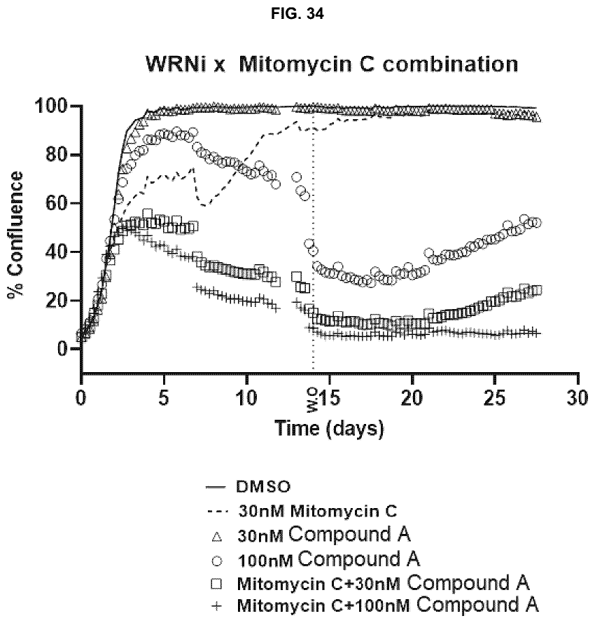

Combination of Compound A and Mitomycin C induces a strong inhibition of cell proliferation when comparing with compound A monotherapy (30 nM), in HCT116 model. A longer-lasting response was observed when comparing with Compound A monotherapy at 100 nM on a long-term proliferation assay up to 28 days (FIG. 34).

### Examples 6 and 7: In Vitro Proliferation Effect of Compound a in Combination with Ionizing Radiation in MSI^{high} Colorectal Cancer (CRC) Cell Models

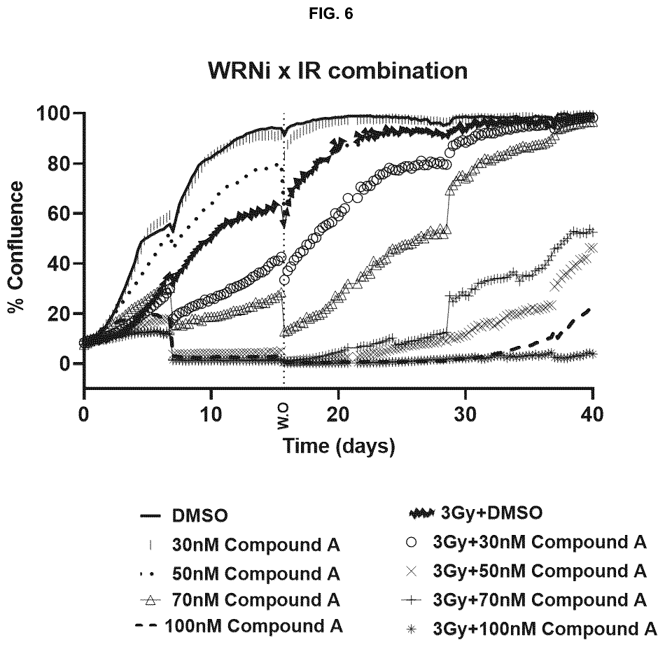

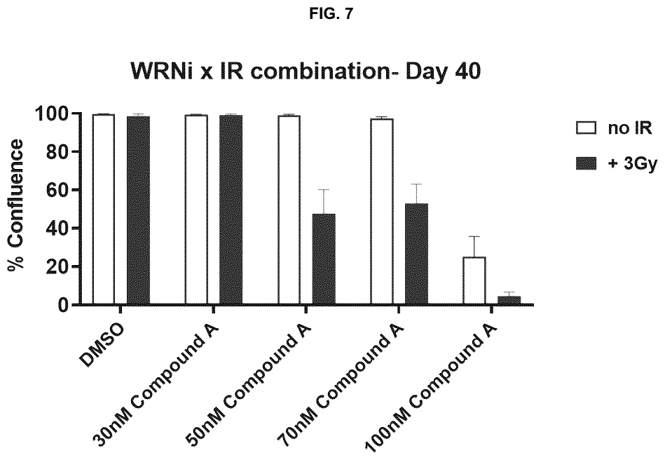

External beam irradiation is extensively used for cancer therapy. We have tested the in vitro combination of Compound A with ionizing radiation (IR) in SW48 model and have observed a dose-dependent increase in cytotoxicity (FIGS. 6 and 7) when comparing with the monotherapy treatments. This was particularly interesting as this increased cytotoxicity was achieved even using sub-efficacious doses of both therapies.

### Examples 12 and 15-18

All animal studies were conducted in accordance to procedures covered by permit number 2275 issued by the Kantonales Veterinäramt Basel-Stadt and strictly adhered to the Eidgenössisches Tierschutzgesetz and the Eidgenössische Tierschutzverordnung. All animals were allowed to adapt for 7 days and housed in a pathogen-controlled environment (5 mice/Type III cage) with access to food and water ad libitum and were identified with transponders.

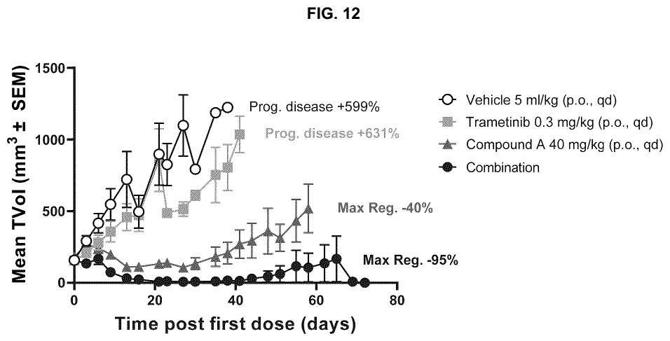

### Example 12 (FIG. 12)

Compound A was made-up fresh on a weekly basis and kept at 4° C. and protected from light. It was dissolved in 20% Hydroxypropyl-β-Cyclodextrine (HP-β-CD, #H107-100G, SIGMA) in water (#10977023, Invitrogen) and administered p.o. Trametinib was made-up fresh every 3 weeks and kept at room temperature and protected from light. It was dissolved in 0.5% hydroxypropylmethylcellulose (HPMC, #09963-500, SIGMA) and 0.2% Tween-80 (#P4780-500 ML, SIGMA) in distilled water.

5.0×10⁶ SW48 colorectal carcinoma cells were implanted subcutaneously in nude mice (Charles River, Germany). Treatment was initiated when tumors reached 150-200 mm³ volume. Efficacy studies and tumor response were measured as above.

Compound A at 40 mg/kg as single agent induced a 40% tumor regression for 30 days followed by relapse in all tumor-bearing mice. As expected, treatment with trametinib at 0.3 mg/kg had no effect on the BRAF^{WT} SW48 tumor growth. Interestingly, the combination of compound A with a clinical relevant dose of trametinib showed a 95% tumor regression sustained until day 40. Afterwards, only 30% of mice showed a relapse on treatment. Unexpectedly, the combination of compound A and trametinib showed a robust benefit and translated into homogeneous regression of a BRAF^{WT} MSI^{High} SW48 model.

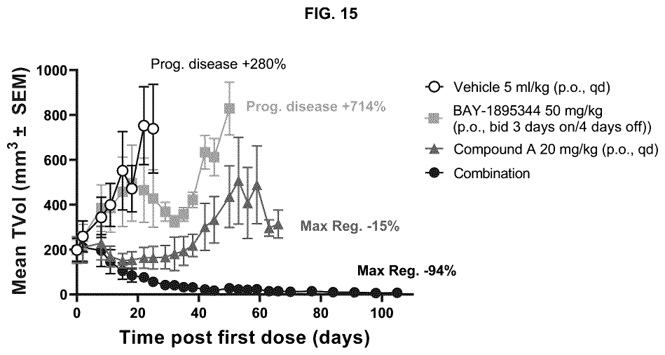

### Example 15 (FIG. 15)

Compound A was made-up fresh on a weekly basis and kept at 4ºC and protected from light. It was dissolved in 20% Hydroxypropyl-β-Cyclodextrine (HP-β-CD, #H107-100G, SIGMA) in water (#10977023, Invitrogen) and administered p.o. BAY-1895344 was made-up fresh weekly and kept at RT and protected from light. It was dissolved in PBS (#10010072, THERMOFISHER), pH was then adjusted to 2-2.5 using 1N HCl (#717631L, FLUKA).

5.0×10⁶ SW48 colorectal carcinoma cells were implanted subcutaneously in nude mice (Charles River, Germany). Treatment was initiated when tumors reached 150-200 mm³ volume. Efficacy studies and tumor response were measured as above.

Treatment with the ATR inhibitor elimusertib (BAY-1895344) at 50 mg/kg had no effect on the SW48 tumor growth. Compound A at 20 mg/kg as single agent induced a 15% tumor regression for 30 days followed by relapse in all tumor-bearing mice. Interestingly, the combination of a sub-optimal dose of compound A with a high and ineffective dose of BAY-1895344 showed a 94% tumor regression with no relapse until day 105. As mechanistically expected, the combination of compound A and BAY-1895344 showed a robust benefit and translated into homogeneous and sustained regression of a MSI^{High} SW48 model.

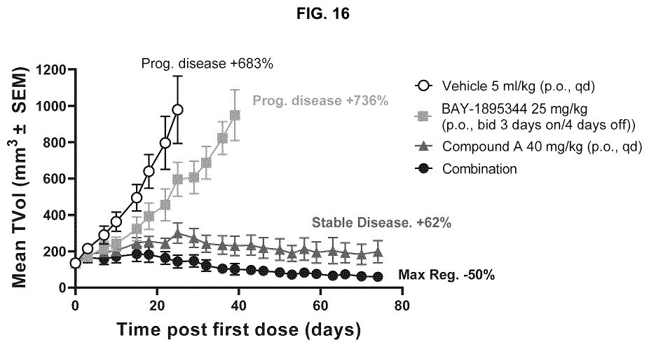

### Example 16 (FIG. 16)

Compound A was made-up fresh on a weekly basis and kept at 4° C. and protected from light. It was dissolved in 20% Hydroxypropyl-β-Cyclodextrine (HP-β-CD, #H107-100G, SIGMA) in water (#10977023, Invitrogen) and administered p.o. BAY-1895344 was made-up fresh weekly and kept at RT and protected from light. It was dissolved in PBS (#10010072, THERMOFISHER), pH is then adjusted to 2-2.5 using 1N HCl (#717631L, FLUKA).

Surgical tumor tissues from treatment-naive cancer patients were implanted in the right flank of nude mice (Charles River, Germany). All samples were anonymized and obtained with informed consent and under the approval of the institutional review boards of the tissue providers and Novartis. PDX models were histologically characterized and genetically profiled using various technology platforms after serial passages in mice. External diagnosis was independently confirmed by in-house pathologists. MSI^{high} CRC HX-2861 tumors was induced and expanded by transplantation as previously described (Gao, 2015). Approximately 20-30 mg of frozen tissue fragments embedded in Matrigel (#354234, Corning) were implanted subcutaneously into right flank region of nude mice using a trocar needle. Successfully engrafted tumor models were then passaged to generate enough tumor-bearing mice to be enrolled in an efficacy experiment. Treatment was initiated when tumors reached 150-200 mm³.

Treatment with the ATR inhibitor elimusertib (BAY-1895344) at 25 mg/kg had no effect on the HX-2861 tumor growth. Compound A at 40 mg/kg as single agent induced an homogenous and sustained stable disease. Interestingly, the combination of compound A with an ineffective dose of BAY-1895344 showed a 50% tumor regression with no relapse until day 74. As mechanistically expected, the combination of compound A and BAY-1895344 showed a robust benefit and translated into homogeneous and sustained regression of a MSI^{High} HX-2861 model.

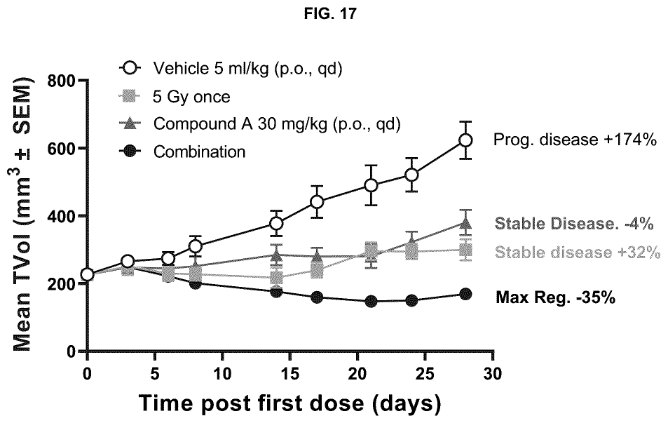

### Example 17 (FIG. 17)

Compound A was made-up fresh on a weekly basis and kept at 4° C. and protected from light. It was dissolved in 20% Hydroxypropyl-β-Cyclodextrine (HP-β-CD, #H107-100G, SIGMA) in water (#10977023, Invitrogen) and administered p.o. Irradiation was performed with a XRad320™ from Precision X-Ray and an adjustable X-ray Collimator (#XD1601-0000, Precision X-Ray). Only the tumor implanted subcutaneously by using specific shields (#XD1907-2012, Precision X-Ray). 1.0×10⁷ IM95 gastric carcinoma cells were implanted subcutaneously in SCID-BEIGE mice (Charles River, Germany). Treatment was initiated when tumors reached 200 mm³ volume. Efficacy studies and tumor response were measured as above.

Irradiation of IM95 tumor at 5 Gy induced a sustained and homogeneous stable disease. Compound A at 30 mg/kg as single agent also induced a stable disease for 21 days but followed by relapse in all tumor-bearing mice. Interestingly, the combination showed a 35% tumor regression with no relapse until day 28. As mechanistically expected, the combination of compound A and irradiation showed an interesting benefit and translated into homogeneous and sustained regression of a MSI^{High} IM95 model.

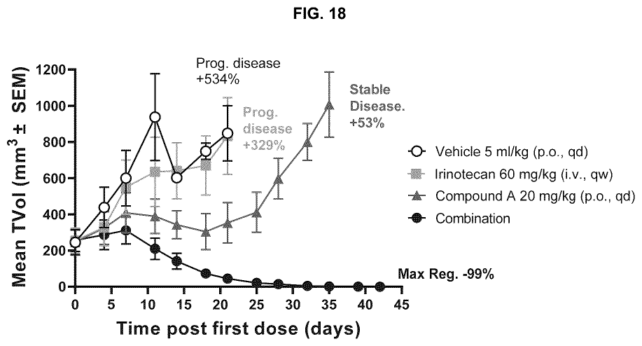

### Example 18 (FIG. 18)

Compound A was made-up fresh on a weekly basis and kept at 4° C. and protected from light. It was dissolved in 20% Hydroxypropyl-β-Cyclodextrine (HP-β-CD, #H107-100G, SIGMA) in water (#10977023, Invitrogen) and administered p.o. Irinotecan was made-up fresh weekly. It was dissolved in NaCl 0.9% (#395158, B.BRAUN).

5.0×10⁶ SW48 colorectal carcinoma cells were implanted subcutaneously in nude mice (Charles River, Germany). Treatment was initiated when tumors reached 150-200 mm³ volume. Efficacy studies and tumor response were measured as above.

Weekly treatment with irinotecan at 60 mg/kg had no effect on the SW48 tumor growth. Compound A at 20 mg/kg as single agent induced a stable disease for 21 days followed by a relapse in all tumor-bearing mice. Interestingly, the combination of a sub-optimal dose of compound A with a high and ineffective dose of irinotecan showed a 99% tumor regression with no relapse until day 42. As mechanistically expected, the combination of compound A and irinotecan showed a robust benefit and translated into homogeneous and sustained regression of a MSI^{High} SW48 model.

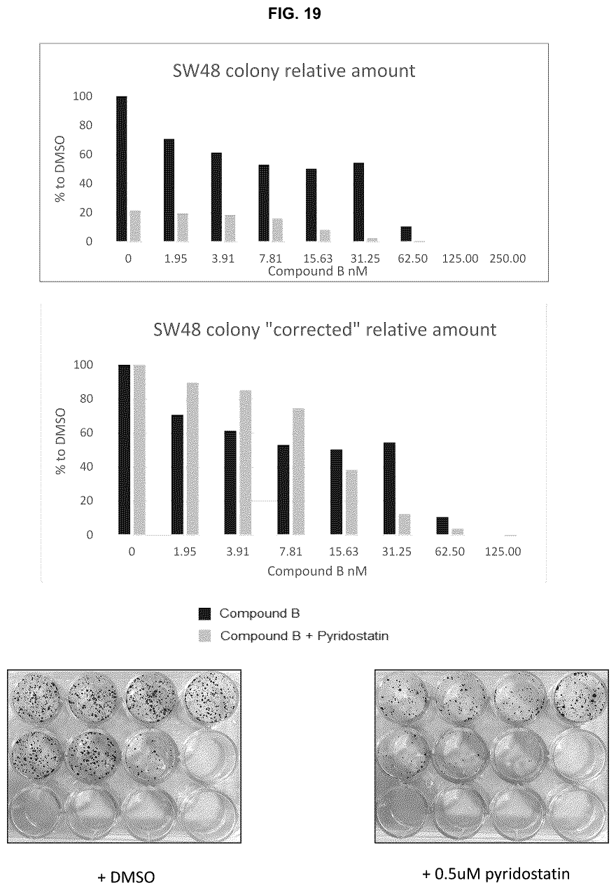

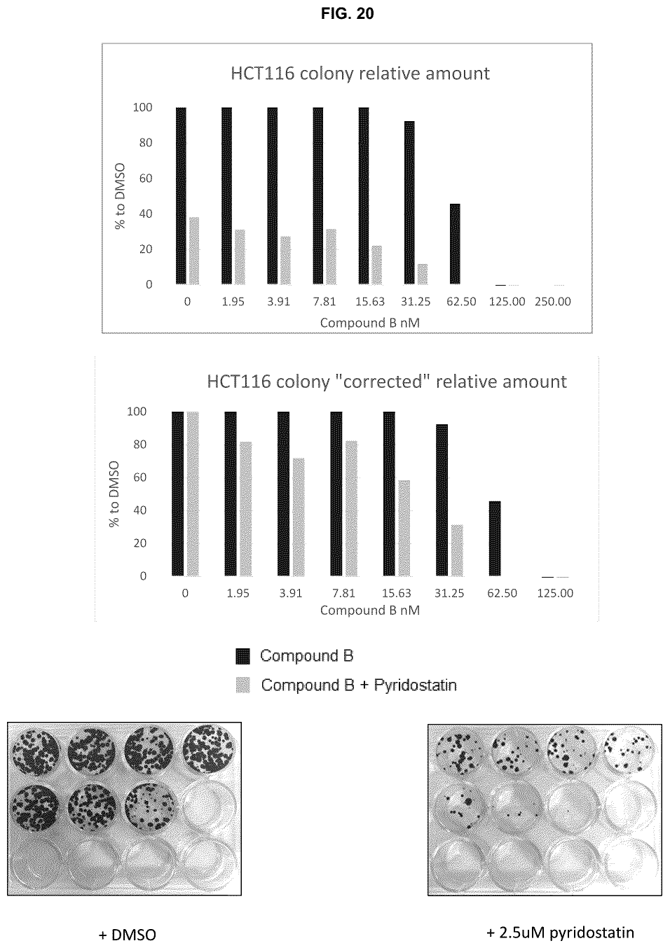

Examples 19 and 20 (FIGS. 19 and 20 respectively): Cell lines were obtained from ATCC and media and culture conditions used as recommended by ATCC. All cells were maintained at 37° C. in a humidified 5% CO2 incubator. Cells were seeded at 400 cells for HCT116 and 300 cells for SW48 per well in a 12-well plate in 1 ml of media. WRN inhibitor example compound B was added with a starting concentration of 2 μM, with or without pyridostatin at a concentration of 0.5 UM for SW48 cells (FIG. 19) and 2.5 μM for HCT116 cells (FIG. 20). Following overnight incubation at 37° C. in a humidified 5% CO2 atmosphere, eleven 2-fold serial dilutions of a given compound stock (obtained at a concentration of 10 mM in DMSO and stored at 4° C.) were dispensed directly into each assay plates using a HP 300D non-contact Digital Dispenser (TECAN). The final concentration of DMSO was normalized to 0.1% in all wells. Cells were left in the incubator for 10-20 days, with medium exchange every 3-4 days. After this time, 100 μl formaldehyde 37% was added directly in each test well and incubated for 15 minutes at room temperature. After rinsing twice with 5 ml of water, 1 ml of Crystal violet (Sigma #HT90132) was added for 15 minutes at room temperature. Wells were rinsed three times with water and pictures of plates were taken. After scanning, 1 mL of 10% acetic acid were added to the plates and shaken until complete color dissolution. 200 μl of this solution was transferred in a 96 well plate and absorbance measured at 600 nM using microtiter plate reader (Synergy HT). FIGS. 19 and 20 depict the images of the stained plates with the colonies with a range of WRN inhibitor compound B from 1.95 nM to 2000 nM in 2-fold increments, with or without pyridostatin. Graphs are a quantification of the signal either relative to DMSO of WRNi alone (top graph each figure), or relative to DMSO within the plate (i.e. in the presence of pyridostatin).

### Example 22

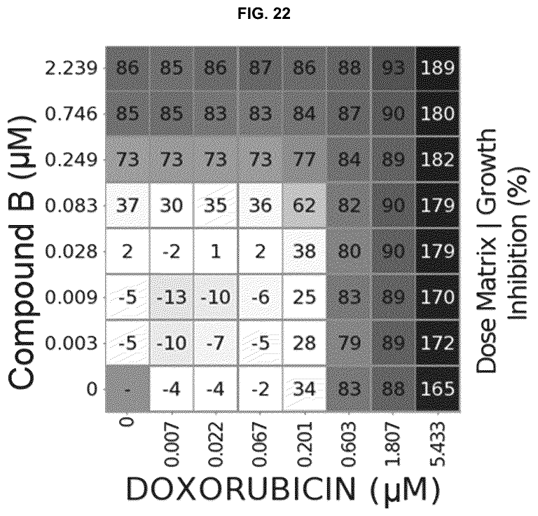

In vitro viability of a colorectal cancer cell line was assessed using the CellTiterGlo following 4-day treatment with the WRN inhibitor Compound B combined with doxorubicin. Proliferation of HCT116 cells was inhibited by Compound B alone and Doxorubicin alone. Further, the combination displayed synergistic growth inhibition (Loewe score of 3.110) compared to either treatment alone (FIG. 22)

### Example 23

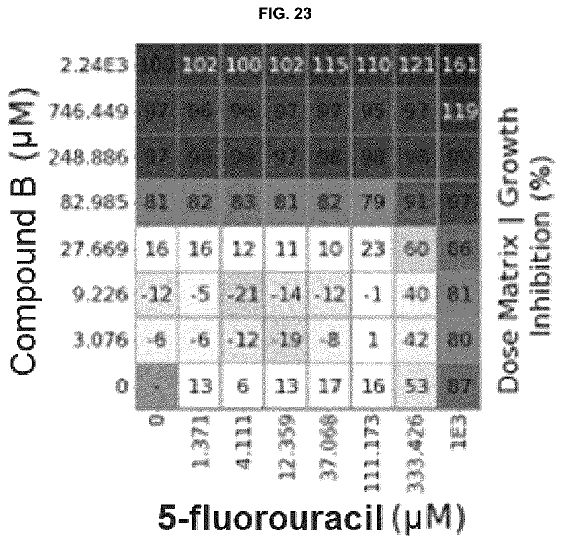

In vitro viability of the colorectal cancer cell line SW48 was assessed using the CellTiterGlo following 4-day treatment with the WRN inhibitor Compound B combined with 5-fluorouracil. Proliferation of SW48 cells was inhibited by Compound B alone and 5-fluorouracil alone. Further, the combination displayed synergistic growth inhibition (Loewe score of 2.370) compared to either treatment alone (FIG. 23).

### Example 24

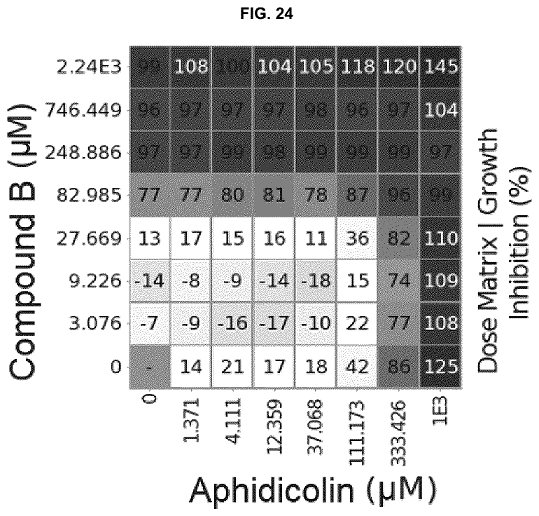

In vitro viability of the colorectal cancer cell line SW48 was assessed using the CellTiterGlo following 4-day treatment with the WRN inhibitor Compound B combined with Aphidicolin. Proliferation of SW48 cells was inhibited by Compound B alone and Aphidicolin alone. Further, the combination displayed slightly synergistic growth inhibition (Loewe score of 1.330) compared to either treatment alone (FIG. 24).

### Example 25

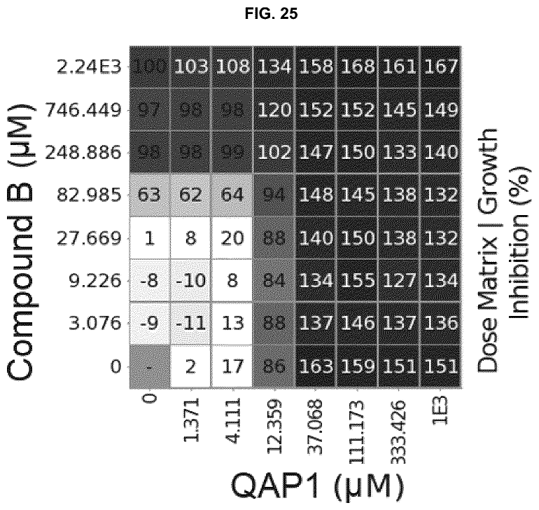

In vitro viability of the colorectal cancer cell line SW48 was assessed using the CellTiterGlo following 4-day treatment with the WRN inhibitor Compound B combined with QAP1. Proliferation of SW48 cells was inhibited by Compound B alone and QAP1 alone. Further, the combination displayed slightly synergistic growth inhibition (Loewe score of 1.188) compared to either treatment alone. (FIG. 25)

### Example 26

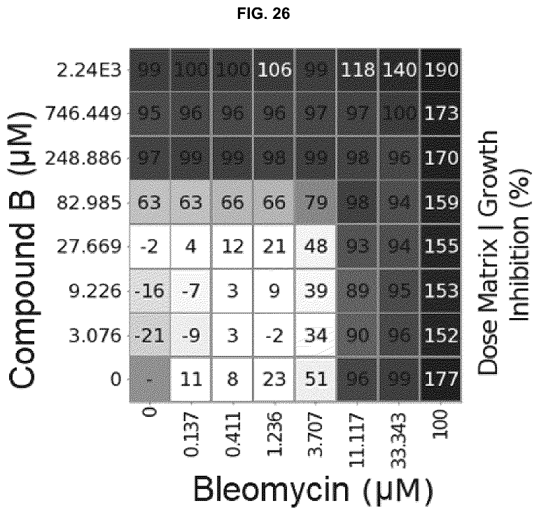

In vitro viability of the colorectal cancer cell line SW48 was assessed using the CellTiterGlo following 4-day treatment with the WRN inhibitor Compound B combined with Bleomycin. Proliferation of SW48 cells was inhibited by Compound B alone and Bleomycin alone. Further, the combination displayed slightly synergistic growth inhibition (Loewe score of 1.560) compared to either treatment alone (FIG. 26).

### Example 27

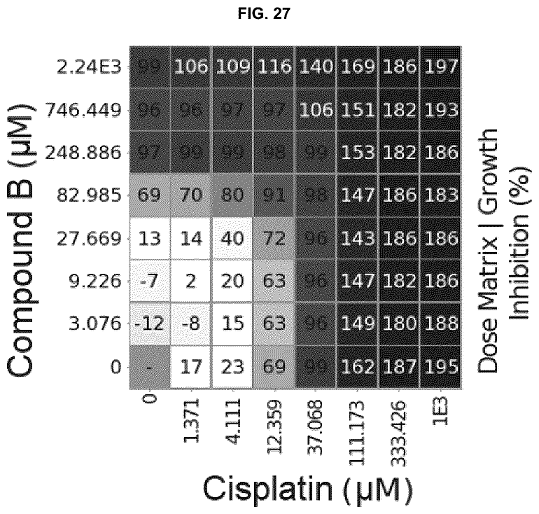

In vitro viability of the colorectal cancer cell line SW48 was assessed using the CellTiterGlo following 4-day treatment with the WRN inhibitor Compound B combined with cisplatin. Proliferation of SW48 cells was inhibited by Compound B alone and cisplatin alone. Further, the combination displayed slightly synergistic growth inhibition (Loewe score of 1.450) compared to either treatment alone (FIG. 27).

### Example 28

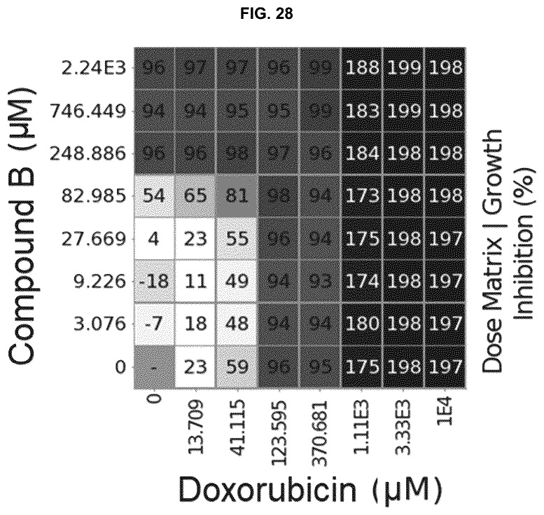

In vitro viability of the colorectal cancer cell line SW48 was assessed using the CellTiterGlo following 4-day treatment with the WRN inhibitor Compound B combined with Doxorubicin. Proliferation of SW48 cells was inhibited by Compound B alone and Doxorubicin alone. Further, the combination displayed synergistic growth inhibition (Loewe score of 5.420) compared to either treatment alone (FIG. 28).

### Example 29

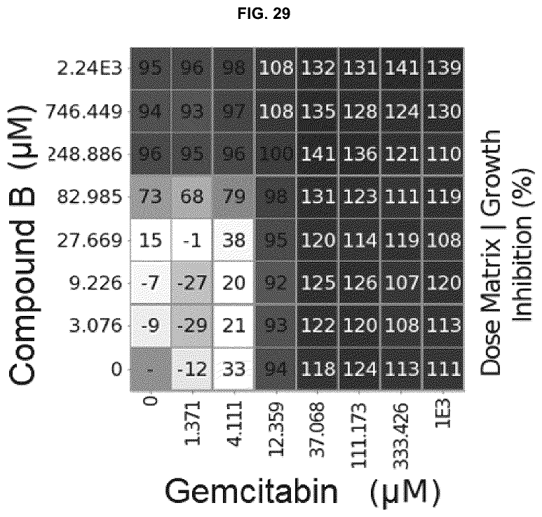

In vitro viability of the colorectal cancer cell line SW48 was assessed using the CellTiterGlo following 4-day treatment with the WRN inhibitor Compound B combined with Gemcitabin. Proliferation of SW48 cells was inhibited by Compound B alone and Gemcitabin alone. Further, the combination displayed synergistic growth inhibition (Loewe score of 4.700) compared to either treatment alone (FIG. 29).

### Example 30

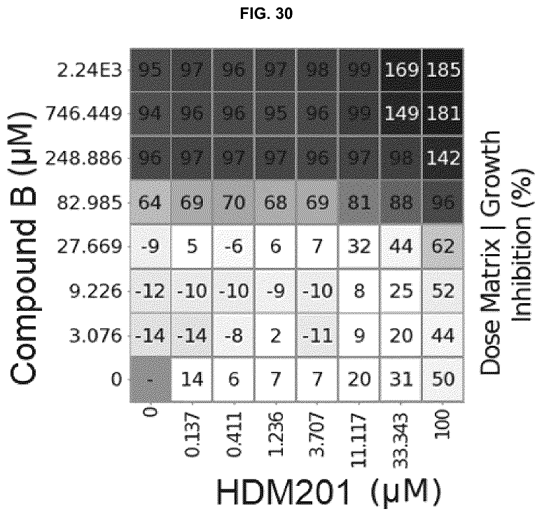

In vitro viability of the colorectal cancer cell line SW48 was assessed using the CellTiterGlo following 4-day treatment with the WRN inhibitor Compound B combined with HDM201. Proliferation of SW48 cells was inhibited by Compound B alone and HDM201 (to some extent) alone. Further, the combination displayed synergistic growth inhibition (Loewe score of 7.940) compared to either treatment alone (FIG. 30).

### Example 31

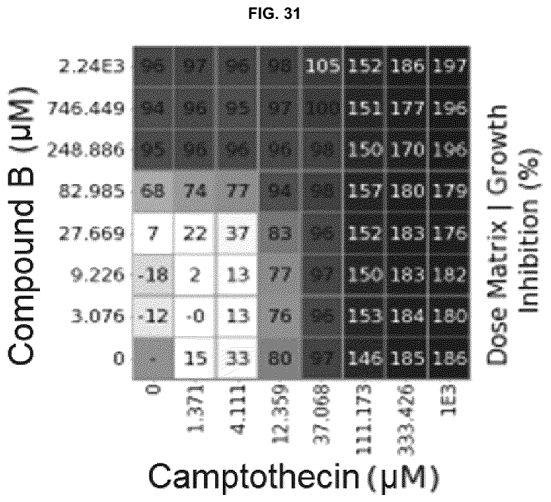

In vitro viability of the colorectal cancer cell line SW48 was assessed using the CellTiterGlo following 4-day treatment with the WRN inhibitor Compound B combined with Camptothecin. Proliferation of SW48 cells was inhibited by Compound B alone and Camptothecin alone. Further, the combination displayed synergistic growth inhibition (Loewe score of 2.580) compared to either treatment alone (FIG. 31).

### Example 32

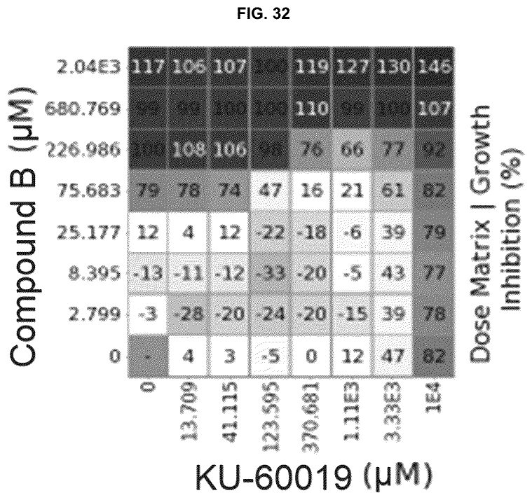

In vitro viability of the colorectal cancer cell line SW48 was assessed using the CellTiterGlo following 4-day treatment with the WRN inhibitor Compound B combined with KU-60019. Proliferation of SW48 cells was inhibited by Compound B alone and KU-60019 alone. Further, the combination displayed slightly synergistic growth inhibition (Loewe score of 1.420) compared to either treatment alone (FIG. 32).

### Example 33

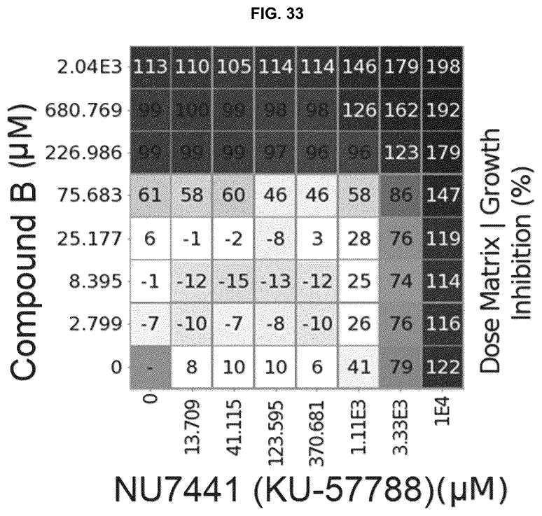

In vitro viability of the colorectal cancer cell line SW48 was assessed using the CellTiterGlo following 4-day treatment with the WRN inhibitor Compound B combined with NU7441 (KU-57788). Proliferation of SW48 cells was inhibited by Compound B alone and NU7441 (KU-57788) alone. Further, the combination displayed synergistic growth inhibition (Loewe score of 9.140) compared to either treatment alone (FIG. 33).

### Example 35: In-Vivo Efficacy of WRN Inhibitor Compound a after Administration in Nude Mice Bearing SW620 Colorectal Carcinoma MSS (Microsatellite Stable) Xenografts, Combination with Temozolomide and/or Irinotecan

Materials and Methods

SW620 (CCL-227, ATCC) [MSS colorectal carcinoma (CRC)] was cultured in DMEM (#1-26F01-I, AMIMED) supplemented with 10% FCS (#2-0-1F30-I, BioConcept), 4 mM L-glutamine (#5-10K50-H, BioConcept) and 1 mM Sodium pyruvate (#5-60F00-H, BioConcept). Cells were passaged by washing first with Dulbecco's PBS without Ca²⁺/Mg²⁺ (#3-05F29-1, AMIMED), trypsinising cells with Trypsin 0.05% in PBS with EDTA (#5-51F00-H, AMIMED), centrifuging in the respective culture media, and splitting cells into fresh media at a ratio of 1:8, 2 times per week.

Athymic nude mice (Charles River, Germany) were allowed to adapt for 7 days upon arrival at the animal facility and housed in a pathogen-controlled environment (5 mice/Type III cage) with access to food and water ad libitum. Animals were identified with transponders. Studies described in this report were performed according to procedures covered by a permit number issued by the Kantonales Veterinäramt Basel-Stadt.

Subcutaneous tumors were initiated by injecting 5.0×10⁶ SW620 tumor cells in 50% Matrigel (#354234, Corning) in HBSS (#H6648, SIGMA) in the right flank of nude mice. The efficacy experiment (n=6), started 11 days post-cell injection.

Compound A and Temozolomide were made-up fresh on a weekly basis and kept at 4° C. and protected from light. Compound A was dissolved in 20% Hydroxypropyl-ß-Cyclodextrine (HP-ß-CD, #H107-100G, SIGMA) in UltraPure™ Water (#10977-035, Invitrogen) and administered p.o. at 10 ml/kg. Temozolomide was dissolved in 30% PEG-400 (#202398-500G, SIGMA) and 70% Dulbecco's PBS without Ca²⁺/Mg²⁺ (#3-05F29-1, AMIMED) and administered p.o. at 10 mL/kg on 5 consecutive days within a 21 days cycle. Irinotecan was made-up fresh for each treatment once a week by diluting with NaCl 0.9% (#3-06S00-I, BioConcept). The solution was administered i.v. at 5 mL/kg.

Tumor volumes (TVol), determined from caliper measurements (using the formula I*w2*π/6) were measured two times per week. Tumor response was quantified by the change in tumor volume (endpoint minus starting value in mm³) as the T/C (the ratio between the tumor volume in the treated group and in the untreated control group). In the case of a tumor regression or to assess the percentage of change in TVol, the tumor response was quantified by the percentage of regression of the starting TVol. The body-weight (BW) of the animal was measured three times per week allowing calculation at any particular time-point relative to the day of initiation of treatment (day 0) of the percentage change in BW (4% BW).

Results

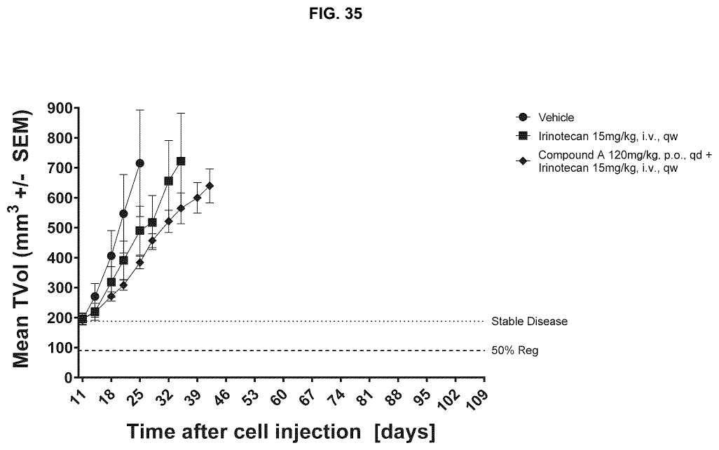

FIG. 35 shows the mean tumor volume in nude mice bearing SW620 (CCL-227, ATCC) [MSS colorectal carcinoma (CRC)] xenografts, following treatment with irinotecan alone, or irinotecan in combination with Compound A. Starting 11 days post-cell injection, to the first group irinotecan was administered intravenously at 15 mg/kg, once weekly for 4 weeks, and to the second group irinotecan was administered intravenously at 15 mg/kg once weekly for 5 weeks in combination with Compound A administered orally at 120 mg/kg daily for 31 days.

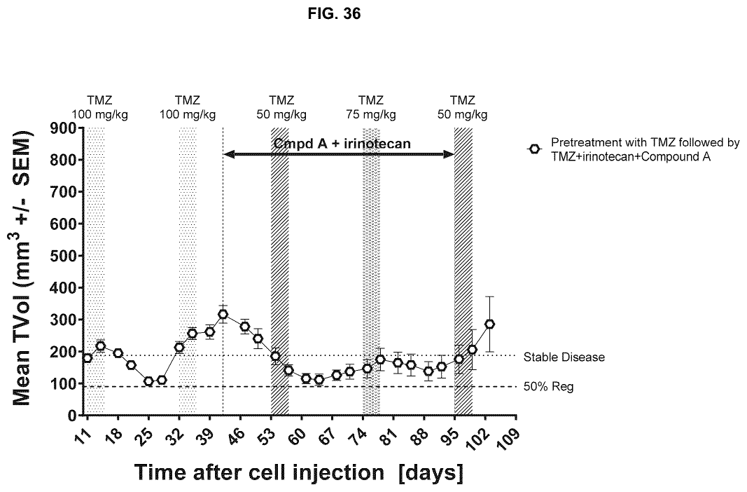

FIG. 36 shows the mean tumor volume in nude mice bearing SW620 (CCL-227, ATCC) [MSS colorectal carcinoma (CRC)] xenografts, following pre-treatment with temozolomide, then followed by treatment with temozolomide, irinotecan and Compound A. Starting 11 days post-cell injection, temozolomide was administered orally at 100 mg/kg on 5 consecutive days (day 11 to day 15 and day 32 to day 36) and administration was continued on 21 day cycles of 5 consecutive days using the dose of temozolomide (TMZ) indicated in the Figure. At day 43 post-cell injection, irinotecan was administered intravenously at 15 mg/kg once weekly for 7 weeks in combination with Compound A, also administered from day 43, orally at 120 mg/kg daily for 52 days.

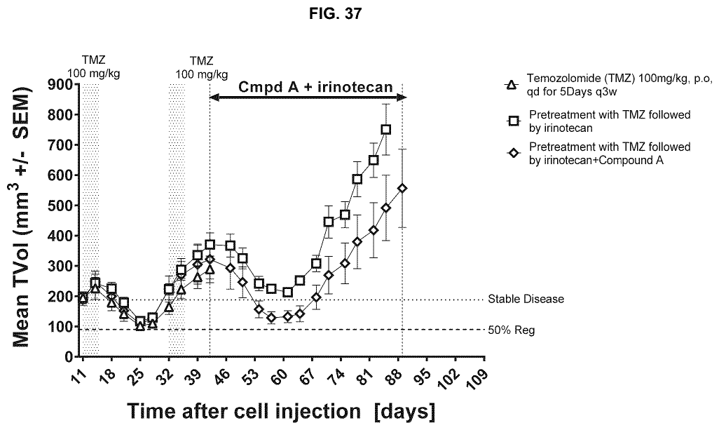

FIG. 37 shows the mean tumor volume in nude mice bearing SW620 (CCL-227, ATCC) [MSS colorectal carcinoma (CRC)] xenografts, following pre-treatment with temozolomide, then followed by treatment with irinotecan alone, or a combination of irinotecan and Compound A only. Starting 11 days post-cell injection, temozolomide was administered orally on a 21 day cycle of 5 consecutive days at 100 mg/kg on 5 consecutive days (day 11 to day 15 and day 32 to day 36). At day 43 post-cell injection, to the first group irinotecan was administered intravenously at 15 mg/kg once weekly for 6 weeks, and to the second group irinotecan was administered intravenously at 15 mg/kg once weekly for 6 weeks in combination with Compound A, also administered from day 43, orally at 120 mg/kg daily for 46 days.

Conclusion:

Treatment of parental SW620 tumors with irinotecan or the combination of irinotecan and compound A induced a delay of the tumor growth. Treatment with 1 cycle of temozolomide (TMZ) induced a 30-50% tumor regression in all treated groups and relapsed after 3 weeks. After the 2^{nd} cycle of TMZ treatment, all tumors were not responding anymore. One group was sacrificed and 2 other groups were treated with either irinotecan or the combination of irinotecan and compound A. Overall, the combination induced a more profound tumor regression with a maximum reached 15-19 days post treatment initiation. In both cases, the tumors relapsed with a subsequent delay when treated with irinotecan and compound A. One additional group was also enrolled with irinotecan and compound A but was still on TMZ therapy. Interestingly, this triple combination maintained the tumor size between stable disease and 50% regression for more than 40 days. Tumors relapsed only when irinotecan and compound A were stopped on day 95, showing a clear benefit from the triple combination.

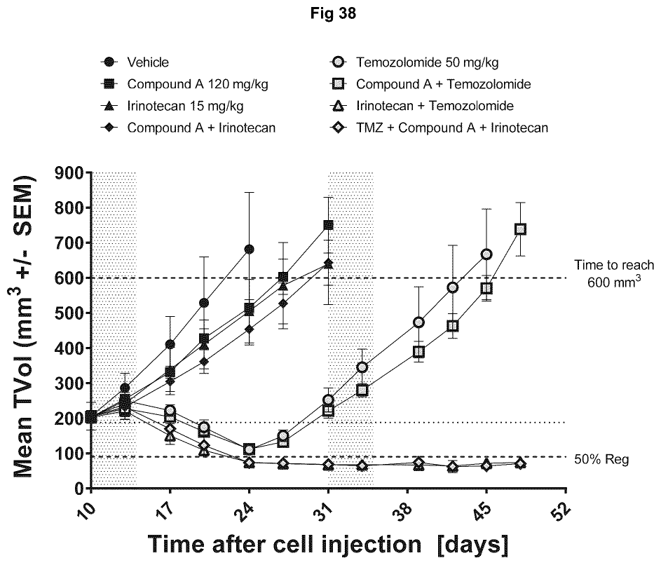

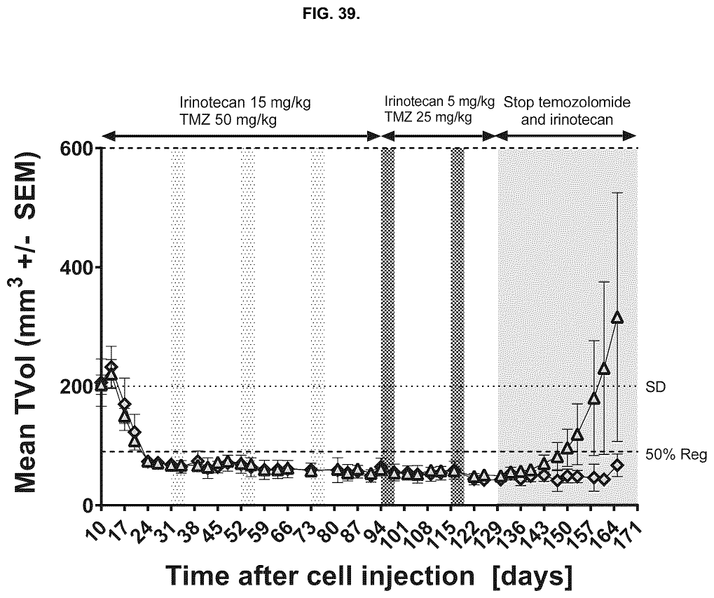

Additional Efficacy Results. (FIGS. 38 and 39)

(FIG. 38) Treatment of parental SW620 tumors with irinotecan, compound A or the combination of both induced a delay of the tumor growth. Treatment with 1 cycle of temozolomide (TMZ) alone or in combination with Compound A induced a 40% tumor regression and relapsed after 3 weeks. After the 2nd cycle of TMZ treatment, all tumors were not responding anymore in both treatment groups.

(FIG. 39) The combination of TMZ with either irinotecan or irinotecan and Compound A induced a 60-70% tumor regression in both treatment groups with no relapse after 3 cycles of TMZ. The decrease of the dose of TMZ at 25 mg/kg for 2 cycles and irinotecan at 5 mg/kg did not affect the tumor regressions in both treatment groups. However, stopping administration of temozolomide and irinotecan on day 129 in both groups induced a clear differentiation. Indeed, the group for which all treatments were stopped (opened triangle) showed relapses within 5 weeks, while the group which was still treated with compound A only remained below 50% tumor regression. These data show that repeated treatment with TMZ and irinotecan sensitized the SW620 tumor to Compound A.

Tolerability

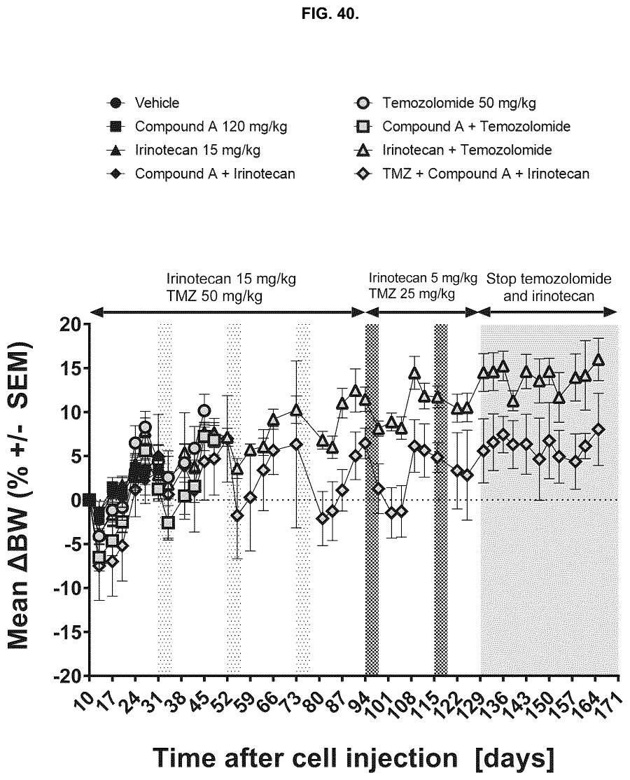

(FIG. 40) Overall, all treatments were well tolerated. Temozolomide (TMZ) in single agent or in the triple combination induced a dose-dependent body weight loss but mice always recovered at the end of the 21-days cycle.

### Example 36: In-Vivo Efficacy of WRN Inhibitor Compound a after Administration in Nude Mice Bearing T84 Colorectal Carcinoma MSS (Microsatellite Stable) Xenografts, Combination with Temozolomide and/or Irinotecan

T84 (CCL-248, ATCC) is MSS colorectal carcinoma (CRC). It was cultured in DMEM (#1-26F01-I, AMIMED) supplemented with 10% FCS (#2-0-1F30-I, BioConcept), 4 mM L-glutamine (#5-10K50-H, BioConcept) and 1 mM Sodium pyruvate (#5-60F00-H, BioConcept). Cells were passaged by washing first with Dulbecco's PBS without Ca²⁺/Mg²⁺ (#3-05F29-1, AMIMED), trypsinising cells with Trypsin 0.05% in PBS with EDTA (#5-51F00-H, AMIMED), centrifuging in the respective culture media, and splitting cells into fresh media at a ratio of 1:8, 2 times per week.

Athymic nude mice (Charles River, Germany) were allowed to adapt for 7 days upon arrival at the animal facility and housed in a pathogen-controlled environment (5 mice/Type III cage) with access to food and water ad libitum. Animals were identified with transponders. Studies described in this report were performed according to procedures covered by permit number 1975 issued by the Kantonales Veterinäramt Basel-Stadt and strictly adhered to the Eidgenössisches Tierschutzgesetz and the Eidgenössische Tierschutzverordnung.

Subcutaneous tumors were initiated by injecting 5.0×10⁶ T84 tumor cells in 50% Matrigel (#354234, Corning) in HBSS (#H6648, SIGMA) in the right flank of nude mice. Efficacy experiment (n=6) started approximatively 7 days post-cell injection.

Compound A and temozolomide were made-up fresh on a weekly basis and kept at 4° C. and protected from light. Compound A was dissolved in 20% Hydroxypropyl-ß-Cyclodextrine (HP-ß-CD, #H107-100G, SIGMA) in UltraPure™ Water (#10977-035, Invitrogen) and administered p.o. at 120 mg/kg and 10 mL/kg. Temozolomide was dissolved in 30% PEG-400 (#202398-500G, SIGMA) and 70% Dulbecco's PBS without Ca²⁺/Mg²⁺ (#3-05F29-1, AMIMED) and administered p.o. at 25 mg/kg and 10 mL/kg on 5 consecutive days within a 28 days cycle. Irinotecan was made-up fresh for each treatment once a week by diluting with NaCl 0.9% (#3-06S00-I, BioConcept). The solution was administered i.v. at 5 mg/kg and 5 mL/kg.

Tumor volumes (TVol), determined from caliper measurements (using the formula I*w2*TT/6) were measured two times per week. Tumor response was quantified by the change in tumor volume (endpoint minus starting value in mm³) as the T/C. In the case of a tumor regression or to assess the percentage of change in TVol, the tumor response was quantified by the percentage of regression of the starting TVol. The body-weight (BW) of the animal was measured three times per week allowing calculation at any particular time-point relative to the day of initiation of treatment (day 0) of the percentage change in BW (4% BW).

Efficacy Results

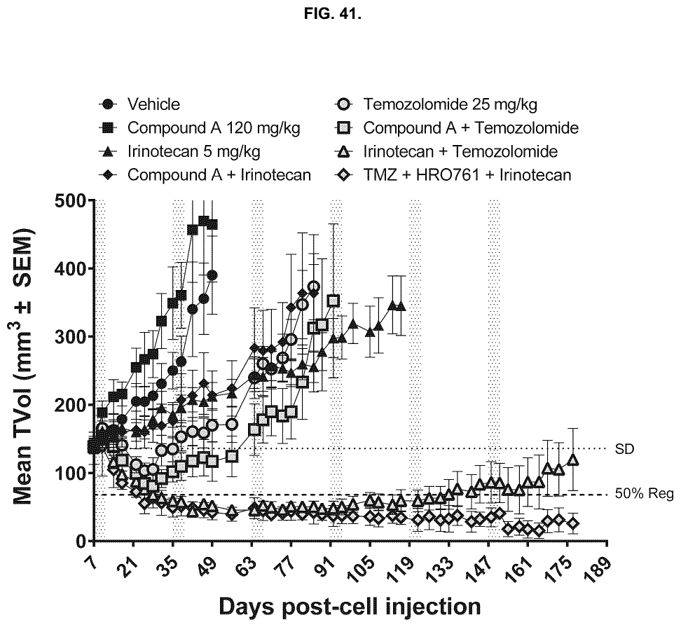

(FIG. 41) Treatment of parental T84 tumors with compound A had no effect on the tumor growth. Treatment with irinotecan or the combination with compound A induced a delay of the tumor growth. Treatment with 1 cycle of temozolomide (TMZ) alone or in combination with Compound A induced a 30-40% tumor regression followed by a quick relapse. After the 2nd cycle of TMZ treatment, all tumors were not responding anymore in both treatment groups.

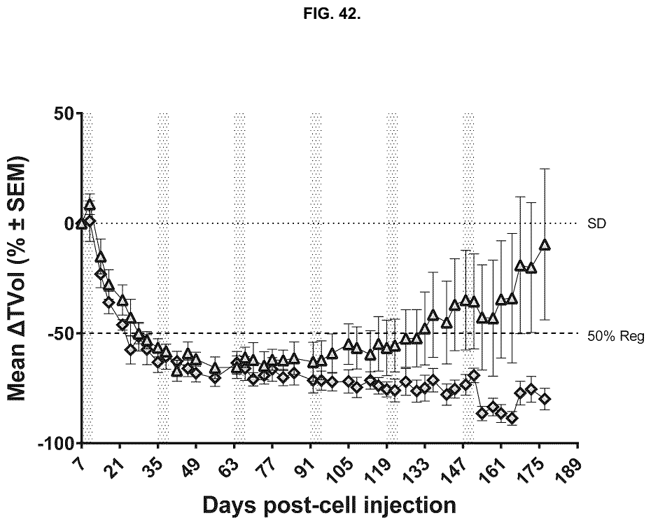

(FIG. 42) The combination of TMZ with either irinotecan or irinotecan and Compound A induced a 60-70% tumor regression in both treatment groups with no relapse after 4 cycles of TMZ. After, this time point, the double combination starts to slowly relapse to be back to baseline level on day 179. However, no relapse could be noted in the triple combination treatment group which 80% tumor regression on day 179. These data show that repeated treatment with TMZ and irinotecan sensitized the T84 tumor to Compound A and differentiation could be reached after a long period of treatment.

Tolerability Results

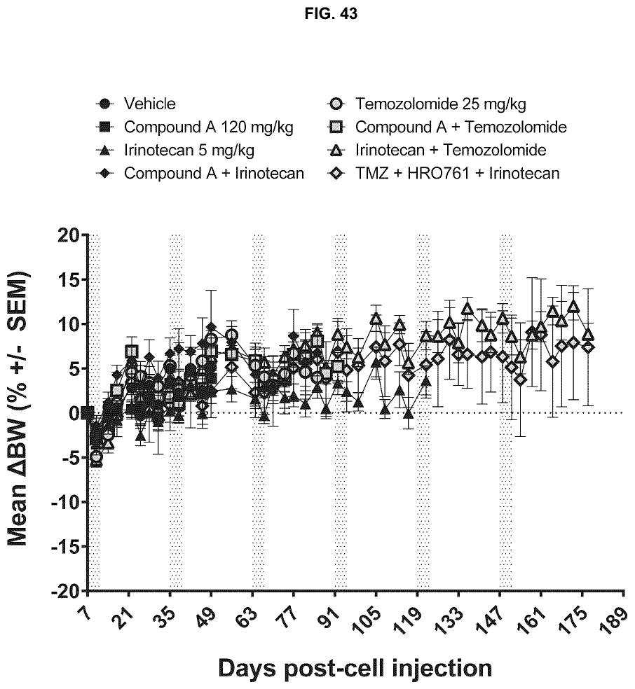

(FIG. 43) Overall, all treatments were well tolerated. Temozolomide (TMZ) in single agent or in the triple combination induced a slight body weight loss but mice always recovered at the end of the 28-days cycle.

### Example 37: In-Vivo Efficacy of WRN Inhibitor Compound a after Administration in Nude Mice Bearing SHP-77 Small Cell Lung Carcinoma (SCLC) MSS (Microsatellite Stable) Xenografts, in Combination with Temozolomide and Irinotecan

SHP-77 (CRL-2195, ATCC) is a MSS small cell lung carcinoma (SCLC). It was cultured in RPMI 1640 Medium, GlutaMAX™ (#61870036, GIBCO) supplemented with 10% FCS (#2-O-1F30-I, BioConcept). Cells were passaged by washing first with Dulbecco's PBS without Ca²⁺/Mg²⁺ (#3-05F29-1, AMIMED), trypsinising cells with Trypsin 0.05% in PBS with EDTA (#5-51F00-H, AMIMED), centrifuging in the respective culture media, and splitting cells into fresh media at a ratio of 1:8, 2 times per week.

Athymic nude mice (Charles River, Germany) were allowed to adapt for 7 days upon arrival at the animal facility and housed in a pathogen-controlled environment (5 mice/Type III cage) with access to food and water ad libitum. Animals were identified with transponders. Studies described in this report were performed according to procedures covered by permit number 1975 issued by the Kantonales Veterinäramt Basel-Stadt and strictly adhered to the Eidgenössisches Tierschutzgesetz and the Eidgenössische Tierschutzverordnung.

Subcutaneous tumors were initiated by injecting 2.0×10⁶ SHP-77 tumor cells in 50% Matrigel (#354234, Corning) in HBSS (#H6648, SIGMA) in the right flank of nude mice. Efficacy experiment (n=6-12) started approximatively 27 days post-cell injection.

Compound A and Temozolomide were made-up fresh on a weekly basis and kept at 4° C. and protected from light. Compound A was dissolved in 20% Hydroxypropyl-ß-Cyclodextrine (HP-β-CD, #H107-100G, SIGMA) in UltraPure™ Water (#10977-035, Invitrogen) and administered p.o. at 120 mg/kg and 10 ml/kg. Temozolomide was dissolved in 30% PEG-400 (#202398-500G, SIGMA) and 70% Dulbecco's PBS without Ca²⁺/Mg²⁺ (#3-05F29-1, AMIMED) and administered p.o. at 25 mg/kg and 10 ml/kg on 5 consecutive days within a 28 days cycle. Irinotecan was made-up fresh for each treatment once a week by diluting with NaCl 0.9% (#3-06S00-I, BioConcept). The solution was administered i.v. at 5 or 15 mg/kg and 5 mL/kg.

Tumor volumes (TVol), determined from caliper measurements (using the formula I*w2*TT/6) were measured two times per week. Tumor response was quantified by the change in tumor volume (endpoint minus starting value in mm³) as the T/C. In the case of a tumor regression or to assess the percentage of change in TVol, the tumor response was quantified by the percentage of regression of the starting TVol. The body-weight (BW) of the animal was measured three times per week allowing calculation at any particular time-point relative to the day of initiation of treatment (day 0) of the percentage change in BW (4% BW).

Efficacy Results

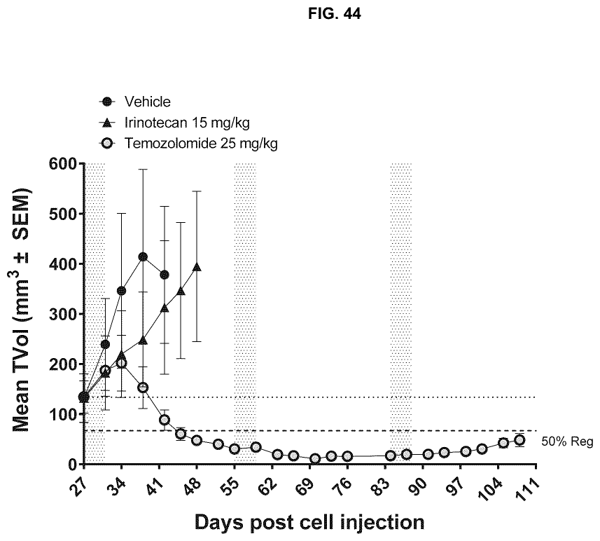

(FIG. 44) Treatment of parental SHP77 tumors with irinotecan had no effect on the tumor growth. Treatment with temozolomide (TMZ) induced a 90% tumor regression after 2 cycles followed by a slow relapse. After the 3^{rd} cycle of TMZ treatment, the tumors that were not responding anymore to TMZ were enrolled in 2 treatments groups, TMZ+irinotecan and TMZ+irinotecan+Compound A.

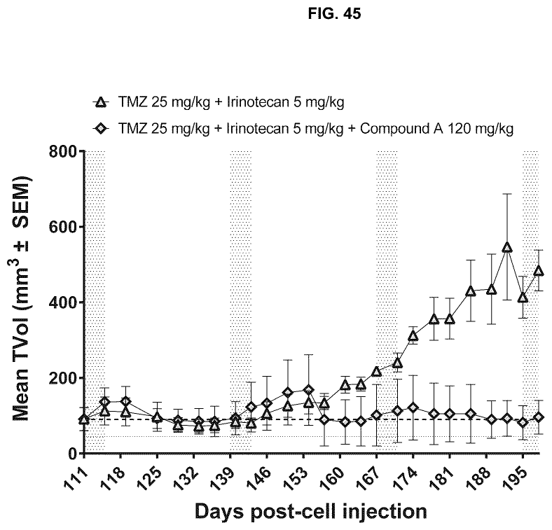

(FIG. 45) For 46 days (day 111 to 157), the addition of irinotecan to TMZ stabilized the tumor growth but followed by a clear relapse. However, the addition of Compound A on top of the double combination showed a stable disease for 87 days (day 111 to 198) and clearly prevented the relapse of the double combination. These data show that repeated treatment with TMZ followed by TMZ+irinotecan sensitized the SHP-77 tumor to Compound A and differentiation could be reached after a long period of treatment

Tolerability Results

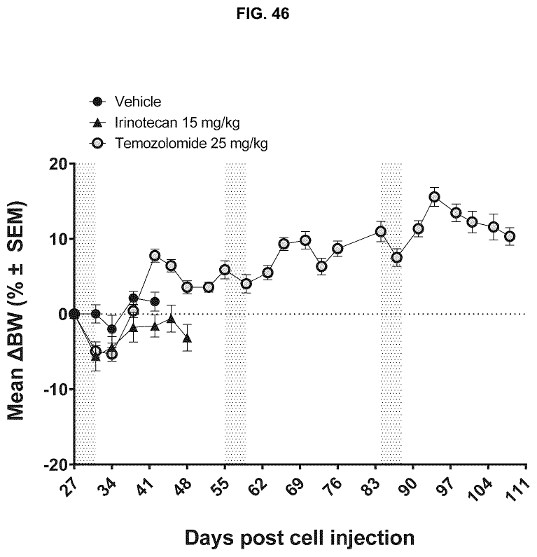

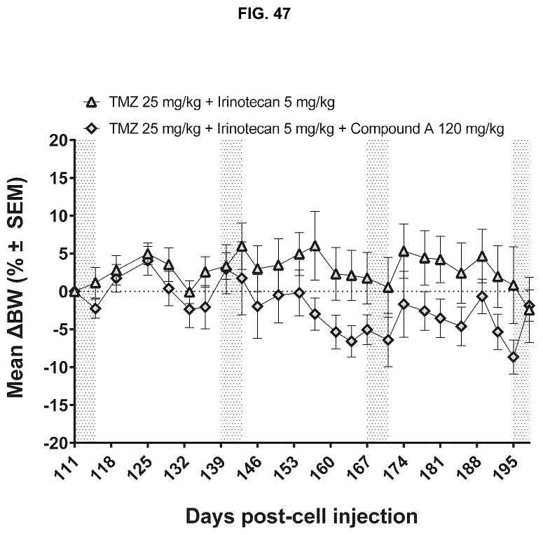

(FIGS. 46 and 47) Temozolomide (TMZ) as single agent was well tolerated, while irinotecan at 15 mg/kg induced a slight body weight loss. The double combination showed stable body weight in average over the time while the triple combination induced a slight body weight loss with some period of recovery.

1. A WRN inhibitor for use in the treatment of cancer, wherein the treatment further comprises administration of:
i. an agent which can
sensitise the cancer cells, for example for an improved response to treatment with a WRN inhibitor,
prime the cancer cells, for example, such priming may include triggering hypermutation status in cancer cells,
create or increase MMR deficiency in cancer cells,
create or increase MSI-H status in cancer cells, or
increase MMR heterogeneity of cancer cells,
and optionally wherein the treatment further comprises administration of:
ii. a chemotherapy, for example irinotecan.

2. A WRN inhibitor for use as claimed in claim 1, wherein the cancer is MSS (microsatellite stable) cancer.

3. A WRN inhibitor for use as claimed in claim 1 or claim 2, wherein the agent i. is selected from temozolomide, cisplatin and 6-thioguanine, or the agent i. is an ionising radiation based therapy selected from i) external beam radiation, ii) brachytherapy and ii) a radiopharmaceutical.

4. A WRN inhibitor for use as claimed in any of claims 1 to 3, wherein the agent i. is temozolomide.

5. A WRN inhibitor for use as claimed in any of claims 1 to 4, wherein the treatment further comprises administration of i. temozolomide and ii. irinotecan.

6. A WRN inhibitor for use as claimed in any of claims 1 to 5, wherein the WRN inhibitor is a compound of formula (1g), or a pharmaceutically acceptable salt thereof: 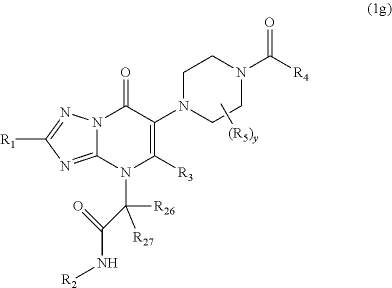
wherein
R₁ is selected from: 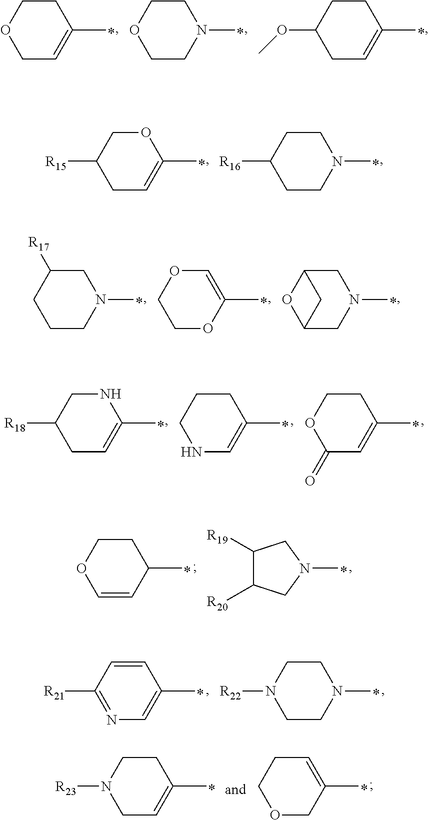
R₁₅ is H or F;
R₁₆ is H or R₂₅(R₂₄)N—;
R₁₇ is H or F;
R₁₈ is H or F;
R₁₉ is H or F;
R₂₀ is H or F;
R₂₁ is H or CH₃;
R₂₂ is H, CF₃, CHF₂CH₂, HOC(O)—CH₂—, H₃C—C(O)—, (H₃C)₃C—O—C(O)—;
R₂₃ is H, CF₃, CHF₂CH₂, (H₃C)₃C—O—C(O)—;
R₂₄ is CH₃;
R₂₅ is CHF₂CH₂—;
R₂₈ is CH₃, H or deuterium;
R₂₇ is H or deuterium;
R₂ is the moiety: 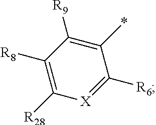
wherein R₆ is selected from H, Cl, CH₃, F, and Br;
R₈ is selected from H, Cl, F and CF₃;
R₉ is selected from H, CH₃ and Cl;
R₂₈ is selected from CF₃, CF₂H, —CH₂CH₃, Cl, SF₅, Br and —C(O)H;
X is selected from C—R₇ and N;
R₇ is selected from H and F;
R₃ is selected from CH₃, CH₂CH₃, cyclopropyl and hydroxyethyl;
R₄ is selected from: 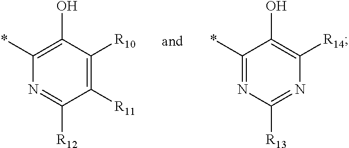
wherein
R₁₀ is selected from H, F, Cl, CH₃ and OCF₃;
R₁₁ is selected from H, Cl, F, and CH₃;
R₁₂ is selected from H, Cl and CH₃;
R₁₃ is selected from H and CH₃;
R₁₄ is selected from H, CH₃, —CH₂CH₃, cyclopropyl, —OCHF₂, OCF₃ and Cl;
y is 0, 1 or 2;
R₅ is CH₃; or alternatively, two R₅ groups on adjacent carbon atoms join, along with the carbon atoms to which they are attached, to form a fused cyclobutyl ring: 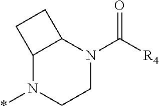
and
* indicates a point of attachment.

7. A WRN inhibitor for use as claimed in any of claims 1 to 6, wherein the WRN inhibitor is Compound A: 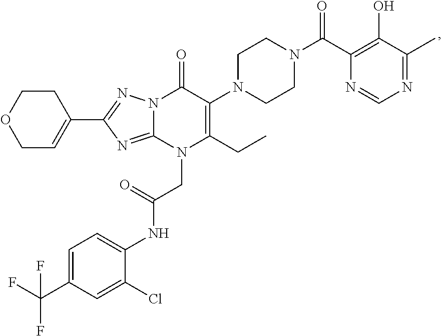 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl]
or a pharmaceutically acceptable salt thereof.

8. A WRN inhibitor for use as claimed in any of claims 1 to 7, wherein the cancer is selected from colorectal cancer (CRC), such as colon adenocarcinoma or rectal adenocarcinoma, gastric cancer, such as stomach adenocarcinoma, prostate cancer, endometrial cancer, adrenocortical cancer, such as adrenocortical carcinoma, cervical cancer, such as cervical squamous cell carcinoma or endocervical adenocarcinoma, uterine cancer, such as uterine corpus endometrial carcinoma and uterine carcinosarcoma, esophageal cancer, such as esophageal carcinoma, breast cancer, such as breast carcinoma or triple negative breast cancer, kidney cancer, such as kidney renal clear cell carcinoma, ovarian cancer, such as ovarian serous cystadenocarcinoma, glioma, glioblastoma, neuroendocrine tumors, melanoma, small cell lung cancer and sarcoma. In particular, colorectal cancer or small cell lung cancer.

9. A WRN inhibitor for use as claimed in any of claims 1 to 8, wherein the cancer is selected from MSS (microsatellite stable) MGMT-defective, or MSS (microsatellite stable) MGMT-deficient, or MSS (microsatellite stable) MGMT-silenced, or MGMT-methylated MSS (microsatellite stable) cancer.

10. A WRN inhibitor for use as claimed in any of claims 1 to 9, wherein the agent i. is administered as:
a pre-treatment agent prior to treatment with the WRN inhibitor,
a combination agent with the WRN inhibitor, without pre-treatment,
both a pre-treatment agent and combination agent with a WRN inhibitor.

11. A WRN inhibitor for use as claimed in any of claims 1 to 10, wherein the agent i. is temozolomide, and temozolomide is administered:
as a pre-treatment before administration of the WRN inhibitor, or
in combination with the WRN inhibitor, without pre-treatment with temozolomide, or
as a pre-treatment before administration of the WRN inhibitor, followed by combination treatment including temozolomide and the WRN inhibitor,
and the treatment preferably further comprises administration of irinotecan.

12. A combination comprising i) a WRN inhibitor and ii) temozolomide, and optionally iii) irinotecan.

13. A combination according to claim 12, for use in the treatment of cancer, wherein the cancer is MSI-H, dMMR or MSS, in particular MSS.

14. A combination according to claim 12, for use according to claim 13, wherein the cancer is selected from colorectal cancer (CRC), such as colon adenocarcinoma or rectal adenocarcinoma, gastric cancer, such as stomach adenocarcinoma, prostate cancer, endometrial cancer, adrenocortical cancer, such as adrenocortical carcinoma, cervical cancer, such as cervical squamous cell carcinoma or endocervical adenocarcinoma, uterine cancer, such as uterine corpus endometrial carcinoma and uterine carcinosarcoma, esophageal cancer, such as esophageal carcinoma, breast cancer, such as breast carcinoma or triple negative breast cancer, kidney cancer, such as kidney renal clear cell carcinoma, ovarian cancer, such as ovarian serous cystadenocarcinoma, glioma, glioblastoma, neuroendocrine tumors, melanoma, small cell lung cancer and sarcoma. In particular, colorectal cancer or small cell lung cancer, especially MSS colorectal cancer or MSS small cell lung cancer.

15. A combination according to claim 12, for use according to claim 13 or 14, wherein the cancer is selected from MSS (microsatellite stable) MGMT-defective, or MSS (microsatellite stable) MGMT-deficient, or MSS (microsatellite stable) MGMT-silenced, or MGMT-methylated MSS (microsatellite stable) cancer.

16. A combination according to claim 12, for use according to any of claims 13 to 15, wherein the WRN inhibitor is a compound of formula (1g), or a pharmaceutically acceptable salt thereof: 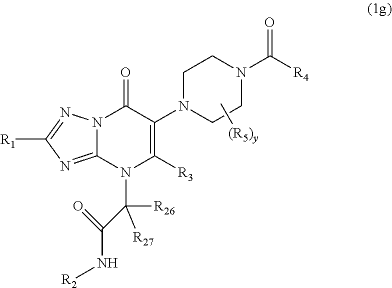
wherein
R₁ is selected from: 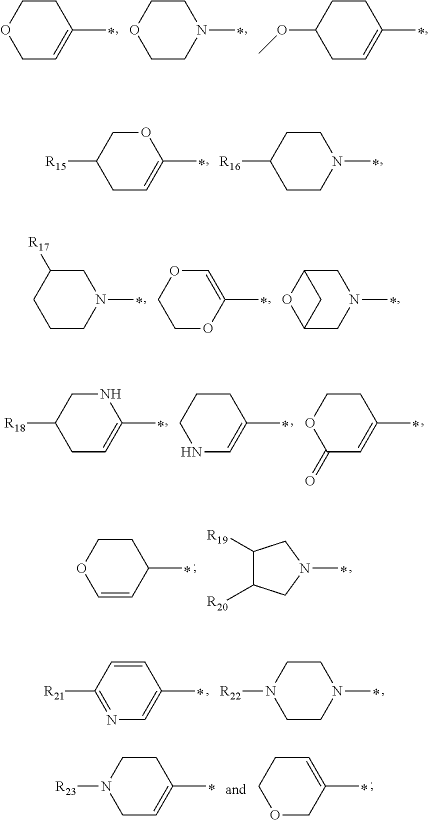
R₁₅ is H or F;
R₁₆ is H or R₂₅(R₂₄)N—;
R₁₇ is H or F;
R₁₈ is H or F;
R₁₉ is H or F;
R₂₀ is H or F;
R₂₁ is H or CH₃;
R₂₂ is H, CF₃, CHF₂CH₂, HOC(O)—CH₂—, H₃C—C(O)—, (H₃C)₃C—O—C(O)—;
R₂₃ is H, CF₃, CHF₂CH₂—, (H₃C)₃C—O—C(O)—;
R₂₄ is CH₃;
R₂₅ is CHF₂CH₂—;
R₂₈ is CH₃, H or deuterium;
R₂₇ is H or deuterium;
R₂ is the moiety: 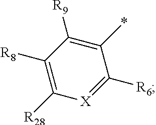
wherein R₆ is selected from H, Cl, CH₃, F, and Br;
R₈ is selected from H, Cl, F and CF₃;
R₉ is selected from H, CH₃ and Cl;
R₂₈ is selected from CF₃, CF₂H, —CH₂CH₃, Cl, SF₅, Br and —C(O)H;
X is selected from C—R₇ and N;
R₇ is selected from H and F;
R₃ is selected from CH₃, CH₂CH₃, cyclopropyl and hydroxyethyl;
R₄ is selected from: 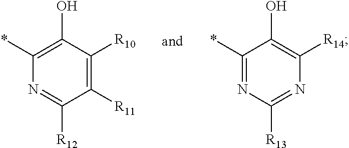
wherein
R₁₀ is selected from H, F, Cl, CH₃ and OCF₃;
R₁₁ is selected from H, Cl, F, and CH₃;
R₁₂ is selected from H, Cl and CH₃;
R₁₃ is selected from H and CH₃;
R₁₄ is selected from H, CH₃, —CH₂CH₃, cyclopropyl, —OCHF₂, OCF₃ and Cl;
y is 0, 1 or 2;
R₅ is CH₃; or alternatively, two R₅ groups on adjacent carbon atoms join, along with the carbon atoms to which they are attached, to form a fused cyclobutyl ring: 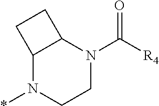
and
* indicates a point of attachment.

17. A combination according to claim 12 or 16, for use according to any of claims 13 to 15, wherein the WRN inhibitor is Compound A: 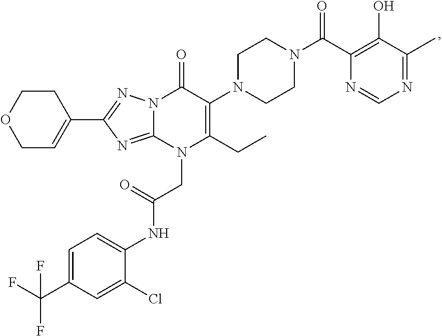 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl]
or a pharmaceutically acceptable salt thereof.

18. A combination according to claim 12, 16 or 17, for use according to any of claims 13 to 15, wherein temozolomide is administered:
as a pre-treatment before administration of the WRN inhibitor, or before administration of irinotecan, or before administration of both the WRN inhibitor and irinotecan, without subsequent combination treatment including temozolide, or
in combination, without pre-treatment with temozolomide, or
as a pre-treatment before administration of the WRN inhibitor, or before administration of irinotecan, or before administration of both the WRN inhibitor and irinotecan, followed by combination treatment including temozolomide.

19. A method of treating cancer in a subject in need thereof, in particular microsatellite instability-high (MSI-H) or mismatch repair deficient (dMMR) cancer, the method comprising administering to the subject a therapeutically effective amount of a WRN inhibitor in combination with a therapeutically effective amount of at least one therapeutically active agent selected from the group consisting of a Wee1 inhibitor, an ATR inhibitor, a DNA-PK inhibitor, ionising radiation based therapy selected from i) external beam radiation, ii) brachytherapy and iii) a radiopharmaceutical, a MEK inhibitor, an MDM2 inhibitor, a G4-quadruplex stabilizer, an ATM inhibitor, a CHK1 or CHK2 inhibitor, including dual CHK1 and CHK2 inhibitor, and a DNA polymerase alpha inhibitor.

20. A WRN inhibitor for use in the treatment of cancer, in particular microsatellite instability-high (MSI-H) or mismatch repair deficient (dMMR) cancer, wherein the treatment further comprises administration of at least one therapeutically active agent selected from the group consisting of a Wee1 inhibitor, an ATR inhibitor, a DNA-PK inhibitor, ionising radiation based therapy selected from i) external beam radiation, ii) brachytherapy and iii) a radiopharmaceutical, a MEK inhibitor, an MDM2 inhibitor, a G4-quadruplex stabilizer, an ATM inhibitor, a CHK1 or CHK2 inhibitor, including dual CHK1 and CHK2 inhibitor, and a DNA polymerase alpha inhibitor.

21. A combination comprising i) a WRN inhibitor, and ii) at least one therapeutically active agent selected from the group consisting of a Wee1 inhibitor, an ATR inhibitor, a DNA-PK inhibitor, ionising radiation selected from i) external beam radiation, ii) brachytherapy and iii) a radiopharmaceutical, a MEK inhibitor, an MDM2 inhibitor, a G4-quadruplex stabilizer, an ATM inhibitor, a CHK1 or CHK2 inhibitor, including dual CHK1 and CHK2 inhibitor, and a DNA polymerase alpha inhibitor.

22. A method of treating cancer in a subject in need thereof, in particular microsatellite instability-high (MSI-H) or mismatch repair deficient (dMMR) cancer, the method comprising administering to the subject a therapeutically effective amount of a WRN inhibitor in combination with a therapeutically effective amount of at least one chemotherapy agent selected from gemcitabine, camptothecin, irinotecan (Camptosar®), docetaxel (Taxotere®), doxorubicin (e.g. doxorubicin hydrochloride) (Adriamycin®, Rubex®), 5-fluorouracil (Adrucil®, Efudex®), capecitabine (Xeloda®), etoposide (Vepesid®), epirubicin (Ellence®, Pharmorubicin®), oxaliplatin (Eloxatin®), mitomycin (e.g. mitomycin A, mitomycin B or mitomycin C, particularly mitomycin C), cisplatin (Platinol®), carboplatin (Paraplatin®), bleomycin and paclitaxel (Taxol®). In particular, the chemotherapy agent is selected from irinotecan (Camptosar®), 5-fluorouracil (Adrucil®, Efudex®), epirubicin (Ellence®, Pharmorubicin®), doxorubicin (e.g. doxorubicin hydrochloride) (Adriamycin®, Rubex®), gemcitabine, camptothecin and mitomycin C.

23. The method according to claim 19 or 22, or the WRN inhibitor for use according to claim 20, wherein the cancer is microsatellite instability-high (MSI-H) or mismatch repair deficient (dMMR).

24. The method according to any of claim 19, 22, or 23, or the WRN inhibitor for use according to claim 20 or 23, or the combination according to claim 21, wherein the (at least one) therapeutically active agent is a WEE1 inhibitor, and the WEE1 inhibitor is adavosertib (also known as AZD1775 and MK-1775).

25. The method according to any of claim 19, 22, or 23, or the WRN inhibitor for use according to claim 20 or 23, or the combination according to claim 21, wherein the (at least one) therapeutically active agent is the ATR inhibitor elimusertib (also known as BAY-1895344).

26. The method according to any of claim 19, 22, or 23, or the WRN inhibitor for use according to claim 20 or 23, or the combination according to claim 21, wherein the (at least one) therapeutically active agent is the DNA-PK inhibitor AZD7648 or NU7441.

27. The method according to any of claim 19, 22, or 23, or the WRN inhibitor for use according to claim 20 or 23, or the combination according to claim 21, wherein the (at least one) therapeutically active agent is an ionising radiation based therapy selected from i) external beam radiation, ii) brachytherapy and iii) a radiopharmaceutical.

28. The method according to any of claim 19, 22, or 23, or the WRN inhibitor for use according to claim 20 or 23, or the combination according to claim 21, wherein the ionising radiation is external beam radiation.

29. The method according to any of claim 19, 22, or 23, or the WRN inhibitor for use according to claim 20 or 23, or the combination according to claim 21, wherein the (at least one) therapeutically active agent is the MEK inhibitor trametinib.

30. The method according to any of claim 19, 22, or 23, or the WRN inhibitor for use according to claim 20 or 23, or the combination according to claim 21, wherein the (at least one) therapeutically active agent is the MDM2 inhibitor HDM201 (also known as siremadlin), or a pharmaceutically acceptable salt thereof.

31. The method according to any of claim 19, 22, or 23, or the WRN inhibitor for use according to claim 20 or 23, or the combination according to claim 21, wherein the (at least one) therapeutically active agent is the G4-quadruplex stabilizer pyridostatin.

32. The method according to any of claim 19, 22, or 23, or the WRN inhibitor for use according to claim 20 or 23, or the combination according to claim 21, wherein the (at least one) therapeutically active agent is the ATM inhibitor KU-60019.

33. The method according to any of claim 19, 22, or 23, or the WRN inhibitor for use according to claim 20 or 23, or the combination according to claim 21, wherein the (at least one) therapeutically active agent is the dual CHK1 and CHK2 inhibitor AZD7762.

34. The method according to any of claim 19 or 22 to 33, or the WRN inhibitor for use according to claim 20 or 23 to 33, wherein the cancer is selected from colorectal cancer (CRC), such as colon adenocarcinoma or rectal adenocarcinoma, gastric cancer, such as stomach adenocarcinoma, prostate cancer, endometrial cancer, adrenocortical cancer, such as adrenocortical carcinoma, cervical cancer, such as cervical squamous cell carcinoma or endocervical adenocarcinoma, uterine cancer, such as uterine corpus endometrial carcinoma and uterine carcinosarcoma, esophageal cancer, such as esophageal carcinoma, breast cancer, such as breast carcinoma or triple negative breast cancer, kidney cancer, such as kidney renal clear cell carcinoma, ovarian cancer, such as ovarian serous cystadenocarcinoma, glioma, glioblastoma, neuroendocrine tumors, melanoma, small cell lung cancer and sarcoma.

35. The method according to claim 34, or the WRN inhibitor for use according to claim 34, wherein the cancer is colorectal cancer (CRC) or gastric cancer, particularly colorectal cancer (CRC).

36. A method of treating colorectal cancer, in particular microsatellite instability-high (MSI-H) or mismatch repair deficient (dMMR) colorectal cancer, the method comprising administering to a subject a therapeutically effective amount of a WRN inhibitor in combination with a therapeutically effective amount of a topoisomerase inhibitor.

37. The method according to claim 36, wherein the topoisomerase inhibitor is QAP1, etoposide, irinotecan or camptothecin.

38. The method according to any of claim 19 or 22 to 35, or the method according to claim 36 or 37, or the WRN inhibitor for use according to claim 20 or 23 to 35, wherein the WRN inhibitor is a compound of formula (1g), or a pharmaceutically acceptable salt thereof: 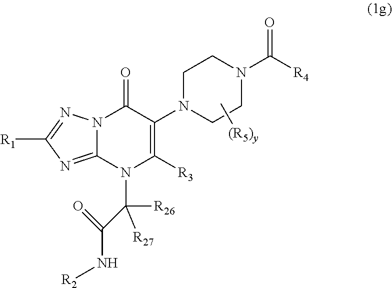
wherein
R₁ is selected from: 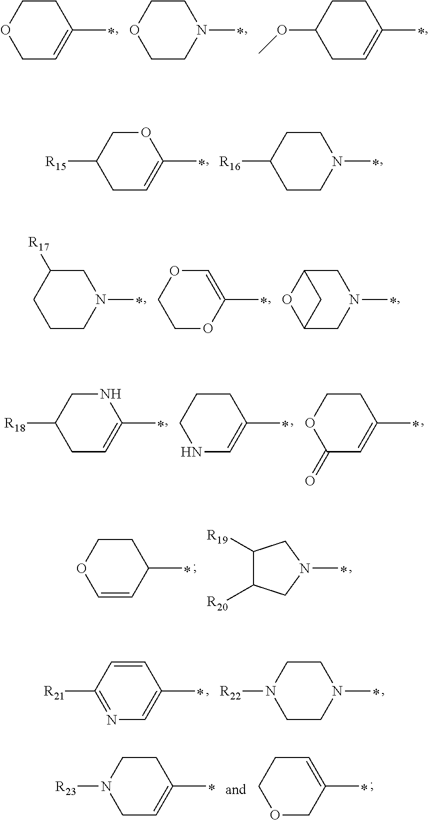
R₁₅ is H or F;
R₁₆ is H or R₂₅(R₂₄)N—;
R₁₇ is H or F;
R₁₈ is H or F;
R₁₉ is H or F;
R₂₀ is H or F;
R₂₁ is H or CH₃;
R₂₂ is H, CF₃, CHF₂CH₂, HOC(O)—CH₂—, H₃C—C(O)—, (H₃C)₃C—O—C(O)—;
R₂₃ is H, CF₃, CHF₂CH₂—, (H₃C)₃C—O—C(O)—;
R₂₄ is CH₃;
R₂₅ is CHF₂CH₂—;
R₂₈ is CH₃, H or deuterium;
R₂₇ is H or deuterium;
R₂ is the moiety: 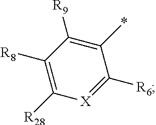
wherein R₆ is selected from H, Cl, CH₃, F, and Br;
R₈ is selected from H, Cl, F and CF₃;
R₉ is selected from H, CH₃ and Cl;
R₂₈ is selected from CF₃, CF₂H, —CH₂CH₃, Cl, SF₅, Br and —C(O)H;
X is selected from C—R₇ and N;
R₇ is selected from H and F;
R₃ is selected from CH₃, CH₂CH₃, cyclopropyl and hydroxyethyl;
R₄ is selected from: 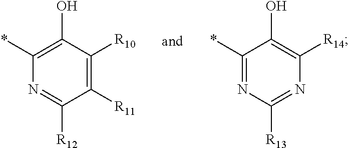
wherein
R₁₀ is selected from H, F, Cl, CH₃ and OCF₃;
R₁₁ is selected from H, Cl, F, and CH₃;
R₁₂ is selected from H, Cl and CH₃;
R₁₃ is selected from H and CH₃;
R₁₄ is selected from H, CH₃, —CH₂CH₃, cyclopropyl, —OCHF₂, OCF₃ and Cl;
y is 0, 1 or 2;
R₅ is CH₃; or alternatively, two R₅ groups on adjacent carbon atoms join, along with the carbon atoms to which they are attached, to form a fused cyclobutyl ring: 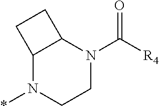
and
* indicates a point of attachment.

39. The method according to claim 38, the WRN inhibitor for use according to claim 38, or the combination according to claim 21, wherein the WRN inhibitor is: 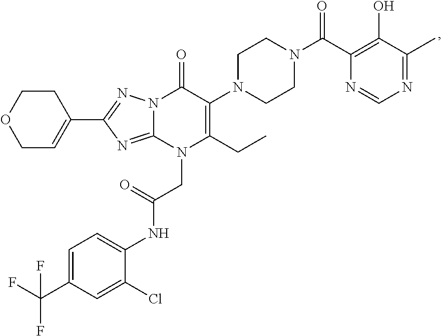 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl]
or a pharmaceutically acceptable salt thereof.
